SEQUENCE LISTING (124 sequences)

SEQ ID NO: 1 - protein, 5 aa, synthetic construct
  source 1..5: note = VH CDR1
DYHIN

SEQ ID NO: 2 - protein, 17 aa, synthetic construct
  source 1..17: note = VH CDR2
WIYPGSGYTKYNEKFKV

SEQ ID NO: 3 - protein, 14 aa, synthetic construct
  source 1..14: note = VH CDR3
SGIYHYVSSYYFDH

SEQ ID NO: 4 - protein, 11 aa, synthetic construct
  source 1..11: note = VL CDR1
KASQDVGTAVA

SEQ ID NO: 5 - protein, 7 aa, synthetic construct
  source 1..7: note = VL CDR2
WASTRHT

SEQ ID NO: 6 - protein, 7 aa, synthetic construct
  source 1..7: note = VL CDR3
QQYSSYT

SEQ ID NO: 7 - protein, 123 aa, synthetic construct
  source 1..123: note = VH AA
QIQLQQSGPELVKPGASVKISCKASGYSFTDYHINWVKQRPGQGLEWIGWIYPGSGYTKYNEKFKVKATLTVDTSSSTAYMHLTSLTSEDSAVYFCARSGIYHYVSSYYFDHWGQGTTLTVSS

SEQ ID NO: 8 - protein, 105 aa, synthetic construct
  source 1..105: note = VL AA
DIVMTQSHTFMSTSVGDRVSITCKASQDVGTAVAWYQQKPGQSPKLLIYWASTRHTGVPDRFTGSGSGTDFTLTISNVQSEDLADYFCQQYSSYTFGGGTKLEIK

SEQ ID NO: 9 - DNA, 369 bp, synthetic construct
  source 1..369: mol_type = other DNA; note = VH DNA
cagattcagctgcagcagagcggccccgagctggtgaagcccggcgcctccgtgaagatcagctgcaaggcctccggctacagcttcaccgactaccacatcaactgggtgaagcagagacccggccaaggcctggagtggatcggctggatctaccccggcagcggctacaccaagtacaacgagaagttcaaggtgaaggccaccctgaccgtcgacacaagcagcagcaccgcctacatgcacctgacaagcctgacaagcgaggacagcgccgtgtacttctgcgctagaagcggcatctaccactacgtgagcagctactacttcgaccactggggccaaggcaccaccctcaccgtgagcagc

SEQ ID NO: 10 - DNA, 315 bp, synthetic construct
  source 1..315: mol_type = other DNA; note = VL DNA
gacatcgtgatgacacagagccacaccttcatgagcacaagcgtgggcgacagagtgagcatcacctgcaaggcctcccaagacgtgggcaccgccgtcgcctggtatcagcagaagcccgggcagagccccaagctgctgatctactgggcctccacaagacacaccggcgtgcccgacagattcaccggcagcggcagcggcaccgacttcaccctgaccatcagcaacgtgcagagcgaggacctggccgactacttctgtcagcagtacagcagctacaccttcggcgggggcaccaagctggagatcaag

SEQ ID NO: 11 - protein, 123 aa, synthetic construct
  source 1..123: note = VH AA
QVQLVQSGAEVKKPGASVKVSCKASGYTFTDYHINWVRQAPGQRLEWMGWIYPGSGYTKYNEKFKVRVTITRDTSASTAYMELSSLRSEDTAVYYCARSGIYHYVSSYYFDHWGKGTTVTVSS

SEQ ID NO: 12 - protein, 105 aa, synthetic construct
  source 1..105: note = VL AA
DIQMTQSPSTLSASVGDRVTITCKASQDVGTAVAWYQQKPGKAPKLLIYWASTRHTGVPSRFSGSGSGTEFTLTISSLQPDDFATYYCQQYSSYTFGGGTKVEIK

SEQ ID NO: 13 - protein, 123 aa, synthetic construct
  source 1..123: note = VH AA
QIQLVQSGAEVKKPGASVKVSCKASGYSFTDYHINWVRQAPGQRLEWMGWIYPGSGYTKYNEKFKVRVTLTVDTSASTAYMELSSLRSEDTAVYFCARSGIYHYVSSYYFDHWGKGTTVTVSS

SEQ ID NO: 14 - protein, 105 aa, synthetic construct
  source 1..105: note = VL AA
DIQMTQSPSTLSASVGDRVTITCKASQDVGTAVAWYQQKPGKSPKLLIYWASTRHTGVPSRFSGSGSGTEFTLTISSVQPDDFATYFCQQYSSYTFGGGTKVEIK

SEQ ID NO: 15 - protein, 123 aa, synthetic construct
  source 1..123: note = VH AA
QVQLVQSGAEVKKPGASVKVSCKASGYSFTDYHINWVRQAPGQRLEWMGWIYPGSGYTKYNEKFKVRVTLTVDTSASTAYMELSSLRSEDTAVYFCARSGIYHYVSSYYFDHWGKGTTVTVSS

SEQ ID NO: 16 - protein, 123 aa, synthetic construct
  source 1..123: note = VH AA
QIQLVQSGAEVKKPGASVKVSCKASGYTFTDYHINWVRQAPGQRLEWMGWIYPGSGYTKYNEKFKVRVTLTVDTSASTAYMELSSLRSEDTAVYFCARSGIYHYVSSYYFDHWGKGTTVTVSS

SEQ ID NO: 17 - protein, 123 aa, synthetic construct
  source 1..123: note = VH AA
QIQLVQSGAEVKKPGASVKVSCKASGYSFTDYHINWVRQAPGQRLEWMGWIYPGSGYTKYNEKFKVRVTITVDTSASTAYMELSSLRSEDTAVYFCARSGIYHYVSSYYFDHWGKGTTVTVSS

SEQ ID NO: 18 - protein, 123 aa, synthetic construct
  source 1..123: note = VH AA
QIQLVQSGAEVKKPGASVKVSCKASGYSFTDYHINWVRQAPGQRLEWMGWIYPGSGYTKYNEKFKVRVTLTRDTSASTAYMELSSLRSEDTAVYFCARSGIYHYVSSYYFDHWGKGTTVTVSS

SEQ ID NO: 19 - protein, 123 aa, synthetic construct
  source 1..123: note = VH AA
QIQLVQSGAEVKKPGASVKVSCKASGYSFTDYHINWVRQAPGQRLEWMGWIYPGSGYTKYNEKFKVRVTLTVDTSASTAYMELSSLRSEDTAVYYCARSGIYHYVSSYYFDHWGKGTTVTVSS

SEQ ID NO: 20 - protein, 105 aa, synthetic construct
  source 1..105: note = VL AA
DIQMTQSPSTLSASVGDRVTITCKASQDVGTAVAWYQQKPGKAPKLLIYWASTRHTGVPSRFSGSGSGTEFTLTISSVQPDDFATYFCQQYSSYTFGGGTKVEIK

SEQ ID NO: 21 - protein, 105 aa, synthetic construct
  source 1..105: note = VL AA
DIQMTQSPSTLSASVGDRVTITCKASQDVGTAVAWYQQKPGKSPKLLIYWASTRHTGVPSRFSGSGSGTEFTLTISSLQPDDFATYFCQQYSSYTFGGGTKVEIK

SEQ ID NO: 22 - protein, 105 aa, synthetic construct
  source 1..105: note = VL AA
DIQMTQSPSTLSASVGDRVTITCKASQDVGTAVAWYQQKPGKSPKLLIYWASTRHTGVPSRFSGSGSGTEFTLTISSVQPDDFATYYCQQYSSYTFGGGTKVEIK

SEQ ID NO: 23 - protein, 17 aa, synthetic construct
  source 1..17: note = VH CDR2
WIYPGSGYTKYNEKFKG

SEQ ID NO: 24 - protein, 123 aa, synthetic construct
  source 1..123: note = VH AA
QVQLVQSGAEVKKPGASVKVSCKASGYSFTDYHINWVRQAPGQRLEWMGWIYPGSGYTKYNEKFKGRVTITRDTSASTAYMELSSLRSEDTAVYYCARSGIYHYVSSYYFDHWGKGTTVTVSS

SEQ ID NO: 25 - protein, 123 aa, synthetic construct
  source 1..123: note = VH AA
QVQLVQSGAEVKKPGASVKVSCKASGYSFTDYHINWVRQAPGQRLEWMGWIYPGSGYTKYNEKFKGRVTITRDTSASTAYMELSSLRSEDTAVYFCARSGIYHYVSSYYFDHWGKGTTVTVSS

SEQ ID NO: 26 - protein, 123 aa, synthetic construct
  source 1..123: note = VH AA
QIQLVQSGAEVKKPGASVKVSCKASGYSFTDYHINWVRQAPGQRLEWMGWIYPGSGYTKYNEKFKGRVTITRDTSASTAYMELSSLRSEDTAVYYCARSGIYHYVSSYYFDHWGKGTTVTVSS

SEQ ID NO: 27 - protein, 123 aa, synthetic construct
  source 1..123: note = VH AA
QVQLVQSGAEVKKPGASVKVSCKASGYSFTDYHINWVRQAPGQRLEWMGWIYPGSGYTKYNEKFKGRVTLTRDTSASTAYMELSSLRSEDTAVYYCARSGIYHYVSSYYFDHWGKGTTVTVSS

SEQ ID NO: 28 - protein, 123 aa, synthetic construct
  source 1..123: note = VH AA
QIQLVQSGAEVKKPGASVKVSCKASGYSFTDYHINWVRQAPGQRLEWMGWIYPGSGYTKYNEKFKGRVTLTRDTSASTAYMELSSLRSEDTAVYYCARSGIYHYVSSYYFDHWGKGTTVTVSS

SEQ ID NO: 29 - protein, 123 aa, synthetic construct
  source 1..123: note = VH AA
QIQLVQSGAEVKKPGASVKVSCKASGYSFTDYHINWVRQAPGQRLEWMGWIYPGSGYTKYNEKFKGRVTITRDTSASTAYMELSSLRSEDTAVYFCARSGIYHYVSSYYFDHWGKGTTVTVSS

SEQ ID NO: 30 - protein, 123 aa, synthetic construct
  source 1..123: note = VH AA
QVQLVQSGAEVKKPGASVKVSCKASGYSFTDYHINWVRQAPGQRLEWMGWIYPGSGYTKYNEKFKGRVTLTRDTSASTAYMELSSLRSEDTAVYFCARSGIYHYVSSYYFDHWGKGTTVTVSS

SEQ ID NO: 31 - protein, 123 aa, synthetic construct
  source 1..123: note = VH AA
QIQLQQSGPELVKPGASVKISCKASGYSFTDYHINWVKQRPGQGLEWIGWIYPGSGYTKYNEKFKVKATLTVDTSSSTAYMHLTSLTSEDSAVYFCARSGIYHYVSSYYFEHWGQGTTLTVSS

SEQ ID NO: 32 - protein, 123 aa, synthetic construct
  source 1..123: note = VH AA
QIQLQQSGPELVKPGASVKISCKASGYSFTDYHINWVKQRPGQGLEWIGWIYPGSGYTKYNEKFKVKATLTVDTSSSTAYMHLTSLTSEDSAVYFCARSGIYHYVSSYYFQHWGQGTTLTVSS

SEQ ID NO: 33 - protein, 123 aa, synthetic construct
  source 1..123: note = VH AA
QIQLQQSGPELVKPGASVKISCKASGYSFTDYHINWVKQRPGQGLEWIGWIYPGSGYTKYNEKFKGKATLTVDTSSSTAYMHLTSLTSEDSAVYFCARSGIYHYVSSYYFDHWGQGTTLTVSS

SEQ ID NO: 34 - protein, 123 aa, synthetic construct
  source 1..123: note = VH AA
QIQLQQSGPELVKPGASVKISCKASGYSFTDYAINWVKQRPGQGLEWIGWIYPGSGYTKYNEKFKVKATLTVDTSSSTAYMHLTSLTSEDSAVYFCARSGIYHYVSSYYFDHWGQGTTLTVSS

SEQ ID NO: 35 - protein, 123 aa, synthetic construct
  source 1..123: note = VH AA
QIQLVQSGAEVKKPGASVKVSCKASGYSFTDYHINWVRQAPGQRLEWMGWIYPGSGYTKYNEKFKVRVTLTVDTSASTAYMELSSLRSEDTAVYFCARSGIYHYVSSYYFEHWGKGTTVTVSS

SEQ ID NO: 36 - protein, 123 aa, synthetic construct
  source 1..123: note = VH AA
QIQLVQSGAEVKKPGASVKVSCKASGYSFTDYHINWVRQAPGQRLEWMGWIYPGSGYTKYNEKFKVRVTLTVDTSASTAYMELSSLRSEDTAVYFCARSGIYHYVSSYYFQHWGKGTTVTVSS

SEQ ID NO: 37 - protein, 123 aa, synthetic construct
  source 1..123: note = VH AA
QIQLVQSGAEVKKPGASVKVSCKASGYSFTDYHINWVRQAPGQRLEWMGWIYPGSGYTKYNEKFKGRVTLTVDTSASTAYMELSSLRSEDTAVYFCARSGIYHYVSSYYFDHWGKGTTVTVSS

SEQ ID NO: 38 - protein, 123 aa, synthetic construct
  source 1..123: note = VH AA
QIQLVQSGAEVKKPGASVKVSCKASGYSFTDYAINWVRQAPGQRLEWMGWIYPGSGYTKYNEKFKVRVTLTVDTSASTAYMELSSLRSEDTAVYFCARSGIYHYVSSYYFDHWGKGTTVTVSS

SEQ ID NO: 39 - protein, 17 aa, synthetic construct
  source 1..17: note = VH CDR2
YIYPGSGHTKYNEKFKG

SEQ ID NO: 40 - protein, 11 aa, synthetic construct
  source 1..11: note = VL CDR1
KASQDAGSAVA

SEQ ID NO: 41 - protein, 123 aa, synthetic construct
EVQLQESGGGLVQPGGSLRLSCAASGYSFTDYHINWVRQAPGKGLEWVSYIYPGSGHTKYNEKFKGRFTISRDTSKNTAYLQMNSLRAEDTAVYYCARSGIYHYVSSYYFDHWGQGTLVTVSS

SEQ ID NO: 42 - protein, 105 aa, synthetic construct
  source 1..105: note = VL AA
DIQMTQSPSTLSASVGDRVTITCKASQDAGSAVAWYQQKPGKAPKLLIYWASTRHTGVPSRFSGSGSGTEFTLTISSLQPDDFATYYCQQYSSYTFGGGTKVEIK

SEQ ID NO: 43 - protein, 123 aa, synthetic construct
  source 1..123: note = VH AA
EVQLQESGGGLVQPGGSLRLSCAASGYSFSDYHINWVRQAPGKGLEWVGYIYPGSGHTKYNEKFKGRFTISRDTSSNTAYLQMNSLRAEDTAVYYCARSGIYHYVSSYYFDHWGQGTLVTVSS

SEQ ID NO: 44 - protein, 105 aa, synthetic construct
  source 1..105: note = VL AA
DIQMTQSPSSLSASVGDRVTITCKASQDAGSAVAWYQQKPGKAPKLLIYWASTRHTGVPSRFSGSGSGTDFTLTISSLQPEDFATYYCQQYSSYTFGGGTKLEIK

SEQ ID NO: 45 - protein, 17 aa, synthetic construct
  source 1..17: note = VH CDR2
YIYPGSGHTKKNEKFKG

SEQ ID NO: 46 - protein, 123 aa, synthetic construct
  source 1..123: note = VH AA
QVQLQQSGAETKKPGASVKVSCKASGYSFTDYHINWVRQASGQRLEWMGYIYPGSGHTKKNEKFKGRVTITRDTSASTAYMELSSLRSEDTAVYYCARSGIYHYVSSYYFDHWGKGTTVTVSS

SEQ ID NO: 47 - protein, 105 aa, synthetic construct
  source 1..105: note = VL AA
DIQMTQSPSTLSASVGDRVTITCKASQDAGSAVAWYQQKPGKAPKLLIKWASTRHTGVPSRFSGSGSGTEFTLTISSLQPDDFATYYCQQYSSYTFGGGTKVEIK

SEQ ID NO: 48 - protein, 5 aa, synthetic construct
  source 1..5: note = HCDR1 (Kabat)
TYAMN

SEQ ID NO: 49 - protein, 19 aa, synthetic construct
  source 1..19: note = HCDR2 (Kabat)
RIRSKYNNYATYYADSVKD

SEQ ID NO: 50 - protein, 14 aa, synthetic construct
  source 1..14: note = HCDR3 (Kabat)
HGNFGNSYVSWFAY

SEQ ID NO: 51 - protein, 14 aa, synthetic construct
  source 1..14: note = LCDR1 (Kabat)
RSSTGAVTTSNYAN

SEQ ID NO: 52 - protein, 7 aa, synthetic construct
  source 1..7: note = LCDR2 (Kabat)
GTNKRAP

SEQ ID NO: 53 - protein, 9 aa, synthetic construct
  source 1..9: note = LCDR3 (Kabat)
ALWYSNLWV

SEQ ID NO: 54 - protein, 125 aa, synthetic construct
  source 1..125: note = VH AA
EVQLVESGGGLVQPKGSLKLSCAASGFTFNTYAMNWVRQAPGKGLEWVARIRSKYNNYATYYADSVKDRFTISRDDSQSILYLQMNNLKTEDTAMYYCVRHGNFGNSYVSWFAYWGQGTLVTVSS

SEQ ID NO: 55 - protein, 109 aa, synthetic construct
  source 1..109: note = VL AA
QAVVTQESALTTSPGETVTLTCRSSTGAVTTSNYANWVQEKPDHLFTGLIGGTNKRAPGVPARFSGSLIGDKAALTITGAQTEDEAIYFCALWYSNLWVFGGGTKLTVL

SEQ ID NO: 56 - DNA, 375 bp, synthetic construct
  source 1..375: mol_type = other DNA; note = VH DNA
gaggtgcagctggtggagtccggcggtggccttgtgcagcccaagggcagcctgaagctgagctgcgccgctagcggcttcaccttcaacacctacgccatgaactgggtgagacaagcccccggcaagggcctggagtgggtggctagaatcagaagcaagtacaacaactacgccacctactacgccgacagcgtgaaggacagattcaccatcagcagagacgactctcagagcatcctgtacctgcagatgaacaacctgaagaccgaggacaccgccatgtactactgcgtgagacacggcaacttcggcaacagctacgtgagctggttcgcctactggggccaaggcaccctggtaacagtaagcagc

SEQ ID NO: 57 - DNA, 327 bp, synthetic construct
  source 1..327: mol_type = other DNA; note = VL DNA
caagccgtggtgacccaagagagcgccctgaccacaagccccggcgagaccgtgaccctgacctgcagaagcagcaccggcgccgtgaccacaagcaactacgccaactgggtgcaagagaagcccgaccacctgttcaccggtctgatcggcggtaccaacaagagagcccccggcgtgcccgctagattcagcggctcactgatcggcgacaaggccgccctgaccatcaccggcgctcagaccgaggacgaggccatctacttctgcgccctgtggtacagcaacctgtgggtgttcggcggcggcaccaagctgaccgtgctg

SEQ ID NO: 58 - protein, 125 aa, synthetic construct
  source 1..125: note = VH AA
EVQLVESGGGLVQPGGSLRLSCAASGFTFNTYAMNWVRQAPGKGLEWVARIRSKYNNYATYYADSVKDRFTISRDDAKSSLYLQMNSLRAEDTAMYYCVRHGNFGNSYVSWFAYWGQGTLVTVSS

SEQ ID NO: 59 - protein, 110 aa, synthetic construct
  source 1..110: note = VL AA
QAVVTQSPATLSVSPGERATLSCRSSTGAVTTSNYANWVQEKPGQLFTGLIGGTNKRAPGIPARFSGSLSGDEATLTISSLQSEDFAIYFCALWYSNLWVFGGGTKVEIK

SEQ ID NO: 60 - DNA, 375 bp, synthetic construct
  source 1..375: mol_type = other DNA; note = VH DNA
gaggtgcagctggtggagtccggcggtggccttgtgcagcccggcggcagtctgagactgagctgcgccgctagcggcttcaccttcaacacctacgccatgaactgggtgagacaagcccccggcaagggcctggagtgggtggctagaatcagaagcaagtacaacaactacgccacctactacgccgacagcgtgaaggacagattcaccatcagcagagacgacgccaagagcagcctgtacctgcagatgaacagcctgagagccgaggacaccgccatgtactactgcgtgagacacggcaacttcggcaacagctacgtgagctggttcgcctactggggccaaggcaccctggtaacggtgagcagc

SEQ ID NO: 61 - DNA, 330 bp, synthetic construct
  source 1..330: mol_type = other DNA; note = VL DNA
caagccgtggtgacccaaagtcccgcgacccttagcgtaagccccggcgagagagctaccctgagctgcagaagcagcaccggcgccgtgaccacaagcaactacgccaactgggtgcaagagaagcccgggcagctgttcaccggcctgatcggcggtaccaacaagagagcccccggcatccccgctagattcagcggcagcctgagcggcgacgaggccaccctgaccatcagcagcctgcagagcgaggacttcgccatctacttctgcgccctgtggtacagcaacctgtgggtgttcggcggcggcaccaaggtggagatcaag

SEQ ID NO: 62 - protein, 125 aa, synthetic construct
  source 1..125: note = VH AA
EVQLVESGGGLVQPGGSLRLSCAASGFTFNTYAMNWVRQAPGKGLEWVARIRSKYNNYATYYADSVKDRFTISRDDAKNSLYLQMNSLRAEDTAVYYCVRHGNFGNSYVSWFAYWGQGTLVTVSS

SEQ ID NO: 63 - protein, 110 aa, synthetic construct
  source 1..110: note = VL AA
EIVLTQSPATLSVSPGERATLSCRSSTGAVTTSNYANWVQQKPGQAPRGLIGGTNKRAPGIPARFSGSLSGDEATLTISSLQSEDFAVYYCALWYSNLWVFGGGTKVEIK

SEQ ID NO: 64 - DNA, 375 bp, synthetic construct
  source 1..375: mol_type = other DNA; note = VH DNA
gaggtgcagctggtggagtccggcggtggccttgtgcagcccggcggcagtctgagactgagctgcgccgctagcggcttcaccttcaacacctacgccatgaactgggtgagacaagcccccggcaagggcctggagtgggtggctagaatcagaagcaagtacaacaactacgccacctactacgccgacagcgtgaaggacagattcaccatcagcagagacgacgccaagaacagcctgtacctgcagatgaacagcctgagagccgaggacaccgccgtgtactactgcgttagacacggcaacttcggcaacagctacgtgagctggttcgcctactggggccaaggcaccctggtaacggtgagcagc

SEQ ID NO: 65 - DNA, 330 bp, synthetic construct
  source 1..330: mol_type = other DNA; note = VL DNA
gaaatcgtgctgacccaaagtcccgcgacccttagcgtaagccccggcgagagagctaccctgagctgcagaagcagcaccggcgccgtgaccacaagcaactacgccaactgggtgcaacagaagcccgggcaggcgccccgcggcctgatcggcggtaccaacaagagagcccccggcatccccgctagattcagcggcagcctgagcggcgacgaggccaccctgaccatcagcagcctgcagagcgaggacttcgccgtctactactgcgccctgtggtacagcaacctgtgggtgttcggcggcggcaccaaggtggagatcaag

SEQ ID NO: 66 - protein, 250 aa, synthetic construct
  source 1..250: note = scFv AA
EVQLVESGGGLVQPGGSLRLSCAASGFTFNTYAMNWVRQAPGKGLEWVARIRSKYNNYATYYADSVKDRFTISRDDAKNSLYLQMNSLRAEDTAVYYCVRHGNFGNSYVSWFAYWGQGTLVTVSSGGGGSGGGGSGGGGSEIVLTQSPATLSVSPGERATLSCRSSTGAVTTSNYANWVQQKPGQAPRGLIGGTNKRAPGIPARFSGSLSGDEATLTISSLQSEDFAVYYCALWYSNLWVFGGGTKVEIK

SEQ ID NO: 67 - DNA, 750 bp, synthetic construct
  source 1..750: mol_type = other DNA; note = scFv DNA
gaggtgcagctggtggagtccggcggtggccttgtgcagcccggcggcagtctgagactgagctgcgccgctagcggcttcaccttcaacacctacgccatgaactgggtgagacaagcccccggcaagggcctggagtgggtggctagaatcagaagcaagtacaacaactacgccacctactacgccgacagcgtgaaggacagattcaccatcagcagagacgacgccaagaacagcctgtacctgcagatgaacagcctgagagccgaggacaccgccgtgtactactgcgttagacacggcaacttcggcaacagctacgtgagctggttcgcctactggggccaaggcaccctggtaacggtgagcagcggcggcggtggcagcggcggaggcggctctggcggcggaggatccgaaatcgtgctgacccaaagtcccgcgacccttagcgtaagccccggcgagagagctaccctgagctgcagaagcagcaccggcgccgtgaccacaagcaactacgccaactgggtgcaacagaagcccgggcaggcgccccgcggcctgatcggcggtaccaacaagagagcccccggcatccccgctagattcagcggcagcctgagcggcgacgaggccaccctgaccatcagcagcctgcagagcgaggacttcgccgtctactactgcgccctgtggtacagcaacctgtgggtgttcggcggcggcaccaaggtggagatcaag

SEQ ID NO: 68 - protein, 110 aa, synthetic construct
  source 1..110: note = VL AA
EIVVTQSPATLSVSPGERATLSCRSSTGAVTTSNYANWVQQKPGQAPRGLIGGTNKRAPGIPARFSGSLSGDEATLTISSLQSEDFAVYYCALWYSNLWVFGGGTKVEIK

SEQ ID NO: 69 - protein, 250 aa, synthetic construct
  source 1..250: note = ScFv AA
EVQLVESGGGLVQPGGSLRLSCAASGFTFNTYAMNWVRQAPGKGLEWVARIRSKYNNYATYYADSVKDRFTISRDDAKNSLYLQMNSLRAEDTAVYYCVRHGNFGNSYVSWFAYWGQGTLVTVSSGGGGSGGGGSGGGGSEIVVTQSPATLSVSPGERATLSCRSSTGAVTTSNYANWVQQKPGQAPRGLIGGTNKRAPGIPARFSGSLSGDEATLTISSLQSEDFAVYYCALWYSNLWVFGGGTKVEIK

SEQ ID NO: 70 - DNA, 750 bp, synthetic construct
  source 1..750: mol_type = other DNA; note = ScFv DNA
gaggtgcagctggtggagtccggcggtggccttgtgcagcccggcggcagtctgagactgagctgcgccgctagcggcttcaccttcaacacctacgccatgaactgggtgagacaagcccccggcaagggcctggagtgggtggctagaatcagaagcaagtacaacaactacgccacctactacgccgacagcgtgaaggacagattcaccatcagcagagacgacgccaagaacagcctgtacctgcagatgaacagcctgagagccgaggacaccgccgtgtactactgcgttagacacggcaacttcggcaacagctacgtgagctggttcgcctactggggccaaggcaccctggtaacggtgagcagcggcggcggtggcagcggcggaggcggctctggcggcggaggatccgaaatcgtggtgacccaaagtcccgcgacccttagcgtaagccccggcgagagagctaccctgagctgcagaagcagcaccggcgccgtgaccacaagcaactacgccaactgggtgcaacagaagcccgggcaggcgccccgcggcctgatcggcggtaccaacaagagagcccccggcatccccgctagattcagcggcagcctgagcggcgacgaggccaccctgaccatcagcagcctgcagagcgaggacttcgccgtctactactgcgccctgtggtacagcaacctgtgggtgttcggcggcggcaccaaggtggagatcaag

SEQ ID NO: 71 - protein, 19 aa, synthetic construct
  source 1..19: note = HCDR2 (Kabat)
RIRSKYNNYATYYADSVKG

SEQ ID NO: 72 - protein, 125 aa, synthetic construct
  source 1..125: note = VH AA
EVQLVESGGGLVQPGGSLRLSCAASGFTFNTYAMNWVRQAPGKGLEWVGRIRSKYNNYATYYADSVKGRFTISRDDAKNSLYLQMNSLRAEDTAVYYCVRHGNFGNSYVSWFAYWGQGTLVTVSS

SEQ ID NO: 73 - protein, 250 aa, synthetic construct
  source 1..250: note = scFv AA
EVQLVESGGGLVQPGGSLRLSCAASGFTFNTYAMNWVRQAPGKGLEWVGRIRSKYNNYATYYADSVKGRFTISRDDAKNSLYLQMNSLRAEDTAVYYCVRHGNFGNSYVSWFAYWGQGTLVTVSSGGGGSGGGGSGGGGSEIVVTQSPATLSVSPGERATLSCRSSTGAVTTSNYANWVQQKPGQAPRGLIGGTNKRAPGIPARFSGSLSGDEATLTISSLQSEDFAVYYCALWYSNLWVFGGGTKVEIK

SEQ ID NO: 74 - DNA, 750 bp, synthetic construct
  source 1..750: mol_type = other DNA; note = scFv DNA
gaggtgcagctggtggagtccggcggtggccttgtgcagcccggcggcagtctgagactgagctgcgccgctagcggcttcaccttcaacacctacgccatgaactgggtgagacaagcccccggcaagggcctggagtgggtgggtagaatcagaagcaagtacaacaactacgccacctactacgccgacagcgtgaagggcagattcaccatcagcagagacgacgccaagaacagcctgtacctgcagatgaacagcctgagagccgaggacaccgccgtgtactactgcgttagacacggcaacttcggcaacagctacgtgagctggttcgcctactggggccaaggcaccctggtaacggtgagcagcggcggcggtggcagcggcggaggcggctctggcggcggaggatccgaaatcgtggtgacccaaagtcccgcgacccttagcgtaagccccggcgagagagctaccctgagctgcagaagcagcaccggcgccgtgaccacaagcaactacgccaactgggtgcaacagaagcccgggcaggcgccccgcggcctgatcggcggtaccaacaagagagcccccggcatccccgctagattcagcggcagcctgagcggcgacgaggccaccctgaccatcagcagcctgcagagcgaggacttcgccgtctactactgcgccctgtggtacagcaacctgtgggtgttcggcggcggcaccaaggtggagatcaag

SEQ ID NO: 75 - protein, 14 aa, synthetic construct
  source 1..14: note = HCDR3 (Kabat)
HGNFGSSYVSWFAY

SEQ ID NO: 76 - protein, 125 aa, synthetic construct
  source 1..125: note = VH AA
EVQLVESGGGLVQPGGSLRLSCAASGFTFSTYAMNWVRQAPGKGLEWVGRIRSKYNNYATYYADSVKGRFTISRDDAKNSLYLQMNSLRAEDTAVYYCVRHGNFGSSYVSWFAYWGQGTLVTVSS

SEQ ID NO: 77 - protein, 250 aa, synthetic construct
  source 1..250: note = scFv AA
EVQLVESGGGLVQPGGSLRLSCAASGFTFSTYAMNWVRQAPGKGLEWVGRIRSKYNNYATYYADSVKGRFTISRDDAKNSLYLQMNSLRAEDTAVYYCVRHGNFGSSYVSWFAYWGQGTLVTVSSGGGGSGGGGSGGGGSEIVVTQSPATLSVSPGERATLSCRSSTGAVTTSNYANWVQQKPGQAPRGLIGGTNKRAPGIPARFSGSLSGDEATLTISSLQSEDFAVYYCALWYSNLWVFGGGTKVEIK

SEQ ID NO: 78 - DNA, 750 bp, synthetic construct
  source 1..750: mol_type = other DNA; note = scFv DNA
gaggtgcagctggtggagtccggcggtggccttgtgcagcccggcggcagtctgagactgagctgcgccgctagcggcttcaccttcagcacctacgccatgaactgggtgagacaagcccccggcaagggcctggagtgggtgggtagaatcagaagcaagtacaacaactacgccacctactacgccgacagcgtgaagggcagattcaccatcagcagagacgacgccaagaacagcctgtacctgcagatgaacagcctgagagccgaggacaccgccgtgtactactgcgttagacacggcaacttcggcagcagctacgtgagctggttcgcctactggggccaaggcaccctggtaacggtgagcagcggcggcggtggcagcggcggaggcggctctggcggcggaggatccgaaatcgtggtgacccaaagtcccgcgacccttagcgtaagccccggcgagagagctaccctgagctgcagaagcagcaccggcgccgtgaccacaagcaactacgccaactgggtgcaacagaagcccgggcaggcgccccgcggcctgatcggcggtaccaacaagagagcccccggcatccccgctagattcagcggcagcctgagcggcgacgaggccaccctgaccatcagcagcctgcagagcgaggacttcgccgtctactactgcgccctgtggtacagcaacctgtgggtgttcggcggcggcaccaaggtggagatcaag

SEQ ID NO: 79 - protein, 481 aa, synthetic construct
  source 1..481: note = Heavy-Knob chain AA
EVQLVESGGGLVQPGGSLRLSCAASGFTFSTYAMNWVRQAPGKGLEWVGRIRSKYNNYATYYADSVKGRFTISRDDAKNSLYLQMNSLRAEDTAVYYCVRHGNFGSSYVSWFAYWGQGTLVTVSSGGGGSGGGGSGGGGSEIVVTQSPATLSVSPGERATLSCRSSTGAVTTSNYANWVQQKPGQAPRGLIGGTNKRAPGIPARFSGSLSGDEATLTISSLQSEDFAVYYCALWYSNLWVFGGGTKVEIKEPKSSDKTHTCPPCPAPPAAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 80 - DNA, 1446 bp, synthetic construct
  source 1..1446: mol_type = other DNA; note = Heavy-Knob chain DNA
gaggtgcaactcgtcgagagcggcgggggcctcgtgcagcctgggggcagcctgagactcagctgcgccgctagcggcttcaccttcagcacctacgccatgaactgggtgagacaagcccccggcaagggcctggagtgggtgggcagaatcagaagcaagtacaacaactacgccacctactacgccgacagcgtgaagggcagattcaccatcagcagagacgacgccaagaacagcctgtacctgcagatgaacagcctgagagccgaggacaccgccgtctactactgcgtgagacacggcaacttcggcagcagctacgtgagctggttcgcttattgggggcaaggcacactggtgaccgtcagcagcggtggaggcggttcaggcggaggtggctctggcggtggcggatcggagattgtggtgacacagagccccgccaccctgagcgtgagccccggcgagagagctacactgagctgcagaagcagcaccggcgccgtgaccacaagcaactacgccaactgggtgcagcagaagcccggccaagcccctagaggcctgatcgggggcaccaacaagagagcccccggcatccccgctagattctccggcagcctgagcggcgacgaggccaccctgaccatcagcagcctgcagagcgaggacttcgccgtgtactactgcgccctgtggtacagcaacctgtgggtgttcggcgggggcaccaaggtcgagatcaaggagcccaaaagcagcgataagacccacacctgtcctccctgccccgcccctcctgccgccggccctagcgtgttcctgttcccccccaagcccaaggacaccctgatgatcagcagaacccccgaggtgacctgcgtggtcgtggacgtgagccacgaggaccccgaggtgaagttcaactggtacgtggacggcgtggaggtgcacaacgccaagaccaagcctagagaggagcagtacaacagcacctacagagtggtgagcgtgctgaccgtgctgcaccaagactggctgaacggcaaggagtacaagtgcaaggtgagcaacaaggccctggccgcccccatcgagaagaccatcagcaaggccaaggggcagcctagagagccccaagtgtacaccctgccccctagcagagacgagctcaccaagaaccaagtgagcctgtggtgcctggtgaagggcttctaccctagcgacatcgccgtggagtgggagagcaacgggcagcccgagaacaactacaagaccaccccccccgtgctggacagcgacggcagcttcttcctgtacagcaagctgaccgtggacaagagcagatggcagcaaggcaacgtgttcagctgcagcgtgatgcacgaggccctgcacaaccactacacacagaagagcctgagcctgagccccggcaagtga

SEQ ID NO: 81 - protein, 452 aa, synthetic construct
  source 1..452: note = Heavy- Hole chain AA
EVQLQESGGGLVQPGGSLRLSCAASGYSFSDYHINWVRQAPGKGLEWVGYIYPGSGHTKYNEKFKGRFTISRDTSSNTAYLQMNSLRAEDTAVYYCARSGIYHYVSSYYFDHWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPAAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 82 - DNA, 1359 bp, synthetic construct
  source 1..1359: mol_type = other DNA; note = Heavy Hole chain DNA
gaagtgcagctgcaagagtccgggggggggctcgtgcaacccgggggctccctgagactgagctgcgccgctagcggctacagcttcagcgactaccacatcaactgggtgagacaagcccccggcaagggcctggagtgggtgggctacatctaccccggcagcggccacaccaagtacaacgagaagttcaagggcagattcaccatcagcagagacacaagcagcaacaccgcctacctgcagatgaacagcctgagagccgaggacaccgccgtgtactactgcgctagaagcggcatctaccactacgtgagcagctactacttcgaccactggggccaaggcaccctggtgacagtgtcctccgcctccacaaaggggcctagcgtgttccccctggcccctagcagcaagagcacaagcggcggcaccgccgccctcggctgcctggtgaaggactatttccccgagcccgtgaccgtgagctggaacagcggcgccctcacaagcggcgtgcacaccttccccgccgtgctgcagagcagcggcctgtacagcctgagcagcgtggtgaccgtgcctagcagcagcctgggcacacagacctacatctgcaacgtgaaccacaagcctagcaacaccaaggtggacaagaaggtggagcctaagagctgcgataagacccacacctgccccccttgtcccgctccccctgctgccgggcctagcgtgttcctgtttcctcccaagcccaaggacaccctgatgatcagcagaacccccgaggtgacctgcgtggtcgtggacgtgagccacgaggaccccgaggtgaagttcaactggtacgtggacggcgtggaggtgcacaacgccaagaccaagcctagagaggagcagtacaacagcacctacagagtggtgagcgtgctgaccgtgctgcaccaagactggctgaacggcaaggagtacaagtgcaaggtgagcaacaaggccctggccgcccccatcgagaagaccatcagcaaggccaaggggcagcctagagagccccaagtgtacaccctgccccctagcagagacgagctgaccaagaaccaagtgagcctgtcctgcgccgtcaagggcttctaccctagcgacatcgccgtggagtgggagagcaacgggcagcccgagaacaactacaagaccaccccccccgtgctggacagcgacggcagcttcttcctggtgagcaagctgaccgtggacaagagcagatggcagcaaggcaacgtgttcagctgcagcgtgatgcacgaggccctgcacaaccactacacacagaagagcctgagcctgagccccggcaagtga

SEQ ID NO: 83 - protein, 212 aa, synthetic construct
  source 1..212: note = Kappa chain AA
DIQMTQSPSSLSASVGDRVTITCKASQDAGSAVAWYQQKPGKAPKLLIYWASTRHTGVPSRFSGSGSGTDFTLTISSLQPEDFATYYCQQYSSYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 84 - DNA, 639 bp, synthetic construct
  source 1..639: mol_type = other DNA; note = Kappa chain DNA
gacattcagatgacacagagccctagcagcctgtccgctagcgtgggcgacagagtgaccatcacctgcaaggctagccaagacgccgggagcgccgtggcctggtatcagcagaagcccggcaaggcccccaagctgctgatctactgggctagcacaagacacaccggcgtgcctagcagattcagcggcagcggcagcggcaccgacttcaccctgaccatcagcagcctgcagcccgaggacttcgccacctactactgtcagcagtacagcagctacaccttcggcgggggcaccaagctggagatcaagcgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttga

SEQ ID NO: 85 - protein, 20 aa, synthetic construct
  source 1..20: note = Linker
GGGGSGGGGSGGGGSGGGGS

SEQ ID NO: 86 - protein, 15 aa, synthetic construct
  source 1..15: note = Linker
GGGGSGGGGSGGGGS

SEQ ID NO: 87 - protein, 220 aa, synthetic construct
  source 1..220: note = CLDN6
MASAGMQILGVVLTLLGWVNGLVSCALPMWKVTAFIGNSIVVAQVVWEGLWMSCVVQSTGQMQCKVYDSLLALPQDLQAARALCVIALLVALFGLLVYLAGAKCTTCVEEKDSKARLVLTSGIVFVISGVLTLIPVCWTAHAIIRDFYNPLVAEAQKRELGASLYLGWAASGLLLLGGGLLCCTCPSGGSQGPSHYMARYSTSAPAISRGPSEYPTKNYV

SEQ ID NO: 88 - protein, 217 aa, synthetic construct
  source 1..217: note = CLDN9
MASTGLELLGMTLAVLGWLGTLVSCALPLWKVTAFIGNSIVVAQVVWEGLWMSCVVQSTGQMQCKVYDSLLALPQDLQAARALCVIALLLALLGLLVAITGAQCTTCVEDEGAKARIVLTAGVILLLAGILVLIPVCWTAHAIIQDFYNPLVAEALKRELGASLYLGWAAAALLMLGGGLLCCTCPPPQVERPRGPRLGYSIPSRSGASGLDKRDYV

SEQ ID NO: 89-93 - skipped (5 entries)

SEQ ID NO: 94 - protein, 4 aa, synthetic construct
  source 1..4: note = Linker
GGGS

SEQ ID NO: 95 - protein, 4 aa, synthetic construct
  source 1..4: note = Linker
SGGG

SEQ ID NO: 96 - protein, 5 aa, synthetic construct
  source 1..5: note = Linker
GGGGS

SEQ ID NO: 97 - protein, 7 aa, synthetic construct
  source 1..7: note = Linker
GGGGSGS

SEQ ID NO: 98 - protein, 8 aa, synthetic construct
  source 1..8: note = Linker
GGGGSGGS

SEQ ID NO: 99 - protein, 10 aa, synthetic construct
  source 1..10: note = Linker
GGGGSGGGGS

SEQ ID NO: 100 - protein, 16 aa, synthetic construct
  source 1..16: note = Linker
AKTTPKLEEGEFSEAR

SEQ ID NO: 101 - protein, 17 aa, synthetic construct
  source 1..17: note = Linker
AKTTPKLEEGEFSEARV

SEQ ID NO: 102 - protein, 9 aa, synthetic construct
  source 1..9: note = Linker
AKTTPKLGG

SEQ ID NO: 103 - protein, 10 aa, synthetic construct
  source 1..10: note = Linker
SAKTTPKLGG

SEQ ID NO: 104 - protein, 6 aa, synthetic construct
  source 1..6: note = Linker
SAKTTP

SEQ ID NO: 105 - protein, 6 aa, synthetic construct
  source 1..6: note = Linker
RADAAP

SEQ ID NO: 106 - protein, 9 aa, synthetic construct
  source 1..9: note = Linker
RADAAPTVS

SEQ ID NO: 107 - protein, 12 aa, synthetic construct
  source 1..12: note = Linker
RADAAAAGGPGS

SEQ ID NO: 108 - protein, 27 aa, synthetic construct
  source 1..27: note = Linker
RADAAAAGGGGSGGGGSGGGGSGGGGS

SEQ ID NO: 109 - protein, 18 aa, synthetic construct
  source 1..18: note = Linker
SAKTTPKLEEGEFSEARV

SEQ ID NO: 110 - protein, 5 aa, synthetic construct
  source 1..5: note = ADAAP
ADAAP

SEQ ID NO: 111 - protein, 12 aa, synthetic construct
  source 1..12: note = Linker
ADAAPTVSIFPP

SEQ ID NO: 112 - protein, 5 aa, synthetic construct
  source 1..5: note = Linker
TVAAP

SEQ ID NO: 113 - protein, 12 aa, synthetic construct
  source 1..12: note = Linker
TVAAPSVFIFPP

SEQ ID NO: 114 - protein, 6 aa, synthetic construct
  source 1..6: note = Linker
QPKAAP

SEQ ID NO: 115 - protein, 13 aa, synthetic construct
  source 1..13: note = Linker
QPKAAPSVTLFPP

SEQ ID NO: 116 - protein, 6 aa, synthetic construct
  source 1..6: note = Linker
AKTTPP

SEQ ID NO: 117 - protein, 13 aa, synthetic construct
  source 1..13: note = Linker
AKTTPPSVTPLAP

SEQ ID NO: 118 - protein, 6 aa, synthetic construct
  source 1..6: note = Linker
AKTTAP

SEQ ID NO: 119 - protein, 13 aa, synthetic construct
  source 1..13: note = Linker
AKTTAPSVYPLAP

SEQ ID NO: 120 - protein, 6 aa, synthetic construct
  source 1..6: note = Linker
ASTKGP

SEQ ID NO: 121 - protein, 13 aa, synthetic construct
  source 1..13: note = Linker
ASTKGPSVFPLAP

SEQ ID NO: 122 - protein, 15 aa, synthetic construct
  source 1..15: note = Linker
GENKVEYAPALMALS

SEQ ID NO: 123 - protein, 15 aa, synthetic construct
  source 1..15: note = Linker
GPAKELTPLKEAKVS

SEQ ID NO: 124 - protein, 15 aa, synthetic construct
  source 1..15: note = Linker
GHEAAAVMQVQYPAS

(19) United States
(12) Patent Application Publication
(10) Pub. No.: US 2024/0301061 A1
(43) Pub. Date: Sep. 12, 2024

(54) ANTI-CLDN6 AND ANTI-CD3 MULTISPECIFIC ANTIBODIES AND METHODS OF USE

(71) Applicant: BeiGene Switzerland GmbH, Basel (CH)

(72) Inventors: Zhitao WAN, Shanghai (CN); Dan LI, Shanghai (CN); Xiaoyan TANG, Beijing (CN); Yun CHEN, Shanghai (CN); Ming LEI, Shanghai (CN); Ting SHAO, Shanghai (CN); Liu XUE, Beijing (CN); Yao DING, Shanghai (CN); Ruyue JI, Shanghai (CN); Qiming XU, Shanghai (CN); Hui GUO, Shanghai (CN)

(73) Assignee: BeiGene Switzerland GmbH, Basel (CH)

(21) Appl. No.: 18/620,208

(22) Filed: Mar. 28, 2024

Related U.S. Application Data
(63) Continuation of application No. PCT/IB2024/052124, filed on Mar. 5, 2024.

(30) Foreign Application Priority Data
Mar. 6, 2023 (WO) PCT/CN2023/079805

(51) Int. Cl. C07K 16/28; A61K 39/00; A61K 47/10; A61K 47/18; A61K 47/26; C12N 15/85

(52) U.S. Cl. CPC C07K 16/2809; A61K 47/10; A61K 47/183; A61K 47/26; C12N 15/85; A61K 2039/505; C07K 2317/10; C07K 2317/24; C07K 2317/31; C07K 2317/33; C07K 2317/41; C07K 2317/565; C07K 2317/622; C07K 2317/732; C07K 2317/734; C07K 2317/92

(57) ABSTRACT

The present disclosure provides for multi-specific antibodies and antigen-binding fragments thereof that bind to human CLDN6 and CD3, a pharmaceutical composition comprising said antibody or antigen-binding fragments thereof, and use of the multi-specific antibody or antigen-binding fragments thereof or the composition for treating a disease, such as cancer.

### CROSS-REFERENCE TO RELATED APPLICATIONS

This application is a continuation of International Patent Application No. PCT/IB2024/052124, filed Mar. 5, 2024, which claims priority to International Patent Application No. PCT/CN2023/079805, filed Mar. 6, 2023, the entire contents of which are incorporated herein by reference in their entirety.

### SEQUENCE LISTING

The instant application contains a Sequence Listing which has been submitted electronically in XML format, and is hereby incorporated by reference in its entirety. Said XML file was created on Mar. 4, 2024, is named 138881_0872_Sequence_Listing.xml, and is 139 bytes in size.

### TECHNICAL FIELD

Disclosed herein are multi-specific antibodies or antigen-binding fragments thereof that bind to human claudin 6 (CLDN6) and human cluster of differentiation 3 (CD3) and methods of producing same. Specifically, the present disclosure provides, among other things, pharmaceutical compositions comprising the antibodies or antigen-binding fragments thereof, as well as methods for the treatment of cancer.

### BACKGROUND

The following description of the background of the present technology is provided simply as an aid in understanding the present technology and is not admitted to describe or constitute prior art to the present technology.

The claudin (CLDN) gene family encodes integral membrane proteins that are crucial structural and functional components of tight junctions (TJ). CLDNs show tetra-span transmembrane topology, with two extracellular loops and both N- and C-termini located in the cytoplasm (Krause et al., Biochimica et Biophysica Acta (BBA)-Biomembranes. 2008). There are 26 human CLDNs expressed in epithelial and endothelial cells in a tissue-specific manner (Günzel et al., Physiol Rev. 2013). By interacting with each other in both cis (intracellular) and trans (intercellular) interactions, CLDNs exert important roles in regulating paracellular permeability and maintaining cell polarity (Tsukita et al., Trends in Biochemical Sciences. 2019). In additional, CLDNs could server as protein scaffolds for assembling complexes at cell junctions, and transmit signals to the cell interior to modulate gene expression and cell behavior (Matter et al., Nat Rev Mol Cell Biol. 2003; Singh et al., Pflugers Arch. 2017).

CLDN6 was first identified and characterized in 2001 (Turksen et al., Developmental Dynamics. 2001). The expression of CLDN6 is dynamically modulated by various factors and mechanisms (Du et al., Mol Med Rep. 2021). CLDN6 is one of the earliest proteins expressed in embryonic stem cells committed to the epithelial fate and a cell-surface-specific marker of human pluripotent stem cells (hPSCs) (Ben-David et al., Nat Commun. 2013). Interestingly, CLDN6 expression could be detected in fetal tissues including the stomach, pancreas, lung, and kidney, but not in the corresponding adult tissue samples (Reinhard et al., Science. 2020; Abuazza et al., Am J Physiol Renal Physiol. 2006; Hashizume et al., Dev Dyn. 2004). Of note, while transcriptionally silenced in normal adult tissues, up-regulation of CLDN6 has been reported in multiple cancer types, including ovarian cancer, endometrial cancer, testicular cancer, lung cancer, gastric cancer, etc. (Kohmoto et al., Gastric Cancer. 2020; Kojima et al., Cancers (Basel). 2020; Micke et al., Int J Cancer. 2014; Sullivan et al., Am J Surg Pathol. 2012; Ushiku et al., Histopathology. 2012). The differential expression CLDN6 between cancer tissue and normal tissues and the membrane localization makes it an attractive target for cancer immunotherapy.

CD3-bispecific antibody (BsAb) is an emerging therapeutic modality in the field of cancer immunotherapy. CD3-BsAbs act by simultaneous binding to a tumor-associated antigen expressed on tumor cells and to CD3 on T cells. Bridging of these two cell types to close proximity by CD3-BsAbs allows the formation of immunological synapse independent of MHC restriction, and leads to T-cell activation and subsequent anti-tumor immune responses (Kamakura et al., Pharmaceuticals (Basel). 2021). Currently, CD3-BsAbs show great potential for hematological malignancies, and early encouraging clinical data has been reported for solid tumors. One key hurdle for development of CD3-BsAb for solid tumors is the identification of cell-surface targets with high cancer-specific expression to allow for efficient tumor eradication and low risk of on-target off-tumor toxicity (Middelburg et al., Cancers (Basel). 2021; Singh et al., Br J Cancer. 2021; Bacuerle et al., Current Opinion in Oncology. 2022). Based on the highly cancer-specific expression pattern, CLDN6 could serve as an promising target for CD3-BsAb development (Stadler et al., Oncoimmunology. 2016; Stadler et al., Nat Med. 2017).

An important consideration of targeting CLDN6 is that many members of the CLDN family have high sequence identity, with claudin 9 (CLDN9) having the highest similarity to CLDN6. CLDN6 and CLDN9 extracellular loops differ at only 3 out of 76 residues. Considering that CLDN9 is highly expressed in some normal tissue, achieving high selectivity for CLDN6 over CLDN9 is critical for any CLDN6 targeting antibody-based therapeutic.

### SUMMARY

The present disclosure provides multi-specific anti-CLDN6×CD3 antibodies and antigen-binding fragments thereof. The present disclosure encompasses the following embodiments.

In some aspects, the present disclosure provides, a multi-specific antibody or antigen-binding fragment thereof, comprising a first antigen binding domain that specifically binds to human claudin 6 (CLDN6) and a second antigen binding domain that specifically binds to human cluster of differentiation 3 (CD3).

In some embodiments, wherein the first antigen binding domain does not bind to other claudin (CLDN) protein family members.

In some embodiments, wherein the first antigen binding domain does not bind to human claudin 9 (CLDN9).

In some embodiments, wherein the first antigen binding domain has high selectivity for human CLDN6 over human CLDN9.

In some embodiments, wherein the first antigen binding domain that specifically binds to human CLDN6 comprises: (a) a heavy chain variable region that comprises: (i) a heavy chain complementarity-determining region (HCDR)1 of SEQ ID NO: 1, (ii) a HCDR2 of SEQ ID NO: 2, (iii) a HCDR3 of SEQ ID NO: 3 and a light chain variable region that comprises:(iv\) a light chain complementarity-determining region (LCDR)1 of SEQ ID NO: 4, (v) a LCDR2 of SEQ ID NO: 5, and (vi) a LCDR3 of SEQ ID NO: 6; (b) a heavy chain variable region that comprises: (i) a HCDR1 of SEQ ID NO: 1, (ii) a HCDR2 of SEQ ID NO: 23, (iii) a HCDR3 of SEQ ID NO: 3; and a light chain variable region that comprises: (iv) a LCDR1 of SEQ ID NO: 4, (v) a LCDR2 of SEQ ID NO: 5, and (vi) a LCDR3 of SEQ ID NO: 6; (c) a heavy chain variable region that comprises (i) a HCDR1 of SEQ ID NO: 1, (ii) a HCDR2 of SEQ ID NO: 39, (iii) a HCDR3 of SEQ ID NO: 3; and a light chain variable region that comprises: (iv) a LCDR1 of SEQ ID NO: 40, (v) a LCDR2 of SEQ ID NO: 5, and (vi) a LCDR3 of SEQ ID NO: 6; or (d) a heavy chain variable region that comprises (i) a HCDR1 of SEQ ID NO: 1, (ii) a HCDR2 of SEQ ID NO: 45, (iii) a HCDR3 of SEQ ID NO: 3; and a light chain variable region that comprises: (iv) a LCDR1 of SEQ ID NO: 40, (v) a LCDR2 of SEQ ID NO: 5, and (vi) a LCDR3 of SEQ ID NO: 6.

In some embodiments, wherein the first antigen binding domain comprises: (a) a heavy chain variable region comprising an amino acid sequence having at least 90, 91, 92, 93, 94, 95, 96, 97, 98 or 99% sequence identity to SEQ ID NO:7, and a light chain variable region comprising an amino acid sequence having at least 90, 91, 92, 93, 94, 95, 96, 97, 98 or 99% sequence identity to SEQ ID NO: 8; (b) a heavy chain variable region comprising an amino acid sequence having at least 90, 91, 92, 93, 94, 95, 96, 97, 98 or 99% sequence identity to SEQ ID NO: 24, and a light chain variable region comprising an amino acid sequence having at least 90, 91, 92, 93, 94, 95, 96, 97, 98 or 99% sequence identity to SEQ ID NO: 12; (c) a heavy chain variable region comprising an amino acid sequence having at least 90, 91, 92, 93, 94, 95, 96, 97, 98 or 99% sequence identity to SEQ ID NO: 41, and a light chain variable region comprising an amino acid sequence having at least 90, 91, 92, 93, 94, 95, 96, 97, 98 or 99% sequence identity to SEQ ID NO: 42; (d) a heavy chain variable region comprising an amino acid sequence having at least 90, 91, 92, 93, 94, 95, 96, 97, 98 or 99% sequence identity to SEQ ID NO: 43, and a light chain variable region comprising an amino acid sequence having at least 90, 91, 92, 93, 94, 95, 96, 97, 98 or 99% sequence identity to SEQ ID NO: 44; (c) a heavy chain variable region comprising an amino acid sequence having at least 90, 91, 92, 93, 94, 95, 96, 97, 98 or 99% sequence identity to SEQ ID NO: 46, and a light chain variable region comprising an amino acid sequence having at least 90, 91, 92, 93, 94, 95, 96, 97, 98 or 99% sequence identity to SEQ ID NO: 47; or (f) a heavy chain variable region comprising an amino acid sequence having at least 90, 91, 92, 93, 94, 95, 96, 97, 98 or 99% sequence identity to SEQ ID NO: 46, and a light chain variable region comprising an amino acid sequence having at least 90, 91, 92, 93, 94, 95, 96, 97, 98 or 99% sequence identity to SEQ ID NO: 42.

In some embodiments, wherein one, two, three, four, five, six, seven, eight, nine, or ten amino acids of SEQ ID NOS: 7, 8, 12, 24, 41, 42, 43, 44, 46, or 47 have been inserted, deleted or substituted.

In some embodiments, wherein the first antigen binding domain comprises: (a) a heavy chain variable region has an amino acid sequence comprising SEQ ID NO:7, and a light chain variable region has an amino acid sequence comprising SEQ ID NO: 8; (b) a heavy chain variable region as an amino acid sequence comprising SEQ ID NO: 24, and a light chain variable region has an amino acid sequence comprising SEQ ID NO: 12; (c) a heavy chain variable region has an amino acid sequence comprising SEQ ID NO: 41, and a light chain variable region has an amino acid sequence comprising SEQ ID NO: 42; (d) a heavy chain variable region has an amino acid sequence comprising SEQ ID NO: 43, and a light chain variable region has an amino acid sequence comprising SEQ ID NO: 44; (e) a heavy chain variable region has an amino acid sequence comprising SEQ ID NO: 46, and a light chain variable region has an amino acid sequence comprising SEQ ID NO: 47; or (f) a heavy chain variable region has an amino acid sequence comprising SEQ ID NO: 46, and a light chain variable region has an amino acid sequence comprising SEQ ID NO: 42.

In some embodiments, wherein the second antigen binding domain that specifically binds to human CD3 comprises: (a) a heavy chain variable region that comprises (i) a HCDR1 having an amino acid sequence of SEQ ID NO: 48, (ii) a HCDR2 having an amino acid sequence of SEQ ID NO: 49, (iii) a HCDR3 having an amino acid sequence of SEQ ID NO: 50, and a light chain variable region that comprises: (iv) a LCDR1 having an amino acid sequence of SEQ ID NO: 51, (v) a LCDR2 having an amino acid sequence of SEQ ID NO: 52, and (vi) a LCDR3 having an amino acid sequence of SEQ ID NO: 53; (b) a heavy chain variable region that comprises (i) a HCDR1 having an amino acid sequence of SEQ ID NO: 48, (ii) a HCDR2 having an amino acid sequence of SEQ ID NO: 71, (iii) a HCDR3 having an amino acid sequence of SEQ ID NO: 50, and a light chain variable region that comprises: (iv) a LCDR1 having an amino acid sequence of SEQ ID NO: 51, (v) a LCDR2 having an amino acid sequence of SEQ ID NO: 52, and (vi) a LCDR3 having an amino acid sequence of SEQ ID NO: 53; or (c) a heavy chain variable region that comprises (i) a HCDR1 having an amino acid sequence of SEQ ID NO: 48, (ii) a HCDR2 having an amino acid sequence of SEQ ID NO: 71, (iii) a HCDR3 having an amino acid sequence of SEQ ID NO: 75, and a light chain variable region that comprises: (iv) a LCDR1 having an amino acid sequence of SEQ ID NO: 51, (v) a LCDR2 having an amino acid sequence of SEQ ID NO: 52, and (vi) a LCDR3 having an amino acid sequence of SEQ ID NO: 53.

In some embodiments, wherein the second antigen binding domain comprises: (a) a heavy chain variable region comprising an amino acid sequence at least 90, 91, 92, 93, 94, 95, 96, 97, 98 or 99% identical to SEQ ID NO: 54, and a light chain variable region comprising an amino acid sequence at least 90, 91, 92, 93, 94, 95, 96, 97, 98 or 99% identical to SEQ ID NO: 55; (b) a heavy chain variable region comprising an amino acid sequence at least 90, 91, 92, 93, 94, 95, 96, 97, 98 or 99% identical to SEQ ID NO: 58, and a light chain variable region comprising an amino acid sequence at least 90, 91, 92, 93, 94, 95, 96, 97, 98 or 99% identical to SEQ ID NO: 59; (c) a heavy chain variable region comprising an amino acid sequence at least 90, 91, 92, 93, 94, 95, 96, 97, 98 or 99% identical to SEQ ID NO: 62, and a light chain variable region comprising an amino acid sequence at least 90, 91, 92, 93, 94, 95, 96, 97, 98 or 99% identical to SEQ ID NO: 63; (d) a heavy chain variable region comprising an amino acid sequence at least 90, 91, 92, 93, 94, 95, 96, 97, 98 or 99% identical to SEQ ID NO: 62, and a light chain variable region comprising an amino acid sequence at least 90, 91, 92, 93, 94, 95, 96, 97, 98 or 99% identical to SEQ ID NO: 68; (c) a heavy chain variable region comprising an amino acid sequence at least 90, 91, 92, 93, 94, 95, 96, 97, 98 or 99% identical to SEQ ID NO: 72, and a light chain variable region comprising an amino acid sequence at least 90, 91, 92, 93, 94, 95, 96, 97, 98 or 99% identical to SEQ ID NO: 68; or (f) a heavy chain variable region comprising an amino acid sequence at least 90, 91, 92, 93, 94, 95, 96, 97, 98 or 99% identical to SEQ ID NO: 76, and a light chain variable region comprising an amino acid sequence at least 90, 91, 92, 93, 94, 95, 96, 97, 98 or 99% identical to SEQ ID NO: 68.

In some embodiments, wherein one, two, three, four, five, six, seven, eight, nine, or ten amino acids within SEQ ID NO: 54, 55, 58, 59, 62, 68, 72 or 76 have been inserted, deleted or substituted.

In some embodiments, wherein the second antigen binding domain comprises: (a) a heavy chain variable region that comprises SEQ ID NO: 54, and a light chain variable region that comprises SEQ ID NO: 55; (b) a heavy chain variable region that comprises SEQ ID NO: 58, and a light chain variable region that comprises SEQ ID NO: 59; (c) a heavy chain variable region that comprises SEQ ID NO: 62, and a light chain variable region that comprises SEQ ID NO: 63; (d) a heavy chain variable region that comprises SEQ ID NO: 62, and a light chain variable region that comprises SEQ ID NO: 68; (c) a heavy chain variable region that comprises SEQ ID NO: 72, and a light chain variable region hat comprises SEQ ID NO: 68; or (f) a heavy chain variable region that comprises SEQ ID NO: 76, and a light chain variable region that comprises SEQ ID NO: 68.

In some embodiments, wherein the second antigen binding domain comprises: (a) a single chain variable fragment (scFv) that comprises SEQ ID NO: 66; (b) a single chain variable fragment (scFv) that comprises SEQ ID NO: 69; (c) a single chain variable fragment (scFv) that comprises SEQ ID NO: 73; or (e) a single chain variable fragment (scFv) that comprises SEQ ID NO: 77.

In some embodiments, wherein: (a) the first antigen binding domain that specifically binds to human CLDN6 comprises: a heavy chain variable region that comprises (i) a HCDR1 of SEQ ID NO: 1, (ii) a HCDR2 of SEQ ID NO: 2, (iii) a HCDR3 of SEQ ID NO: 3 and a light chain variable region that comprises: (iv) a LCDR1 of SEQ ID NO: 4, (v) a LCDR2 of SEQ ID NO: 5, and (vii) a LCDR3 of SEQ ID NO: 6; and the second antigen binding domain that specifically binds to human CD3 comprises: a heavy chain variable region that comprises (i) a HCDR1 of SEQ ID NO: 48, (b) a HCDR2 of SEQ ID NO: 49, (c) a HCDR3 of SEQ ID NO: 50 and a light chain variable region that comprises: (d) a LCDR1 of SEQ ID NO: 51, (c) a LCDR2 of SEQ ID NO: 52, and (f) a LCDR3 of SEQ ID NO: 53; (b) the first antigen binding domain that specifically binds to human CLDN6 comprises: a heavy chain variable region that comprises (i) a HCDR1 of SEQ ID NO: 1, (ii) a HCDR2 of SEQ ID NO: 2, (iii) a HCDR3 of SEQ ID NO: 3 and a light chain variable region that comprises: (iv) a LCDR1 of SEQ ID NO: 4, (v) a LCDR2 of SEQ ID NO: 5, and (vi) a LCDR3 of SEQ ID NO: 6; and the second antigen binding domain that specifically binds to human CD3 comprises: a heavy chain variable region that comprises (i) a HCDR1 of SEQ ID NO: 48, (ii) a HCDR2 of SEQ ID NO: 71, (iii) a HCDR3 of SEQ ID NO: 50 and a light chain variable region that comprises: (iv) a LCDR1 of SEQ ID NO: 51, (v) a LCDR2 of SEQ ID NO: 52, and (vi) a LCDR3 of SEQ ID NO: 53; or (c) the first antigen binding domain that specifically binds to human CLDN6 comprises: a heavy chain variable region that comprises (i) a HCDR1 of SEQ ID NO: 1, (ii) a HCDR2 of SEQ ID NO: 2, (iii) a HCDR3 of SEQ ID NO: 3 and a light chain variable region that comprises: (iv) a LCDR1 of SEQ ID NO: 4, (v) a LCDR2 of SEQ ID NO: 5, and (vi) a LCDR3 of SEQ ID NO: 6; and the second antigen binding domain that specifically binds to human CD3 comprises: a heavy chain variable region that comprises (i) a HCDR1 of SEQ ID NO: 48, (ii) a HCDR2 of SEQ ID NO: 71, (iii) a HCDR3 of SEQ ID NO: 75 and a light chain variable region that comprises: (iv) a LCDR1 of SEQ ID NO: 51, (v) a LCDR2 of SEQ ID NO: 52, and (vi) a LCDR3 of SEQ ID NO: 53.

In some embodiments, wherein: (a) the first antigen binding domain that specifically binds to human CLDN6 comprises: a heavy chain variable region that comprises (i) a HCDR1 of SEQ ID NO: 1, (ii) a HCDR2 of SEQ ID NO: 23, (iii) a HCDR3 of SEQ ID NO: 3 and a light chain variable region that comprises: (iv) a LCDR1 of SEQ ID NO: 4, (v) a LCDR2 of SEQ ID NO: 5, and (vi) a LCDR3 of SEQ ID NO: 6; and the second antigen binding domain that specifically binds to human CD3 comprises: a heavy chain variable region that comprises (i) a HCDR1 of SEQ ID NO: 48, (ii) a HCDR2 of SEQ ID NO: 49, (iii) a HCDR3 of SEQ ID NO: 50 and a light chain variable region that comprises: (iv) a LCDR1 of SEQ ID NO: 51, (v) a LCDR2 of SEQ ID NO: 52, and (vi) a LCDR3 of SEQ ID NO: 53; (b) the first antigen binding domain that specifically binds to human CLDN6 comprises: a heavy chain variable region that comprises (i) a HCDR1 of SEQ ID NO: 1, (ii) a HCDR2 of SEQ ID NO: 23, (iii) a HCDR3 of SEQ ID NO: 3 and a light chain variable region that comprises: (iv) a LCDR1 of SEQ ID NO: 4, (v) a LCDR2 of SEQ ID NO: 5, and (vi) a LCDR3 of SEQ ID NO: 6; and the second antigen binding domain that specifically binds to human CD3 comprises: a heavy chain variable region that comprises (i) a HCDR1 of SEQ ID NO: 48, (ii) a HCDR2 of SEQ ID NO: 71, (iii) a HCDR3 of SEQ ID NO: 50 and a light chain variable region that comprises: (iv) a LCDR1 of SEQ ID NO: 51, (v) a LCDR2 of SEQ ID NO: 52, and (vi) a LCDR3 of SEQ ID NO: 53; or (c) the first antigen binding domain that specifically binds to human CLDN6 comprises: a heavy chain variable region that comprises (i) a HCDR1 of SEQ ID NO: 1, (ii) a HCDR2 of SEQ ID NO: 23, (iii) a HCDR3 of SEQ ID NO: 3 and a light chain variable region that comprises: (iv) a LCDR1 of SEQ ID NO: 4, (v) a LCDR2 of SEQ ID NO: 5, and (vi) a LCDR3 of SEQ ID NO: 6; and the second antigen binding domain that specifically binds to human CD3 comprises: a heavy chain variable region that comprises (i) a HCDR1 of SEQ ID NO: 48, (ii) a HCDR2 of SEQ ID NO: 71, (iii) a HCDR3 of SEQ ID NO: 75 and a light chain variable region that comprises: (iv) a LCDR1 of SEQ ID NO: 51, (v) a LCDR2 of SEQ ID NO: 52, and (vi) a LCDR3 of SEQ ID NO: 53.

In some embodiments, wherein: (a) the first antigen binding domain that specifically binds to human CLDN6 comprises: a heavy chain variable region that comprises (i) a HCDR1 of SEQ ID NO: 1, (ii) a HCDR2 of SEQ ID NO: 39, (iii) a HCDR3 of SEQ ID NO: 3 and a light chain variable region that comprises: (iv) a LCDR1 of SEQ ID NO: 40, (v) a LCDR2 of SEQ ID NO: 5, and (vi) a LCDR3 of SEQ ID NO: 6; and the second antigen binding domain that specifically binds to human CD3 comprises: a heavy chain variable region that comprises (i) a HCDR1 of SEQ ID NO: 48, (ii) a HCDR2 of SEQ ID NO: 49, (iii) a HCDR3 of SEQ ID NO: 50 and a light chain variable region that comprises: (iv) a LCDR1 of SEQ ID NO: 51, (v) a LCDR2 of SEQ ID NO: 52, and (vi) a LCDR3 of SEQ ID NO: 53; (b) the first antigen binding domain that specifically binds to human CLDN6 comprises: a heavy chain variable region that comprises (i) a HCDR1 of SEQ ID NO: 1, (ii) a HCDR2 of SEQ ID NO: 39, (iii) a HCDR3 of SEQ ID NO: 3 and a light chain variable region that comprises: (iv) a LCDR1 of SEQ ID NO: 40, (v) a LCDR2 of SEQ ID NO: 5, and (vi) a LCDR3 of SEQ ID NO: 6; and the second antigen binding domain that specifically binds to human CD3 comprises: a heavy chain variable region that comprises (i) a HCDR1 of SEQ ID NO: 48, (ii) a HCDR2 of SEQ ID NO: 71, (iii) a HCDR3 of SEQ ID NO: 50 and a light chain variable region that comprises: (iv) a LCDR1 of SEQ ID NO: 51, (v) a LCDR2 of SEQ ID NO: 52, and (vi) a LCDR3 of SEQ ID NO: 53; or (c) the first antigen binding domain that specifically binds to human CLDN6 comprises: a heavy chain variable region that comprises (i) a HCDR1 of SEQ ID NO: 1, (ii) a HCDR2 of SEQ ID NO: 39, (iii) a HCDR3 of SEQ ID NO: 3 and a light chain variable region that comprises: (iv) a LCDR1 of SEQ ID NO: 40, (v) a LCDR2 of SEQ ID NO: 5, and (vi) a LCDR3 of SEQ ID NO: 6; and the second antigen binding domain that specifically binds to human CD3 comprises: a heavy chain variable region that comprises (i) a HCDR1 of SEQ ID NO: 48, (ii) a HCDR2 of SEQ ID NO: 71, (iii) a HCDR3 of SEQ ID NO: 75 and a light chain variable region that comprises: (iv) a LCDR1 of SEQ ID NO: 51, (v) a LCDR2 of SEQ ID NO: 52, and (vi) a LCDR3 of SEQ ID NO: 53.

In some embodiments, wherein: the first antigen binding domain that specifically binds to human CLDN6 comprises: a heavy chain variable region that comprises (i) a HCDR1 of SEQ ID NO: 1, (ii) a HCDR2 of SEQ ID NO: 39, (iii) a HCDR3 of SEQ ID NO: 3 and a light chain variable region that comprises: (iv) a LCDR1 of SEQ ID NO: 40, (v) a LCDR2 of SEQ ID NO: 5, and (vi) a LCDR3 of SEQ ID NO: 6; and the second antigen binding domain that specifically binds to human CD3 comprises: a heavy chain variable region that comprises (i) a HCDR1 of SEQ ID NO: 48, (ii) a HCDR2 of SEQ ID NO: 71, (iii) a HCDR3 of SEQ ID NO: 75 and a light chain variable region that comprises: (iv) a LCDR1 of SEQ ID NO: 51, (v) a LCDR2 of SEQ ID NO: 52, and (vi) a LCDR3 of SEQ ID NO: 53.

In some embodiments, wherein: (a) the first antigen binding domain that specifically binds to human CLDN6 comprises: a heavy chain variable region that comprises (i) a HCDR1 of SEQ ID NO: 1, (ii) a HCDR2 of SEQ ID NO: 45, (iii) a HCDR3 of SEQ ID NO: 3 and a light chain variable region that comprises: (iv) a LCDR1 of SEQ ID NO: 40, (v) a LCDR2 of SEQ ID NO: 5, and (vi) a LCDR3 of SEQ ID NO: 6; and the second antigen binding domain that specifically binds to human CD3 comprises: a heavy chain variable region that comprises (i) a HCDR1 of SEQ ID NO: 48, (ii) a HCDR2 of SEQ ID NO: 49, (iii) a HCDR3 of SEQ ID NO: 50 and a light chain variable region that comprises: (iv) a LCDR1 of SEQ ID NO: 51, (v) a LCDR2 of SEQ ID NO: 52, and (vi) a LCDR3 of SEQ ID NO: 53; (b) the first antigen binding domain that specifically binds to human CLDN6 comprises: a heavy chain variable region that comprises (i) a HCDR1 of SEQ ID NO: 1, (ii) a HCDR2 of SEQ ID NO: 45, (iii) a HCDR3 of SEQ ID NO: 3 and a light chain variable region that comprises: (iv) a LCDR1 of SEQ ID NO: 40, (v) a LCDR2 of SEQ ID NO: 5, and (vi) a LCDR3 of SEQ ID NO: 6; and the second antigen binding domain that specifically binds to human CD3 comprises: a heavy chain variable region that comprises (i) a HCDR1 of SEQ ID NO: 48, (ii) a HCDR2 of SEQ ID NO: 71, (iii) a HCDR3 of SEQ ID NO: 50 and a light chain variable region that comprises: (iv) a LCDR1 of SEQ ID NO: 51, (v) a LCDR2 of SEQ ID NO: 52, and (vii) a LCDR3 of SEQ ID NO: 53; or (c) the first antigen binding domain that specifically binds to human CLDN6 comprises: a heavy chain variable region that comprises (i) a HCDR1 of SEQ ID NO: 1, (ii) a HCDR2 of SEQ ID NO: 45, (iii) a HCDR3 of SEQ ID NO: 3 and a light chain variable region that comprises: (iv) a LCDR1 of SEQ ID NO: 40, (v) a LCDR2 of SEQ ID NO: 5, and (vi) a LCDR3 of SEQ ID NO: 6; and the second antigen binding domain that specifically binds to human CD3 comprises: a heavy chain variable region that comprises (i) a HCDR1 of SEQ ID NO: 48, (ii) a HCDR2 of SEQ ID NO: 71, (iii) a HCDR3 of SEQ ID NO: 75 and a light chain variable region that comprises: (iv) a LCDR1 of SEQ ID NO: 51, (v) a LCDR2 of SEQ ID NO: 52, and (vi) a LCDR3 of SEQ ID NO: 53.

In some embodiments, wherein: (a) the first antigen binding domain that specifically binds to human CLDN6 comprises: a heavy chain variable region that comprises (i) a HCDR1 of SEQ ID NO: 1, (ii) a HCDR2 of SEQ ID NO: 45, (iii) a HCDR3 of SEQ ID NO: 3 and a light chain variable region that comprises: (iv) a LCDR1 of SEQ ID NO: 40, (v) a LCDR2 of SEQ ID NO: 5, and (vi) a LCDR3 of SEQ ID NO: 6; and the second antigen binding domain that specifically binds to human CD3 comprises: a heavy chain variable region that comprises (i) a HCDR1 of SEQ ID NO: 48, (ii) a HCDR2 of SEQ ID NO: 71, (iii) a HCDR3 of SEQ ID NO: 75 and a light chain variable region that comprises: (iv) a LCDR1 of SEQ ID NO: 51, (v) a LCDR2 of SEQ ID NO: 52, and (vi) a LCDR3 of SEQ ID NO: 53.

In some embodiments, wherein: (a) the first antigen binding domain that specifically binds to human CLDN6 comprises: (i) a heavy chain variable region that comprises (1) a HCDR1 of SEQ ID NO: 1, (2) a HCDR2 of SEQ ID NO: 2, (3) a HCDR3 of SEQ ID NO: 3; and a light chain variable region that comprises: (4) a LCDR1 of SEQ ID NO: 4, (5) a LCDR2 of SEQ ID NO: 5, and (6) a LCDR3 of SEQ ID NO: 6; (ii) a heavy chain variable region that comprises (1) a HCDR1 of SEQ ID NO: 1, (2) a HCDR2 of SEQ ID NO: 23, (3) a HCDR3 of SEQ ID NO: 3 and a light chain variable region that comprises: (4) a LCDR1 of SEQ ID NO: 4, (5) a LCDR2 of SEQ ID NO: 5, and (6) a LCDR3 of SEQ ID NO: 6; (iii) a heavy chain variable region that comprises (1) a HCDR1 of SEQ ID NO: 1, (2) a HCDR2 of SEQ ID NO: 39, (3) a HCDR3 of SEQ ID NO: 3 and a light chain variable region that comprises: (4) a LCDR1 of SEQ ID NO: 40, (5) a LCDR2 of SEQ ID NO: 5, and (6) a LCDR3 of SEQ ID NO: 6; or (iv) the first antigen binding domain that specifically binds to human CLDN6 comprises: a heavy chain variable region that comprises (1) a HCDR1 of SEQ ID NO: 1, (2) a HCDR2 of SEQ ID NO: 45, (3) a HCDR3 of SEQ ID NO: 3 and a light chain variable region that comprises: (4) a LCDR1 of SEQ ID NO: 40, (5) a LCDR2 of SEQ ID NO: 5, and (6) a LCDR3 of SEQ ID NO: 6; and (b) the second antigen binding domain that specifically binds to human CD3 comprises: (i) a heavy chain variable region (VH) that comprises SEQ ID NO: 54, and a light chain variable region (VL) that comprises SEQ ID NO: 55; (ii) a heavy chain variable region (VH) that comprises SEQ ID NO: 58, and a light chain variable region (VL) that comprises SEQ ID NO: 59; (iii) a heavy chain variable region (VH) that comprises SEQ ID NO: 62, and a light chain variable region (VL) that comprises SEQ ID NO: 63; (iv) a heavy chain variable region (VH) that comprises SEQ ID NO: 62, and a light chain variable region (VL) that comprises SEQ ID NO: 68; (v) a heavy chain variable region (VH) that comprises SEQ ID NO: 72, and a light chain variable region (VL) that comprises SEQ ID NO: 68; or (vi) a heavy chain variable region (VH) that comprises SEQ ID NO: 76, and a light chain variable region (VL) that comprises SEQ ID NO: 68.

In some embodiments, wherein: (a) the first antigen binding domain that specifically binds to human CLDN6 comprises: (i) a heavy chain variable region that comprises (1) a HCDR1 of SEQ ID NO: 1, (2) a HCDR2 of SEQ ID NO: 39, (3) a HCDR3 of SEQ ID NO: 3 and a light chain variable region that comprises: (4) a LCDR1 of SEQ ID NO: 40, (5) a LCDR2 of SEQ ID NO: 5, and (6) a LCDR3 of SEQ ID NO: 6; or and (b) the second antigen binding domain that specifically binds to human CD3 comprises: (i) a heavy chain variable region (VH) that comprises SEQ ID NO: 54, and a light chain variable region (VL) that comprises SEQ ID NO: 55; (ii) a heavy chain variable region (VH) that comprises SEQ ID NO: 58, and a light chain variable region (VL) that comprises SEQ ID NO: 59; (iii) a heavy chain variable region (VH) that comprises SEQ ID NO: 62, and a light chain variable region (VL) that comprises SEQ ID NO: 63; (iv) a heavy chain variable region (VH) that comprises SEQ ID NO: 62, and a light chain variable region (VL) that comprises SEQ ID NO: 68; (v) a heavy chain variable region (VH) that comprises SEQ ID NO: 72, and a light chain variable region (VL) that comprises SEQ ID NO: 68; or (vi) a heavy chain variable region (VH) that comprises SEQ ID NO: 76, and a light chain variable region (VL) that comprises SEQ ID NO: 68.

In some embodiments, wherein: (a) the first antigen binding domain that specifically binds to human CLDN6 comprises: (i) a heavy chain variable region that comprises (1) a HCDR1 of SEQ ID NO: 1, (2) a HCDR2 of SEQ ID NO: 23, (3) a HCDR3 of SEQ ID NO: 3 and a light chain variable region that comprises: (4) a LCDR1 of SEQ ID NO: 4, (5) a LCDR2 of SEQ ID NO: 5, and (6) a LCDR3 of SEQ ID NO: 6; or (ii) a heavy chain variable region that comprises (1) a HCDR1 of SEQ ID NO: 1, (2) a HCDR2 of SEQ ID NO: 39, (3) a HCDR3 of SEQ ID NO: 3 and a light chain variable region that comprises: (4) a LCDR1 of SEQ ID NO: 40, (5) a LCDR2 of SEQ ID NO: 5, and (6) a LCDR3 of SEQ ID NO: 6; or (iii) the first antigen binding domain that specifically binds to human CLDN6 comprises: a heavy chain variable region that comprises (1) a HCDR1 of SEQ ID NO: 1, (2) a HCDR2 of SEQ ID NO: 45, (3) a HCDR3 of SEQ ID NO: 3 and a light chain variable region that comprises: (4) a LCDR1 of SEQ ID NO: 40, (5) a LCDR2 of SEQ ID NO: 5, and (6) a LCDR3 of SEQ ID NO: 6; and (b) the second antigen binding domain that specifically binds to human CD3 comprises: (i) a single chain variable fragment (scFv) that comprises SEQ ID NO: 66; (ii) a single chain variable fragment (scFv) that comprises SEQ ID NO: 69; (iii) a single chain variable fragment (scFv) that comprises SEQ ID NO: 73; or (iv) a single chain variable fragment (scFv) that comprises SEQ ID NO: 77.

In some embodiments, wherein: (a) the first antigen binding domain that specifically binds to human CLDN6 comprises: (i) a heavy chain variable region (VH) that comprises SEQ ID NO: 24, and a light chain variable region (VL) that comprises SEQ ID NO: 12; (ii) a heavy chain variable region (VH) that comprises SEQ ID NO: 41, and a light chain variable region ( ) that comprises SEQ ID NO: 42; (iii) a heavy chain variable region (VH) that comprises SEQ ID NO: 43, and a light chain variable region (VL) that comprises SEQ ID NO: 44; (iv) a heavy chain variable region (VH) that comprises SEQ ID NO: 46, and a light chain variable region (VL) that comprises SEQ ID NO: 47; or (v) a heavy chain variable region (VH) that comprises SEQ ID NO: 46, and a light chain variable region (VL) that comprises SEQ ID NO: 42 and (ii) the second antigen binding domain that specifically binds to human CD3 comprises:(i) a heavy chain variable region (VH) that comprises SEQ ID NO: 54, and a light chain variable region (VL) that comprises SEQ ID NO: 55; (ii) a heavy chain variable region (VH) that comprises SEQ ID NO: 58, and a light chain variable region (VL) that comprises SEQ ID NO: 59; (iii) a heavy chain variable region (VH) that comprises SEQ ID NO: 62, and a light chain variable region (VL) that comprises SEQ ID NO: 63; (iv) a heavy chain variable region (VH) that comprises SEQ ID NO: 62, and a light chain variable region (VL) that comprises SEQ ID NO: 68; (v) a heavy chain variable region (VH) that comprises SEQ ID NO: 72, and a light chain variable region (VL) that comprises SEQ ID NO: 68; or (vi) a heavy chain variable region (VH) that comprises SEQ ID NO: 76, and a light chain variable region (VL) that comprises SEQ ID NO: 68.

In some embodiments, wherein: (a) the first antigen binding domain that specifically binds to human CLDN6 comprises: (i) a heavy chain variable region (VH) that comprises SEQ ID NO: 24, and a light chain variable region (VL) that comprises SEQ ID NO: 12; (ii) a heavy chain variable region (VH) that comprises SEQ ID NO: 41, and a light chain variable region (VL) that comprises SEQ ID NO: 42; (iii) a heavy chain variable region (VH) that comprises SEQ ID NO: 43, and a light chain variable region (VL) that comprises SEQ ID NO: 44; (iv) a heavy chain variable region (VH) that comprises SEQ ID NO: 46, and a light chain variable region (VL) that comprises SEQ ID NO: 47; or v) a heavy chain variable region (VH) that comprises SEQ ID NO: 46, and a light chain variable region (VL) that comprises SEQ ID NO: 42; and (a) the second antigen binding domain that specifically binds to human CD3 comprises: a heavy chain variable region (VH) that comprises SEQ ID NO: 72, and a light chain variable region (VL) that comprises SEQ ID NO: 68.

In some embodiments, wherein: (a) the first antigen binding domain that specifically binds to human CLDN6 comprises: (i) a heavy chain variable region (VH) that comprises SEQ ID NO: 24, and a light chain variable region (VL) that comprises SEQ ID NO: 12; (ii) a heavy chain variable region (VH) that comprises SEQ ID NO: 41, and a light chain variable region (VL) that comprises SEQ ID NO: 42; (iii) a heavy chain variable region (VH) that comprises SEQ ID NO: 43, and a light chain variable region (VL) that comprises SEQ ID NO: 44; (iv) a heavy chain variable region (VH) that comprises SEQ ID NO: 46, and a light chain variable region (VL) that comprises SEQ ID NO: 47; or (v) a heavy chain variable region (VH) that comprises SEQ ID NO: 46, and a light chain variable region (VL) that comprises SEQ ID NO: 42; and (b) the second antigen binding domain that specifically binds to human CD3 comprises: a heavy chain variable region (VH) that comprises SEQ ID NO: 76, and a light chain variable region (VL) that comprises SEQ ID NO: 68.

In some embodiments, wherein: the first antigen binding domain that specifically binds to human CLDN6 comprises: a heavy chain variable region (VH) that comprises SEQ ID NO: 43, and a light chain variable region (VL) that comprises SEQ ID NO: 44; and the second antigen binding domain that specifically binds to human CD3 comprises: (i) a heavy chain variable region (VH) that comprises SEQ ID NO: 54, and a light chain variable region (VL) that comprises SEQ ID NO: 55; (ii) a heavy chain variable region (VH) that comprises SEQ ID NO: 58, and a light chain variable region (VL) that comprises SEQ ID NO: 59; (iii) a heavy chain variable region (VH) that comprises SEQ ID NO: 62, and a light chain variable region (VL) that comprises SEQ ID NO: 63; (iv) a heavy chain variable region (VH) that comprises SEQ ID NO: 62, and a light chain variable region (VL) that comprises SEQ ID NO: 68; (v) a heavy chain variable region (VH) that comprises SEQ ID NO: 72, and a light chain variable region (VL) that comprises SEQ ID NO: 68; or (vi) a heavy chain variable region (VH) that comprises SEQ ID NO: 76, and a light chain variable region (VL) that comprises SEQ ID NO: 68.

In some embodiments, wherein the first antigen binding domain that specifically binds to human CLDN6 comprises: a heavy chain variable region (VH) that comprises SEQ ID NO: 43, and a light chain variable region (VL) that comprises SEQ ID NO: 44; and the second antigen binding domain that specifically binds to human CD3 comprises: a heavy chain variable region (VH) that comprises SEQ ID NO: 72, and a light chain variable region (VL) that comprises SEQ ID NO: 68.

In some embodiments, wherein: the first antigen binding domain that specifically binds to human CLDN6 comprises: a heavy chain variable region (VH) that comprises SEQ ID NO: 43, and a light chain variable region (VL) that comprises SEQ ID NO: 44; and the second antigen binding domain that specifically binds to human CD3 comprises: a heavy chain variable region (VH) that comprises SEQ ID NO: 76, and a light chain variable region (VL) that comprises SEQ ID NO: 68.

In some embodiments, wherein: (a) the first antigen binding domain that specifically binds to human CLDN6 comprises: (i) a heavy chain variable region (VH) that comprises SEQ ID NO: 24, and a light chain variable region (VL) that comprises SEQ ID NO: 12; (ii) a heavy chain variable region (VH) that comprises SEQ ID NO: 41, and a light chain variable region (VL) that comprises SEQ ID NO: 42; (iii) a heavy chain variable region (VH) that comprises SEQ ID NO: 43, and a light chain variable region (VL) that comprises SEQ ID NO: 44; (iv) a heavy chain variable region (VH) that comprises SEQ ID NO: 46, and a light chain variable region (VL) that comprises SEQ ID NO: 47; or (v) a heavy chain variable region (VH) that comprises SEQ ID NO: 46, and a light chain variable region (VL) that comprises SEQ ID NO: 42; and (b) the second antigen binding domain that specifically binds to human CD3 comprises:(i) a single chain variable fragment (scFv) that comprises SEQ ID NO: 66; (ii) a single chain variable fragment (scFv) that comprises SEQ ID NO: 69; (iii) a single chain variable fragment (scFv) that comprises SEQ ID NO: 73; or (iv) a single chain variable fragment (scFv) that comprises SEQ ID NO: 77.

In some embodiments, wherein: the first antigen binding domain that specifically binds to human CLDN6 comprises: a heavy chain variable region (VH) that comprises SEQ ID NO: 43, and a light chain variable region (VL) that comprises SEQ ID NO: 44; and the second antigen binding domain that specifically binds to human CD3 comprises: (a) a single chain variable fragment (scFv) that comprises SEQ ID NO: 66; (b) a single chain variable fragment (scFv) that comprises SEQ ID NO: 69; (c) a single chain variable fragment (scFv) that comprises SEQ ID NO: 73; or (d) a single chain variable fragment (scFv) that comprises SEQ ID NO: 77.

In some embodiments, wherein: the first antigen binding domain that specifically binds to human CLDN6 comprises: a heavy chain variable region (VH) that comprises SEQ ID NO: 43, and a light chain variable region (VL) that comprises SEQ ID NO: 44; and the second antigen binding domain that specifically binds to human CD3 comprises: a single chain variable fragment (scFv) that comprises SEQ ID NO: 66.

In some embodiments, wherein: the first antigen binding domain that specifically binds to human CLDN6 comprises: a heavy chain variable region (VH) that comprises SEQ ID NO: 43, and a light chain variable region (VL) that comprises SEQ ID NO: 44; and the second antigen binding domain that specifically binds to human CD3 comprises: a single chain variable fragment (scFv) that comprises SEQ ID NO: 69.

In some embodiments, wherein: the first antigen binding domain that specifically binds to human CLDN6 comprises: a heavy chain variable region (VH) that comprises SEQ ID NO: 43, and a light chain variable region (VL) that comprises SEQ ID NO: 44; and the second antigen binding domain that specifically binds to human CD3 comprises: a single chain variable fragment (scFv) that comprises SEQ ID NO: 73.

In some embodiments, wherein: the first antigen binding domain that specifically binds to human CLDN6 comprises: a heavy chain variable region (VH) that comprises SEQ ID NO: 43, and a light chain variable region (VL) that comprises SEQ ID NO: 44; and the second antigen binding domain that specifically binds to human CD3 comprises: a single chain variable fragment (scFv) that comprises SEQ ID NO: 77.

In some embodiments, which is a monoclonal antibody, a chimeric antibody, a humanized antibody, a human engineered antibody, a single chain antibody (scFv), a Fab fragment, a Fab′ fragment, or a F(ab′)2 fragment.

In some embodiments, wherein the multi-specific antibody is a bispecific antibody.

In some embodiments, wherein the multi-specific antibody is BG143P and has a sequence comprising SEQ ID NO: 80, SEQ ID NO: 82, or SEQ ID NO: 84.

In some embodiments, wherein the antibody or antigen-binding fragment thereof has antibody dependent cellular cytotoxicity (ADCC) or complement dependent cytotoxicity (CDC).

In some embodiments, wherein the antibody or antigen-binding fragment thereof has reduced glycosylation or no glycosylation or is hypofucosylated.

In some embodiments, wherein the antibody or antigen-binding fragment thereof comprises increased bisecting GlcNac structures.

In some embodiments, wherein the Fc domain is an IgG1 with reduced effector function.

In some embodiments, wherein the Fc domain is an IgG4.

In some aspects, the present disclosure provides a pharmaceutical composition comprising the multi-specific antibody or antigen-binding fragment as disclosed herein

In some embodiments, the pharmaceutical composition further comprising a pharmaceutically acceptable carrier.

In some embodiments, the pharmaceutical composition comprising a histidine/histidine HCl, a trehalose dihydrate, and/or a polysorbate 20.

In some aspect, the present disclosure provide a method of treating cancer comprising administering to a patient in need an effective amount of the multi-specific antibody or antigen-binding fragment as disclose herein.

In some embodiments, wherein the cancer is a solid tumor.

In some embodiments, wherein the cancer is selected from gastric cancer, colon cancer, pancreatic cancer, breast cancer, head and neck cancer, kidney cancer, liver cancer, lung cancer, small cell lung cancer, non-small cell lung cancer, ovarian cancer, skin cancer, mesothelioma, lymphoma, leukemia, myeloma, sarcoma, brain cancer, colorectal cancer, prostate cancer, cervical cancer, testicular cancer, endometrial cancer, bladder cancer, rhabdoid tumor and/or glioma.

In some embodiments, wherein the multi-specific antibody or antigen-binding fragment is administered in combination with one or more additional therapeutic agent.

In some embodiments, wherein the one or more therapeutic agent is selected from paclitaxel or a paclitaxel agent, docetaxel, carboplatin, topotecan, cisplatin, irinotecan, doxorubicin, lenalidomide or 5-azacytidine.

In some embodiments, wherein the one or more therapeutic agent is a paclitaxel agent, lenalidomide or 5-azacytidine.

In some embodiments, wherein at least one of the one or more therapeutic agent an anti-PD1 or anti-PDL1 antibody.

In some embodiments, wherein the anti-PD1 antibody is Tislelizumab.

In some aspects, the present disclosure provides an isolated nucleic acid that encodes the multi-specific antibody or antigen-binding fragment as disclosed herein.

In some aspects, the present disclosure provides a vector comprising the nucleic acid as disclosed herein.

In some aspects, the present disclosure provides a host cell comprising the nucleic acid or the vector as disclosed herein.

In some aspects, the present disclosure provides a process for producing the multi-specific antibody or antigen-binding fragment as disclosed herein comprising cultivating the host cell as disclosed herein and recovering the antibody or antigen-binding fragment from the culture.

In some aspects, the present disclosure provides, the multi-specific antibody or antigen-binding fragment as disclosed herein for use in a method for treating cancer.

In some aspects, the present disclosure provides, the multi-specific antibody or antigen-binding fragment as disclosed herein in the manufacture of a medicament for the treatment of cancer.

In some aspects, the present disclosure provides a pharmaceutical composition for use in a method for treating cancer.

A multi-specific antibody or antigen-binding fragment thereof, comprising a first antigen binding domain that specifically binds to human CLDN6 and a second antigen binding domain that specifically binds to human CD3.

The multi-specific antibody or antigen-binding fragment, wherein the first antigen binding domain does not bind to other CLDN family members.

The multi-specific antibody or antigen-binding fragment, wherein the first antigen binding domain does not bind to human CLDN9.

The multi-specific antibody or antigen-binding fragment, wherein the first antigen binding domain has high selectivity over human CLDN9.

The multi-specific antibody or antigen-binding fragment, wherein the first antigen binding domain that specifically binds to human CLDN6 comprises:
(i). a heavy chain variable region that comprises (a) a HCDR1 of SEQ ID NO: 1, (b) a HCDR2 of SEQ ID NO: 2, (c) a HCDR3 of SEQ ID NO: 3 and a light chain variable region that comprises: (d) a LCDR1 of SEQ ID NO: 4, (c) a LCDR2 of SEQ ID NO: 5, and (f) a LCDR3 of SEQ ID NO: 6;
(ii). a heavy chain variable region that comprises (a) a HCDR1 of SEQ ID NO: 1, (b) a HCDR2 of SEQ ID NO: 23, (c) a HCDR3 of SEQ ID NO: 3; and a light chain variable region that comprises: (d) a LCDR1 of SEQ ID NO: 4, (c) a LCDR2 of SEQ ID NO: 5, and (f) a LCDR3 of SEQ ID NO: 6;
(iii). a heavy chain variable region that comprises (a) a HCDR1 of SEQ ID NO: 1, (b) a HCDR2 of SEQ ID NO: 39, (c) a HCDR3 of SEQ ID NO: 3; and a light chain variable region that comprises: (d) a LCDR1 of SEQ ID NO: 40, (c) a LCDR2 of SEQ ID NO: 5, and (f) a LCDR3 of SEQ ID NO: 6; or
(iv). a heavy chain variable region that comprises (a) a HCDR1 of SEQ ID NO: 1, (b) a HCDR2 of SEQ ID NO: 45, (c) a HCDR3 of SEQ ID NO: 3; and a light chain variable region that comprises: (d) a LCDR1 of SEQ ID NO: 40, (c) a LCDR2 of SEQ ID NO: 5, and (f) a LCDR3 of SEQ ID NO: 6.

The multi-specific antibody or antigen-binding fragment, wherein the first antigen binding domain comprises:
(i). a heavy chain variable region (VH) comprising an amino acid sequence at least 90, 91, 92, 93, 94, 95, 96, 97, 98 or 99% identical to SEQ ID NO:7, and a light chain variable region (VL) comprising an amino acid sequence at least 90, 91, 92, 93, 94, 95, 96, 97, 98 or 99% identical to SEQ ID NO: 8;
(ii). a heavy chain variable region (VH) comprising an amino acid sequence at least 90, 91, 92, 93, 94, 95, 96, 97, 98 or 99% identical to SEQ ID NO: 24, and a light chain variable region (VL) comprising an amino acid sequence at least 90, 91, 92, 93, 94, 95, 96, 97, 98 or 99% identical to SEQ ID NO: 12;
(iii). a heavy chain variable region (VH) comprising an amino acid sequence at least 90, 91, 92, 93, 94, 95, 96, 97, 98 or 99% identical to SEQ ID NO: 41, and a light chain variable region (VL) comprising an amino acid sequence at least 90, 91, 92, 93, 94, 95, 96, 97, 98 or 99% identical to SEQ ID NO: 42;
(iv). a heavy chain variable region (VH) comprising an amino acid sequence at least 90, 91, 92, 93, 94, 95, 96, 97, 98 or 99% identical to SEQ ID NO: 43, and a light chain variable region (VL) comprising an amino acid sequence at least 90, 91, 92, 93, 94, 95, 96, 97, 98 or 99% identical to SEQ ID NO: 44;
(v). a heavy chain variable region (VH) comprising an amino acid sequence at least 90, 91, 92, 93, 94, 95, 96, 97, 98 or 99% identical to SEQ ID NO: 46, and a light chain variable region (VL) comprising an amino acid sequence at least 90, 91, 92, 93, 94, 95, 96, 97, 98 or 99% identical to SEQ ID NO: 47; or
(vi). a heavy chain variable region (VH) comprising an amino acid sequence at least 90, 91, 92, 93, 94, 95, 96, 97, 98 or 99% identical to SEQ ID NO: 46, and a light chain variable region (VL) comprising an amino acid sequence at least 90, 91, 92, 93, 94, 95, 96, 97, 98 or 99% identical to SEQ ID NO: 42.

The multi-specific antibody or antigen-binding fragment, wherein one, two, three, four, five, six, seven, eight, nine, or ten amino acids within SEQ ID NO: 7, 8, 12, 24, 41, 42, 43, 44, 46, or 47 have been inserted, deleted or substituted.

The multi-specific antibody or antigen-binding fragment, wherein the first antigen binding domain comprises:
(i). a heavy chain variable region (VH) that comprises SEQ ID NO:7, and a light chain variable region (VL) that comprises SEQ ID NO: 8;
(ii). a heavy chain variable region (VH) that comprises SEQ ID NO: 24, and a light chain variable region (VL) that comprises SEQ ID NO: 12;
(iii). a heavy chain variable region (VH) that comprises SEQ ID NO: 41, and a light chain variable region (VL) that comprises SEQ ID NO: 42;
(iv). a heavy chain variable region (VH) that comprises SEQ ID NO: 43, and a light chain variable region (VL) that comprises SEQ ID NO: 44;
(v). a heavy chain variable region (VH) that comprises SEQ ID NO: 46, and a light chain variable region (VL) that comprises SEQ ID NO: 47; or
(vi). a heavy chain variable region (VH) that comprises SEQ ID NO: 46, and a light chain variable region (VL) that comprises SEQ ID NO: 42.

The multi-specific antibody or antigen-binding fragment, wherein the second antigen binding domain that specifically binds to human CD3 comprises:
(i). a heavy chain variable region that comprises (a) a HCDR1 of SEQ ID NO: 48, (b) a HCDR2 of SEQ ID NO: 49, (c) a HCDR3 of SEQ ID NO: 50 and a light chain variable region that comprises: (d) a LCDR1 of SEQ ID NO: 51, (e) a LCDR2 of SEQ ID NO: 52, and (f) a LCDR3 of SEQ ID NO: 53;
(ii). a heavy chain variable region that comprises (a) a HCDR1 of SEQ ID NO: 48, (b) a HCDR2 of SEQ ID NO: 71, (c) a HCDR3 of SEQ ID NO: 50 and a light chain variable region that comprises: (d) a LCDR1 of SEQ ID NO: 51, (e) a LCDR2 of SEQ ID NO: 52, and (f) a LCDR3 of SEQ ID NO: 53; or
(iii). a heavy chain variable region that comprises (a) a HCDR1 of SEQ ID NO: 48, (b) a HCDR2 of SEQ ID NO: 71, (c) a HCDR3 of SEQ ID NO: 75 and a light chain variable region that comprises: (d) a LCDR1 of SEQ ID NO: 51, (e) a LCDR2 of SEQ ID NO: 52, and (f) a LCDR3 of SEQ ID NO: 53.

The multi-specific antibody or antigen-binding fragment, wherein the second antigen binding domain comprises:
a heavy chain variable region (VH) comprising an amino acid sequence at least 90, 91, 92, 93, 94, 95, 96, 97, 98 or 99% identical to SEQ ID NO: 54, and a light chain variable region (VL) comprising an amino acid sequence at least 90, 91, 92, 93, 94, 95, 96, 97, 98 or 99% identical to SEQ ID NO: 55;
a heavy chain variable region (VH) comprising an amino acid sequence at least 90, 91, 92, 93, 94, 95, 96, 97, 98 or 99% identical to SEQ ID NO: 58, and a light chain variable region (VL) comprising an amino acid sequence at least 90, 91, 92, 93, 94, 95, 96, 97, 98 or 99% identical to SEQ ID NO: 59;
a heavy chain variable region (VH) comprising an amino acid sequence at least 90, 91, 92, 93, 94, 95, 96, 97, 98 or 99% identical to SEQ ID NO: 62, and a light chain variable region (VL) comprising an amino acid sequence at least 90, 91, 92, 93, 94, 95, 96, 97, 98 or 99% identical to SEQ ID NO: 63;
a heavy chain variable region (VH) comprising an amino acid sequence at least 90, 91, 92, 93, 94, 95, 96, 97, 98 or 99% identical to SEQ ID NO: 62, and a light chain variable region (VL) comprising an amino acid sequence at least 90, 91, 92, 93, 94, 95, 96, 97, 98 or 99% identical to SEQ ID NO: 68;

a heavy chain variable region (VH) comprising an amino acid sequence at least 90, 91, 92, 93, 94, 95, 96, 97, 98 or 99% identical to SEQ ID NO: 72, and a light chain variable region (VL) comprising an amino acid sequence at least 90, 91, 92, 93, 94, 95, 96, 97, 98 or 99% identical to SEQ ID NO: 68; or

a heavy chain variable region (VH) comprising an amino acid sequence at least 90, 91, 92, 93, 94, 95, 96, 97, 98 or 99% identical to SEQ ID NO: 76, and a light chain variable region (VL) comprising an amino acid sequence at least 90, 91, 92, 93, 94, 95, 96, 97, 98 or 99% identical to SEQ ID NO: 68.

The multi-specific antibody or antigen-binding fragment, wherein one, two, three, four, five, six, seven, eight, nine, or ten amino acids within SEQ ID NO: 58, 59, 62 or 63 have been inserted, deleted or substituted.

The multi-specific antibody or antigen-binding fragment, wherein the second antigen binding domain comprises:
(i). a heavy chain variable region (VH) that comprises SEQ ID NO: 54, and a light chain variable region (VL) that comprises SEQ ID NO: 55;
(ii). a heavy chain variable region (VH) that comprises SEQ ID NO: 58, and a light chain variable region (VL) that comprises SEQ ID NO: 59;
(iii). a heavy chain variable region (VH) that comprises SEQ ID NO: 62, and a light chain variable region (VL) that comprises SEQ ID NO: 63;
(iv). a heavy chain variable region (VH) that comprises SEQ ID NO: 62, and a light chain variable region (VL) that comprises SEQ ID NO: 68;
(v). a heavy chain variable region (VH) that comprises SEQ ID NO: 72, and a light chain variable region (VL) that comprises SEQ ID NO: 68; or
(vi). a heavy chain variable region (VH) that comprises SEQ ID NO: 76, and a light chain variable region (VL) that comprises SEQ ID NO: 68.

The multi-specific antibody or antigen-binding fragment, wherein the second antigen binding domain comprises:
(a) a single chain variable fragment (scFv) that comprises SEQ ID NO: 66;
(b) a single chain variable fragment (scFv) that comprises SEQ ID NO: 69;
(c) a single chain variable fragment (scFv) that comprises SEQ ID NO: 73; or
(d) a single chain variable fragment (scFv) that comprises SEQ ID NO: 77.

The multi-specific antibody or antigen-binding fragment, wherein: (i) the first antigen binding domain that specifically binds to human CLDN6 comprises: a heavy chain variable region that comprises (a) a HCDR1 of SEQ ID NO: 1, (b) a HCDR2 of SEQ ID NO: 2, (c) a HCDR3 of SEQ ID NO: 3 and a light chain variable region that comprises: (d) a LCDR1 of SEQ ID NO: 4, (c) a LCDR2 of SEQ ID NO: 5, and (f) a LCDR3 of SEQ ID NO: 6; and the second antigen binding domain that specifically binds to human CD3 comprises: a heavy chain variable region that comprises (a) a HCDR1 of SEQ ID NO: 48, (b) a HCDR2 of SEQ ID NO: 49, (c) a HCDR3 of SEQ ID NO: 50 and a light chain variable region that comprises: (d) a LCDR1 of SEQ ID NO: 51, (c) a LCDR2 of SEQ ID NO: 52, and (f) a LCDR3 of SEQ ID NO: 53; (ii) the first antigen binding domain that specifically binds to human CLDN6 comprises: a heavy chain variable region that comprises (a) a HCDR1 of SEQ ID NO: 1, (b) a HCDR2 of SEQ ID NO: 2, (c) a HCDR3 of SEQ ID NO: 3 and a light chain variable region that comprises: (d) a LCDR1 of SEQ ID NO: 4, (c) a LCDR2 of SEQ ID NO: 5, and (f) a LCDR3 of SEQ ID NO: 6; and the second antigen binding domain that specifically binds to human CD3 comprises: a heavy chain variable region that comprises (a) a HCDR1 of SEQ ID NO: 48, (b) a HCDR2 of SEQ ID NO: 71, (c) a HCDR3 of SEQ ID NO: 50 and a light chain variable region that comprises: (d) a LCDR1 of SEQ ID NO: 51, (c) a LCDR2 of SEQ ID NO: 52, and (f) a LCDR3 of SEQ ID NO: 53; or (iii) the first antigen binding domain that specifically binds to human CLDN6 comprises: a heavy chain variable region that comprises (a) a HCDR1 of SEQ ID NO: 1, (b) a HCDR2 of SEQ ID NO: 2, (c) a HCDR3 of SEQ ID NO: 3 and a light chain variable region that comprises: (d) a LCDR1 of SEQ ID NO: 4, (c) a LCDR2 of SEQ ID NO: 5, and (f) a LCDR3 of SEQ ID NO: 6; and (iv) the second antigen binding domain that specifically binds to human CD3 comprises: a heavy chain variable region that comprises (a) a HCDR1 of SEQ ID NO: 48, (b) a HCDR2 of SEQ ID NO: 71, (c) a HCDR3 of SEQ ID NO: 75 and a light chain variable region that comprises: (d) a LCDR1 of SEQ ID NO: 51, (c) a LCDR2 of SEQ ID NO: 52, and (f) a LCDR3 of SEQ ID NO: 53.

The multi-specific antibody or antigen-binding fragment, wherein:
(i). the first antigen binding domain that specifically binds to human CLDN6 comprises: a heavy chain variable region that comprises (a) a HCDR1 of SEQ ID NO: 1, (b) a HCDR2 of SEQ ID NO: 23, (c) a HCDR3 of SEQ ID NO: 3 and a light chain variable region that comprises: (d) a LCDR1 of SEQ ID NO: 4, (c) a LCDR2 of SEQ ID NO: 5, and (f) a LCDR3 of SEQ ID NO: 6; and the second antigen binding domain that specifically binds to human CD3 comprises: a heavy chain variable region that comprises (a) a HCDR1 of SEQ ID NO: 48, (b) a HCDR2 of SEQ ID NO: 49, (c) a HCDR3 of SEQ ID NO: 50 and a light chain variable region that comprises: (d) a LCDR1 of SEQ ID NO: 51, (c) a LCDR2 of SEQ ID NO: 52, and (f) a LCDR3 of SEQ ID NO: 53;
(ii). the first antigen binding domain that specifically binds to human CLDN6 comprises: a heavy chain variable region that comprises (a) a HCDR1 of SEQ ID NO: 1, (b) a HCDR2 of SEQ ID NO: 23, (c) a HCDR3 of SEQ ID NO: 3 and a light chain variable region that comprises: (d) a LCDR1 of SEQ ID NO: 4, (c) a LCDR2 of SEQ ID NO: 5, and (f) a LCDR3 of SEQ ID NO: 6; and the second antigen binding domain that specifically binds to human CD3 comprises: a heavy chain variable region that comprises (a) a HCDR1 of SEQ ID NO: 48, (b) a HCDR2 of SEQ ID NO: 71, (c) a HCDR3 of SEQ ID NO: 50 and a light chain variable region that comprises: (d) a LCDR1 of SEQ ID NO: 51, (c) a LCDR2 of SEQ ID NO: 52, and (f) a LCDR3 of SEQ ID NO: 53; or
(iii). the first antigen binding domain that specifically binds to human CLDN6 comprises: a heavy chain variable region that comprises (a) a HCDR1 of SEQ ID NO: 1, (b) a HCDR2 of SEQ ID NO: 23, (c) a HCDR3 of SEQ ID NO: 3 and a light chain variable region that comprises: (d) a LCDR1 of SEQ ID NO: 4, (c) a LCDR2 of SEQ ID NO: 5, and (f) a LCDR3 of SEQ ID NO: 6; and the second antigen binding domain that specifically binds to human CD3 comprises: a heavy chain variable region that comprises (a) a HCDR1 of SEQ ID NO: 48, (b) a HCDR2 of SEQ ID NO: 71, (c) a HCDR3 of SEQ ID NO: 75 and a light chain variable region that comprises: (d) a LCDR1 of SEQ ID NO: 51, (e) a LCDR2 of SEQ ID NO: 52, and (f) a LCDR3 of SEQ ID NO: 53.

The multi-specific antibody or antigen-binding fragment, wherein:
(i). The multi-specific antibody or antigen-binding fragment, wherein: the first antigen binding domain that specifically binds to human CLDN6 comprises: a heavy chain variable region that comprises (a) a HCDR1 of SEQ ID NO: 1, (b) a HCDR2 of SEQ ID NO: 39, (c) a HCDR3 of SEQ ID NO: 3 and a light chain variable region that comprises: (d) a LCDR1 of SEQ ID NO: 40, (c) a LCDR2 of SEQ ID NO: 5, and (f) a LCDR3 of SEQ ID NO: 6; and the second antigen binding domain that specifically binds to human CD3 comprises: a heavy chain variable region that comprises (a) a HCDR1 of SEQ ID NO: 48, (b) a HCDR2 of SEQ ID NO: 49, (c) a HCDR3 of SEQ ID NO: 50 and a light chain variable region that comprises: (d) a LCDR1 of SEQ ID NO: 51, (c) a LCDR2 of SEQ ID NO: 52, and (f) a LCDR3 of SEQ ID NO: 53;
(ii). the first antigen binding domain that specifically binds to human CLDN6 comprises: a heavy chain variable region that comprises (a) a HCDR1 of SEQ ID NO: 1, (b) a HCDR2 of SEQ ID NO: 39, (c) a HCDR3 of SEQ ID NO: 3 and a light chain variable region that comprises: (d) a LCDR1 of SEQ ID NO: 40, (e) a LCDR2 of SEQ ID NO: 5, and (f) a LCDR3 of SEQ ID NO: 6; and the second antigen binding domain that specifically binds to human CD3 comprises: a heavy chain variable region that comprises (a) a HCDR1 of SEQ ID NO: 48, (b) a HCDR2 of SEQ ID NO: 71, (c) a HCDR3 of SEQ ID NO: 50 and a light chain variable region that comprises: (d) a LCDR1 of SEQ ID NO: 51, (c) a LCDR2 of SEQ ID NO: 52, and (f) a LCDR3 of SEQ ID NO: 53; or
(iii). the first antigen binding domain that specifically binds to human CLDN6 comprises: a heavy chain variable region that comprises (a) a HCDR1 of SEQ ID NO: 1, (b) a HCDR2 of SEQ ID NO: 39, (c) a HCDR3 of SEQ ID NO: 3 and a light chain variable region that comprises: (d) a LCDR1 of SEQ ID NO: 40, (c) a LCDR2 of SEQ ID NO: 5, and (f) a LCDR3 of SEQ ID NO: 6; and the second antigen binding domain that specifically binds to human CD3 comprises: a heavy chain variable region that comprises (a) a HCDR1 of SEQ ID NO: 48, (b) a HCDR2 of SEQ ID NO: 71, (c) a HCDR3 of SEQ ID NO: 75 and a light chain variable region that comprises: (d) a LCDR1 of SEQ ID NO: 51, (e) a LCDR2 of SEQ ID NO: 52, and (f) a LCDR3 of SEQ ID NO: 53.

The multi-specific antibody or antigen-binding fragment, wherein: the first antigen binding domain that specifically binds to human CLDN6 comprises: a heavy chain variable region that comprises (a) a HCDR1 of SEQ ID NO: 1, (b) a HCDR2 of SEQ ID NO: 39, (c) a HCDR3 of SEQ ID NO: 3 and a light chain variable region that comprises: (d) a LCDR1 of SEQ ID NO: 40, (c) a LCDR2 of SEQ ID NO: 5, and (f) a LCDR3 of SEQ ID NO: 6; and the second antigen binding domain that specifically binds to human CD3 comprises: a heavy chain variable region that comprises (a) a HCDR1 of SEQ ID NO: 48, (b) a HCDR2 of SEQ ID NO: 71, (c) a HCDR3 of SEQ ID NO: 75 and a light chain variable region that comprises: (d) a LCDR1 of SEQ ID NO: 51, (c) a LCDR2 of SEQ ID NO: 52, and (f) a LCDR3 of SEQ ID NO: 53.

The multi-specific antibody or antigen-binding fragment, wherein:
(i). the first antigen binding domain that specifically binds to human CLDN6 comprises: a heavy chain variable region that comprises (a) a HCDR1 of SEQ ID NO: 1, (b) a HCDR2 of SEQ ID NO: 45, (c) a HCDR3 of SEQ ID NO: 3 and a light chain variable region that comprises: (d) a LCDR1 of SEQ ID NO: 40, (c) a LCDR2 of SEQ ID NO: 5, and (f) a LCDR3 of SEQ ID NO: 6; and the second antigen binding domain that specifically binds to human CD3 comprises: a heavy chain variable region that comprises (a) a HCDR1 of SEQ ID NO: 48, (b) a HCDR2 of SEQ ID NO: 49, (c) a HCDR3 of SEQ ID NO: 50 and a light chain variable region that comprises: (d) a LCDR1 of SEQ ID NO: 51, (c) a LCDR2 of SEQ ID NO: 52, and (f) a LCDR3 of SEQ ID NO: 53;
(ii). the first antigen binding domain that specifically binds to human CLDN6 comprises: a heavy chain variable region that comprises (a) a HCDR1 of SEQ ID NO: 1, (b) a HCDR2 of SEQ ID NO: 45, (c) a HCDR3 of SEQ ID NO: 3 and a light chain variable region that comprises: (d) a LCDR1 of SEQ ID NO: 40, (c) a LCDR2 of SEQ ID NO: 5, and (f) a LCDR3 of SEQ ID NO: 6; and the second antigen binding domain that specifically binds to human CD3 comprises: a heavy chain variable region that comprises (a) a HCDR1 of SEQ ID NO: 48, (b) a HCDR2 of SEQ ID NO: 71, (c) a HCDR3 of SEQ ID NO: 50 and a light chain variable region that comprises: (d) a LCDR1 of SEQ ID NO: 51, (c) a LCDR2 of SEQ ID NO: 52, and (f) a LCDR3 of SEQ ID NO: 53; or
(iii). the first antigen binding domain that specifically binds to human CLDN6 comprises: a heavy chain variable region that comprises (a) a HCDR1 of SEQ ID NO: 1, (b) a HCDR2 of SEQ ID NO: 45, (c) a HCDR3 of SEQ ID NO: 3 and a light chain variable region that comprises: (d) a LCDR1 of SEQ ID NO: 40, (c) a LCDR2 of SEQ ID NO: 5, and (f) a LCDR3 of SEQ ID NO: 6; and the second antigen binding domain that specifically binds to human CD3 comprises: a heavy chain variable region that comprises (a) a HCDR1 of SEQ ID NO: 48, (b) a HCDR2 of SEQ ID NO: 71, (c) a HCDR3 of SEQ ID NO: 75 and a light chain variable region that comprises: (d) a LCDR1 of SEQ ID NO: 51, (c) a LCDR2 of SEQ ID NO: 52, and (f) a LCDR3 of SEQ ID NO: 53.

The multi-specific antibody or antigen-binding fragment, wherein: the first antigen binding domain that specifically binds to human CLDN6 comprises: a heavy chain variable region that comprises (a) a HCDR1 of SEQ ID NO: 1, (b) a HCDR2 of SEQ ID NO: 45, (c) a HCDR3 of SEQ ID NO: 3 and a light chain variable region that comprises: (d) a LCDR1 of SEQ ID NO: 40, (c) a LCDR2 of SEQ ID NO: 5, and (f) a LCDR3 of SEQ ID NO: 6; and the second antigen binding domain that specifically binds to human CD3 comprises: a heavy chain variable region that comprises (a) a HCDR1 of SEQ ID NO: 48, (b) a HCDR2 of SEQ ID NO: 71, (c) a HCDR3 of SEQ ID NO: 75 and a light chain variable region that comprises: (d) a LCDR1 of SEQ ID NO: 51, (c) a LCDR2 of SEQ ID NO: 52, and (f) a LCDR3 of SEQ ID NO: 53.

The multi-specific antibody or antigen-binding fragment, wherein:
(i). the first antigen binding domain that specifically binds to human CLDN6 comprises:
   a) a heavy chain variable region that comprises (a) a HCDR1 of SEQ ID NO: 1, (b) a HCDR2 of SEQ ID NO: 2, (c) a HCDR3 of SEQ ID NO: 3 and a light chain variable region that comprises: (d) a LCDR1 of SEQ ID NO: 4, (c) a LCDR2 of SEQ ID NO: 5, and (f) a LCDR3 of SEQ ID NO: 6;
   b) a heavy chain variable region that comprises (a) a HCDR1 of SEQ ID NO: 1, (b) a HCDR2 of SEQ ID NO: 23, (c) a HCDR3 of SEQ ID NO: 3 and a light chain variable region that comprises: (d) a LCDR1 of SEQ ID NO: 4, (c) a LCDR2 of SEQ ID NO: 5, and (f) a LCDR3 of SEQ ID NO: 6;
   c) a heavy chain variable region that comprises (a) a HCDR1 of SEQ ID NO: 1, (b) a HCDR2 of SEQ ID NO: 39, (c) a HCDR3 of SEQ ID NO: 3 and a light chain variable region that comprises: (d) a LCDR1 of SEQ ID NO: 40, (e) a LCDR2 of SEQ ID NO: 5, and (f) a LCDR3 of SEQ ID NO: 6; or
   d) the first antigen binding domain that specifically binds to human CLDN6 comprises: a heavy chain variable region that comprises (a) a HCDR1 of SEQ ID NO: 1, (b) a HCDR2 of SEQ ID NO: 45, (c) a HCDR3 of SEQ ID NO: 3 and a light chain variable region that comprises: (d) a LCDR1 of SEQ ID NO: 40, (e) a LCDR2 of SEQ ID NO: 5, and (f) a LCDR3 of SEQ ID NO: 6;
and
(ii). the second antigen binding domain that specifically binds to human CD3 comprises:
   a) a heavy chain variable region that comprises (a) a HCDR1 of SEQ ID NO: 48, (b) a HCDR2 of SEQ ID NO: 49, (c) a HCDR3 of SEQ ID NO: 50 and a light chain variable region that comprises: (d) a LCDR1 of SEQ ID NO: 51, (c) a LCDR2 of SEQ ID NO: 52, and (f) a LCDR3 of SEQ ID NO: 53;
   b) a heavy chain variable region that comprises (a) a HCDR1 of SEQ ID NO: 48, (b) a HCDR2 of SEQ ID NO: 71, (c) a HCDR3 of SEQ ID NO: 50 and a light chain variable region that comprises: (d) a LCDR1 of SEQ ID NO: 51, (c) a LCDR2 of SEQ ID NO: 52, and (f) a LCDR3 of SEQ ID NO: 53; or
   c) a heavy chain variable region that comprises (a) a HCDR1 of SEQ ID NO: 48, (b) a HCDR2 of SEQ ID NO: 71, (c) a HCDR3 of SEQ ID NO: 75 and a light chain variable region that comprises: (d) a LCDR1 of SEQ ID NO: 51, (c) a LCDR2 of SEQ ID NO: 52, and (f) a LCDR3 of SEQ ID NO: 53.

The multi-specific antibody or antigen-binding fragment, wherein:
(i). the first antigen binding domain that specifically binds to human CLDN6 comprises:
a) a heavy chain variable region that comprises (a) a HCDR1 of SEQ ID NO: 1, (b) a HCDR2 of SEQ ID NO: 2, (c) a HCDR3 of SEQ ID NO: 3 and a light chain variable region that comprises: (d) a LCDR1 of SEQ ID NO: 4, (c) a LCDR2 of SEQ ID NO: 5, and (f) a LCDR3 of SEQ ID NO: 6;
b) a heavy chain variable region that comprises (a) a HCDR1 of SEQ ID NO: 1, (b) a HCDR2 of SEQ ID NO: 23, (c) a HCDR3 of SEQ ID NO: 3 and a light chain variable region that comprises: (d) a LCDR1 of SEQ ID NO: 4, (e) a LCDR2 of SEQ ID NO: 5, and (f) a LCDR3 of SEQ ID NO: 6;
c) a heavy chain variable region that comprises (a) a HCDR1 of SEQ ID NO: 1, (b) a HCDR2 of SEQ ID NO: 39, (c) a HCDR3 of SEQ ID NO: 3 and a light chain variable region that comprises: (d) a LCDR1 of SEQ ID NO: 40, (e) a LCDR2 of SEQ ID NO: 5, and (f) a LCDR3 of SEQ ID NO: 6; or
d) the first antigen binding domain that specifically binds to human CLDN6 comprises: a heavy chain variable region that comprises (a) a HCDR1 of SEQ ID NO: 1, (b) a HCDR2 of SEQ ID NO: 45, (c) a HCDR3 of SEQ ID NO: 3 and a light chain variable region that comprises: (d) a LCDR1 of SEQ ID NO: 40, (e) a LCDR2 of SEQ ID NO: 5, and (f) a LCDR3 of SEQ ID NO: 6;
   and
(ii). the second antigen binding domain that specifically binds to human CD3 comprises:
a) a heavy chain variable region (VH) that comprises SEQ ID NO: 54, and a light chain variable region (VL) that comprises SEQ ID NO: 55;
b) a heavy chain variable region (VH) that comprises SEQ ID NO: 58, and a light chain variable region (VL) that comprises SEQ ID NO: 59;
c) a heavy chain variable region (VH) that comprises SEQ ID NO: 62, and a light chain variable region (VL) that comprises SEQ ID NO: 63;
d) a heavy chain variable region (VH) that comprises SEQ ID NO: 62, and a light chain variable region (VL) that comprises SEQ ID NO: 68;
e) a heavy chain variable region (VH) that comprises SEQ ID NO: 72, and a light chain variable region (VL) that comprises SEQ ID NO: 68; or
f) a heavy chain variable region (VH) that comprises SEQ ID NO: 76, and a light chain variable region (VL) that comprises SEQ ID NO: 68.

The multi-specific antibody or antigen-binding fragment, wherein:
(i). the first antigen binding domain that specifically binds to human CLDN6 comprises:
   a) a heavy chain variable region that comprises (a) a HCDR1 of SEQ ID NO: 1, (b) a HCDR2 of SEQ ID NO: 39, (c) a HCDR3 of SEQ ID NO: 3 and a light chain variable region that comprises: (d) a LCDR1 of SEQ ID NO: 40, (e) a LCDR2 of SEQ ID NO: 5, and (f) a LCDR3 of SEQ ID NO: 6; or
and
(ii). the second antigen binding domain that specifically binds to human CD3 comprises:
   a) a heavy chain variable region (VH) that comprises SEQ ID NO: 54, and a light chain variable region (VL) that comprises SEQ ID NO: 55;
   b) a heavy chain variable region (VH) that comprises SEQ ID NO: 58, and a light chain variable region (VL) that comprises SEQ ID NO: 59;
   c) a heavy chain variable region (VH) that comprises SEQ ID NO: 62, and a light chain variable region (VL) that comprises SEQ ID NO: 63;
   d) a heavy chain variable region (VH) that comprises SEQ ID NO: 62, and a light chain variable region (VL) that comprises SEQ ID NO: 68;
   e) a heavy chain variable region (VH) that comprises SEQ ID NO: 72, and a light chain variable region (VL) that comprises SEQ ID NO: 68; or
   f) a heavy chain variable region (VH) that comprises SEQ ID NO: 76, and a light chain variable region (VL) that comprises SEQ ID NO: 68.

The multi-specific antibody or antigen-binding fragment, wherein:
(i). the first antigen binding domain that specifically binds to human CLDN6 comprises:
   a) a heavy chain variable region that comprises (a) a HCDR1 of SEQ ID NO: 1, (b) a HCDR2 of SEQ ID NO: 23, (c) a HCDR3 of SEQ ID NO: 3 and a light chain variable region that comprises: (d) a LCDR1 of SEQ ID NO: 4, (e) a LCDR2 of SEQ ID NO: 5, and (f) a LCDR3 of SEQ ID NO: 6; or
   b) a heavy chain variable region that comprises (a) a HCDR1 of SEQ ID NO: 1, (b) a HCDR2 of SEQ ID NO: 39, (c) a HCDR3 of SEQ ID NO: 3 and a light chain variable region that comprises: (d) a LCDR1 of SEQ ID NO: 40, (e) a LCDR2 of SEQ ID NO: 5, and (f) a LCDR3 of SEQ ID NO: 6; or
   c) the first antigen binding domain that specifically binds to human CLDN6 comprises: a heavy chain variable region that comprises (a) a HCDR1 of SEQ ID NO: 1, (b) a HCDR2 of SEQ ID NO: 45, (c) a HCDR3 of SEQ ID NO: 3 and a light chain variable region that comprises: (d) a LCDR1 of SEQ ID NO: 40, (e) a LCDR2 of SEQ ID NO: 5, and (f) a LCDR3 of SEQ ID NO: 6;
and
(ii). the second antigen binding domain that specifically binds to human CD3 comprises:
   a) a single chain variable fragment (scFv) that comprises SEQ ID NO: 66;
   b) a single chain variable fragment (scFv) that comprises SEQ ID NO: 69;
   c) a single chain variable fragment (scFv) that comprises SEQ ID NO: 73; or
   d) a single chain variable fragment (scFv) that comprises SEQ ID NO: 77.

The multi-specific antibody or antigen-binding fragment, wherein:
(i). the first antigen binding domain that specifically binds to human CLDN6 comprises:
   a) a heavy chain variable region (VH) that comprises SEQ ID NO: 24, and a light chain variable region (VL) that comprises SEQ ID NO: 12;
   b) a heavy chain variable region (VH) that comprises SEQ ID NO: 41, and a light chain variable region (VL) that comprises SEQ ID NO: 42;
   c) a heavy chain variable region (VH) that comprises SEQ ID NO: 43, and a light chain variable region (VL) that comprises SEQ ID NO: 44;
   d) a heavy chain variable region (VH) that comprises SEQ ID NO: 46, and a light chain variable region (VL) that comprises SEQ ID NO: 47; or
   e) a heavy chain variable region (VH) that comprises SEQ ID NO: 46, and a light chain variable region (VL) that comprises SEQ ID NO: 42;
and
(ii). the second antigen binding domain that specifically binds to human CD3 comprises:
   a) a heavy chain variable region (VH) that comprises SEQ ID NO: 54, and a light chain variable region (VL) that comprises SEQ ID NO: 55;
   b) a heavy chain variable region (VH) that comprises SEQ ID NO: 58, and a light chain variable region (VL) that comprises SEQ ID NO: 59;
   c) a heavy chain variable region (VH) that comprises SEQ ID NO: 62, and a light chain variable region (VL) that comprises SEQ ID NO: 63;
   d) a heavy chain variable region (VH) that comprises SEQ ID NO: 62, and a light chain variable region (VL) that comprises SEQ ID NO: 68;
   e) a heavy chain variable region (VH) that comprises SEQ ID NO: 72, and a light chain variable region (VL) that comprises SEQ ID NO: 68; or
   f) a heavy chain variable region (VH) that comprises SEQ ID NO: 76, and a light chain variable region (VL) that comprises SEQ ID NO: 68.

The multi-specific antibody or antigen-binding fragment, wherein:
(i). the first antigen binding domain that specifically binds to human CLDN6 comprises:
   a) a heavy chain variable region (VH) that comprises SEQ ID NO: 24, and a light chain variable region (VL) that comprises SEQ ID NO: 12;
   b) a heavy chain variable region (VH) that comprises SEQ ID NO: 41, and a light chain variable region (VL) that comprises SEQ ID NO: 42;
   c) a heavy chain variable region (VH) that comprises SEQ ID NO: 43, and a light chain variable region (VL) that comprises SEQ ID NO: 44;
   d) a heavy chain variable region (VH) that comprises SEQ ID NO: 46, and a light chain variable region (VL) that comprises SEQ ID NO: 47; or
   e) a heavy chain variable region (VH) that comprises SEQ ID NO: 46, and a light chain variable region (VL) that comprises SEQ ID NO: 42;
and
(ii). the second antigen binding domain that specifically binds to human CD3 comprises: a heavy chain variable region (VH) that comprises SEQ ID NO: 72, and a light chain variable region (VL) that comprises SEQ ID NO: 68.

The multi-specific antibody or antigen-binding fragment, wherein:
(i). the first antigen binding domain that specifically binds to human CLDN6 comprises:
   a) a heavy chain variable region (VH) that comprises SEQ ID NO: 24, and a light chain variable region (VL) that comprises SEQ ID NO: 12;
   b) a heavy chain variable region (VH) that comprises SEQ ID NO: 41, and a light chain variable region (VL) that comprises SEQ ID NO: 42;
   c) a heavy chain variable region (VH) that comprises SEQ ID NO: 43, and a light chain variable region (VL) that comprises SEQ ID NO: 44;
   d) a heavy chain variable region (VH) that comprises SEQ ID NO: 46, and a light chain variable region (VL) that comprises SEQ ID NO: 47; or
   e) a heavy chain variable region (VH) that comprises SEQ ID NO: 46, and a light chain variable region (VL) that comprises SEQ ID NO: 42;
and
(ii). the second antigen binding domain that specifically binds to human CD3 comprises: a heavy chain variable region (VH) that comprises SEQ ID NO: 76, and a light chain variable region (VL) that comprises SEQ ID NO: 68.

The multi-specific antibody or antigen-binding fragment, wherein: the first antigen binding domain that specifically binds to human CLDN6 comprises: a heavy chain variable region (VH) that comprises SEQ ID NO: 43, and a light chain variable region (VL) that comprises SEQ ID NO: 44; and the second antigen binding domain that specifically binds to human CD3 comprises:
a) a heavy chain variable region (VH) that comprises SEQ ID NO: 54, and a light chain variable region (VL) that comprises SEQ ID NO: 55;
b) a heavy chain variable region (VH) that comprises SEQ ID NO: 58, and a light chain variable region (VL) that comprises SEQ ID NO: 59;
c) a heavy chain variable region (VH) that comprises SEQ ID NO: 62, and a light chain variable region (VL) that comprises SEQ ID NO: 63;
d) a heavy chain variable region (VH) that comprises SEQ ID NO: 62, and a light chain variable region (VL) that comprises SEQ ID NO: 68;
e) a heavy chain variable region (VH) that comprises SEQ ID NO: 72, and a light chain variable region (VL) that comprises SEQ ID NO: 68; or
f) a heavy chain variable region (VH) that comprises SEQ ID NO: 76, and a light chain variable region (VL) that comprises SEQ ID NO: 68.

The multi-specific antibody or antigen-binding fragment, wherein: the first antigen binding domain that specifically binds to human CLDN6 comprises: a heavy chain variable region (VH) that comprises SEQ ID NO: 43, and a light chain variable region (VL) that comprises SEQ ID NO: 44; and the second antigen binding domain that specifically binds to human CD3 comprises: a heavy chain variable region (VH) that comprises SEQ ID NO: 72, and a light chain variable region (VL) that comprises SEQ ID NO: 68.

The multi-specific antibody or antigen-binding fragment, wherein: the first antigen binding domain that specifically binds to human CLDN6 comprises: a heavy chain variable region (VH) that comprises SEQ ID NO: 43, and a light chain variable region (VL) that comprises SEQ ID NO: 44; and the second antigen binding domain that specifically binds to human CD3 comprises: a heavy chain variable region (VH) that comprises SEQ ID NO: 76, and a light chain variable region (VL) that comprises SEQ ID NO: 68.

The multi-specific antibody or antigen-binding fragment, wherein:
(i). the first antigen binding domain that specifically binds to human CLDN6 comprises:
   a) a heavy chain variable region (VH) that comprises SEQ ID NO: 24, and a light chain variable region (VL) that comprises SEQ ID NO: 12;
   b) a heavy chain variable region (VH) that comprises SEQ ID NO: 41, and a light chain variable region (VL) that comprises SEQ ID NO: 42;
   c) a heavy chain variable region (VH) that comprises SEQ ID NO: 43, and a light chain variable region (VL) that comprises SEQ ID NO: 44;
   d) a heavy chain variable region (VH) that comprises SEQ ID NO: 46, and a light chain variable region (VL) that comprises SEQ ID NO: 47; or
   e) a heavy chain variable region (VH) that comprises SEQ ID NO: 46, and a light chain variable region (VL) that comprises SEQ ID NO: 42.
and
(ii). the second antigen binding domain that specifically binds to human CD3 comprises:
   a) a single chain variable fragment (scFv) that comprises SEQ ID NO: 66;
   b) a single chain variable fragment (scFv) that comprises SEQ ID NO: 69;
   c) a single chain variable fragment (scFv) that comprises SEQ ID NO: 73; or
   d) a single chain variable fragment (scFv) that comprises SEQ ID NO: 77.

The multi-specific antibody or antigen-binding fragment, wherein: the first antigen binding domain that specifically binds to human CLDN6 comprises: a heavy chain variable region (VH) that comprises SEQ ID NO: 43, and a light chain variable region (VL) that comprises SEQ ID NO: 44; and the second antigen binding domain that specifically binds to human CD3 comprises:
a) a single chain variable fragment (scFv) that comprises SEQ ID NO: 66;
b) a single chain variable fragment (scFv) that comprises SEQ ID NO: 69;
c) a single chain variable fragment (scFv) that comprises SEQ ID NO: 73; or
d) a single chain variable fragment (scFv) that comprises SEQ ID NO: 77.

The multi-specific antibody or antigen-binding fragment, wherein: the first antigen binding domain that specifically binds to human CLDN6 comprises: a heavy chain variable region (VH) that comprises SEQ ID NO: 43, and a light chain variable region (VL) that comprises SEQ ID NO: 44; and the second antigen binding domain that specifically binds to human CD3 comprises: a single chain variable fragment (scFv) that comprises SEQ ID NO: 66.

The multi-specific antibody or antigen-binding fragment, wherein: the first antigen binding domain that specifically binds to human CLDN6 comprises: a heavy chain variable region (VH) that comprises SEQ ID NO: 43, and a light chain variable region (VL) that comprises SEQ ID NO: 44; and the second antigen binding domain that specifically binds to human CD3 comprises: a single chain variable fragment (scFv) that comprises SEQ ID NO: 69.

The multi-specific antibody or antigen-binding fragment, wherein: the first antigen binding domain that specifically binds to human CLDN6 comprises: a heavy chain variable region (VH) that comprises SEQ ID NO: 43, and a light chain variable region (VL) that comprises SEQ ID NO: 44; and the second antigen binding domain that specifically binds to human CD3 comprises: a single chain variable fragment (scFv) that comprises SEQ ID NO: 73.

The multi-specific antibody or antigen-binding fragment, wherein: the first antigen binding domain that specifically binds to human CLDN6 comprises: a heavy chain variable region (VH) that comprises SEQ ID NO: 43, and a light chain variable region (VL) that comprises SEQ ID NO: 44; and the second antigen binding domain that specifically binds to human CD3 comprises: a single chain variable fragment (scFv) that comprises SEQ ID NO: 77.

The multi-specific antibody or antigen-binding fragment, which is a monoclonal antibody, a chimeric antibody, a humanized antibody, a human engineered antibody, a single chain antibody (scFv), a Fab fragment, a Fab′ fragment, or a F(ab′)2 fragment.

The multi-specific antibody, wherein the multi-specific antibody is a bispecific antibody.

The multi-specific antibody, wherein the multi-specific antibody is BG143P (SEQ ID NO: 80, SEQ ID NO: 82, and SEQ ID NO: 84).

The multi-specific antibody or antigen-binding fragment, wherein the antibody or antigen-binding fragment thereof has antibody dependent cellular cytotoxicity (ADCC) or complement dependent cytotoxicity (CDC).

The multi-specific antibody or antigen-binding fragment, wherein the antibody or antigen-binding fragment thereof has reduced glycosylation or no glycosylation or is hypofucosylated.

The multi-specific antibody or antigen-binding fragment, wherein the antibody or antigen-binding fragment thereof comprises increased bisecting GlcNac structures.

The multi-specific antibody or antigen-binding fragment, wherein the Fc domain is an IgG1 with reduced effector function.

The multi-specific antibody or antigen-binding fragment, wherein the Fc domain is an IgG4.

A pharmaceutical composition comprising the multi-specific antibody or antigen-binding fragment thereof further comprising a pharmaceutically acceptable carrier.

The pharmaceutical composition, further comprising histidine/histidine HCl, trehalose dihydrate, and polysorbate 20.

A method of treating cancer comprising administering to a patient in need an effective amount of the multi-specific antibody or antigen-binding fragment.

The method, wherein the cancer is gastric cancer, breast cancer, lung cancer, small cell lung cancer, non-small cell lung cancer, ovarian cancer, testicular cancer, endometrial cancer, bladder cancer, rhabdoid tumor and glioma.

The method, wherein the multi-specific antibody or antigen-binding fragment is administered in combination with another therapeutic agent.

The method, wherein the therapeutic agent is paclitaxel or a paclitaxel agent, docetaxel, carboplatin, topotecan, cisplatin, irinotecan, doxorubicin, lenalidomide or 5-azacytidine.

The method, wherein the therapeutic agent is a paclitaxel agent, lenalidomide or 5-azacytidine.

The method, wherein the therapeutic agent an anti-PD1 or anti-PDL1 antibody.

The method, wherein the anti-PD1 antibody is Tislelizumab.

An isolated nucleic acid that encodes the multi-specific antibody or antigen-binding fragment.

A vector comprising the nucleic acid.

A host cell comprising the nucleic acid or the vector.

A process for producing a multi-specific antibody or antigen-binding fragment thereof comprising cultivating the host cell and recovering the antibody or antigen-binding fragment from the culture.

In one embodiment, the multi-specific antibody or an antigen-binding fragment thereof comprises one or more complementarity determining regions (CDRs) comprising an amino acid sequence selected from a group consisting of SEQ ID NO: 1, SEQ ID NO: 2, SEQ ID NO: 3, SEQ ID NO: 23, SEQ ID NO: 39, SEQ ID NO: 48, SEQ ID NO: 49, SEQ ID NO: 50, SEQ ID NO: 71, SEQ ID NO: 75, SEQ ID NO: 4, SEQ ID NO: 5, SEQ ID NO: 6, SEQ ID NO: 40, SEQ ID NO: 51, SEQ ID NO: 52, SEQ ID NO: 53.

In another embodiment, the multi-specific antibody or an antigen-binding fragment thereof comprises: (a) a heavy chain variable region comprising one or more complementarity determining regions (HCDRs) comprising an amino acid sequence selected from the group consisting of SEQ ID NO: 1, SEQ ID NO: 2, SEQ ID NO: 3, SEQ ID NO: 23, SEQ ID NO: 39, SEQ ID NO: 48, SEQ ID NO: 49, SEQ ID NO: 50, SEQ ID NO: 71, SEQ ID NO: 75 and/or (b) a light chain variable region comprising one or more complementarity determining regions (LCDRs) having an amino acid sequence selected from the group consisting of SEQ ID NO: 4, SEQ ID NO: 5, SEQ ID NO: 6, SEQ ID NO: 40, SEQ ID NO: 51, SEQ ID NO: 52, SEQ ID NO: 53.

In another embodiment, the multi-specific antibody or an antigen-binding fragment thereof comprises: (a) a heavy chain variable region comprising three complementarity determining regions (HCDRs) which are HCDR1 comprising an amino acid sequence of SEQ ID NO: 1; SEQ ID NO: 48, HCDR2 comprising an amino acid sequence of SEQ ID NO: 2, SEQ ID NO: 23, SEQ ID NO: 39, SEQ ID NO: 45, SEQ ID NO: 49, SEQ ID NO: 71, and HCDR3 comprising an amino acid sequence of SEQ ID NO: 3, SEQ ID NO: 50, SEQ ID NO: 75, and/or (b) a light chain variable region comprising three complementarity determining regions (LCDRs) which are LCDR1 comprising an amino acid sequence of SEQ ID NO: 4, SEQ ID NO: 40, or SEQ ID NO: 51, LCDR2 comprising an amino acid sequence of SEQ ID NO: 5, SEQ ID NO: 52; and LCDR3 comprising an amino acid sequence of SEQ ID NO: 6, SEQ ID NO: 53.

In another embodiment, the multi-specific antibody or an antigen-binding fragment thereof comprises:
(a) a heavy chain variable region comprising three complementarity determining regions (HCDRs) which are
HCDR1 comprising an amino acid sequence of SEQ ID NO: 1, HCDR2 comprising an amino acid sequence of SEQ ID NO: 2, and HCDR3 comprising an amino acid sequence of SEQ ID NO: 3;
HCDR1 comprising an amino acid sequence of SEQ ID NO: 1, HCDR2 comprising an amino acid sequence of SEQ ID NO: 23, and HCDR3 comprising an amino acid sequence of SEQ ID NO: 3;
HCDR1 comprising an amino acid sequence of SEQ ID NO: 1, HCDR2 comprising an amino acid sequence of SEQ ID NO: 39, and HCDR3 comprising an amino acid sequence of SEQ ID NO: 3; or
HCDR1 comprising an amino acid sequence of SEQ ID NO: 15, HCDR2 comprising an amino acid sequence of SEQ ID NO: 45, and HCDR3 comprising an amino acid sequence of SEQ ID NO: 3;
and/or (b) a light chain variable region comprising three complementarity determining regions (LCDRs) which are
LCDR1 comprising an amino acid sequence of SEQ ID NO: 4, LCDR2 comprising an amino acid sequence of SEQ ID NO: 5, and LCDR3 comprising an amino acid sequence of SEQ ID NO: 6; or
LCDR1 comprising an amino acid sequence of SEQ ID NO: 40, LCDR2 comprising an amino acid sequence of SEQ ID NO: 5, and LCDR3 comprising an amino acid sequence of SEQ ID NO: 6.

In another embodiment, the multi-specific antibody or the antigen-binding fragment comprises: a first antigen binding domain comprising: a heavy chain variable region that comprises (a) a HCDR1 of SEQ ID NO: 1, (b) a HCDR2 of SEQ ID NO: 2, (c) a HCDR3 of SEQ ID NO: 3 and a light chain variable region that comprises: (d) a LCDR1 of SEQ ID NO: 4, (c) a LCDR2 of SEQ ID NO: 5, and (f) a LCDR3 of SEQ ID NO: 6;

and a second antigen binding domain comprising: a heavy chain variable region that comprises (a) a HCDR1 of SEQ ID NO: 48, (b) a HCDR2 of SEQ ID NO: 49, (c) a HCDR3 of SEQ ID NO: 50 and a light chain variable region that comprises: (d) a LCDR1 of SEQ ID NO: 51, (c) a LCDR2 of SEQ ID NO: 52, and (f) a LCDR3 of SEQ ID NO: 53.

In another embodiment, the multi-specific antibody or the antigen-binding fragment comprises: a first antigen binding domain comprising: a heavy chain variable region that comprises (a) a HCDR1 of SEQ ID NO: 1, (b) a HCDR2 of SEQ ID NO: 23, (c) a HCDR3 of SEQ ID NO: 3 and a light chain variable region that comprises: (d) a LCDR1 of SEQ ID NO: 4, (c) a LCDR2 of SEQ ID NO: 5, and (f) a LCDR3 of SEQ ID NO: 6;

and a second antigen binding domain comprising: a heavy chain variable region that comprises (a) a HCDR1 of SEQ ID NO: 48, (b) a HCDR2 of SEQ ID NO: 49, (c) a HCDR3 of SEQ ID NO: 50 and a light chain variable region that comprises: (d) a LCDR1 of SEQ ID NO: 51, (e) a LCDR2 of SEQ ID NO: 52, and (f) a LCDR3 of SEQ ID NO: 53.

In another embodiment, the multi-specific antibody or the antigen-binding fragment comprises: a first antigen binding domain comprising: a heavy chain variable region that comprises (a) a HCDR1 of SEQ ID NO: 1, (b) a HCDR2 of SEQ ID NO: 39, (c) a HCDR3 of SEQ ID NO: 3 and a light chain variable region that comprises: (d) a LCDR1 of SEQ ID NO: 40, (c) a LCDR2 of SEQ ID NO: 5, and (f) a LCDR3 of SEQ ID NO: 6;

and a second antigen binding domain comprising: a heavy chain variable region that comprises (a) a HCDR1 of SEQ ID NO: 48, (b) a HCDR2 of SEQ ID NO: 49, (c) a HCDR3 of SEQ ID NO: 50 and a light chain variable region that comprises: (d) a LCDR1 of SEQ ID NO: 51, (c) a LCDR2 of SEQ ID NO: 52, and (f) a LCDR3 of SEQ ID NO: 53.

In another embodiment, the multi-specific antibody or the antigen-binding fragment comprises: a first antigen binding domain comprising: a heavy chain variable region that comprises (a) a HCDR1 of SEQ ID NO: 1, (b) a HCDR2 of SEQ ID NO: 45, (c) a HCDR3 of SEQ ID NO: 3 and a light chain variable region that comprises: (d) a LCDR1 of SEQ ID NO: 40, (c) a LCDR2 of SEQ ID NO: 5, and (f) a LCDR3 of SEQ ID NO: 6;

and a second antigen binding domain comprising: a heavy chain variable region that comprises (a) a HCDR1 of SEQ ID NO: 48, (b) a HCDR2 of SEQ ID NO: 49, (c) a HCDR3 of SEQ ID NO: 50 and a light chain variable region that comprises: (d) a LCDR1 of SEQ ID NO: 51, (c) a LCDR2 of SEQ ID NO: 52, and (f) a LCDR3 of SEQ ID NO: 53.

In another embodiment, the multi-specific antibody or the antigen-binding fragment comprises: a first antigen binding domain comprising: a heavy chain variable region that comprises (a) a HCDR1 of SEQ ID NO: 1, (b) a HCDR2 of SEQ ID NO: 2, (c) a HCDR3 of SEQ ID NO: 3 and a light chain variable region that comprises: (d) a LCDR1 of SEQ ID NO: 4, (c) a LCDR2 of SEQ ID NO: 5, and (f) a LCDR3 of SEQ ID NO: 6;

and a second antigen binding domain comprising: a heavy chain variable region that comprises (a) a HCDR1 of SEQ ID NO: 48, (b) a HCDR2 of SEQ ID NO: 71, (c) a HCDR3 of SEQ ID NO: 50 and a light chain variable region that comprises: (d) a LCDR1 of SEQ ID NO: 51, (c) a LCDR2 of SEQ ID NO: 52, and (f) a LCDR3 of SEQ ID NO: 53.

In another embodiment, the multi-specific antibody or the antigen-binding fragment comprises: a first antigen binding domain comprising: a heavy chain variable region that comprises (a) a HCDR1 of SEQ ID NO: 1, (b) a HCDR2 of SEQ ID NO: 23, (c) a HCDR3 of SEQ ID NO: 3 and a light chain variable region that comprises: (d) a LCDR1 of SEQ ID NO: 4, (c) a LCDR2 of SEQ ID NO: 5, and (f) a LCDR3 of SEQ ID NO: 6;

and a second antigen binding domain comprising: a heavy chain variable region that comprises (a) a HCDR1 of SEQ ID NO: 48, (b) a HCDR2 of SEQ ID NO: 71, (c) a HCDR3 of SEQ ID NO: 50 and a light chain variable region that comprises: (d) a LCDR1 of SEQ ID NO: 51, (c) a LCDR2 of SEQ ID NO: 52, and (f) a LCDR3 of SEQ ID NO: 53.

In another embodiment, the multi-specific antibody or the antigen-binding fragment comprises: a first antigen binding domain comprising: a heavy chain variable region that comprises (a) a HCDR1 of SEQ ID NO: 1, (b) a HCDR2 of SEQ ID NO: 39, (c) a HCDR3 of SEQ ID NO: 3 and a light chain variable region that comprises: (d) a LCDR1 of SEQ ID NO: 40, (c) a LCDR2 of SEQ ID NO: 5, and (f) a LCDR3 of SEQ ID NO: 6;

and a second antigen binding domain comprising: a heavy chain variable region that comprises (a) a HCDR1 of SEQ ID NO: 48, (b) a HCDR2 of SEQ ID NO: 71, (c) a HCDR3 of SEQ ID NO: 50 and a light chain variable region that comprises: (d) a LCDR1 of SEQ ID NO: 51, (c) a LCDR2 of SEQ ID NO: 52, and (f) a LCDR3 of SEQ ID NO: 53.

In another embodiment, the multi-specific antibody or the antigen-binding fragment comprises: a first antigen binding domain comprising: a heavy chain variable region that comprises (a) a HCDR1 of SEQ ID NO: 1, (b) a HCDR2 of SEQ ID NO: 45, (c) a HCDR3 of SEQ ID NO: 3 and a light chain variable region that comprises: (d) a LCDR1 of SEQ ID NO: 40, (c) a LCDR2 of SEQ ID NO: 5, and (f) a LCDR3 of SEQ ID NO: 6;

and a second antigen binding domain comprising: a heavy chain variable region that comprises (a) a HCDR1 of SEQ ID NO: 48, (b) a HCDR2 of SEQ ID NO: 71, (c) a HCDR3 of SEQ ID NO: 50 and a light chain variable region that comprises: (d) a LCDR1 of SEQ ID NO: 51, (c) a LCDR2 of SEQ ID NO: 52, and (f) a LCDR3 of SEQ ID NO: 53.

In another embodiment, the multi-specific antibody or the antigen-binding fragment comprises: a first antigen binding domain comprising: a heavy chain variable region that comprises (a) a HCDR1 of SEQ ID NO: 1, (b) a HCDR2 of SEQ ID NO: 2, (c) a HCDR3 of SEQ ID NO: 3 and a light chain variable region that comprises: (d) a LCDR1 of SEQ ID NO: 4, (c) a LCDR2 of SEQ ID NO: 5, and (f) a LCDR3 of SEQ ID NO: 6;

and a second antigen binding domain comprising: a heavy chain variable region that comprises (a) a HCDR1 of SEQ ID NO: 48, (b) a HCDR2 of SEQ ID NO: 71, (c) a HCDR3 of SEQ ID NO: 75 and a light chain variable region that comprises: (d) a LCDR1 of SEQ ID NO: 51, (c) a LCDR2 of SEQ ID NO: 52, and (f) a LCDR3 of SEQ ID NO: 53.

In another embodiment, the multi-specific antibody or the antigen-binding fragment comprises: a first antigen binding domain comprising: a heavy chain variable region that comprises (a) a HCDR1 of SEQ ID NO: 1, (b) a HCDR2 of SEQ ID NO: 23, (c) a HCDR3 of SEQ ID NO: 3 and a light chain variable region that comprises: (d) a LCDR1 of SEQ ID NO: 4, (c) a LCDR2 of SEQ ID NO: 5, and (f) a LCDR3 of SEQ ID NO: 6;

and a second antigen binding domain comprising: a heavy chain variable region that comprises (a) a HCDR1 of SEQ ID NO: 48, (b) a HCDR2 of SEQ ID NO: 71, (c) a HCDR3 of SEQ ID NO: 75 and a light chain variable region that comprises: (d) a LCDR1 of SEQ ID NO: 51, (c) a LCDR2 of SEQ ID NO: 52, and (f) a LCDR3 of SEQ ID NO: 53.

In another embodiment, the multi-specific antibody or the antigen-binding fragment comprises: a first antigen binding domain comprising: a heavy chain variable region that comprises (a) a HCDR1 of SEQ ID NO: 1, (b) a HCDR2 of SEQ ID NO: 39, (c) a HCDR3 of SEQ ID NO: 3 and a light chain variable region that comprises: (d) a LCDR1 of SEQ ID NO: 40, (c) a LCDR2 of SEQ ID NO: 5, and (f) a LCDR3 of SEQ ID NO: 6;

and a second antigen binding domain comprising: a heavy chain variable region that comprises (a) a HCDR1 of SEQ ID NO: 48, (b) a HCDR2 of SEQ ID NO: 71, (c) a HCDR3 of SEQ ID NO: 75 and a light chain variable region that comprises: (d) a LCDR1 of SEQ ID NO: 51, (c) a LCDR2 of SEQ ID NO: 52, and (f) a LCDR3 of SEQ ID NO: 53.

In another embodiment, the multi-specific antibody or the antigen-binding fragment comprises: a first antigen binding domain comprising: a heavy chain variable region that comprises (a) a HCDR1 of SEQ ID NO: 1, (b) a HCDR2 of SEQ ID NO: 45, (c) a HCDR3 of SEQ ID NO: 3 and a light chain variable region that comprises: (d) a LCDR1 of SEQ ID NO: 40, (c) a LCDR2 of SEQ ID NO: 5, and (f) a LCDR3 of SEQ ID NO: 6;

and a second antigen binding domain comprising: a heavy chain variable region that comprises (a) a HCDR1 of SEQ ID NO: 48, (b) a HCDR2 of SEQ ID NO: 71, (c) a HCDR3 of SEQ ID NO: 75 and a light chain variable region that comprises: (d) a LCDR1 of SEQ ID NO: 51, (c) a LCDR2 of SEQ ID NO: 52, and (f) a LCDR3 of SEQ ID NO: 53.

In one embodiment, the antibody of the present disclosure or an antigen-binding fragment thereof comprises: (a) a heavy chain variable region having an amino acid sequence of HCDRs or VHs listed in Table 1 and 2; and/or (b) a light chain variable region comprising an amino acid sequence of LCDRs or VLs listed in Table 1 and 2.

In another embodiment, the multi-specific antibody of the present disclosure or an antigen-binding fragment thereof comprises: (a) an amino acid sequence comprising one, two, or three amino acid substitutions in the amino acid sequence of HCDRs or VHs listed in Table 1 and 2; and/or (b) a light chain variable region comprising an amino acid sequence comprising one, two, three, four, or five amino acid substitutions in the amino acid of LCDRs or VLs listed in Table 1 and 2. In another embodiment, the amino acid substitutions are conservative amino acid substitutions.

In one embodiment, the multi-specific antibody of the present disclosure is of IgG1, IgG2, IgG3, or IgG4 isotype. In a more specific embodiment, the antibody of the present disclosure comprises Fc domain of wild-type human IgG1 (also referred as human IgG1 wt or huIgG1) or IgG2.

In one embodiment, the multi-specific antibody of the present disclosure binds to CLDN6 with a binding affinity (K_{D}) of from 1×10⁻⁶ M to 1×10⁻¹⁰ M. In another embodiment, the antibody of the present disclosure binds to CLDN6 with a binding affinity (K_{D}) of about 1×10⁻⁶ M, about 1×10⁻⁷ M, about 1×10⁻⁸ M, about 1×10⁻⁹ M or about 1×10⁻¹⁰ M.

In another embodiment, the anti-human CLDN6 multi-specific antibody of the present disclosure shows a cross-species binding activity to cynomolgus CLDN6.

In one embodiment, antibodies of the present disclosure have strong Fc-mediated effector functions. The antibodies mediate antibody-dependent cellular cytotoxicity (ADCC) against CLDN6 expressing target cells.

In another aspect, the present disclosure relates to a pharmaceutical composition comprising the CLDN6×CD3 multi-specific antibody or antigen-binding fragment thereof, and optionally a pharmaceutically acceptable excipient.

In yet another aspect, the present disclosure relates to a method of treating a disease in a subject, which comprises administering the CLDN6×CD3 multi-specific antibody or antigen-binding fragment thereof, or a CLDN6×CD3 multi-specific antibody pharmaceutical composition in a therapeutically effective amount to a subject in need thereof. In another embodiment the disease to be treated by the antibody or the antigen-binding fragment is cancer.

The current disclosure relates to use of the CLDN6×CD3 multi-specific antibody or the antigen-binding fragment thereof, or a CLDN6×CD3 multi-specific antibody pharmaceutical composition for treating a disease, such as cancer.

### BRIEF DESCRIPTION OF THE DRAWINGS

FIGS. 1A, 1B, IC, 1D, 1E, IF, 1G, 1H, 1I, and 1J show the cell binding activity of chBG87P engineered variants. FIG. 1A shows cell binding activity of the 1^{st} round of BG87P humanization back mutation variants (BG87P-z0, BG87P-Bz0, BG87P-Bz1, BG87P-Bz2, BG87P-B23, BG87P-Bz4, BG87P-Bz5, BG87P-B26, BG87P-Bz7, and BG87P-Bz8) compared with anti-CLDN6 chimeric BG87P (chBG87P) against the HEK293T/human CLDN6. FIG. 1B shows cell binding activity of combination humanized variants (BG87P-21, BG87P-22, BG87P-23, and BG87P-24) compared with anti-CLDN6 chimeric BG87P (chBG87P) against the HEK293T/human CLDN6. FIG. 1C shows cell binding activity of combination humanized variants (BG87P-25, BG87P-26, and BG87P-27) compared with anti-CLDN6 chimeric BG87P (chBG87P) against the HEK293T/human CLDN6. FIG. 1D shows cell binding activity of combination humanized variants (BG87P-21, BG87P-22, BG87P-23, and BG87P-24) compared with anti-CLDN6 chimeric BG87P (chBG87P) against the cancer cell line PA-1. FIG. 1E shows cell binding activity of combination humanized variants (BG87P-25, BG87P-26, and BG87P-27) compared with anti-CLDN6 chimeric BG87P (chBG87P) against the cancer cell line PA-1. FIG. 1F shows cell binding activity of post translational modifications (PTM) removal engineering variants (BG87P-m1, BG87P-m2, BG87P-m3, BG87P-m4, BG87P-m5, BG87P-m6, BG87P-m7, and BG87P-m8) compared to anti-CLDN6 chimeric BG87P (chBG87P) and BG87P-B20 against the HEK293T/human CLDN6. FIG. 1G shows cell binding activity of BG87P solubility engineering variants (BG87P-21, BG87P-34, and BG87P-33) compared to anti-CLDN6 chimeric BG87P (chBG87P) against the HEK293T/human CLDN6. FIG. 1H shows non-specific binding activity of solubility engineering variants (BG87P-21, BG87P-34, and BG87P-33) compared to anti-CLDN6 chimeric BG87P (chBG87P) against the HEK293T-human CLDN9. FIG. 1I shows cross-reactivity of humanized variants (BG87P-21, BG87P-34, and BG87P-33) compared to anti-CLDN6 chimeric BG87P (chBG87P) against CHOK1-cyno CLDN6. FIG. 1J shows cross-reactivity of humanized variants (BG87P-21, BG87P-34, and BG87P-33) compared to anti-CLDN6 chimeric BG87P (chBG87P) against CHOK1-mouse CLDN6.

FIG. 2 depicts the predicated a hydrophobic patch in a homology model of chimeric BG87P by Schroedinger. 197-Y98-Y100-V100a of HCDR3 are predicated to form an exposed hydrophobic patch, along with Y49-W50 of HCDR2 (Y49 is the last residue of FR2 of variable region of light chain while W50 is the first residue of LCDR2).

FIG. 3 shows the hydrophobicity of selected humanized BG87P variants after engineering (BG87-33, BG87-34, BG87P-21) as determined by HIC-HPLC.

FIGS. 4A and 4B show the comparison of binding activity between chimeric and humanized sp34s in Hut78 cells. FIG. 4A shows comparison of binding affinities between chimeric sp34 (ch-sp34) and humanized sp34 BG53P (BG53P) measured by melt flow index (MFI) in Hut78 cells. FIG. 4B shows the comparison of binding affinities among chimeric sp34 (ch-sp34), humanized sp34 BG53P (BG53P) and BG56P.

FIG. 5 shows the comparison of binding activity between humanized sp34 BG56P (BG56P) and humanized sp34 scFv BG561p (BG561P).

FIGS. 6A, 6B, and 6C show the comparison of binding affinities among humanized sp34 scFvs in Hut78 cells. FIG. 6A shows comparison of binding affinities between humanized sp34 scFv BG561p (BG561P) and humanized scFv BG562P (BG562P) in Hut78 cells. FIG. 6B shows comparison of binding affinities between humanized scFv BG562P (BG562P) and humanized scFv BG563P (BG563P) in Hut78 cells. FIG. 6C shows comparison of binding affinities between humanized scFv BG563P (BG563P) and humanized scFv BG564P (BG564P) in Hut78 cells.

FIG. 7 shows a schematic representation of CLDN6×CD3 BsAb BG143P.

FIGS. 8A and 8B show the target-binding activity of CLDN6×CD3 BsAb BG143P. FIG. 8A shows CD3 binding activity of BG143P in CD3-expressing Jurkat cells. FIG. 8B shows CLDN6 binding activity of BG143P in CLDN6-expressing PA-1 cells.

FIGS. 9A, 9B, and 9C shows on-target functional activity of CLDN6×CD3 BsAb BG143P in tumor cell lines with different CLDN6 expression. FIG. 9A shows redirected T cell cytotoxicity of BG143P in CLDN6-expressing PA-1 cells, Hutu80 cells, AGS cells, and NCI-H1299 cells by cell lysis assay. FIG. 9B shows IFN-γ induction activity of BG143P in CLDN6-expressing PA-1 cells, Hutu80 cells, AGS cells, and NCI-H1299 cells. FIG. 9C shows IL-2 induction activity of BG143P in CLDN6-expressing PA-1 cells, Hutu80 cells, AGS cells, and NCI-H1299 cells.

FIGS. 10A, 10B, and 10C show functional specificity of CLDN6×CD3 BsAb BG143P against human CLDN6 and CLDN9. FIG. 10A shows binding specificity of BG143P against human CLDN6 (left graph) and CLDN9 (right graph) in NCI-H1299 cells. FIG. 10B shows killing specificity (cell lysis activity) of BG143P against human CLDN6 (left graph) and CLDN9 (right graph) in NCI-H1299 cells. FIG. 10C shows cytokine (IFN-γ) induction of BG143P against human CLDN6 (left graph) and CLDN9 (right graph) in NCI-H1299 cells.

FIGS. 11A and 11B show the in vivo efficacy of CLDN6×CD3 BsAb BG143P in an OV-90 xenograft model in PBMC-humanized mice. FIG. 11A shows the tumor volume along with time. Mice were left untreated (no PBMC), treated with PBS (PBS i.p QW), treated with 0.01 mg/kg of BG143P (BG143P-0.01 mg/kg, i.p), treated with 0.03 mg/kg of BG143P (BG143P-0.01 mg/kg, i.p) or treated with 0.1 mg/kg of BG143P (BG143P-0.1 mg/kg, i.p). Treatment was once a week and represented on the X-axis by the triangles. FIG. 11B shows the percentage of hCD45+ cells in the peripheral blood of the mice left untreated (no PBMC), treated with PBS (PBS i.p QW), treated with 0.01 mg/kg of BG143P (BG143P-0.01 mg/kg, i.p), treated with 0.03 mg/kg of BG143P (BG143P-0.01 mg/kg, i.p) or treated with 0.1 mg/kg of BG143P (BG143P-0.1 mg/kg, i.p) at day 13, day 21, and day 27 post PBMC injection, indicating human PBMC reconstitution.

FIGS. 12A and 12B show the in vivo efficacy of CLDN6×CD3 BsAb BG143P in aB16F10/human CLDN6 syngeneic model in hCD3EDG-transgenic mice. FIG. 12A shows the tumor volume along with time. Mice were treated with PBS (PBS i.p QW), treated with 0.01 mg/kg of BG143P (BG143P-0.01 mg/kg, i.p), treated with 0.03 mg/kg of BG143P (BG143P-0.01 mg/kg, i.p) or treated with 0.1 mg/kg of BG143P (BG143P-0.1 mg/kg, i.p). Treatment was once a week and represented on the X-axis by the triangles. FIG. 12B shows the body weight of the mice treated with PBS (PBS i.p QW), treated with 0.01 mg/kg of BG143P (BG143P-0.01 mg/kg, i.p), treated with 0.03 mg/kg of BG143P (BG143P-0.01 mg/kg, i.p) or treated with 0.1 mg/kg of BG143P (BG143P-0.1 mg/kg, i.p) over the course of day 11 to 27 post inoculation, as an indicator of tolerability of the mice to the antibody.

### DEFINITIONS

Unless specifically defined elsewhere in this document, all other technical and scientific terms used herein have the meaning commonly understood by one of ordinary skill in the art.

As used herein, including the appended claims, the singular forms of words such as “a,” “an,” and “the,” include their corresponding plural references unless the context clearly dictates otherwise.

As used herein, the term “or” is used to mean, and is used interchangeably with, the term “and/or” unless the context clearly dictates otherwise. As also used herein, the term “and/or” refers to and encompasses any and all possible combinations of one or more of the associated listed items, as well as the lack of combinations when interpreted in the alternative (“or”).

As used herein, “about” when used with a numerical value means the numerical value stated as well as plus or minus 10% of the numerical value. For example, “about 10” should be understood as both “10” and “9-11.”

As used herein, a phrase in the form “A/B” or in the form “A and/or B” means (A), (B), or (A and B); a phrase in the form “at least one of A, B, and C” means (A), (B), (C), (A and B), (A and C), (B and C), or (A, B, and C).

The term “anti-cancer agent” as used herein refers to any agent that can be used to treat a cell proliferative disorder such as cancer, including but not limited to, cytotoxic agents, chemotherapeutic agents, radiotherapy and radiotherapeutic agents, targeted anti-cancer agents, and immunotherapeutic agents.

The term “claudin 6” or “CLDN6” refers to a member of the CLDN family. CLDN6 has a molecular weight of 23 kDa. CLDN6 has four transmembrane domains and a PDZ-binding region at the carboxyl end of the cytoplasm. The amino acid sequence of human CLDN6 can be found at UniPort ID P56747. An exemplary human CLDN6 sequence is SEQ ID NO: 87.

The term “claudin 9” or “CLDN9” refers to another member of the CLDN family. CLDN9 has a molecular weight of 23 kDa, and the amino acid sequence of which can be found at UniPort ID 095484. An exemplary human CLDN9 sequence is SEQ ID NO: 88.

The term “cluster of differentiation 3” or “CD3,” as used herein, refers to any native CD3 from any vertebrate source, including mammals such as primates (e.g. humans) and rodents (e.g., mice and rats), unless otherwise indicated, including, for example, CD3ε, CD3γ, CD3α, and CD3β chains. The term encompasses “full-length,” unprocessed CD3 (e.g., unprocessed or unmodified CD3ε or CD3γ), as well as any form of CD3 that results from processing in the cell. The term also encompasses naturally occurring variants of CD3, including, for example, splice variants or allelic variants. CD3 includes, for example, human CD3ε protein (NCBI RefSeq No. NP_000724), which is 207 amino acids in length, and human CD3γ protein (NCBI RefSeq No. NP_000064), which is 182 amino acids in length.

The terms “administration,” “administering,” “treating,” and “treatment” as used herein, when applied to an animal, human, experimental subject, cell, tissue, organ, or biological fluid, means contact of an exogenous pharmaceutical, therapeutic, diagnostic agent, or composition to the animal, human, subject, cell, tissue, organ, or biological fluid. Treatment of a cell encompasses contact of a reagent to the cell, as well as contact of a reagent to a fluid, where the fluid is in contact with the cell. The term “administration” and “treatment” also means in vitro and ex vivo treatments, e.g., of a cell, by a reagent, diagnostic, binding compound, or by another cell. The term “subject” herein includes any organism. Non-limiting examples include an animal. In any embodiment, the animal is a mammal (e.g., primate, higher primate, human, rat, mouse, dog, cat, rabbit). In any embodiment, the mammal isa human. In any embodiment, the subject is a patient comprising, or at risk of having, a disorder described herein. In any embodiment, treating any disease or disorder refers to ameliorating the disease or disorder (i.e., slowing or arresting or reducing the development of the disease or at least one of the clinical symptoms thereof). In another aspect, “treat,” “treating,” or “treatment” refers to alleviating or ameliorating at least one physical parameter including those which may not be discernible by the patient. In yet another aspect, “treat,” “treating,” or “treatment” refers to modulating the disease or disorder, either physically, (e.g., stabilization of a discernible symptom), physiologically, (e.g., stabilization of a physical parameter), or both. In yet another aspect, “treat,” “treating,” or “treatment” refers to preventing or delaying the onset or development or progression of the disease or disorder. In an aspect, the terms “prevent,” “preventing” or “prevention” as used herein with reference to a cancer refer to precluding or reducing the risk of developing cancer. Prevention may also refer to the prevention of recurrence or a secondary cancer once an initial cancer has been treated or cured.

The terms “individual,” “subject,” and “patient” are used interchangeably herein, and refer to any individual mammalian subject, e.g., bovine, canine, feline, equine, or human. In specific embodiments, the subject, individual, or patient is a human.

The term “affinity” as used herein refers to the strength of interaction between an antibody and antigen. Within the antigen, the variable regions of the antibody interacts through non-covalent forces with the antigen at numerous sites. In general, the more interactions, the stronger the affinity.

The term “antibody” as used herein refers to a polypeptide of the immunoglobulin family that can bind a corresponding antigen non-covalently, reversibly, and in a specific manner. For example, a naturally occurring IgG antibody is a tetramer comprising at least two heavy (H) chains and two light (L) chains inter-connected by disulfide bonds. Each heavy chain is comprised of a heavy chain variable region (abbreviated herein as VH) and a heavy chain constant region. The heavy chain constant region is comprised of three domains, CH1, CH2 and CH3. Each light chain is comprised of a light chain variable region (abbreviated herein as VL or Vκ) and a light chain constant region. The light chain constant region is comprised of one domain, CL. The VH and VL regions can be further subdivided into regions of hypervariability, termed complementarity determining regions (CDRs), interspersed with regions that are more conserved, termed framework regions (FR). Each VH and VL is composed of three CDRs and four framework regions (FRs) arranged from amino-terminus to carboxyl-terminus in the following order: FR1, CDR1, FR2, CDR2, FR3, CDR3, and FR4. The variable regions of the heavy and light chains contain a binding domain that interacts with an antigen. The constant regions of the antibodies can mediate the binding of the immunoglobulin to host tissues or factors, including various cells of the immune system (e.g., effector cells) and the first component (Clq) of the classical complement system.

The term “antibody” includes, but is not limited to, monoclonal antibodies, human antibodies, humanized antibodies, chimeric antibodies, and anti-idiotypic (anti-Id) antibodies. The antibodies can be of any isotype/class (e.g., IgG, IgE, IgM, IgD, IgA and IgY), or subclass (e.g., IgG1, IgG2, IgG3, IgG4, IgA1 and IgA2).

The term “chimeric” antibody refers to an antibody in which a portion of the heavy and/or light chain is derived from a particular source or species, while the remainder of the heavy and/or light chain is derived from a different source or species.

The terms “full-length antibody,” “intact antibody,” and “whole antibody” are used herein interchangeably to refer to an antibody having a structure substantially similar to a native antibody structure or having heavy chains that contain an Fc region.

In some embodiments, the anti-CLDN6 antibodies comprise at least one antigen-binding site, at least a variable region. In some embodiments, the anti-CLDN6 antibodies comprise an antigen-binding fragment from an CLDN6 antibody described herein. In some embodiments, the anti-CLDN6 antibody is isolated or recombinant.

In some embodiments, the anti-CD3 antibodies comprise at least one antigen-binding site, at least a variable region. In some embodiments, the anti-CD3 antibodies comprise an antigen-binding fragment from an CD3 antibody described herein. In some embodiments, the anti-CD3 antibody is isolated or recombinant.

The term “monoclonal antibody” or “mAb” or “Mab” herein means a population of substantially homogeneous antibodies, i.e., the antibody molecules comprised in the population are identical in amino acid sequence except for possible naturally occurring mutations that can be present in minor amounts. In contrast, conventional (polyclonal) antibody preparations typically include a multitude of different antibodies having different amino acid sequences in their variable domains, particularly their complementarity determining regions (CDRs), which are often specific for different epitopes. The modifier “monoclonal” indicates the character of the antibody as being obtained from a substantially homogeneous population of antibodies and is not to be construed as requiring production of the antibody by any particular method. Monoclonal antibodies (mAbs) can be obtained by methods known to those skilled in the art. See, for example Kohler et al., Nature 1975 256:495-497; U.S. Pat. No. 4,376,110; Ausubel et al., CURRENT PROTOCOLS IN MOLECULAR BIOLOGY 1992; Harlow et al., ANTIBODIES: A LABORATORY MANUAL, Cold spring Harbor Laboratory 1988; and Colligan et al., CURRENT PROTOCOLS IN IMMUNOLOGY 1993. The antibodies disclosed herein can be of any immunoglobulin class including IgG, IgM, IgD, IgE, IgA, and any subclass thereof such as IgG1, IgG2, IgG3, IgG4. A hybridoma producing a monoclonal antibody can be cultivated in vitro or in vivo. High titers of monoclonal antibodies can be obtained in in vivo production where cells from the individual hybridomas are injected intraperitoneally into mice, such as pristine-primed Balb/c mice to produce ascites fluid containing high concentrations of the desired antibodies. Monoclonal antibodies of isotype IgM or IgG can be purified from such ascites fluids, or from culture supernatants, using column chromatography methods well known to those of skill in the art.

In general, the basic antibody structural unit comprises a tetramer. Each tetramer includes two identical pairs of polypeptide chains, each pair having one “light chain” (about 25 kDa) and one “heavy chain” (about 50-70 kDa). The amino-terminal portion of each chain includes a variable region of about 100 to 110 or more amino acids primarily responsible for antigen recognition. The carboxy-terminal portion of the heavy chain can define a constant region primarily responsible for effector function. Typically, human light chains are classified as kappa and lambda light chains. Furthermore, human heavy chains are typically classified as α, δ, ε, γ, or μ, and define the antibody's isotypes as IgA, IgD, IgE, IgG, and IgM, respectively. Within light and heavy chains, the variable and constant regions are joined by a “J” region of about 12 or more amino acids, with the heavy chain also including a “D” region of about 10 more amino acids.

The variable regions of each light/heavy chain (VL/VH) pair form the antibody binding site. Thus, in general, an intact antibody has two binding sites. Except in bifunctional or bispecific antibodies, the two binding sites are, in general, the same in primary sequence.

Typically, the variable domains of both the heavy and light chains comprise three hypervariable regions, also called “complementarity determining regions” or “CDRs,” which are located between relatively conserved framework regions (FR). The CDRs are usually aligned by the framework regions, enabling binding to a specific epitope. In general, from N-terminal to C-terminal, both light and heavy chain variable domains comprise FR-1 (or FR1), CDR-1 (or CDR1), FR-2 (FR2), CDR-2 (CDR2), FR-3 (or FR3), CDR-3 (CDR3), and FR-4 (or FR4). The positions of the CDRs and framework regions can be determined using various well known definitions in the art, e.g., Kabat, Chothia, AbM and IMGT (see, e.g., Johnson et al., Nucleic Acids Res., 29:205-206 (2001); Chothia and Lesk, J. Mol. Biol., 196:901-917 (1987); Chothia et al., Nature, 342:877-883 (1989); Chothia et al., J. Mol. Biol., 227:799-817 (1992); Al-Lazikani et al., J. Mol. Biol., 273:927-748 (1997) ImMunoGenTics (IMGT) numbering (Lefranc, M.-P., The Immunologist, 7, 132-136 (1999); Lefranc, M.-P. et al., Dev. Comp. Immunol., 27, 55-77 (2003) (“IMGT” numbering scheme)). Definitions of antigen combining sites are also described in the following: Ruiz et al., Nucleic Acids Res., 28:219-221 (2000); and Lefranc, M. P., Nucleic Acids Res., 29:207-209 (2001); MacCallum et al., J. Mol. Biol., 262:732-745 (1996); and Martin et al., Proc. Natl. Acad. Sci. USA, 86:9268-9272 (1989); Martin et al., Methods Enzymol., 203:121-153 (1991); and Rees et al., In Sternberg M. J. E. (ed.), Protein Structure Prediction, Oxford University Press, Oxford, 141-172 (1996). For example, under Kabat, the CDR amino acid residues in the heavy chain variable domain (VH) are numbered 31-35 (HCDR1), 50-65 (HCDR2), and 95-102 (HCDR3); and the CDR amino acid residues in the light chain variable domain (VL) are numbered 24-34 (LCDR1), 50-56 (LCDR2), and 89-97 (LCDR3). Under Chothia the CDR amino acids in the VH are numbered 26-32 (HCDR1), 52-56 (HCDR2), and 95-102 (HCDR3); and the amino acid residues in VL are numbered 26-32 (LCDR1), 50-52 (LCDR2), and 91-96 (LCDR3). By combining the CDR definitions of both Kabat and Chothia, the CDRs consist of amino acid residues 26-35 (HCDR1), 50-65 (HCDR2), and 95-102 (HCDR3) in human VH and amino acid residues 24-34 (LCDR1), 50-56 (LCDR2), and 89-97 (LCDR3) in human VL. Under IMGT the CDR amino acid residues in the VH are numbered approximately 26-35 (HCDR1), 51-57 (HCDR2) and 93-102 (HCDR3), and the CDR amino acid residues in the VL are numbered approximately 27-32 (LCDR1), 50-52 (LCDR2), and 89-97 (LCDR3) (numbering according to Kabat). Under IMGT, the CDR regions of an antibody can be determined using the program IMGT/DomainGap Align.

The term “hypervariable region” means the amino acid residues of an antibody that are responsible for antigen-binding. The hypervariable region comprises amino acid residues from a “CDR” (e.g., LCDR1, LCDR2 and LCDR3 in the light chain variable domain and HCDR1, HCDR2 and HCDR3 in the heavy chain variable domain). Sec, Kabat et al., (1991) Sequences of Proteins of Immunological Interest, 5th Ed. Public Health Service, National Institutes of Health, Bethesda, Md. (defining the CDR regions of an antibody by sequence); see also Chothia and Lesk (1987) J. Mol. Biol. 196: 901-917 (defining the CDR regions of an antibody by structure). The term “framework” or “FR” residues means those variable domain residues other than the hypervariable region residues defined herein as CDR residues.

Unless otherwise indicated, an “antigen-binding fragment” means antigen-binding fragments of antibodies, i.e. antibody fragments that retain the ability to bind specifically to the antigen bound by the full-length antibody, e.g., fragments that retain one or more CDR regions. Examples of antigen-binding fragments include, but not limited to, Fab, Fab′, F(ab′)2, and Fv fragments; diabodies; linear antibodies; single-chain antibody molecules, e.g., single chain Fv (ScFv); nanobodies and multi-specific antibodies formed from antibody fragments.

As used herein, an antibody “specifically binds” to a target protein, meaning the antibody exhibits preferential binding to that target as compared to other proteins, but this specificity does not require absolute binding specificity. An antibody “specifically binds” or “selectively binds,” is used in the context of describing the interaction between an antigen (e.g., a protein) and an antibody, or antigen binding antibody fragment, refers to a binding reaction that is determinative of the presence of the antigen in a heterogeneous population of proteins and other biologics, for example, in a biological sample, blood, serum, plasma or tissue sample. Thus, under certain designated immunoassay conditions, the antibodies or antigen-binding fragments thereof specifically bind to a particular antigen at least two times when compared to the background level and do not specifically bind in a significant amount to other antigens present in the sample. In one aspect, under designated immunoassay conditions, the antibody or antigen-binding fragment thereof, specifically bind to a particular antigen at least ten (10) times when compared to the background level of binding and does not specifically bind in a significant amount to other antigens present in the sample.

“Antigen-binding domain” as used herein, comprise at least three CDRs and specifically bind to an epitope. An “antigen-binding domain” of a multi-specific antibody (e.g., a bispecific antibody) comprises a first antigen binding domain that specifically binds to a first epitope and a second antigen binding domain also comprised of at least three CDRs specifically binds to a second epitope. Multi-specific antibodies can be bispecific, trispecific, tetraspecific etc., with antigen binding domains directed to each specific epitope. Multi-specific antibodies can be multivalent (e.g., a bispecific tetravalent antibody) that comprises multiple antigen binding domains, for example, 2, 3, 4 or more antigen binding domains that specifically bind to a first epitope and 2, 3, 4 or more antigen binding domains that specifically bind a second epitope.

The term “human antibody” herein means an antibody that comprises human immunoglobulin protein sequences only. A human antibody can contain murine carbohydrate chains if produced in a mouse, in a mouse cell, or in a hybridoma derived from a mouse cell. Similarly, “mouse antibody” or “rat antibody” mean an antibody that comprises only mouse or rat immunoglobulin protein sequences, respectively.

The term “humanized” or “humanized antibody” means forms of antibodies that contain sequences from non-human (e.g., murine) antibodies as well as human antibodies. Such antibodies contain minimal sequence derived from non-human immunoglobulin. In general, the humanized antibody will comprise substantially all of at least one, and typically two, variable domains, in which all or substantially all of the hypervariable loops correspond to those of a non-human immunoglobulin and all or substantially all of the FR regions are those of a human immunoglobulin sequence. The humanized antibody optionally also will comprise at least a portion of an immunoglobulin constant region (Fc), typically that of a human immunoglobulin. The prefix “hum,” “hu,” “Hu,” or “h” is added to antibody clone designations when necessary to distinguish humanized antibodies from parental rodent antibodies. The humanized forms of rodent antibodies will generally comprise the same CDR sequences of the parental rodent antibodies, although certain amino acid substitutions can be included to increase affinity, increase stability of the humanized antibody, remove a post-translational modification or for other reasons.

The term “epitope” refers to the particular site on an antigen to which an antibody binds. The particular site on an antigen to which an antibody binds can be determined, for example, by crystallography. Methods such as hydroxyl radical protein footprinting and alanine scanning mutagenesis can also be used but may provide less resolution.

The term “monospecific antibody” refers to an antibody that specifically binds to only one antigen. A monospecific antibody can bind to only one epitope of an antigen or can bind to two or more epitopes of an antigen. A monospecific antibody that binds to two or more epitopes of an antigen is a monospecific polyepitopic antibody.

The term “multispecific antibody” or “multi-specific antibody” refers to an antibody that specifically binds two or more antigens (e.g., a bispecific antibody, a trispecific antibody, etc.). Non-limiting examples of multispecific antibodies include, but are not limited to, an antibody comprising a heavy chain variable domain (VH) and a light chain variable domain (VL), where the VH/VL unit has polyepitopic specificity, antibodies having two or more VL and VH domains with each VH/VL unit binding to a different epitope, antibodies having two or more single variable domains with each single variable domain binding to a different epitope, diabodies, triabodies, etc., as well as full-length antibodies and/or antibody fragments that have been linked covalently or non-covalently.

The terms “polyepitopic antibody” and “antibody having polyepitopic specificity” are used herein interchangeably to refer to an antibody that binds to two or more epitopes on the same or different antigen.

The term “Fc region” is used herein to define a C-terminal region of an immunoglobulin heavy chain, including native sequence Fc regions and variant Fc regions. Although the boundaries of the Fc region of an immunoglobulin heavy chain might vary, the human IgG heavy chain Fc region is usually defined to stretch from an amino acid residue at position Cys226, or from Pro230, to the carboxyl-terminus thereof. The C-terminal lysine (residue 447 according to the Eu numbering system) of the Fc region may be removed, for example, during production or purification of the antibody, or by recombinantly engineering the nucleic acid encoding a heavy chain of the antibody. Accordingly, a composition of intact antibodies may comprise antibody populations with all Lys447 residues removed, antibody populations with no Lys447 residues removed, and antibody populations having a mixture of antibodies with and without the Lys447 residue.

A “functional Fc region” possesses an effector function of a native sequence Fc region. Exemplary effector functions include Clq binding; complement dependent cytotoxicity (CDC); Fc receptor binding; antibody-dependent cell-mediated cytotoxicity (ADCC); phagocytosis; down regulation of cell surface receptors (e.g., B cell receptor; BCR), etc. Such effector functions generally require the Fc region to be combined with a binding domain (e.g., an antibody variable domain) and can be assessed using various assays disclosed herein or otherwise known in the art. A functional Fc region may possess effector function substantially similar to a wild-type IgG, reduced effector function compared to a wild-type IgG, or enhanced effector function compared to a wild-type IgG. For antibodies comprising a human Fc region, the comparison is typically to a wild-type human IgG1.

A “native sequence Fc region” comprises an amino acid sequence identical to the amino acid sequence of an Fc region found in nature. Native sequence human Fc regions include a native sequence human IgG1 Fc region (non-A and A allotypes); native sequence human lgG2 Fc region; native sequence human lgG3 Fc region; and native sequence human lgG4 Fc region as well as naturally occurring variants thereof.

A “variant Fc region” comprises an amino acid sequence which differs from that of a native sequence Fc region by virtue of at least one amino acid modification (e.g., from about one to about ten amino acid modifications, and in some embodiments from about one to about five amino acid modifications), preferably one or more amino acid substitution(s). The variant Fc region herein will preferably possess at least about 80% homology with a native sequence Fc region and/or with an Fc region of a parent polypeptide, preferably at least about 90% homology therewith, or preferably at least about 95% homology therewith. In some embodiments, variant Fc regions may possess reduced or enhanced effector function, as compared to a wild-type IgG. For antibodies comprising a human Fc region, the comparison is typically to a wild-type human IgG1.

The term “Fc component” as used herein refers to a hinge region, a CH2 domain or a CH3 domain of an Fc region.

The term “Hinge region” is generally defined as stretching from about residue 216 to 230 of an IgG (Eu numbering), from about residue 226 to 243 of an IgG (Kabat numbering), or from about residue 1 to 15 of an IgG (IMGT unique numbering).

The term “antibody fragment” refers to a molecule other than an intact antibody that comprises a portion of an intact antibody that binds the antigen to which the intact antibody binds. Examples of antigen-binding fragment include, without limitation, a diabody, a Fab, a Fab′, a F(ab′)₂, a F(ab)_{c}, an Fv fragment, a disulfide stabilized Fv fragment (dsFv), a (dsFv)₂, a bispecific dsFv (dsFv-dsFv′), a disulfide stabilized diabody (ds diabody), a triabody, a tetrabody, a single-chain antibody, an scFv, an scFv dimer, a single domain antibody, a single-domain antibody, and a multivalent domain antibody. Typically, binding fragments compete with the intact antibody from which they were derived for specific binding. Binding fragments can be produced by recombinant DNA techniques, or by enzymatic or chemical separation of intact immunoglobulins.

The term “Fab” refers to that portion of an antibody consisting of a single light chain (both variable and constant regions) bound to the variable region and first constant region of a single heavy chain by a disulfide bond.

The term “Fab” refers to a Fab fragment that includes a portion of the hinge region.

The term “F(ab′)₂” refers to a dimer of Fab′. F(ab′)₂ antibody fragments originally were produced as pairs of Fab′ fragments which have hinge cysteines between them. Other chemical couplings of antibody fragments are also known.

The term “Fv” refers to the smallest fragment of an antibody to bear the complete antigen binding site. An Fv fragment consists of the variable region of a single light chain bound to the variable region of a single heavy chain.

The term “single-chain antibody” refers to an antibody consisting of a heavy chain variable region and a light chain variable region connected by a linker. In most instances, but not all, the linker may be a peptide. The length of the linker varies depending upon the type of single-chain antibody. Covalently or non-covalently linking two or more single-chain antibodies together results in higher order forms. Single-chain antibodies, and their higher order forms, may include, but are not limited to, single-domain antibodies, multivalent domain antibodies, single chain variant fragments (scFvs), divalent scFvs (di-scFvs), trivalent scFvs (tri-scFvs), tetravalent scFvs (tetra-scFvs), diabodies, and triabodies and tetrabodies.

The terms “single-chain Fv antibody” and “scFv” are used herein interchangeably to refer to a single-chain antibody consisting of heavy variable region and a light chain variable region connected by a linker. In most instances, but not all, the linker may be a peptide. The linker peptide is preferably from about 5 to 30 amino acids in length, or from about 10 to 25 amino acids in length. Typically, the linker allows for stabilization of the variable domains without interfering with the proper folding and creation of an active binding site. In preferred embodiments, a linker peptide is rich in glycine, as well as serine or threonine. Covalently or non-covalently linking two or more scFvs together results in higher order forms di-scFvs, tri-scFvs, tetra-scFvs, etc. The antigen-binding sites of each scFv in a higher order form can target the same or different antigen or epitope.

The term “single-chain Fv-Fc antibody” or “scFv-Fc” refers to a full-length antibody consisting of a scFv connected to an Fc region.

A “diabody” is a higher order variant of a single-chain antibody consisting of two single-chain antibodies. For each single-chain antibody, a linker is used that is too short to allow pairing between the two domains on the same chain, forcing the domains to pair with the complementary domains of another chain, thereby creating two antigen-binding sites. In most instances, but not all, the linker may be a peptide. The antigen-binding sites can target the same or different antigens or epitopes. Triabodies (three single chain antibodies assembled to form three antigen-binding sites), tetrabodies (four single chain antibodies assembled to form four antigen-binding sites), and higher order variants can similarly be produced. See, for example, Holliger P. et al., Proc Natl Acad Sci USA. July 15; 90(14):6444-8 (1993); EP404097; WO93/11161.

A “single-domain antibody” refers to an antibody fragment containing only the variable region of a heavy chain or the variable region of a light chain. In certain instances, two or more VH domains are covalently joined with a peptide linker to create a multivalent domain antibody. The two or more VH domains of a multivalent domain antibody can target the same or different antigens or epitopes.

The term “heavy chain antibody” refers to an antibody that consists of two heavy chains. A heavy chain antibody may be an IgG-like antibody from camels, llamas, alpacas, sharks, etc., or an IgNAR from a cartilaginous fish. See, for example, Riechmann L. and Muyldermans S., J Immunol Methods. December 10; 231 (1-2): 25-38 (1999); Muyldermans S., J Biotechnol. June; 74(4):277-302 (2001); WO94/04678; WO94/25591; or U.S. Pat. No. 6,005,079. Heavy chain antibodies were originally derived from Camelidae (camels, dromedaries, and llamas). Although devoid of light chains, camelized antibodies have an authentic antigen-binding repertoire (Hamers-Casterman C. et al., Nature. June 3; 363(6428):446-8 (1993); Nguyen V. K. et al. “Heavy-chain antibodies in Camelidae; a case of evolutionary innovation,” Immunogenetics. April; 54(1):39-47 (2002); Nguyen V. K. et al. Immunology. May; 109(1):93-101 (2003)). The variable domain of a heavy chain antibody (VHH domain) represents the smallest known antigen-binding unit generated by adaptive immune responses (Koch-Nolte F. et al., FASEB J. November; 21(13):3490-8. Epub 2007 Jun. 15 (2007)).

The term “corresponding human germline sequence” refers to the nucleic acid sequence encoding a human variable region amino acid sequence or subsequence that shares the highest determined amino acid sequence identity with a reference variable region amino acid sequence or subsequence in comparison to all other known variable region amino acid sequences encoded by human germline immunoglobulin variable region sequences. The corresponding human germline sequence can also refer to the human variable region amino acid sequence or subsequence with the highest amino acid sequence identity with a reference variable region amino acid sequence or subsequence in comparison to all other evaluated variable region amino acid sequences. The corresponding human germline sequence can be framework regions only, complementarity determining regions only, framework and complementary determining regions, a variable segment (as defined above), or other combinations of sequences or subsequences that comprise a variable region. Sequence identity can be determined using the methods described herein, for example, aligning two sequences using BLAST, ALIGN, or another alignment algorithm known in the art. The corresponding human germline nucleic acid or amino acid sequence can have at least about 90%, 91, 92%, 93%, 94%, 95%, 96%, 97%, 98%, 99%, or 100% sequence identity with the reference variable region nucleic acid or amino acid sequence. In addition, if the antibody contains a constant region, the constant region also is derived from such human sequences, e.g., human germline sequences, or mutated versions of human germline sequences or antibody containing consensus framework sequences derived from human framework sequences analysis, for example, as described in Knappik et al., J. Mol. Biol. 296:57-86, 2000.

The term “equilibrium dissociation constant (KD, M)” refers to the dissociation rate constant (kd, time-1) divided by the association rate constant (ka, time-1, M-1). Equilibrium dissociation constants can be measured using any known method in the art. The antibodies of the present disclosure generally will have an equilibrium dissociation constant of less than about 10-7 or 10-8 M, for example, less than about 10-9 M or 10-10 M, in some aspects, less than about 10-11 M, 10-12 M or 10-13 M.

The terms “cancer” or “tumor” herein has the broadest meaning as understood in the art and refers to the physiological condition in mammals that is typically characterized by unregulated cell growth. In the context of the present disclosure, the cancer is not limited to certain type or location.

In the context of the present disclosure, when reference is made to an amino acid sequence, the term “conservative substitution” means substitution of the original amino acid by a new amino acid that does not substantially alter the chemical, physical and/or functional properties of the antibody or fragment, e.g., its binding affinity to CLDN6 or to CD3. Specifically, common conservative substations of amino acids are well known in the art.

The term “knob-into-hole” technology as used herein refers to amino acids that direct the pairing of two polypeptides together either in vitro or in vivo by introducing a spatial protuberance (knob) into one polypeptide and a socket or cavity (hole) into the other polypeptide at an interface in which they interact. For example, knob-into-holes have been introduced in the Fc:Fc binding interfaces, CL:CHI interfaces or VH/VL interfaces of antibodies (see, e.g., US 2011/0287009, US2007/0178552, WO 96/027011, WO 98/050431, and Zhu et al, 1997, Protein Science 6:781-788). In some embodiments, knob-into-holes insure the correct pairing of two different heavy chains together during the manufacture of multi-specific antibodies. For example, multi-specific antibodies having knob-into-hole amino acids in their Fc regions can further comprise single variable domains linked to each Fc region, or further comprise different heavy chain variable domains that pair with similar or different light chain variable domains. Knob-into-hole technology can also be used in the VH or VL regions to also insure correct pairing.

The term “knob” as used herein in the context of “knob-into-hole” technology refers to an amino acid change that introduces a protuberance (knob) into a polypeptide at an interface in which the polypeptide interacts with another polypeptide. In some embodiments, the other polypeptide has a hole mutation.

The term “hole” as used herein in the context of “knob-into-hole” refers to an amino acid change that introduces a socket or cavity (hole) into a polypeptide at an interface in which the polypeptide interacts with another polypeptide. In some embodiments, the other polypeptide has a knob mutation.

Examples of algorithms that are suitable for determining percent sequence identity and sequence similarity are the BLAST algorithms, which are described in Altschul et al, Nuc. Acids Res. 25:3389-3402, 1977; and Altschul et al., J. Mol. Biol. 215:403-410, 1990, respectively. Software for performing BLAST analyses is publicly available through the National Center for Biotechnology Information. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold. These initial neighborhood word hits act as values for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a word length (W) of 11, an expectation (E) or 10, M=5, N=−4 and a comparison of both strands. For amino acid sequences, the BLAST program uses as defaults a word length of 3, and expectation (E) of 10, and the BLOSUM62 scoring matrix (see Henikoff and Henikoff, (1989) Proc. Natl. Acad. Sci. USA 89: 10915) alignments (B) of 50, expectation (E) of 10, M=5, N=−4, and a comparison of both strands.

The BLAST algorithm also performs a statistical analysis of the similarity between two sequences (see, e.g., Karlin and Altschul, Proc. Natl. Acad. Sci. USA 90:5873-5787, 1993). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01, and most preferably less than about 0.001.

The percent identity between two amino acid sequences can also be determined using the algorithm of E. Meyers and W. Miller, Comput. Appl. Biosci. 4: 11-17, (1988), which has been incorporated into the ALIGN program (version 2.0), using a PAM120 weight residue table, a gap length penalty of 12 and a gap penalty of 4. In addition, the percent identity between two amino acid sequences can be determined using the Needleman and Wunsch, J. Mol. Biol. 48:444-453, (1970), algorithm which has been incorporated into the GAP program in the GCG software package using either a BLOSUM62 matrix or a PAM250 matrix, and a gap weight of 16, 14, 12, 10, 8, 6, or 4 and a length weight of 1, 2, 3, 4, 5, or 6.

The term “nucleic acid” is used herein interchangeably with the term “polynucleotide” and refers to deoxyribonucleotides or ribonucleotides and polymers thereof in either single- or double-stranded form. The term encompasses nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, without limitation, phosphorothioates, phosphoramidates, methyl phosphonates, chiral-methyl phosphonates, 2-O-methyl ribonucleotides, peptide-nucleic acids (PNAs).

The term “operably linked” in the context of nucleic acids refers to a functional relationship between two or more polynucleotide (e.g., DNA) segments. Typically, it refers to the functional relationship of a transcriptional regulatory sequence to a transcribed sequence. For example, a promoter or enhancer sequence is operably linked to a coding sequence if it stimulates or modulates the transcription of the coding sequence in an appropriate host cell or other expression system. Generally, promoter transcriptional regulatory sequences that are operably linked to a transcribed sequence are physically contiguous to the transcribed sequence, i.e., they are cis-acting. However, some transcriptional regulatory sequences, such as enhancers, need not be physically contiguous or located in close proximity to the coding sequences whose transcription they enhance.

In some aspects, the present disclosure provides compositions, e.g., pharmaceutically acceptable compositions, which include anti-CLDN6×CD3 multi-specific antibodies as described herein, formulated together with at least one pharmaceutically acceptable excipient. As used herein, the term “pharmaceutically acceptable excipient” includes any and all solvents, dispersion media, isotonic and absorption delaying agents, and the like that are physiologically compatible. The excipient can be suitable for intravenous, intramuscular, subcutaneous, parenteral, rectal, spinal or epidermal administration (e.g. by injection or infusion).

The compositions disclosed herein can be in a variety of forms. These include, for example, liquid, semi-solid and solid dosage forms, such as liquid solutions (e.g., injectable and infusion solutions), dispersions or suspensions, liposomes, and suppositories. A suitable form depends on the intended mode of administration and therapeutic application. Typical suitable compositions are in the form of injectable or infusion solutions. One suitable mode of administration is parenteral (e.g., intravenous, subcutaneous, intraperitoneal, intramuscular). In some embodiments, the antibody is administered by intravenous infusion or injection. In certain embodiments, the antibody is administered by intramuscular or subcutaneous injection.

The term “therapeutically effective amount” as herein used, refers to the amount of an antibody that, when administered to a subject for treating a disease, or at least one of the clinical symptoms of a disease or disorder, is sufficient to effect such treatment for the disease, disorder, or symptom. The “therapeutically effective amount” can vary with the antibody, the disease, disorder, and/or symptoms of the disease or disorder, severity of the disease, disorder, and/or symptoms of the disease or disorder, the age of the subject to be treated, and/or the weight of the subject to be treated. An appropriate amount in any given instance can be apparent to those skilled in the art or can be determined by routine experiments. In the case of combination therapy, the “therapeutically effective amount” refers to the total amount of the combination objects for the effective treatment of a disease, a disorder or a condition.

The term “combination therapy” refers to the administration of two or more therapeutic agents to treat a therapeutic condition or disorder described in the present disclosure. Such administration encompasses co-administration of these therapeutic agents in a substantially simultaneous manner. Such administration also encompasses co-administration in multiple, or in separate containers (e.g., capsules, powders, and liquids) for each active ingredient. Powders and/or liquids can be reconstituted or diluted to a desired dose prior to administration. In addition, such administration also encompasses use of each type of therapeutic agent in a sequential manner, either at approximately the same time or at different times. In either case, the treatment regimen will provide beneficial effects of the drug combination in treating the conditions or disorders described herein.

As used herein, the phrase “in combination with” means that an anti-CLDN6×CD3 multi-specific antibody is administered to the subject at the same time as, just before, or just after administration of an additional therapeutic agent. In certain embodiments, an anti-CLDN6×CD3 multi-specific antibody is administered as a co-formulation with an additional therapeutic agent.

### DETAILED DESCRIPTION

The present disclosure provides for antibodies, antigen-binding fragments, and anti-CLDN6×CD3 multi-specific antibodies. Furthermore, the present disclosure provides antibodies that have desirable pharmacokinetic characteristics and other desirable attributes, and thus can be used for reducing the likelihood of cancer or used for treating cancer. The present disclosure further provides pharmaceutical compositions comprising the antibodies and methods of making and using such pharmaceutical compositions for the prevention and treatment of cancer and associated disorders.

### Anti-CLDN6 Antibodies

The present disclosure provides for antibodies or antigen-binding fragments thereof that specifically bind to CLDN6. Antibodies or antigen-binding fragments of the present disclosure include, but are not limited to, the antibodies or antigen-binding fragments thereof, generated as described, below.

The present disclosure provides antibodies or antigen-binding fragments that specifically bind to CLDN6, wherein said antibodies or antibody fragments (e.g., antigen-binding fragments) comprise a VH domain having an amino acid sequences listed in Table 1. The present disclosure also provides antibodies or antigen-binding fragments that specifically bind CLDN6, wherein said antibodies or antigen-binding fragments comprise a HCDR having an amino acid sequence of any one of the HCDRs listed in Table 1. In one aspect, the present disclosure provides antibodies or antigen-binding fragments that specifically bind to CLDN6, wherein said antibodies comprise (or alternatively, consist of) one, two, three, or more HCDRs having an amino acid sequence of any of the HCDRs listed in Table 1.

The present disclosure provides for antibodies or antigen-binding fragments that specifically bind to CLDN6, wherein said antibodies or antigen-binding fragments comprise a VL domain having an amino acid sequences listed in Table 1. The present disclosure also provides antibodies or antigen-binding fragments that specifically bind to CLDN6, wherein said antibodies or antigen-binding fragments comprise a LCDR having an amino acid sequence of any one of the LCDRs listed in Table 1. In particular, the disclosure provides for antibodies or antigen-binding fragments that specifically bind to CLDN6, said antibodies or antigen-binding fragments comprise (or alternatively, consist of) one, two, three or more LCDRs having an amino acid sequence of any of the LCDRs listed in Table 1.

Other antibodies or antigen-binding fragments thereof of the present disclosure include amino acids that have been changed, yet have at least 60%, 70%, 80%, 90%, 95% or 99% identity in the CDR regions with the CDR regions disclosed in Table 1. In some embodiments, the amino acid sequence has at least 90, 91, 92, 93, 94, 95, 96, 97, 98 or 99% sequence identity. In some aspects, it includes amino acid changes wherein no more than 1, 2, 3, 4 or 5 amino acids have been changed in the CDR regions when compared with the CDR regions depicted in the sequence described in Table 1.

Other antibodies of the present disclosure include those where the amino acids or nucleic acids encoding the amino acids have been changed; yet have at least 60%, 70%, 80%, 90%, 95% or 99% identity to the sequences described in Table 1. In some embodiments, the amino acid sequence has at least 90, 91, 92, 93, 94, 95, 96, 97, 98 or 99% sequence identity. In some aspects, it includes changes in the amino acid sequences wherein no more than 1, 2, 3, 4 or 5 amino acids have been changed in the variable regions when compared with the variable regions depicted in the sequence described in Table 1, while retaining substantially the same therapeutic activity.

The present disclosure also provides nucleic acid sequences that encode VH, VL, the full length heavy chain, and the full length light chain of the antibodies that specifically bind to CLDN6. Such nucleic acid sequences can be optimized for expression in mammalian cells.

The present disclosure provides antibodies and antigen-binding fragments thereof that bind to an epitope of human CLDN6. In certain aspects the antibodies and antigen-binding fragments can bind to the same epitope of CLDN6.

The present disclosure also provides for antibodies and antigen-binding fragments thereof that bind to the same epitope as do the anti-CLDN6 antibodies described in Table 1. Additional antibodies and antigen-binding fragments thereof can therefore be identified based on their ability to cross-compete (e.g., to competitively inhibit the binding of, in a statistically significant manner) with other antibodies in binding assays. The ability of a test antibody to inhibit the binding of antibodies and antigen-binding fragments thereof of the present disclosure to CLDN6 demonstrates that the test antibody can compete with that antibody or antigen-binding fragments thereof for binding to CLDN6. Such an antibody can, without being bound to any one theory, bind to the same or a related (e.g., a structurally similar or spatially proximal) epitope on CLDN6 as the antibody or antigen-binding fragments thereof with which it competes. In a certain aspect, the antibody that binds to the same epitope on CLDN6 as the antibodies or antigen-binding fragments thereof of the present disclosure is a human or humanized monoclonal antibody. Such human or humanized monoclonal antibodies can be prepared and isolated as described herein.

### Anti-CD3 Antibodies

The present disclosure provides for antibodies or antigen-binding fragments thereof that specifically bind to CD3. Antibodies or antigen-binding fragments of the present disclosure include, but are not limited to, the antibodies or antigen-binding fragments thereof, generated as described, below.

The present disclosure provides antibodies or antigen-binding fragments that specifically bind to CD3, wherein said antibodies or antibody fragments (e.g., antigen-binding fragments) comprise a VH domain having an amino acid sequence listed in Table 2. The present disclosure also provides antibodies or antigen-binding fragments that specifically bind CD3, wherein said antibodies or antigen-binding fragments comprise a HCDR having an amino acid sequence of any one of the HCDRs listed in Table 2. In one aspect, the present disclosure provides antibodies or antigen-binding fragments that specifically bind to CD3, wherein said antibodies comprise (or alternatively, consist of) one, two, three, or more HCDRs having an amino acid sequence of any of the HCDRs listed in Table 2.

Other antibodies or antigen-binding fragments thereof of the present disclosure include amino acids that have been changed, yet have at least 60%, 70%, 80%, 90%, 95% or 99% identity in the CDR regions with the CDR regions disclosed in Table 2. In some embodiments, the amino acid sequence has at least 90, 91, 92, 93, 94, 95, 96, 97, 98 or 99% sequence identity. In some aspects, it includes amino acid changes wherein no more than 1, 2, 3, 4 or 5 amino acids have been changed in the CDR regions when compared with the CDR regions depicted in the sequence described in Table 2.

Other antibodies of the present disclosure include those where the amino acids or nucleic acids encoding the amino acids have been changed; yet have at least 60%, 70%, 80%, 90%, 95% or 99% identity to the sequences described in Table 2. In some embodiments, the amino acid sequence has at least 90, 91, 92, 93, 94, 95, 96, 97, 98 or 99% sequence identity. In some aspects, it includes changes in the amino acid sequences wherein no more than 1, 2, 3, 4 or 5 amino acids have been changed in the variable regions when compared with the variable regions depicted in the sequence described in Table 2, while retaining substantially the same therapeutic activity.

The present disclosure also provides nucleic acid sequences that encode VH, VL, the full length heavy chain, and the full length light chain of the antibodies that specifically bind to CD3. Such nucleic acid sequences can be optimized for expression in mammalian cells.

The present disclosure provides antibodies and antigen-binding fragments thereof that bind to an epitope of human CD3. In certain aspects the antibodies and antigen-binding fragments can bind to the same epitope of CD3.

The present disclosure also provides for antibodies and antigen-binding fragments thereof that bind to the same epitope as do the anti-CD3 antibodies described in Table 2. Additional antibodies and antigen-binding fragments thereof can therefore be identified based on their ability to cross-compete (e.g., to competitively inhibit the binding of, in a statistically significant manner) with other antibodies in binding assays. The ability of a test antibody to inhibit the binding of antibodies and antigen-binding fragments thereof of the present disclosure to CD3 demonstrates that the test antibody can compete with that antibody or antigen-binding fragments thereof for binding to CD3. Such an antibody can, without being bound to any one theory, bind to the same or a related (e.g., a structurally similar or spatially proximal) epitope on CD3 as the antibody or antigen-binding fragments thereof with which it competes. In a certain aspect, the antibody that binds to the same epitope on CD3 as the antibodies or antigen-binding fragments thereof of the present disclosure is a human or humanized monoclonal antibody. Such human or humanized monoclonal antibodies can be prepared and isolated as described herein.

### Anti-CLDN6×CD3 Multi-Specific Antibodies

In one embodiment, the anti-CLDN6 and anti-CD3 antibodies as disclosed herein can be incorporated into an anti-CLDN6×CD3 multi-specific antibody. An antibody molecule is a multi-specific antibody molecule, for example, it comprises a number of antigen binding domains, wherein at least one antigen binding domain sequence specifically binds CLDN6 as a first epitope and a second antigen binding domain sequence specifically binds CD3 as a second epitope. In one embodiment, the multi-specific antibody comprises a third, fourth or fifth antigen binding domain. In one embodiment, the multi-specific antibody is a bispecific antibody, a tri-specific antibody, or tetra-specific antibody. In each example, the multi-specific antibody comprises at least one anti-CLDN6 antigen binding domain and at least one anti-CD3 antigen binding domain.

In one embodiment, the multi-specific antibody is a bispecific antibody. As used herein, a bispecific antibody specifically binds only two antigens. The bispecific antibody comprises a first antigen binding domain which specifically binds CLDN6 and a second antigen binding domain that specifically binds CD3. This includes a bispecific antibody comprising a heavy chain variable domain and a light chain variable domain which specifically bind CLDN6 as a first epitope and a heavy chain variable domain which specifically bind CD3 as a second epitope. In another embodiment, the bispecific antibody comprises an antigen binding fragment of an antibody that specifically binds CLDN6 and an antigen binding fragment that specially binds CD3. The bispecific antibody that comprises antigen binding fragments, the antigen-binding fragment can be a Fab, F(ab′)2, Fv, or a single chain Fv (ScFv) or a scFv.

Previous experimentation (Coloma and Morrison Nature Biotech. 15: 159-163 (1997)) described a tetravalent bispecific antibody which was engineered by fusing DNA encoding a single chain anti-dansyl antibody Fv (scFv) after the C terminus (CH3-scFv) or after the hinge (hinge-scFv) of an IgG3 anti-dansyl antibody. The present disclosure provides multivalent antibodies (e.g. tetravalent antibodies) with at least two antigen binding domains, which can be readily produced by recombinant expression of nucleic acid encoding the polypeptide chains of the antibody. The multivalent antibody herein comprises three to eight, but preferably four, antigen binding domains, which specifically bind at least two antigens.

### Linkers

It is also understood that the domains and/or regions of the polypeptide chains of the bispecific tetravalent antibody can be separated by linker regions of various lengths. In some embodiments, the antigen binding domains are separated from each other, a CL, CH1, hinge, CH2, CH3, or the entire Fc region by a linker region. For example, VL1-CL-(linker) VH2-CH1, VH-linker-VL. Such linker region may comprise a random assortment of amino acids, or a restricted set of amino acids. Such linker regions can be flexible or rigid (see US2009/0155275).

Multi-specific antibodies have been constructed by genetically fusing two single chain Fv (scFv) or Fab fragments with or without the use of flexible linkers (Mallender et al., J. Biol. Chem. 1994 269:199-206; Macket al., Proc. Natl. Acad. Sci. USA. 1995 92:7021-5; Zapata et al., Protein Eng. 1995 8.1057-62), via a dimerization device such as leucine Zipper (Kostelny et al., J. Immunol. 1992148:1547-53; de Kruifetal J. Biol. Chem. 1996 271:7630-4) and Ig C/CH1 domains (Muller et al., FEBS Lett. 422:259-64); by diabody (Holliger et al., (1993) Proc. Nat. Acad. Sci. USA. 1998 90:6444-8; Zhu et al., Bio/Technology (NY) 1996 14:192-6); Fab-scFv fusion (Schoonjans et al., J. Immunol. 2000 165:7050-7); and mini antibody formats (Packet al., Biochemistry 1992.31:1579-84; Packet al., Bio/Technology 1993 11:1271-7).

The bispecific tetravalent antibodies as disclosed herein comprise a linker region of at least 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, or more amino acid residues between one or more of its antigen binding domains, CL domains, CH1 domains, Hinge region, CH2 domains, CH3 domains, or Fc regions. In some embodiments, the amino acids glycine and serine comprise the amino acids within the linker region. In another embodiment, the linker can be GS, GGS, GSG, SGG, GGG, GGGS, SGGG, GGGGS, GGGGSGS, GGGGSGS, GGGGSGGS, GGGGSGGGGS, GGGGSGGGGSGGGGS, AKTTPKLEEGEFSEAR, AKTTPKLEEGEFSEARV, AKTTPKLGG, SAKTTPKLGG, AKTTPKLEEGEFSEARV, SAKTTP, SAKTTPKLGG, RADAAP, RADAAPTVS, RADAAAAGGPGS, RADAAAA(G4S)4, SAKTTP, SAKTTPKLGG, SAKTTPKLEEGEFSEARV, ADAAP, ADAAPTVSIFPP, TVAAP, TVAAPSVFIFPP, QPKAAP, QPKAAPSVTLFPP, AKTTPP, AKTTPPSVTPLAP, AKTTAP, AKTTAPSVYPLAP, ASTKGP, ASTKGPSVFPLAP, GENKVEYAPALMALS, GPAKELTPLKEAKVS, and GHEAAAVMQVQYPAS or any combination thereof (see WO2007/024715).

### Dimerization Specific Amino Acids

In one embodiment, the multivalent antibody comprises at least one dimerization specific amino acid change. The dimerization specific amino acid changes result in “knobs into holes” interactions, and increases the assembly of correct multivalent antibodies. The dimerization specific amino acids can be within the CH1 domain or the CL domain or combinations thereof. The dimerization specific amino acids used to pair CH1 domains with other CH1 domains (CH1-CH1) and CL domains with other CL domains (CL-CL) and can be found at least in the disclosures of WO2014082179, WO2015181805 family and WO2017059551. The dimerization specific amino acids can also be within the Fc domain and can be in combination with dimerization specific amino acids within the CH1 or CL domains. In one embodiment, the disclosure provides a bispecific antibody comprising at least one dimerization specific amino acid pair.

### Further Alteration of the Framework of Fc Region

In yet other aspects, the Fc region is altered by replacing at least one amino acid residue with a different amino acid residue to alter the effector functions of the antibody. For example, one or more amino acids can be replaced with a different amino acid residue such that the antibody has an altered affinity for an effector ligand but retains the antigen-binding ability of the parent antibody. The effector ligand to which affinity is altered can be, for example, an Fc receptor or the Cl component of complement. This approach is described in, e.g., U.S. Pat. Nos. 5,624,821 and 5,648,260, both by Winter et al.

In another aspect, one or more amino acid residues can be replaced with one or more different amino acid residues such that the antibody has altered Clq binding and/or reduced or abolished complement dependent cytotoxicity (CDC). This approach is described in, e.g., U.S. Pat. No. 6,194,551 by Idusogie et al.

In yet another aspect, one or more amino acid residues are changed to thereby alter the ability of the antibody to fix complement. This approach is described in, e.g., the publication WO 94/29351 by Bodmer et al. In a specific aspect, one or more amino acids of an antibody or antigen-binding fragment thereof of the present disclosure are replaced by one or more allotypic amino acid residues, for the IgG1 subclass and the kappa isotype. Allotypic amino acid residues also include, but are not limited to, the constant region of the heavy chain of the IgG1, IgG2, and IgG3 subclasses as well as the constant region of the light chain of the kappa isotype as described by Jefferis et al., MAbs. 1:332-338 (2009).

In another aspect, the Fc region is modified to increase the ability of the antibody to mediate antibody dependent cellular cytotoxicity (ADCC) and/or to increase the affinity of the antibody for an Fcγ receptor by modifying one or more amino acids. This approach is described in, e.g., the publication WO00/42072 by Presta. Moreover, the binding sites on human IgG1 for FcγRI, FcγRII, FcγRIII and FcRn have been mapped and variants with improved binding have been described (see Shields et al., J. Biol. Chem. 276:6591-6604, 2001).

In still another aspect, the glycosylation of the multi-specific antibody is modified. For example, an aglycosylated antibody can be made (i.e., the antibody lacks or has reduced glycosylation). Glycosylation can be altered to, for example, increase the affinity of the antibody for “antigen.” Such carbohydrate modifications can be accomplished by, for example, altering one or more sites of glycosylation within the antibody sequence. For example, one or more amino acid substitutions can be made that result in elimination of one or more variable region framework glycosylation sites to thereby eliminate glycosylation at that site. Such aglycosylation can increase the affinity of the antibody for antigen. Such an approach is described in, e.g., U.S. Pat. Nos. 5,714,350 and 6,350,861 by Co et al.

Additionally, or alternatively, an antibody can be made that has an altered type of glycosylation, such as a hypofucosylated antibody having reduced amounts of fucosyl residues or an antibody having increased bisecting GlcNac structures. Such altered glycosylation patterns have been demonstrated to increase the ADCC ability of antibodies. Such carbohydrate modifications can be accomplished by, for example, expressing the antibody in a host cell with an altered glycosylation pathway. Cells with altered glycosylation pathways have been described in the art and can be used as host cells in which to express recombinant antibodies to thereby produce an antibody with altered glycosylation. For example, EP 1,176,195 by Hang et al., describes a cell line with a functionally disrupted FUT8 gene, which encodes a fucosyl transferase, such that antibodies expressed in such a cell line exhibit hypofucosylation. Publication WO 03/035835 by Presta describes a variant CHO cell line, Lecl3 cells, with reduced ability to attach fucose to Asn (297)-linked carbohydrates, also resulting in hypofucosylation of antibodies expressed in that host cell (see also Shields et al., (2002) J. Biol. Chem. 277:26733-26740). WO99/54342 by Umana et al., describes cell lines engineered to express glycoprotein-modifying glycosyl transferases (e.g., beta(1,4)-N acetylglucosaminyltransferase III (GnTIII)) such that antibodies expressed in the engineered cell lines exhibit increased bisecting GlcNac structures which results in increased ADCC activity of the antibodies (see also Umana et al., Nat. Biotech. 17:176-180, 1999).

In another aspect, if a reduction of ADCC is desired, human antibody subclass IgG4 was shown in many previous reports to have only modest ADCC and almost no CDC effector function (Moore G L, et al., 2010 MAbs, 2:181-189). However, natural IgG4 was found less stable in stress conditions such as in acidic buffer or under increasing temperature (Angal, S. 1993 Mol Immunol, 30:105-108; Dall'Acqua, W. et al., 1998 Biochemistry, 37:9266-9273; Aalberse et al., 2002 Immunol, 105:9-19). Reduced ADCC can be achieved by operably linking the antibody to an IgG4 Fc engineered with combinations of alterations that reduce FcγR binding or Clq binding activities, thereby reducing or eliminating ADCC and CDC effector functions. Considering the physicochemical properties of antibody as a biological drug, one of the less desirable, intrinsic properties of IgG4 is dynamic separation of its two heavy chains in solution to form half antibody, which lead to bi-specific antibodies generated in vivo via a process called “Fab arm exchange” (Van der Neut Kolfschoten M, et al., 2007 Science, 317:1554-157). The mutation of serine to proline at position 228 (EU numbering system) appeared inhibitory to the IgG4 heavy chain separation (Angal, S. 1993 Mol Immunol, 30:105-108; Aalberse et al., 2002 Immunol, 105:9-19). Some of the amino acid residues in the hinge and γFc region were reported to have impact on antibody interaction with Fcγ receptors (Chappel S M, et al., 1991 Proc. Natl. Acad. Sci. USA, 88:9036-9040; Mukherjee, J. et al., 1995 FASEB J, 9:115-119; Armour, K. L. et al., 1999 Eur J Immunol, 29:2613-2624; Clynes, R. A. et al., 2000 Nature Medicine, 6:443-446; Arnold J. N., 2007 Annu Rev immunol, 25:21-50). Furthermore, some rarely occurring IgG4 isoforms in human population can also elicit different physicochemical properties (Brusco, A. et al., 1998 Eur J Immunogenet, 25:349-55; Aalberse et al., 2002 Immunol, 105:9-19). To generate multi-specific antibodies with low ADCC and CDC but with good stability, it is possible to modify the hinge and Fc region of human IgG4 and introduce a number of alterations. These modified IgG4 Fc molecules can be found in U.S. Pat. No. 8,735,553 to Li et al, which is incorporated by reference herein.

### Antibody Production

Antibodies and antigen-binding fragments thereof can be produced by any means known in the art, including but not limited to, recombinant expression, chemical synthesis, and enzymatic digestion of antibody tetramers, whereas full-length monoclonal antibodies can be obtained by, e.g., hybridoma or recombinant production. Recombinant expression can be from any appropriate host cells known in the art, for example, mammalian host cells, bacterial host cells, yeast host cells, insect host cells, etc.

The disclosure further provides polynucleotides encoding the antibodies described herein, e.g., polynucleotides encoding heavy or light chain variable regions or segments comprising the complementarity determining regions as described herein. In some aspects, the polynucleotide encoding the heavy chain variable regions has at least 85%, 89%, 90%, 91%, 92%, 93%, 94%, 95%, 96%, 97%, 98%, 99%, or 100% nucleic acid sequence identity with a polynucleotide selected from the group consisting of SEQ ID NO: 9, SEQ ID NO: 56, SEQ ID NO: 60, and SEQ ID NO: 64. In some aspects, the polynucleotide encoding the light chain variable regions has at least 85%, 89%, 90%, 91%, 92%, 93%, 94%, 95%, 96%, 97%, 98%, 99%, or 100% nucleic acid sequence identity with a polynucleotide selected from the group consisting of SEQ ID NO: 10, 57, 61 or 65.

The polynucleotides of the present disclosure can encode the variable region sequence of an anti-CLDN6×CD3 antibody. They can also encode both a variable region and a constant region of the antibody. Some of the polynucleotide sequences encode a polypeptide that comprises variable regions of both the heavy chain and the light chain of the exemplified anti-CLDN6×CD3 antibodies.

Also provided in the present disclosure are expression vectors and host cells for producing the anti-CLDN6×CD3 antibodies. The choice of expression vector depends on the intended host cells in which the vector is to be expressed. Typically, the expression vectors contain a promoter and other regulatory sequences (e.g., enhancers) that are operably linked to the polynucleotides encoding an anti-CLDN6×CD3 antibody chain or antigen-binding fragment. In some aspects, an inducible promoter is employed to prevent expression of inserted sequences except under the control of inducing conditions. Inducible promoters include, e.g., arabinose, lacZ, metallothionein promoter or a heat shock promoter. Cultures of transformed organisms can be expanded under non-inducing conditions without biasing the population for coding sequences whose expression products are better tolerated by the host cells. In addition to promoters, other regulatory elements can also be required or desired for efficient expression of an anti-CLDN6×CD3 antibody or antigen-binding fragment. These elements typically include an ATG initiation codon and adjacent ribosome binding site or other sequences. In addition, the efficiency of expression can be enhanced by the inclusion of enhancers appropriate to the cell system in use (see, e.g., Scharf et al., Results Probl. Cell Differ. 20:125, 1994; and Bittner et al., Meth. Enzymol., 153:516, 1987). For example, the SV40 enhancer or CMV enhancer can be used to increase expression in mammalian host cells.

The host cells for harboring and expressing the anti-CLDN6×CD3 antibody chains can be either prokaryotic or eukaryotic. E. coli is one prokaryotic host useful for cloning and expressing the polynucleotides of the present disclosure. Other microbial hosts suitable for use include bacilli, such as Bacillus subtilis, and other enterobacteriaceae, such as Salmonella, Serratia, and various Pseudomonas species. In these prokaryotic hosts, one can also make expression vectors, which typically contain expression control sequences compatible with the host cell (e.g., an origin of replication). In addition, any number of a variety of well-known promoters will be present, such as the lactose promoter system, a tryptophan (trp) promoter system, a beta-lactamase promoter system, or a promoter system from phage lambda. The promoters typically control expression, optionally with an operator sequence, and have ribosome binding site sequences and the like, for initiating and completing transcription and translation. Other microbes, such as yeast, can also be employed to express anti-CLDN6×CD3 antibodies. Insect cells in combination with baculovirus vectors can also be used. In other aspects, mammalian host cells are used to express and produce the anti-CLDN6×CD3 antibodies of the present disclosure. For example, they can be either a hybridoma cell line expressing endogenous immunoglobulin genes or a mammalian cell line harboring an exogenous expression vector. These include any normal mortal or normal or abnormal immortal animal or human cells. For example, several suitable host cell lines capable of secreting intact immunoglobulins have been developed, including the CHO cell lines, various COS cell lines, HEK 293 cells, myeloma cell lines, transformed B-cells and hybridomas. The use of mammalian tissue cell culture to express polypeptides is discussed generally in, e.g., Winnacker, From Genes to Clones, VCH Publishers, NY, N.Y., 1987. Expression vectors for mammalian host cells can include expression control sequences, such as an origin of replication, a promoter, and an enhancer (see, e.g., Queen et al., Immunol. Rev. 89:49-68, 1986), and necessary processing information sites, such as ribosome binding sites, RNA splice sites, polyadenylation sites, and transcriptional terminator sequences. These expression vectors usually contain promoters derived from mammalian genes or from mammalian viruses. Suitable promoters can be constitutive, cell type-specific, stage-specific, and/or modulatable or regulatable. Useful promoters include, but are not limited to, the metallothionein promoter, the constitutive adenovirus major late promoter, the dexamethasone-inducible MMTV promoter, the SV40 promoter, the MRP polIII promoter, the constitutive MPSV promoter, the tetracycline-inducible CMV promoter (such as the human immediate-early CMV promoter), the constitutive CMV promoter, and promoter-enhancer combinations known in the art.

### Production of Bispecific Antibodies

The current standard for an engineered heterodimeric antibody Fc domain is the knobs-into-holes (KiH) design, which introduced mutations at the core CH3 domain interface. The resulted heterodimers have a reduced CH3 melting temperature (69° C. or less). On the contrary, the ZW heterodimeric Fc design has a thermal stability of 81.5° C., which is comparable to the wild-type CH3 domain.

### Methods of Detection and Diagnosis

The antibodies or antigen-binding fragments of the present disclosure are useful in a variety of applications including, but not limited to, methods for the detection of CLDN6. In one aspect, the antibodies or antigen-binding fragments are useful for detecting the presence of CLDN6 in a biological sample. The term “detecting” as used herein includes quantitative or qualitative detection. In certain aspects, a biological sample comprises a cell or tissue. In other aspects, such tissues include normal and/or cancerous tissues that express CLDN6 at higher levels relative to other tissues.

In one aspect, the present disclosure provides a method of detecting the presence of CLDN6 in a biological sample. In certain aspects, the method comprises contacting the biological sample with an anti-CLDN6×CD3 antibody under conditions permissive for binding of the antibody to the antigen and detecting whether a complex is formed between the antibody and the antigen. The biological sample can include, without limitation, urine, tissue, sputum or blood samples.

Also included is a method of diagnosing a disorder associated with expression of CLDN6. In certain aspects, the method comprises contacting a test cell with an anti-CLDN6×CD3 antibody; determining the level of expression (either quantitatively or qualitatively) of CLDN6 expressed by the test cell by detecting binding of the anti-CLDN6×CD3 antibody to the CLDN6 polypeptide; and comparing the level of expression by the test cell with the level of CLDN6 expression in a control cell (e.g., a normal cell of the same tissue origin as the test cell or a non-CLDN6 expressing cell), wherein a higher level of CLDN6 expression in the test cell as compared to the control cell indicates the presence of a disorder associated with expression of CLDN6.

### Methods of Treatment

The antibodies or antigen-binding fragments of the present disclosure are useful in a variety of applications including, but not limited to, methods for the treatment of a CLDN6-associated disorder or disease. In one aspect, the CLDN6-associated disorder or disease is a cancer.

In one aspect, the present disclosure provides a method of treating cancer. In certain aspects, the method comprises administering to a patient in need thereof an effective amount of an anti-CLDN6×CD3 antibody or antigen-binding fragment. In some embodiments, the cancer is a solid tumor. The cancer can include, without limitation, gastric cancer, colon cancer, pancreatic cancer, breast cancer, head and neck cancer, kidney cancer, liver cancer, small cell lung cancer, non-small cell lung cancer, ovarian cancer, skin cancer, mesothelioma, lymphoma, leukemia, myeloma, sarcoma, brain cancer, colorectal cancer, prostate cancer, cervical cancer, testicular cancer, endometrial cancer, bladder cancer, rhabdoid tumor, and/or glioma.

The antibody or antigen-binding fragment as disclosed herein can be administered by any suitable means, including parenteral, intrapulmonary, and intranasal, and, if desired for local treatment, intralesional administration. Parenteral infusions include intramuscular, intravenous, intraarterial, intraperitoneal, or subcutaneous administration. Dosing can be by any suitable route, e.g. by injections, such as intravenous or subcutaneous injections, depending in part on whether the administration is brief or chronic. Various dosing schedules including but not limited to single or multiple administrations over various time-points, bolus administration, and pulse infusion are contemplated herein.

Antibodies or antigen-binding fragments of the disclosure can be formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners. The antibody need not be, but is optionally formulated with one or more agents currently used to prevent or treat the disorder in question. The effective amount of such other agents depends on the amount of antibody present in the formulation, the type of disorder or treatment, and other factors discussed above. These are generally used in the same dosages and with administration routes as described herein, or about from 1 to 99% of the dosages described herein, or in any dosage and by any route that is empirically/clinically determined to be appropriate.

For the prevention or treatment of disease, the appropriate dosage of an antibody or antigen-binding fragment of the disclosure will depend on the type of disease to be treated, the type of antibody, the severity and course of the disease, whether the antibody is administered for preventive or therapeutic purposes, previous therapy, the patient's clinical history and response to the antibody, and the discretion of the attending physician. The antibody is suitably administered to the patient at one time or over a series of treatments. Depending on the type and severity of the disease, about 1 μg/kg to 100 mg/kg of antibody can be an initial candidate dosage for administration to the patient, whether, for example, by one or more separate administrations, or by continuous infusion. One typical daily dosage might range from about 1 μg/kg to 100 mg/kg or more, depending on the factors mentioned above. For repeated administrations over several days or longer, depending on the condition, the treatment would generally be sustained until a desired suppression of disease symptoms occurs. Such doses can be administered intermittently, e.g. every week or every three weeks (e.g. such that the patient receives from about two to about twenty, or e.g. about six doses of the antibody). An initial higher loading dose, followed by one or more lower doses can be administered. However, other dosage regimens can be useful. The progress of this therapy is easily monitored by conventional techniques and assays.

### Combination Therapy

In one aspect, anti-CLDN6×CD3 antibodies of the present disclosure can be used in combination with other therapeutic agents. Other therapeutic agents that can be used with the anti-CLDN6×CD3 antibodies of the present disclosure include: but are not limited to, a chemotherapeutic agent (e.g., paclitaxel or a paclitaxel agent; (e.g. Abraxane®), docetaxel; carboplatin; topotecan; cisplatin; irinotecan, doxorubicin, lenalidomide, 5-azacytidine, ifosfamide, oxaliplatin, pemetrexed disodium, cyclophosphamide, etoposide, decitabine, fludarabine, vincristine, bendamustine, chlorambucil, busulfan, gemcitabine, melphalan, pentostatin, mitoxantrone, pemetrexed disodium), tyrosine kinase inhibitor (e.g., EGFR inhibitor (e.g., erlotinib), multikinase inhibitor (e.g., MGCD265, RGB-286638), CD-20 targeting agent (e.g., rituximab, ofatumumab, RO5072759, LFB-R603), CD52 targeting agent (e.g., alemtuzumab), prednisolone, darbepoetin alfa, lenalidomide, Bcl-2 inhibitor (e.g., oblimersen sodium), aurora kinase inhibitor (e.g., MLN8237, TAK-901), proteasome inhibitor (e.g., bortezomib), CD-19 targeting agent (e.g., MEDI-551, MOR208), MEK inhibitor (e.g., ABT-348), JAK-2 inhibitor (e.g., INCB018424), mTOR inhibitor (e.g., temsirolimus, everolimus), BCR/ABL inhibitor (e.g., imatinib), ET-A receptor antagonist (e.g., ZD4054), TRAIL receptor 2 (TR-2) agonist (e.g., CS-1008), EGEN-001, Polo-like kinase 1 inhibitor (e.g., BI 672).

Anti-CLDN6×CD3 antibodies of the present disclosure can be used in combination with other therapeutics, for example, immune checkpoint antibodies. Such immune checkpoint antibodies can include anti-PD1 antibodies. Anti-PD1 antibodies can include, without limitation, Tislelizumab, Pembrolizumab or Nivolumab. Tislelizumab is disclosed in U.S. Pat. No. 8,735,553. Pembrolizumab (formerly MK-3475), is disclosed in U.S. Pat. Nos. 8,354,509 and 8,900,587 and is a humanized lgG4-K immunoglobulin which targets the PD1 receptor and inhibits binding of the PD1 receptor ligands PD-L1 and PD-L2. Pembrolizumab has been approved for the indications of metastatic melanoma and metastatic non-small cell lung cancer (NSCLC) and is under clinical investigation for the treatment of head and neck squamous cell carcinoma (HNSCC), and refractory Hodgkin's lymphoma (cHL). Nivolumab (as disclosed by Bristol-Meyers Squibb) is a fully human lgG4-K monoclonal antibody. Nivolumab (clone 5C4) is disclosed in US Patent No. U.S. Pat. No. 8,008,449 and WO 2006/121168. Nivolumab is approved for the treatment of melanoma, lung cancer, kidney cancer, and Hodgkin's lymphoma.

Other immune checkpoint antibodies for combination with anti-CLDN6×CD3 antibodies can include anti-TIGIT antibodies. Such anti-TIGIT antibodies can include without limitation, anti-TIGIT antibodies as disclosed in WO2019/129261.

Other immune checkpoint antibodies for combination with anti-CLDN6×CD3 antibodies can include anti-OX40 antibodies. Such anti-OX40 antibodies can include without limitation, anti-OX40 antibodies as disclosed in WO2019/223733.

Other immune checkpoint antibodies for combination with anti-CLDN6×CD3 antibodies can include anti-TIM3 antibodies. Such anti-TIM3 antibodies can include without limitation, anti-TIM3 antibodies as disclosed in WO2018/036561.

### Pharmaceutical Compositions and Formulations

Also provided are compositions, including pharmaceutical formulations, comprising an anti-CLDN6×CD3 antibody or antigen-binding fragment thereof, or polynucleotides comprising sequences encoding an anti-CLDN6×CD3 antibody or antigen-binding fragment. In certain embodiments, compositions comprise one or more anti-CLDN6×CD3 antibodies or antigen-binding fragments, or one or more polynucleotides comprising sequences encoding one or more anti-CLDN6×CD3 antibodies or antigen-binding fragments. These compositions can further comprise suitable carriers, such as pharmaceutically acceptable excipients including buffers, which are well known in the art.

Pharmaceutical formulations of an anti-CLDN6×CD3 antibody or antigen-binding fragment as described herein are prepared by mixing such antibody or antigen-binding fragment having the desired degree of purity with one or more optional pharmaceutically acceptable carriers (Remington's Pharmaceutical Sciences 16th edition, Osol, A. Ed. (1980)), in the form of lyophilized formulations or aqueous solutions. Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG). Exemplary pharmaceutically acceptable carriers herein further include interstitial drug dispersion agents such as soluble neutral-active hyaluronidase glycoproteins (sHASEGP), for example, human soluble PH-20 hyaluronidase glycoproteins, such as rHuPH20 (HYLENEX®, Baxter International, Inc.). Certain exemplary sHASEGPs and methods of use, including rHuPH20, are described in US Patent Nos. U.S. Pat. No. 7,871,607 and 2006/0104968. In one aspect, a sHASEGP is combined with one or more additional glycosaminoglycanases such as chondroitinases.

In one embodiment, the formulation is composed of L-histidine/L-histidine hydrochloride monohydrate, trehalose and polysorbate 20. In another embodiment the concentration of the anti-CLDN6×CD3 antibody drug product, after constitution with sterile water for injection, is an isotonic solution consisting of 10 mg/mL anti-CLDN6×CD3 antibody, 20 mM histidine/histidine HCl, 240 mM trehalose dihydrate, and 0.02% polysorbate 20, at a pH of approximately 5.5.

Exemplary lyophilized antibody formulations are described in U.S. Pat. No. 6,267,958. Aqueous antibody formulations include those described in U.S. Pat. No. 6,171,586 and WO2006/044908, the latter formulations including a histidine-acetate buffer.

Sustained-release preparations can be prepared. Suitable examples of sustained-release preparations include semipermeable matrices of solid hydrophobic polymers containing the antibody, which matrices are in the form of shaped articles, e.g. films, or microcapsules.

The formulations to be used for in vivo administration are generally sterile. Sterility can be readily accomplished, e.g., by filtration through sterile filtration membranes.

### Sequence Lists

The sequences list of the present disclosure is provided below in Table 1˜ 3.

TABLE 1
Sequences list (Kabat Numbering)

| Antibody | SEQ ID NO | Note | Sequence |
|---|---|---|---|
| BG87P | SEQ ID NO: 1 | VH CDR1 | DYHIN |
| | SEQ ID NO: 2 | VH CDR2 | WIYPGSGYTKYNEKFKV |
| | SEQ ID NO: 3 | VH CDR3 | SGIYHYVSSYYFDH |
| | SEQ ID NO: 4 | VL CDR1 | KASQDVGTAVA |
| | SEQ ID NO: 5 | VL CDR2 | WASTRHT |
| | SEQ ID NO: 6 | VL CDR3 | QQYSSYT |
| | SEQ ID NO: 7 | VH AA | QIQLQQSGPELVKPGASVKISCKASGYSFTDYHI |
| | | | NWVKQRPGQGLEWIGWIYPGSGYTKYNEKFKV |
| | | | KATLTVDTSSSTAYMHLTSLTSEDSAVYFCARSGI |
| | | | YHYVSSYYFDHWGQGTTLTVSS |
| | SEQ ID NO: 8 | VL AA | DIVMTQSHTFMSTSVGDRVSITCKASQDVGTAV |
| | | | AWYQQKPGQSPKLLIYWASTRHTGVPDRFTGSG |
| | | | SGTDFTLTISNVQSEDLADYFCQQYSSYTFGGGT |
| | | | KLEIK |
| | SEQ ID NO: 9 | VH DNA | CAGATTCAGCTGCAGCAGAGCGGCCCCGAGC |
| | | | TGGTGAAGCCCGGCGCCTCCGTGAAGATCAGC |
| | | | TGCAAGGCCTCCGGCTACAGCTTCACCGACTA |
| | | | CCACATCAACTGGGTGAAGCAGAGACCCGGC |
| | | | CAAGGCCTGGAGTGGATCGGCTGGATCTACCC |
| | | | CGGCAGCGGCTACACCAAGTACAACGAGAAG |
| | | | TTCAAGGTGAAGGCCACCCTGACCGTCGACA |
| | | | CAAGCAGCAGCACCGCCTACATGCACCTGACA |
| | | | AGCCTGACAAGCGAGGACAGCGCCGTGTACT |
| | | | TCTGCGCTAGAAGCGGCATCTACCACTACGTG |
| | | | AGCAGCTACTACTTCGACCACTGGGGCCAAGG |
| | | | CACCACCCTCACCGTGAGCAGC |
| | SEQ ID NO: 10 | VL DNA | GACATCGTGATGACACAGAGCCACACCTTCAT |
| | | | GAGCACAAGCGTGGGCGACAGAGTGAGCATC |
| | | | ACCTGCAAGGCCTCCCAAGACGTGGGCACCG |
| | | | CCGTCGCCTGGTATCAGCAGAAGCCCGGGCAG |
| | | | AGCCCCAAGCTGCTGATCTACTGGGCCTCCAC |
| | | | AAGACACACCGGCGTGCCCGACAGATTCACC |
| | | | GGCAGCGGCAGCGGCACCGACTTCACCCTGA |
| | | | CCATCAGCAACGTGCAGAGCGAGGACCTGGC |
| | | | CGACTACTTCTGTCAGCAGTACAGCAGCTACA |
| | | | CCTTCGGCGGGGGCACCAAGCTGGAGATCAA |
| | | | G |
| ChBG87P | SEQ ID NO: 1 | VH CDR1 | DYHIN |
| | SEQ ID NO: 2 | VH CDR2 | WIYPGSGYTKYNEKFKV |
| | SEQ ID NO: 3 | VH CDR3 | SGIYHYVSSYYFDH |
| | SEQ ID NO: 4 | VL CDR1 | KASQDVGTAVA |
| | SEQ ID NO: 5 | VL CDR2 | WASTRHT |
| | SEQ ID NO: 6 | VL CDR3 | QQYSSYT |
| BG87P-z0 | SEQ ID NO: 11 | VH AA | QVQLVQSGAEVKKPGASVKVSCKASGYTFTDY |
| | | | HINWVRQAPGQRLEWMGWIYPGSGYTKYNEK |
| | | | FKVRVTITRDTSASTAYMELSSLRSEDTAVYYC |
| | | | ARSGIYHYVSSYYFDHWGKGTTVTVSS |
| | SEQ ID NO: 12 | VL AA | DIQMTQSPSTLSASVGDRVTITCKASQDVGTAV |
| | | | AWYQQKPGKAPKLLIYWASTRHTGVPSRFSGS |
| | | | GSGTEFTLTISSLQPDDFATYYCQQYSSYTFGGG |
| | | | TKVEIK |
| BG87P- | SEQ ID NO: 13 | VH AA | QIQLVQSGAEVKKPGASVKVSCKASGYSFTDYH |
| Bz0 | | | INWVRQAPGQRLEWMGWIYPGSGYTKYNEKF |
| | | | KVRVTLTVDTSASTAYMELSSLRSEDTAVYFCA |
| | | | RSGIYHYVSSYYFDHWGKGTTVTVSS |
| | SEQ ID NO: 14 | VL AA | DIQMTQSPSTLSASVGDRVTITCKASQDVGTAV |
| | | | AWYQQKPGKSPKLLIYWASTRHTGVPSRFSGSG |
| | | | SGTEFTLTISSVQPDDFATYFCQQYSSYTFGGGT |
| | | | KVEIK |
| BG87P- | SEQ ID NO: 15 | VH AA | QVQLVQSGAEVKKPGASVKVSCKASGYSFTDY |
| Bz1 | | | HINWVRQAPGQRLEWMGWIYPGSGYTKYNEK |
| | | | FKVRVTLTVDTSASTAYMELSSLRSEDTAVYFC |
| | | | ARSGIYHYVSSYYFDHWGKGTTVTVSS |
| | SEQ ID NO: 14 | VL AA | DIQMTQSPSTLSASVGDRVTITCKASQDVGTAV |
| | | | AWYQQKPGKSPKLLIYWASTRHTGVPSRFSGSG |
| | | | SGTEFTLTISSVQPDDFATYFCQQYSSYTFGGGT |
| | | | KVEIK |
| BG87P- | SEQ ID NO: 16 | VH AA | QIQLVQSGAEVKKPGASVKVSCKASGYTFTDY |
| Bz2 | | | HINWVRQAPGQRLEWMGWIYPGSGYTKYNEK |
| | | | FKVRVTLTVDTSASTAYMELSSLRSEDTAVYFC |
| | | | ARSGIYHYVSSYYFDHWGKGTTVTVSS |
| | SEQ ID NO: 14 | VL AA | DIQMTQSPSTLSASVGDRVTITCKASQDVGTAV |
| | | | AWYQQKPGKSPKLLIYWASTRHTGVPSRFSGSG |
| | | | SGTEFTLTISSVQPDDFATYFCQQYSSYTFGGGT |
| | | | KVEIK |
| BG87P- | SEQ ID NO: 17 | VH AA | QIQLVQSGAEVKKPGASVKVSCKASGYSFTDYH |
| Bz3 | | | INWVRQAPGQRLEWMGWIYPGSGYTKYNEKF |
| | | | KVRVTITVDTSASTAYMELSSLRSEDTAVYFCA |
| | | | RSGIYHYVSSYYFDHWGKGTTVTVSS |
| | SEQ ID NO: 14 | VL AA | DIQMTQSPSTLSASVGDRVTITCKASQDVGTAV |
| | | | AWYQQKPGKSPKLLIYWASTRHTGVPSRFSGSG |
| | | | SGTEFTLTISSVQPDDFATYFCQQYSSYTFGGGT |
| | | | KVEIK |
| BG87P- | SEQ ID NO: 18 | VH AA | QIQLVQSGAEVKKPGASVKVSCKASGYSFTDYH |
| Bz4 | | | INWVRQAPGQRLEWMGWIYPGSGYTKYNEKF |
| | | | KVRVTLTRDTSASTAYMELSSLRSEDTAVYFCA |
| | | | RSGIYHYVSSYYFDHWGKGTTVTVSS |
| | SEQ ID NO: 14 | VL AA | DIQMTQSPSTLSASVGDRVTITCKASQDVGTAV |
| | | | AWYQQKPGKSPKLLIYWASTRHTGVPSRFSGSG |
| | | | SGTEFTLTISSVQPDDFATYFCQQYSSYTFGGGT |
| | | | KVEIK |
| BG87P- | SEQ ID NO: 19 | VH AA | QIQLVQSGAEVKKPGASVKVSCKASGYSFTDYH |
| Bz5 | | | INWVRQAPGQRLEWMGWIYPGSGYTKYNEKF |
| | | | KVRVTLTVDTSASTAYMELSSLRSEDTAVYYCA |
| | | | RSGIYHYVSSYYFDHWGKGTTVTVSS |
| | SEQ ID NO: 14 | VL AA | DIQMTQSPSTLSASVGDRVTITCKASQDVGTAV |
| | | | AWYQQKPGKSPKLLIYWASTRHTGVPSRFSGSG |
| | | | SGTEFTLTISSVQPDDFATYFCQQYSSYTFGGGT |
| | | | KVEIK |
| BG87P- | SEQ ID NO: 13 | VH AA | QIQLVQSGAEVKKPGASVKVSCKASGYSFTDYH |
| Bz6 | | | INWVRQAPGQRLEWMGWIYPGSGYTKYNEKF |
| | | | KVRVTLTVDTSASTAYMELSSLRSEDTAVYFCA |
| | | | RSGIYHYVSSYYFDHWGKGTTVTVSS |
| | SEQ ID NO: 20 | VL AA | DIQMTQSPSTLSASVGDRVTITCKASQDVGTAV |
| | | | AWYQQKPGKAPKLLIYWASTRHTGVPSRFSGS |
| | | | GSGTEFTLTISSVQPDDFATYFCQQYSSYTFGGG |
| | | | TKVEIK |
| BG87P- | SEQ ID NO: 13 | VH AA | QIQLVQSGAEVKKPGASVKVSCKASGYSFTDYH |
| Bz7 | | | INWVRQAPGQRLEWMGWIYPGSGYTKYNEKF |
| | | | KVRVTLTVDTSASTAYMELSSLRSEDTAVYFCA |
| | | | RSGIYHYVSSYYFDHWGKGTTVTVSS |
| | SEQ ID NO: 21 | VL AA | DIQMTQSPSTLSASVGDRVTITCKASQDVGTAV |
| | | | AWYQQKPGKSPKLLIYWASTRHTGVPSRFSGSG |
| | | | SGTEFTLTISSLQPDDFATYFCQQYSSYTFGGGT |
| | | | KVEIK |
| BG87P- | SEQ ID NO: 13 | VH AA | QIQLVQSGAEVKKPGASVKVSCKASGYSFTDYH |
| Bz8 | | | INWVRQAPGQRLEWMGWIYPGSGYTKYNEKF |
| | | | KVRVTLTVDTSASTAYMELSSLRSEDTAVYFCA |
| | | | RSGIYHYVSSYYFDHWGKGTTVTVSS |
| | SEQ ID NO: 22 | VL AA | DIQMTQSPSTLSASVGDRVTITCKASQDVGTAV |
| | | | AWYQQKPGKSPKLLIYWASTRHTGVPSRFSGSG |
| | | | SGTEFTLTISSVQPDDFATYYCQQYSSYTFGGGT |
| | | | KVEIK |
| BG87P-21 | SEQ ID NO: 1 | VH CDR1 | DYHIN |
| | SEQ ID NO: 23 | VH CDR2 | WIYPGSGYTKYNEKFKG |
| | SEQ ID NO: 3 | VH CDR3 | SGIYHYVSSYYFDH |
| | SEQ ID NO: 4 | VL CDR1 | KASQDVGTAVA |
| | SEQ ID NO: 5 | VL CDR2 | WASTRHT |
| | SEQ ID NO: 6 | VL CDR3 | QQYSSYT |
| | SEQ ID NO: 24 | VH AA | QVQLVQSGAEVKKPGASVKVSCKASGYSFTDY |
| | | | HINWVRQAPGQRLEWMGWIYPGSGYTKYNEK |
| | | | FKGRVTITRDTSASTAYMELSSLRSEDTAVYYC |
| | | | ARSGIYHYVSSYYFDHWGKGTTVTVSS |
| | SEQ ID NO: 12 | VL AA | DIQMTQSPSTLSASVGDRVTITCKASQDVGTAV |
| | | | AWYQQKPGKAPKLLIYWASTRHTGVPSRFSGS |
| | | | GSGTEFTLTISSLQPDDFATYYCQQYSSYTFGGG |
| | | | TKVEIK |
| BG87P-22 | SEQ ID NO: 1 | VH CDR1 | DYHIN |
| | SEQ ID NO: 23 | VH CDR2 | WIYPGSGYTKYNEKFKG |
| | SEQ ID NO: 3 | VH CDR3 | SGIYHYVSSYYFDH |
| | SEQ ID NO: 4 | VL CDR1 | KASQDVGTAVA |
| | SEQ ID NO: 5 | VL CDR2 | WASTRHT |
| | SEQ ID NO: 6 | VL CDR3 | QQYSSYT |
| | SEQ ID NO: 25 | VH AA | QVQLVQSGAEVKKPGASVKVSCKASGYSFTDY |
| | | | HINWVRQAPGQRLEWMGWIYPGSGYTKYNEK |
| | | | FKGRVTITRDTSASTAYMELSSLRSEDTAVYFC |
| | | | ARSGIYHYVSSYYFDHWGKGTTVTVSS |
| | SEQ ID NO: 12 | VL AA | DIQMTQSPSTLSASVGDRVTITCKASQDVGTAV |
| | | | AWYQQKPGKAPKLLIYWASTRHTGVPSRFSGS |
| | | | GSGTEFTLTISSLQPDDFATYYCQQYSSYTFGGG |
| | | | TKVEIK |
| BG87P-23 | SEQ ID NO: 1 | VH CDR1 | DYHIN |
| | SEQ ID NO: 23 | VH CDR2 | WIYPGSGYTKYNEKFKG |
| | SEQ ID NO: 3 | VH CDR3 | SGIYHYVSSYYFDH |
| | SEQ ID NO: 4 | VL CDR1 | KASQDVGTAVA |
| | SEQ ID NO: 5 | VL CDR2 | WASTRHT |
| | SEQ ID NO: 6 | VL CDR3 | QQYSSYT |
| | SEQ ID NO: 26 | VH AA | QIQLVQSGAEVKKPGASVKVSCKASGYSFTDYH |
| | | | INWVRQAPGQRLEWMGWIYPGSGYTKYNEKF |
| | | | KGRVTITRDTSASTAYMELSSLRSEDTAVYYCA |
| | | | RSGIYHYVSSYYFDHWGKGTTVTVSS |
| | SEQ ID NO: 12 | VL AA | DIQMTQSPSTLSASVGDRVTITCKASQDVGTAV |
| | | | AWYQQKPGKAPKLLIYWASTRHTGVPSRFSGS |
| | | | GSGTEFTLTISSLQPDDFATYYCQQYSSYTFGGG |
| | | | TKVEIK |
| BG87P-24 | SEQ ID NO: 1 | VH CDR1 | DYHIN |
| | SEQ ID NO: 23 | VH CDR2 | WIYPGSGYTKYNEKFKG |
| | SEQ ID NO: 3 | VH CDR3 | SGIYHYVSSYYFDH |
| | SEQ ID NO: 4 | VL CDR1 | KASQDVGTAVA |
| | SEQ ID NO: 5 | VL CDR2 | WASTRHT |
| | SEQ ID NO: 6 | VL CDR3 | QQYSSYT |
| | SEQ ID NO: 27 | VH AA | QVQLVQSGAEVKKPGASVKVSCKASGYSFTDY |
| | | | HINWVRQAPGQRLEWMGWIYPGSGYTKYNEK |
| | | | FKGRVTLTRDTSASTAYMELSSLRSEDTAVYYC |
| | | | ARSGIYHYVSSYYFDHWGKGTTVTVSS |
| | SEQ ID NO: 12 | VL AA | DIQMTQSPSTLSASVGDRVTITCKASQDVGTAV |
| | | | AWYQQKPGKAPKLLIYWASTRHTGVPSRFSGS |
| | | | GSGTEFTLTISSLQPDDFATYYCQQYSSYTFGGG |
| | | | TKVEIK |
| BG87P-25 | SEQ ID NO: 1 | VH CDR1 | DYHIN |
| | SEQ ID NO: 23 | VH CDR2 | WIYPGSGYTKYNEKFKG |
| | SEQ ID NO: 3 | VH CDR3 | SGIYHYVSSYYFDH |
| | SEQ ID NO: 4 | VL CDR1 | KASQDVGTAVA |
| | SEQ ID NO: 5 | VL CDR2 | WASTRHT |
| | SEQ ID NO: 6 | VL CDR3 | QQYSSYT |
| | SEQ ID NO: 28 | VH AA | QIQLVQSGAEVKKPGASVKVSCKASGYSFTDYH |
| | | | INWVRQAPGQRLEWMGWIYPGSGYTKYNEKF |
| | | | KGRVTLTRDTSASTAYMELSSLRSEDTAVYYCA |
| | | | RSGIYHYVSSYYFDHWGKGTTVTVSS |
| | SEQ ID NO: 12 | VL AA | DIQMTQSPSTLSASVGDRVTITCKASQDVGTAV |
| | | | AWYQQKPGKAPKLLIYWASTRHTGVPSRFSGS |
| | | | GSGTEFTLTISSLQPDDFATYYCQQYSSYTFGGG |
| | | | TKVEIK |
| BG87P-26 | SEQ ID NO: 1 | VH CDR1 | DYHIN |
| | SEQ ID NO: 23 | VH CDR2 | WIYPGSGYTKYNEKFKG |
| | SEQ ID NO: 3 | VH CDR3 | SGIYHYVSSYYFDH |
| | SEQ ID NO: 4 | VL CDR1 | KASQDVGTAVA |
| | SEQ ID NO: 5 | VL CDR2 | WASTRHT |
| | SEQ ID NO: 6 | VL CDR3 | QQYSSYT |
| | SEQ ID NO: 29 | VH AA | QIQLVQSGAEVKKPGASVKVSCKASGYSFTDYH |
| | | | INWVRQAPGQRLEWMGWIYPGSGYTKYNEKF |
| | | | KGRVTITRDTSASTAYMELSSLRSEDTAVYFCA |
| | | | RSGIYHYVSSYYFDHWGKGTTVTVSS |
| | SEQ ID NO: 12 | VL AA | DIQMTQSPSTLSASVGDRVTITCKASQDVGTAV |
| | | | AWYQQKPGKAPKLLIYWASTRHTGVPSRFSGS |
| | | | GSGTEFTLTISSLQPDDFATYYCQQYSSYTFGGG |
| | | | TKVEIK |
| BG87P-27 | SEQ ID NO: 1 | VH CDR1 | DYHIN |
| | SEQ ID NO: 23 | VH CDR2 | WIYPGSGYTKYNEKFKG |
| | SEQ ID NO: 3 | VH CDR3 | SGIYHYVSSYYFDH |
| | SEQ ID NO: 4 | VL CDR1 | KASQDVGTAVA |
| | SEQ ID NO: 5 | VL CDR2 | WASTRHT |
| | SEQ ID NO: 6 | VL CDR3 | QQYSSYT |
| | SEQ ID NO: 30 | VH AA | QVQLVQSGAEVKKPGASVKVSCKASGYSFTDY |
| | | | HINWVRQAPGQRLEWMGWIYPGSGYTKYNEK |
| | | | FKGRVTLTRDTSASTAYMELSSLRSEDTAVYFC |
| | | | ARSGIYHYVSSYYFDHWGKGTTVTVSS |
| | SEQ ID NO: 12 | VL AA | DIQMTQSPSTLSASVGDRVTITCKASQDVGTAV |
| | | | AWYQQKPGKAPKLLIYWASTRHTGVPSRFSGS |
| | | | GSGTEFTLTISSLQPDDFATYYCQQYSSYTFGGG |
| | | | TKVEIK |
| BG87P- | SEQ ID NO: 31 | VH AA | QIQLQQSGPELVKPGASVKISCKASGYSFTDYHI |
| m1 | | | NWVKQRPGQGLEWIGWIYPGSGYTKYNEKFKV |
| | | | KATLTVDTSSSTAYMHLTSLTSEDSAVYFCARS |
| | | | GIYHYVSSYYFEHWGQGTTLTVSS |
| | SEQ ID NO: 8 | VL AA | DIVMTQSHTFMSTSVGDRVSITCKASQDVGTAV |
| | | | AWYQQKPGQSPKLLIYWASTRHTGVPDRFTGS |
| | | | GSGTDFTLTISNVQSEDLADYFCQQYSSYTFGG |
| | | | GTKLEIK |
| BG87P- | SEQ ID NO: 32 | VH AA | QIQLQQSGPELVKPGASVKISCKASGYSFTDYHI |
| m2 | | | NWVKQRPGQGLEWIGWIYPGSGYTKYNEKFKV |
| | | | KATLTVDTSSSTAYMHLTSLTSEDSAVYFCARS |
| | | | GIYHYVSSYYFQHWGQGTTLTVSS |
| | SEQ ID NO: 8 | VL AA | DIVMTQSHTFMSTSVGDRVSITCKASQDVGTAV |
| | | | AWYQQKPGQSPKLLIYWASTRHTGVPDRFTGS |
| | | | GSGTDFTLTISNVQSEDLADYFCQQYSSYTFGG |
| | | | GTKLEIK |
| BG87P- | SEQ ID NO: 33 | VH AA | QIQLQQSGPELVKPGASVKISCKASGYSFTDYHI |
| m3 | | | NWVKQRPGQGLEWIGWIYPGSGYTKYNEKFKG |
| | | | KATLTVDTSSSTAYMHLTSLTSEDSAVYFCARS |
| | | | GIYHYVSSYYFDHWGQGTTLTVSS |
| | SEQ ID NO: 8 | VL AA | DIVMTQSHTFMSTSVGDRVSITCKASQDVGTAV |
| | | | AWYQQKPGQSPKLLIYWASTRHTGVPDRFTGS |
| | | | GSGTDFTLTISNVQSEDLADYFCQQYSSYTFGG |
| | | | GTKLEIK |
| BG87P- | SEQ ID NO: 34 | VH AA | QIQLQQSGPELVKPGASVKISCKASGYSFTDYAI |
| m4 | | | NWVKQRPGQGLEWIGWIYPGSGYTKYNEKFKV |
| | | | KATLTVDTSSSTAYMHLTSLTSEDSAVYFCARS |
| | | | GIYHYVSSYYFDHWGQGTTLTVSS |
| | SEQ ID NO: 8 | VL AA | DIVMTQSHTFMSTSVGDRVSITCKASQDVGTAV |
| | | | AWYQQKPGQSPKLLIYWASTRHTGVPDRFTGS |
| | | | GSGTDFTLTISNVQSEDLADYFCQQYSSYTFGG |
| | | | GTKLEIK |
| BG87P- | SEQ ID NO: 35 | VH AA | QIQLVQSGAEVKKPGASVKVSCKASGYSFTDYH |
| m5 | | | INWVRQAPGQRLEWMGWIYPGSGYTKYNEKF |
| | | | KVRVTLTVDTSASTAYMELSSLRSEDTAVYFCA |
| | | | RSGIYHYVSSYYFEHWGKGTTVTVSS |
| | SEQ ID NO: 14 | VL AA | DIQMTQSPSTLSASVGDRVTITCKASQDVGTAV |
| | | | AWYQQKPGKSPKLLIYWASTRHTGVPSRFSGSG |
| | | | SGTEFTLTISSVQPDDFATYFCQQYSSYTFGGGT |
| | | | KVEIK |
| BG87P- | SEQ ID NO: 36 | VH AA | QIQLVQSGAEVKKPGASVKVSCKASGYSFTDYH |
| m6 | | | INWVRQAPGQRLEWMGWIYPGSGYTKYNEKF |
| | | | KVRVTLTVDTSASTAYMELSSLRSEDTAVYFCA |
| | | | RSGIYHYVSSYYFQHWGKGTTVTVSS |
| | SEQ ID NO: 14 | VL AA | DIQMTQSPSTLSASVGDRVTITCKASQDVGTAV |
| | | | AWYQQKPGKSPKLLIYWASTRHTGVPSRFSGSG |
| | | | SGTEFTLTISSVQPDDFATYFCQQYSSYTFGGGT |
| | | | KVEIK |
| BG87P- | SEQ ID NO: 37 | VH AA | QIQLVQSGAEVKKPGASVKVSCKASGYSFTDYH |
| m7 | | | INWVRQAPGQRLEWMGWIYPGSGYTKYNEKF |
| | | | KGRVTLTVDTSASTAYMELSSLRSEDTAVYFCA |
| | | | RSGIYHYVSSYYFDHWGKGTTVTVSS |
| | SEQ ID NO: 14 | VL AA | DIQMTQSPSTLSASVGDRVTITCKASQDVGTAV |
| | | | AWYQQKPGKSPKLLIYWASTRHTGVPSRFSGSG |
| | | | SGTEFTLTISSVQPDDFATYFCQQYSSYTFGGGT |
| | | | KVEIK |
| BG87P- | SEQ ID NO: 38 | VH AA | QIQLVQSGAEVKKPGASVKVSCKASGYSFTDYA |
| m8 | | | INWVRQAPGQRLEWMGWIYPGSGYTKYNEKF |
| | | | KVRVTLTVDTSASTAYMELSSLRSEDTAVYFCA |
| | | | RSGIYHYVSSYYFDHWGKGTTVTVSS |
| | SEQ ID NO: 14 | VLAA | DIQMTQSPSTLSASVGDRVTITCKASQDVGTAV |
| | | | AWYQQKPGKSPKLLIYWASTRHTGVPSRFSGSG |
| | | | SGTEFTLTISSVQPDDFATYFCQQYSSYTFGGGT |
| | | | KVEIK |
| BG87P-33 | SEQ ID NO: 1 | VH CDR1 | DYHIN |
| | SEQ ID NO: 39 | VH CDR2 | YIYPGSGHTKYNEKFKG |
| | SEQ ID NO: 3 | VH CDR3 | SGIYHYVSSYYFDH |
| | SEQ ID NO: 40 | VL CDR1 | KASQDAGSAVA |
| | SEQ ID NO: 5 | VL CDR2 | WASTRHT |
| | SEQ ID NO: 6 | VL CDR3 | QQYSSYT |
| | SEQ ID NO: 41 | VH AA | EVQLQESGGGLVQPGGSLRLSCAASGYSFTDYH |
| | | | INWVRQAPGKGLEWVSYIYPGSGHTKYNEKFK |
| | | | GRFTISRDTSKNTAYLQMNSLRAEDTAVYYCAR |
| | | | SGIYHYVSSYYFDHWGQGTLVTVSS |
| | SEQ ID NO: 42 | VL AA | DIQMTQSPSTLSASVGDRVTITCKASQDAGSAV |
| | | | AWYQQKPGKAPKLLIYWASTRHTGVPSRFSGS |
| | | | GSGTEFTLTISSLQPDDFATYYCQQYSSYTFGGG |
| | | | TKVEIK |
| BG87P-34 | SEQ ID NO: 1 | VH CDR1 | DYHIN |
| | SEQ ID NO: 39 | VH CDR2 | YIYPGSGHTKYNEKFKG |
| | SEQ ID NO: 3 | VH CDR3 | SGIYHYVSSYYFDH |
| | SEQ ID NO: 40 | VL CDR1 | KASQDAGSAVA |
| | SEQ ID NO: 5 | VL CDR2 | WASTRHT |
| | SEQ ID NO: 6 | VL CDR3 | QQYSSYT |
| | SEQ ID NO: 43 | VH AA | EVQLQESGGGLVQPGGSLRLSCAASGYSFSDYH |
| | | | INWVRQAPGKGLEWVGYIYPGSGHTKYNEKFK |
| | | | GRFTISRDTSSNTAYLQMNSLRAEDTAVYYCAR |
| | | | SGIYHYVSSYYFDHWGQGTLVTVSS |
| | SEQ ID NO: 44 | VL AA | DIQMTQSPSSLSASVGDRVTITCKASQDAGSAV |
| | | | AWYQQKPGKAPKLLIYWASTRHTGVPSRFSGS |
| | | | GSGTDFTLTISSLQPEDFATYYCQQYSSYTFGGG |
| | | | TKLEIK |
| BG87P-31 | SEQ ID NO: 1 | VH CDR1 | DYHIN |
| | SEQ ID NO: 45 | VH CDR2 | YIYPGSGHTKKNEKFKG |
| | SEQ ID NO: 3 | VH CDR3 | SGIYHYVSSYYFDH |
| | SEQ ID NO: 40 | VL CDR1 | KASQDAGSAVA |
| | SEQ ID NO: 5 | VL CDR2 | WASTRHT |
| | SEQ ID NO: 6 | VL CDR3 | QQYSSYT |
| | SEQ ID NO: 46 | VH AA | QVQLQQSGAETKKPGASVKVSCKASGYSFTDY |
| | | | HINWVRQASGQRLEWMGYIYPGSGHTKKNEKF |
| | | | KGRVTITRDTSASTAYMELSSLRSEDTAVYYCA |
| | | | RSGIYHYVSSYYFDHWGKGTTVTVSS |
| | SEQ ID NO: 47 | VL AA | DIQMTQSPSTLSASVGDRVTITCKASQDAGSAV |
| | | | AWYQQKPGKAPKLLIKWASTRHTGVPSRFSGS |
| | | | GSGTEFTLTISSLQPDDFATYYCQQYSSYTFGGG |
| | | | TKVEIK |
| BG87P-32 | SEQ ID NO: 1 | VH CDR1 | DYHIN |
| | SEQ ID NO: 45 | VH CDR2 | YIYPGSGHTKKNEKFKG |
| | SEQ ID NO: 3 | VH CDR3 | SGIYHYVSSYYFDH |
| | SEQ ID NO: 40 | VL CDR1 | KASQDAGSAVA |
| | SEQ ID NO: 5 | VL CDR2 | WASTRHT |
| | SEQ ID NO: 6 | VL CDR3 | QQYSSYT |
| | SEQ ID NO: 46 | VH AA | QVQLQQSGAETKKPGASVKVSCKASGYSFTDY |
| | | | HINWVRQASGQRLEWMGYIYPGSGHTKKNEKF |
| | | | KGRVTITRDTSASTAYMELSSLRSEDTAVYYCA |
| | | | RSGIYHYVSSYYFDHWGKGTTVTVSS |
| | SEQ ID NO: 42 | VL AA | DIQMTQSPSTLSASVGDRVTITCKASQDAGSAV |
| | | | AWYQQKPGKAPKLLIYWASTRHTGVPSRFSGS |
| | | | GSGTEFTLTISSLQPDDFATYYCQQYSSYTFGGG |
| | | | TKVEIK |

TABLE 2
Sequences list (Kabat Numbering)

| | | | |
|---|---|---|---|
| Sp34 | SEQ ID NO: 48 | HCDR1 | TYAMN |
| | | (Kabat) | |
| | SEQ ID NO: 49 | HCDR2 | RIRSKYNNYATYYADSVKD |
| | | (Kabat) | |
| | SEQ ID NO: 50 | HCDR3 | HGNFGNSYVSWFAY |
| | | (Kabat) | |
| | SEQ ID NO: 51 | LCDR1 | RSSTGAVTTSNYAN |
| | | (Kabat) | |
| | SEQ ID NO: 52 | LCDR2 | GTNKRAP |
| | | (Kabat) | |
| | SEQ ID NO: 53 | LCDR3 | ALWYSNLWV |
| | | (Kabat) | |
| | SEQ ID NO: 54 | VH AA | EVQLVESGGGLVQPKGSLKLSCAASGFTFNTYA |
| | | | MNWVRQAPGKGLEWVARIRSKYNNYATYYADS |
| | | | VKDRFTISRDDSQSILYLQMNNLKTEDTAMYYC |
| | | | VRHGNFGNSYVSWFAYWGQGTLVTVSS |
| | SEQ ID NO: 55 | VL AA | QAVVTQESALTTSPGETVTLTCRSSTGAVTTSNY |
| | | | ANWVQEKPDHLFTGLIGGTNKRAPGVPARFSGS |
| | | | LIGDKAALTITGAQTEDEAIYFCALWYSNLWVFG |
| | | | GGTKLTVL |
| | SEQ ID NO: 56 | VH DNA | GAGGTGCAGCTGGTGGAGTCCGGCGGTGGCCT |
| | | | TGTGCAGCCCAAGGGCAGCCTGAAGCTGAGCT |
| | | | GCGCCGCTAGCGGCTTCACCTTCAACACCTAC |
| | | | GCCATGAACTGGGTGAGACAAGCCCCCGGCAA |
| | | | GGGCCTGGAGTGGGTGGCTAGAATCAGAAGCA |
| | | | AGTACAACAACTACGCCACCTACTACGCCGAC |
| | | | AGCGTGAAGGACAGATTCACCATCAGCAGAGA |
| | | | CGACTCTCAGAGCATCCTGTACCTGCAGATGA |
| | | | ACAACCTGAAGACCGAGGACACCGCCATGTAC |
| | | | TACTGCGTGAGACACGGCAACTTCGGCAACAG |
| | | | CTACGTGAGCTGGTTCGCCTACTGGGGCCAAG |
| | | | GCACCCTGGTAACAGTAAGCAGC |
| | SEQ ID NO: 57 | VL DNA | CAAGCCGTGGTGACCCAAGAGAGCGCCCTGA |
| | | | CCACAAGCCCCGGCGAGACCGTGACCCTGACC |
| | | | TGCAGAAGCAGCACCGGCGCCGTGACCACAA |
| | | | GCAACTACGCCAACTGGGTGCAAGAGAAGCC |
| | | | CGACCACCTGTTCACCGGTCTGATCGGCGGTA |
| | | | CCAACAAGAGAGCCCCCGGCGTGCCCGCTAGA |
| | | | TTCAGCGGCTCACTGATCGGCGACAAGGCCGC |
| | | | CCTGACCATCACCGGCGCTCAGACCGAGGACG |
| | | | AGGCCATCTACTTCTGCGCCCTGTGGTACAGCA |
| | | | ACCTGTGGGTGTTCGGCGGCGGCACCAAGCTG |
| | | | ACCGTGCTG |
| BG53P | SEQ ID NO: 48 | HCDR1 | TYAMN |
| | | (Kabat) | |
| | SEQ ID NO: 49 | HCDR2 | RIRSKYNNYATYYADSVKD |
| | | (Kabat) | |
| | SEQ ID NO: 50 | HCDR3 | HGNFGNSYVSWFAY |
| | | (Kabat) | |
| | SEQ ID NO: 51 | LCDR1 | RSSTGAVTTSNYAN |
| | | (Kabat) | |
| | SEQ ID NO: 52 | LCDR2 | GTNKRAP |
| | | (Kabat) | |
| | SEQ ID NO: 53 | LCDR3 | ALWYSNLWV |
| | | (Kabat) | |
| | SEQ ID NO: 58 | VH AA | EVQLVESGGGLVQPGGSLRLSCAASGFTFNTYA |
| | | | MNWVRQAPGKGLEWVARIRSKYNNYATYYADS |
| | | | VKDRFTISRDDAKSSLYLQMNSLRAEDTAMYYC |
| | | | VRHGNFGNSYVSWFAYWGQGTLVTVSS |
| | SEQ ID NO: 59 | VL AA | QAVVTQSPATLSVSPGERATLSCRSSTGAVTTSNY |
| | | | ANWVQEKPGQLFTGLIGGTNKRAPGIPARFSGSL |
| | | | SGDEATLTISSLQSEDFAIYFCALWYSNLWVFGG |
| | | | GTKVEIK |
| | SEQ ID NO: 60 | VH DNA | GAGGTGCAGCTGGTGGAGTCCGGCGGTGGCCT |
| | | | TGTGCAGCCCGGCGGCAGTCTGAGACTGAGCT |
| | | | GCGCCGCTAGCGGCTTCACCTTCAACACCTAC |
| | | | GCCATGAACTGGGTGAGACAAGCCCCCGGCAA |
| | | | GGGCCTGGAGTGGGTGGCTAGAATCAGAAGCA |
| | | | AGTACAACAACTACGCCACCTACTACGCCGAC |
| | | | AGCGTGAAGGACAGATTCACCATCAGCAGAGA |
| | | | CGACGCCAAGAGCAGCCTGTACCTGCAGATGA |
| | | | ACAGCCTGAGAGCCGAGGACACCGCCATGTAC |
| | | | TACTGCGTGAGACACGGCAACTTCGGCAACAG |
| | | | CTACGTGAGCTGGTTCGCCTACTGGGGCCAAG |
| | | | GCACCCTGGTAACGGTGAGCAGC |
| | SEQ ID NO: 61 | VL DNA | CAAGCCGTGGTGACCCAAAGTCCCGCGACCCT |
| | | | TAGCGTAAGCCCCGGCGAGAGAGCTACCCTGA |
| | | | GCTGCAGAAGCAGCACCGGCGCCGTGACCAC |
| | | | AAGCAACTACGCCAACTGGGTGCAAGAGAAG |
| | | | CCCGGGCAGCTGTTCACCGGCCTGATCGGCGG |
| | | | TACCAACAAGAGAGCCCCCGGCATCCCCGCTA |
| | | | GATTCAGCGGCAGCCTGAGCGGCGACGAGGCC |
| | | | ACCCTGACCATCAGCAGCCTGCAGAGCGAGGA |
| | | | CTTCGCCATCTACTTCTGCGCCCTGTGGTACAG |
| | | | CAACCTGTGGGTGTTCGGCGGCGGCACCAAGG |
| | | | TGGAGATCAAG |
| BG56P | SEQ ID NO: 48 | HCDR1 | TYAMN |
| | | (Kabat) | |
| | SEQ ID NO: 49 | HCDR2 | RIRSKYNNYATYYADSVKD |
| | | (Kabat) | |
| | SEQ ID NO: 50 | HCDR3 | HGNFGNSYVSWFAY |
| | | (Kabat) | |
| | SEQ ID NO: 51 | LCDR1 | RSSTGAVTTSNYAN |
| | | (Kabat) | |
| | SEQ ID NO: 52 | LCDR2 | GTNKRAP |
| | | (Kabat) | |
| | SEQ ID NO: 53 | LCDR3 | ALWYSNLWV |
| | | (Kabat) | |
| | SEQ ID NO: 62 | VH | EVQLVESGGGLVQPGGSLRLSCAASGFTFNTYA |
| | | AA | MNWVRQAPGKGLEWVARIRSKYNNYATYYADS |
| | | | VKDRFTISRDDAKNSLYLQMNSLRAEDTAVYYC |
| | | | VRHGNFGNSYVSWFAYWGQGTLVTVSS |
| | SEQ ID NO: 63 | VL | EIVLTQSPATLSVSPGERATLSCRSSTGAVTTSNYA |
| | | AA | NWVQQKPGQAPRGLIGGTNKRAPGIPARFSGSLS |
| | | | GDEATLTISSLQSEDFAVYYCALWYSNLWVFGGG |
| | | | TKVEIK |
| | SEQ ID NO: 64 | VH | GAGGTGCAGCTGGTGGAGTCCGGCGGTGGCCT |
| | | DNA | TGTGCAGCCCGGCGGCAGTCTGAGACTGAGCT |
| | | | GCGCCGCTAGCGGCTTCACCTTCAACACCTAC |
| | | | GCCATGAACTGGGTGAGACAAGCCCCCGGCAA |
| | | | GGGCCTGGAGTGGGTGGCTAGAATCAGAAGCA |
| | | | AGTACAACAACTACGCCACCTACTACGCCGAC |
| | | | AGCGTGAAGGACAGATTCACCATCAGCAGAGA |
| | | | CGACGCCAAGAACAGCCTGTACCTGCAGATGA |
| | | | ACAGCCTGAGAGCCGAGGACACCGCCGTGTAC |
| | | | TACTGCGTTAGACACGGCAACTTCGGCAACAG |
| | | | CTACGTGAGCTGGTTCGCCTACTGGGGCCAAG |
| | | | GCACCCTGGTAACGGTGAGCAGC |
| | SEQ ID NO: 65 | VL | GAAATCGTGCTGACCCAAAGTCCCGCGACCCT |
| | | DNA | TAGCGTAAGCCCCGGCGAGAGAGCTACCCTGA |
| | | | GCTGCAGAAGCAGCACCGGCGCCGTGACCAC |
| | | | AAGCAACTACGCCAACTGGGTGCAACAGAAG |
| | | | CCCGGGCAGGCGCCCCGCGGCCTGATCGGCGG |
| | | | TACCAACAAGAGAGCCCCCGGCATCCCCGCTA |
| | | | GATTCAGCGGCAGCCTGAGCGGCGACGAGGCC |
| | | | ACCCTGACCATCAGCAGCCTGCAGAGCGAGGA |
| | | | CTTCGCCGTCTACTACTGCGCCCTGTGGTACAG |
| | | | CAACCTGTGGGTGTTCGGCGGCGGCACCAAGG |
| | | | TGGAGATCAAG |
| BG561P | SEQ ID NO: 48 | HCDR1 | TYAMN |
| | | (Kabat) | |
| | SEQ ID NO: 49 | HCDR2 | RIRSKYNNYATYYADSVKD |
| | | (Kabat) | |
| | SEQ ID NO: 50 | HCDR3 | HGNFGNSYVSWFAY |
| | | (Kabat) | |
| | SEQ ID NO: 51 | LCDR1 | RSSTGAVTTSNYAN |
| | | (Kabat) | |
| | SEQ ID NO: 52 | LCDR2 | GTNKRAP |
| | | (Kabat) | |
| | SEQ ID NO: 53 | LCDR3 | ALWYSNLWV |
| | | (Kabat) | |
| | SEQ ID NO: 62 | VH | EVQLVESGGGLVQPGGSLRLSCAASGFTFNTYA |
| | | AA | MNWVRQAPGKGLEWVARIRSKYNNYATYYADS |
| | | | VKDRFTISRDDAKNSLYLQMNSLRAEDTAVYYC |
| | | | VRHGNFGNSYVSWFAYWGQGTLVTVSS |
| | SEQ ID NO: 63 | VL | EIVLTQSPATLSVSPGERATLSCRSSTGAVTTSNYA |
| | | AA | NWVQQKPGQAPRGLIGGTNKRAPGIPARFSGSLS |
| | | | GDEATLTISSLQSEDFAVYYCALWYSNLWVFGGG |
| | | | TKVEIK |
| | SEQ ID NO: 66 | scFv | EVQLVESGGGLVQPGGSLRLSCAASGFTFNTYA |
| | | AA | MNWVRQAPGKGLEWVARIRSKYNNYATYYADS |
| | | | VKDRFTISRDDAKNSLYLQMNSLRAEDTAVYYC |
| | | | VRHGNFGNSYVSWFAYWGQGTLVTVSSGGGGS |
| | | | GGGGSGGGGSEIVLTQSPATLSVSPGERATLSCRS |
| | | | STGAVTTSNYANWVQQKPGQAPRGLIGGTNKRA |
| | | | PGIPARFSGSLSGDEATLTISSLQSEDFAVYYCAL |
| | | | WYSNLWVFGGGTKVEIK |
| | SEQ ID NO: 67 | scFv | GAGGTGCAGCTGGTGGAGTCCGGCGGTGGCCT |
| | | DNA | TGTGCAGCCCGGCGGCAGTCTGAGACTGAGCT |
| | | | GCGCCGCTAGCGGCTTCACCTTCAACACCTAC |
| | | | GCCATGAACTGGGTGAGACAAGCCCCCGGCAA |
| | | | GGGCCTGGAGTGGGTGGCTAGAATCAGAAGCA |
| | | | AGTACAACAACTACGCCACCTACTACGCCGAC |
| | | | AGCGTGAAGGACAGATTCACCATCAGCAGAGA |
| | | | CGACGCCAAGAACAGCCTGTACCTGCAGATGA |
| | | | ACAGCCTGAGAGCCGAGGACACCGCCGTGTAC |
| | | | TACTGCGTTAGACACGGCAACTTCGGCAACAG |
| | | | CTACGTGAGCTGGTTCGCCTACTGGGGCCAAG |
| | | | GCACCCTGGTAACGGTGAGCAGCGGCGGCGGT |
| | | | GGCAGCGGCGGAGGCGGCTCTGGCGGCGGAG |
| | | | GATCCGAAATCGTGCTGACCCAAAGTCCCGCG |
| | | | ACCCTTAGCGTAAGCCCCGGCGAGAGAGCTAC |
| | | | CCTGAGCTGCAGAAGCAGCACCGGCGCCGTG |
| | | | ACCACAAGCAACTACGCCAACTGGGTGCAACA |
| | | | GAAGCCCGGGCAGGCGCCCCGCGGCCTGATCG |
| | | | GCGGTACCAACAAGAGAGCCCCCGGCATCCCC |
| | | | GCTAGATTCAGCGGCAGCCTGAGCGGCGACGA |
| | | | GGCCACCCTGACCATCAGCAGCCTGCAGAGCG |
| | | | AGGACTTCGCCGTCTACTACTGCGCCCTGTGGT |
| | | | ACAGCAACCTGTGGGTGTTCGGCGGCGGCACC |
| | | | AAGGTGGAGATCAAG |
| BG562P | SEQ ID NO: 48 | HCDR1 | TYAMN |
| | | (Kabat) | |
| | SEQ ID NO: 49 | HCDR2 | RIRSKYNNYATYYADSVKD |
| | | (Kabat) | |
| | SEQ ID NO: 50 | HCDR3 | HGNFGNSYVSWFAY |
| | | (Kabat) | |
| | SEQ ID NO: 51 | LCDR1 | RSSTGAVTTSNYAN |
| | | (Kabat) | |
| | SEQ ID NO: 52 | LCDR2 | GTNKRAP |
| | | (Kabat) | |
| | SEQ ID NO: 53 | LCDR3 | ALWYSNLWV |
| | | (Kabat) | |
| | SEQ ID NO: 62 | VH | EVQLVESGGGLVQPGGSLRLSCAASGFTFNTYA |
| | | AA | MNWVRQAPGKGLEWVARIRSKYNNYATYYADS |
| | | | VKDRFTISRDDAKNSLYLQMNSLRAEDTAVYYC |
| | | | VRHGNFGNSYVSWFAYWGQGTLVTVSS |
| | SEQ ID NO: 68 | VL | EIVVTQSPATLSVSPGERATLSCRSSTGAVTTSNY |
| | | AA | ANWVQQKPGQAPRGLIGGTNKRAPGIPARFSGS |
| | | | LSGDEATLTISSLQSEDFAVYYCALWYSNLWVFG |
| | | | GGTKVEIK |
| | SEQ ID NO: 69 | ScFv | EVQLVESGGGLVQPGGSLRLSCAASGFTFNTYA |
| | | AA | MNWVRQAPGKGLEWVARIRSKYNNYATYYADS |
| | | | VKDRFTISRDDAKNSLYLQMNSLRAEDTAVYYC |
| | | | VRHGNFGNSYVSWFAYWGQGTLVTVSSGGGGS |
| | | | GGGGSGGGGSEIVVTQSPATLSVSPGERATLSCRS |
| | | | STGAVTTSNYANWVQQKPGQAPRGLIGGTNKRA |
| | | | PGIPARFSGSLSGDEATLTISSLQSEDFAVYYCAL |
| | | | WYSNLWVFGGGTKVEIK |
| | SEQ ID NO: 70 | ScFv | GAGGTGCAGCTGGTGGAGTCCGGCGGTGGCCT |
| | | DNA | TGTGCAGCCCGGCGGCAGTCTGAGACTGAGCT |
| | | | GCGCCGCTAGCGGCTTCACCTTCAACACCTAC |
| | | | GCCATGAACTGGGTGAGACAAGCCCCCGGCAA |
| | | | GGGCCTGGAGTGGGTGGCTAGAATCAGAAGCA |
| | | | AGTACAACAACTACGCCACCTACTACGCCGAC |
| | | | AGCGTGAAGGACAGATTCACCATCAGCAGAGA |
| | | | CGACGCCAAGAACAGCCTGTACCTGCAGATGA |
| | | | ACAGCCTGAGAGCCGAGGACACCGCCGTGTAC |
| | | | TACTGCGTTAGACACGGCAACTTCGGCAACAG |
| | | | CTACGTGAGCTGGTTCGCCTACTGGGGCCAAG |
| | | | GCACCCTGGTAACGGTGAGCAGCGGCGGCGGT |
| | | | GGCAGCGGCGGAGGCGGCTCTGGCGGCGGAG |
| | | | GATCCGAAATCGTGGTGACCCAAAGTCCCGCG |
| | | | ACCCTTAGCGTAAGCCCCGGCGAGAGAGCTAC |
| | | | CCTGAGCTGCAGAAGCAGCACCGGCGCCGTG |
| | | | ACCACAAGCAACTACGCCAACTGGGTGCAACA |
| | | | GAAGCCCGGGCAGGCGCCCCGCGGCCTGATCG |
| | | | GCGGTACCAACAAGAGAGCCCCCGGCATCCCC |
| | | | GCTAGATTCAGCGGCAGCCTGAGCGGCGACGA |
| | | | GGCCACCCTGACCATCAGCAGCCTGCAGAGCG |
| | | | AGGACTTCGCCGTCTACTACTGCGCCCTGTGGT |
| | | | ACAGCAACCTGTGGGTGTTCGGCGGCGGCACC |
| | | | AAGGTGGAGATCAAG |
| BG563P | SEQ ID NO: 48 | HCDR1 | TYAMN |
| | | (Kabat) | |
| | SEQ ID NO: 71 | HCDR2 | RIRSKYNNYATYYADSVKG |
| | | (Kabat) | |
| | SEQ ID NO: 50 | HCDR3 | HGNFGNSYVSWFAY |
| | | (Kabat) | |
| | SEQ ID NO: 51 | LCDR1 | RSSTGAVTTSNYAN |
| | | (Kabat) | |
| | SEQ ID NO: 52 | LCDR2 | GTNKRAP |
| | | (Kabat) | |
| | SEQ ID NO: 53 | LCDR3 | ALWYSNLWV |
| | | (Kabat) | |
| | SEQ ID NO: 72 | VH | EVQLVESGGGLVQPGGSLRLSCAASGFTFNTYA |
| | | AA | MNWVRQAPGKGLEWVGRIRSKYNNYATYYADS |
| | | | VKGRFTISRDDAKNSLYLQMNSLRAEDTAVYYC |
| | | | VRHGNFGNSYVSWFAYWGQGTLVTVSS |
| | SEQ ID NO: 68 | VL | EIVVTQSPATLSVSPGERATLSCRSSTGAVTTSNY |
| | | AA | ANWVQQKPGQAPRGLIGGTNKRAPGIPARFSGS |
| | | | LSGDEATLTISSLQSEDFAVYYCALWYSNLWVFG |
| | | | GGTKVEIK |
| | SEQ ID NO: 73 | scFv | EVQLVESGGGLVQPGGSLRLSCAASGFTFNTYA |
| | | AA | MNWVRQAPGKGLEWVGRIRSKYNNYATYYADS |
| | | | VKGRFTISRDDAKNSLYLQMNSLRAEDTAVYYC |
| | | | VRHGNFGNSYVSWFAYWGQGTLVTVSSGGGGS |
| | | | GGGGSGGGGSEIVVTQSPATLSVSPGERATLSCRS |
| | | | STGAVTTSNYANWVQQKPGQAPRGLIGGTNKRA |
| | | | PGIPARFSGSLSGDEATLTISSLQSEDFAVYYCAL |
| | | | WYSNLWVFGGGTKVEIK |
| | SEQ ID NO: 74 | scFv | GAGGTGCAGCTGGTGGAGTCCGGCGGTGGCCT |
| | | DNA | TGTGCAGCCCGGCGGCAGTCTGAGACTGAGCT |
| | | | GCGCCGCTAGCGGCTTCACCTTCAACACCTAC |
| | | | GCCATGAACTGGGTGAGACAAGCCCCCGGCAA |
| | | | GGGCCTGGAGTGGGTGGGTAGAATCAGAAGC |
| | | | AAGTACAACAACTACGCCACCTACTACGCCGA |
| | | | CAGCGTGAAGGGCAGATTCACCATCAGCAGAG |
| | | | ACGACGCCAAGAACAGCCTGTACCTGCAGATG |
| | | | AACAGCCTGAGAGCCGAGGACACCGCCGTGT |
| | | | ACTACTGCGTTAGACACGGCAACTTCGGCAAC |
| | | | AGCTACGTGAGCTGGTTCGCCTACTGGGGCCA |
| | | | AGGCACCCTGGTAACGGTGAGCAGCGGCGGC |
| | | | GGTGGCAGCGGCGGAGGCGGCTCTGGCGGCG |
| | | | GAGGATCCGAAATCGTGGTGACCCAAAGTCCC |
| | | | GCGACCCTTAGCGTAAGCCCCGGCGAGAGAGC |
| | | | TACCCTGAGCTGCAGAAGCAGCACCGGCGCCG |
| | | | TGACCACAAGCAACTACGCCAACTGGGTGCAA |
| | | | CAGAAGCCCGGGCAGGCGCCCCGCGGCCTGAT |
| | | | CGGCGGTACCAACAAGAGAGCCCCCGGCATCC |
| | | | CCGCTAGATTCAGCGGCAGCCTGAGCGGCGAC |
| | | | GAGGCCACCCTGACCATCAGCAGCCTGCAGAG |
| | | | CGAGGACTTCGCCGTCTACTACTGCGCCCTGTG |
| | | | GTACAGCAACCTGTGGGTGTTCGGCGGCGGCA |
| | | | CCAAGGTGGAGATCAAG |
| BG564P | SEQ ID NO: 48 | HCDR1 | TYAMN |
| | | (Kabat) | |
| | SEQ ID NO: 71 | HCDR2 | RIRSKYNNYATYYADSVKG |
| | | (Kabat) | |
| | SEQ ID NO: 75 | HCDR3 | HGNFGSSYVSWFAY |
| | | (Kabat) | |
| | SEQ ID NO: 51 | LCDR1 | RSSTGAVTTSNYAN |
| | | (Kabat) | |
| | SEQ ID NO: 52 | LCDR2 | GTNKRAP |
| | | (Kabat) | |
| | SEQ ID NO: 53 | LCDR3 | ALWYSNLWV |
| | | (Kabat) | |
| | SEQ ID NO: 76 | VH | EVQLVESGGGLVQPGGSLRLSCAASGFTFSTYAM |
| | | AA | NWVRQAPGKGLEWVGRIRSKYNNYATYYADSV |
| | | | KGRFTISRDDAKNSLYLQMNSLRAEDTAVYYCV |
| | | | RHGNFGSSYVSWFAYWGQGTLVTVSS |
| | SEQ ID NO: 68 | VL | EIVVTQSPATLSVSPGERATLSCRSSTGAVTTSNY |
| | | AA | ANWVQQKPGQAPRGLIGGTNKRAPGIPARFSGS |
| | | | LSGDEATLTISSLQSEDFAVYYCALWYSNLWVFG |
| | | | GGTKVEIK |
| | SEQ ID NO: 77 | scFv | EVQLVESGGGLVQPGGSLRLSCAASGFTFSTYAM |
| | | AA | NWVRQAPGKGLEWVGRIRSKYNNYATYYADSV |
| | | | KGRFTISRDDAKNSLYLQMNSLRAEDTAVYYCV |
| | | | RHGNFGSSYVSWFAYWGQGTLVTVSSGGGGSG |
| | | | GGGSGGGGSEIVVTQSPATLSVSPGERATLSCRSS |
| | | | TGAVTTSNYANWVQQKPGQAPRGLIGGTNKRAP |
| | | | GIPARFSGSLSGDEATLTISSLQSEDFAVYYCALW |
| | | | YSNLWVFGGGTKVEIK |
| | SEQ ID NO: 78 | scFv | GAGGTGCAGCTGGTGGAGTCCGGCGGTGGCCT |
| | | DNA | TGTGCAGCCCGGCGGCAGTCTGAGACTGAGCT |
| | | | GCGCCGCTAGCGGCTTCACCTTCAGCACCTAC |
| | | | GCCATGAACTGGGTGAGACAAGCCCCCGGCAA |
| | | | GGGCCTGGAGTGGGTGGGTAGAATCAGAAGC |
| | | | AAGTACAACAACTACGCCACCTACTACGCCGA |
| | | | CAGCGTGAAGGGCAGATTCACCATCAGCAGAG |
| | | | ACGACGCCAAGAACAGCCTGTACCTGCAGATG |
| | | | AACAGCCTGAGAGCCGAGGACACCGCCGTGT |
| | | | ACTACTGCGTTAGACACGGCAACTTCGGCAGC |
| | | | AGCTACGTGAGCTGGTTCGCCTACTGGGGCCA |
| | | | AGGCACCCTGGTAACGGTGAGCAGCGGCGGC |
| | | | GGTGGCAGCGGCGGAGGCGGCTCTGGCGGCG |
| | | | GAGGATCCGAAATCGTGGTGACCCAAAGTCCC |
| | | | GCGACCCTTAGCGTAAGCCCCGGCGAGAGAGC |
| | | | TACCCTGAGCTGCAGAAGCAGCACCGGCGCCG |
| | | | TGACCACAAGCAACTACGCCAACTGGGTGCAA |
| | | | CAGAAGCCCGGGCAGGCGCCCCGCGGCCTGAT |
| | | | CGGCGGTACCAACAAGAGAGCCCCCGGCATCC |
| | | | CCGCTAGATTCAGCGGCAGCCTGAGCGGCGAC |
| | | | GAGGCCACCCTGACCATCAGCAGCCTGCAGAG |
| | | | CGAGGACTTCGCCGTCTACTACTGCGCCCTGTG |
| | | | GTACAGCAACCTGTGGGTGTTCGGCGGCGGCA |
| | | | CCAAGGTGGAGATCAAG |

TABLE 3
Sequences list (Kabat Numbering)

| | | | |
|---|---|---|---|
| BG143P | SEQ ID NO: 79 | Heavy-Knob | EVQLVESGGGLVQPGGSLRLSCAASGFTFSTYAM |
| | | chain AA | NWVRQAPGKGLEWVGRIRSKYNNYATYYADSV |
| | | | KGRFTISRDDAKNSLYLQMNSLRAEDTAVYYCV |
| | | | RHGNFGSSYVSWFAYWGQGTLVTVSSGGGGSG |
| | | | GGGSGGGGSEIVVTQSPATLSVSPGERATLSCRSS |
| | | | TGAVTTSNYANWVQQKPGQAPRGLIGGTNKRAP |
| | | | GIPARFSGSLSGDEATLTISSLQSEDFAVYYCALW |
| | | | YSNLWVFGGGTKVEIK |
| | | | EPKSSDKTHTCPPCPAPPAAGPSVFLFPPKPKDTL |
| | | | MISRTPEVTCVVVDVSHEDPEVKFNWYVDGVE |
| | | | VHNAKTKPREEQYNSTYRVVSVLTVLHQDWLN |
| | | | GKEYKCKVSNKALAAPIEKTISKAKGQPREPQV |
| | | | YTLPPSRDELTKNQVSLWCLVKGFYPSDIAVEWE |
| | | | SNGQPENNYKTTPPVLDSDGSFFLYSKLTVDKSR |
| | | | WQQGNVFSCSVMHEALHNHYTQKSLSLSPGK |
| BG143P | SEQ ID NO: 80 | Heavy-Knob | GAGGTGCAACTCGTCGAGAGCGGCGGGGGCC |
| | | chain DNA | TCGTGCAGCCTGGGGGCAGCCTGAGACTCAGC |
| | | | TGCGCCGCTAGCGGCTTCACCTTCAGCACCTAC |
| | | | GCCATGAACTGGGTGAGACAAGCCCCCGGCAA |
| | | | GGGCCTGGAGTGGGTGGGCAGAATCAGAAGC |
| | | | AAGTACAACAACTACGCCACCTACTACGCCGA |
| | | | CAGCGTGAAGGGCAGATTCACCATCAGCAGAG |
| | | | ACGACGCCAAGAACAGCCTGTACCTGCAGATG |
| | | | AACAGCCTGAGAGCCGAGGACACCGCCGTCTA |
| | | | CTACTGCGTGAGACACGGCAACTTCGGCAGCA |
| | | | GCTACGTGAGCTGGTTCGCTTATTGGGGGCAA |
| | | | GGCACACTGGTGACCGTCAGCAGCGGTGGAG |
| | | | GCGGTTCAGGCGGAGGTGGCTCTGGCGGTGGC |
| | | | GGATCGGAGATTGTGGTGACACAGAGCCCCGC |
| | | | CACCCTGAGCGTGAGCCCCGGCGAGAGAGCTA |
| | | | CACTGAGCTGCAGAAGCAGCACCGGCGCCGT |
| | | | GACCACAAGCAACTACGCCAACTGGGTGCAGC |
| | | | AGAAGCCCGGCCAAGCCCCTAGAGGCCTGATC |
| | | | GGGGGCACCAACAAGAGAGCCCCCGGCATCC |
| | | | CCGCTAGATTCTCCGGCAGCCTGAGCGGCGAC |
| | | | GAGGCCACCCTGACCATCAGCAGCCTGCAGAG |
| | | | CGAGGACTTCGCCGTGTACTACTGCGCCCTGT |
| | | | GGTACAGCAACCTGTGGGTGTTCGGCGGGGGC |
| | | | ACCAAGGTCGAGATCAAGGAGCCCAAAAGCA |
| | | | GCGATAAGACCCACACCTGTCCTCCCTGCCCC |
| | | | GCCCCTCCTGCCGCCGGCCCTAGCGTGTTCCTG |
| | | | TTCCCCCCCAAGCCCAAGGACACCCTGATGAT |
| | | | CAGCAGAACCCCCGAGGTGACCTGCGTGGTCG |
| | | | TGGACGTGAGCCACGAGGACCCCGAGGTGAA |
| | | | GTTCAACTGGTACGTGGACGGCGTGGAGGTGC |
| | | | ACAACGCCAAGACCAAGCCTAGAGAGGAGCA |
| | | | GTACAACAGCACCTACAGAGTGGTGAGCGTGC |
| | | | TGACCGTGCTGCACCAAGACTGGCTGAACGGC |
| | | | AAGGAGTACAAGTGCAAGGTGAGCAACAAGG |
| | | | CCCTGGCCGCCCCCATCGAGAAGACCATCAGC |
| | | | AAGGCCAAGGGGCAGCCTAGAGAGCCCCAAG |
| | | | TGTACACCCTGCCCCCTAGCAGAGACGAGCTC |
| | | | ACCAAGAACCAAGTGAGCCTGTGGTGCCTGGT |
| | | | GAAGGGCTTCTACCCTAGCGACATCGCCGTGG |
| | | | AGTGGGAGAGCAACGGGCAGCCCGAGAACAA |
| | | | CTACAAGACCACCCCCCCCGTGCTGGACAGCG |
| | | | ACGGCAGCTTCTTCCTGTACAGCAAGCTGACC |
| | | | GTGGACAAGAGCAGATGGCAGCAAGGCAACG |
| | | | TGTTCAGCTGCAGCGTGATGCACGAGGCCCTG |
| | | | CACAACCACTACACACAGAAGAGCCTGAGCCT |
| | | | GAGCCCCGGCAAGTGA |
| BG143P | SEQ ID NO: 81 | Heavy- Hole | EVQLQESGGGLVQPGGSLRLSCAASGYSFSDYHI |
| | | chain AA | NWVRQAPGKGLEWVGYIYPGSGHTKYNEKFKG |
| | | | RFTISRDTSSNTAYLQMNSLRAEDTAVYYCARSG |
| | | | IYHYVSSYYFDHWGQGTLVTVSSASTKGPSVFPL |
| | | | APSSKSTSGGTAALGCLVKDYFPEPVTVSWNSG |
| | | | ALTSGVHTFPAVLQSSGLYSLSSVVTVPSSSLGTQ |
| | | | TYICNVNHKPSNTKVDKKVEPKSCDKTHTCPPC |
| | | | PAPPAAGPSVFLFPPKPKDTLMISRTPEVTCVVVD |
| | | | VSHEDPEVKFNWYVDGVEVHNAKTKPREEQYN |
| | | | STYRVVSVLTVLHQDWLNGKEYKCKVSNKALA |
| | | | APIEKTISKAKGQPREPQVYTLPPSRDELTKNQVS |
| | | | LSCAVKGFYPSDIAVEWESNGQPENNYKTTPPVL |
| | | | DSDGSFFLVSKLTVDKSRWQQGNVFSCSVMHEA |
| | | | LHNHYTQKSLSLSPGK |
| BG143P | SEQ ID NO: 82 | Heavy Hole | GAAGTGCAGCTGCAAGAGTCCGGGGGGGGGC |
| | | chain DNA | TCGTGCAACCCGGGGGCTCCCTGAGACTGAGC |
| | | | TGCGCCGCTAGCGGCTACAGCTTCAGCGACTA |
| | | | CCACATCAACTGGGTGAGACAAGCCCCCGGCA |
| | | | AGGGCCTGGAGTGGGTGGGCTACATCTACCCC |
| | | | GGCAGCGGCCACACCAAGTACAACGAGAAGT |
| | | | TCAAGGGCAGATTCACCATCAGCAGAGACACA |
| | | | AGCAGCAACACCGCCTACCTGCAGATGAACAG |
| | | | CCTGAGAGCCGAGGACACCGCCGTGTACTACT |
| | | | GCGCTAGAAGCGGCATCTACCACTACGTGAGC |
| | | | AGCTACTACTTCGACCACTGGGGCCAAGGCAC |
| | | | CCTGGTGACAGTGTCCTCCGCCTCCACAAAGG |
| | | | GGCCTAGCGTGTTCCCCCTGGCCCCTAGCAGC |
| | | | AAGAGCACAAGCGGCGGCACCGCCGCCCTCG |
| | | | GCTGCCTGGTGAAGGACTATTTCCCCGAGCCC |
| | | | GTGACCGTGAGCTGGAACAGCGGCGCCCTCAC |
| | | | AAGCGGCGTGCACACCTTCCCCGCCGTGCTGC |
| | | | AGAGCAGCGGCCTGTACAGCCTGAGCAGCGTG |
| | | | GTGACCGTGCCTAGCAGCAGCCTGGGCACACA |
| | | | GACCTACATCTGCAACGTGAACCACAAGCCTA |
| | | | GCAACACCAAGGTGGACAAGAAGGTGGAGCC |
| | | | TAAGAGCTGCGATAAGACCCACACCTGCCCCC |
| | | | CTTGTCCCGCTCCCCCTGCTGCCGGGCCTAGCG |
| | | | TGTTCCTGTTTCCTCCCAAGCCCAAGGACACC |
| | | | CTGATGATCAGCAGAACCCCCGAGGTGACCTG |
| | | | CGTGGTCGTGGACGTGAGCCACGAGGACCCCG |
| | | | AGGTGAAGTTCAACTGGTACGTGGACGGCGTG |
| | | | GAGGTGCACAACGCCAAGACCAAGCCTAGAG |
| | | | AGGAGCAGTACAACAGCACCTACAGAGTGGTG |
| | | | AGCGTGCTGACCGTGCTGCACCAAGACTGGCT |
| | | | GAACGGCAAGGAGTACAAGTGCAAGGTGAGC |
| | | | AACAAGGCCCTGGCCGCCCCCATCGAGAAGAC |
| | | | CATCAGCAAGGCCAAGGGGCAGCCTAGAGAG |
| | | | CCCCAAGTGTACACCCTGCCCCCTAGCAGAGA |
| | | | CGAGCTGACCAAGAACCAAGTGAGCCTGTCCT |
| | | | GCGCCGTCAAGGGCTTCTACCCTAGCGACATC |
| | | | GCCGTGGAGTGGGAGAGCAACGGGCAGCCCG |
| | | | AGAACAACTACAAGACCACCCCCCCCGTGCTG |
| | | | GACAGCGACGGCAGCTTCTTCCTGGTGAGCAA |
| | | | GCTGACCGTGGACAAGAGCAGATGGCAGCAA |
| | | | GGCAACGTGTTCAGCTGCAGCGTGATGCACGA |
| | | | GGCCCTGCACAACCACTACACACAGAAGAGCC |
| | | | TGAGCCTGAGCCCCGGCAAGTGA |
| BG143P | SEQ ID NO: 83 | Kappa chain | DIQMTQSPSSLSASVGDRVTITCKASQDAGSAVA |
| | | AA | WYQQKPGKAPKLLIYWASTRHTGVPSRFSGSGS |
| | | | GTDFTLTISSLQPEDFATYYCQQYSSYTFGGGTKL |
| | | | EIKRTVAAPSVFIFPPSDEQLKSGTASVVCLLNNF |
| | | | YPREAKVQWKVDNALQSGNSQESVTEQDSKDS |
| | | | TYSLSSTLTLSKADYEKHKVYACEVTHQGLSSPV |
| | | | TKSFNRGEC |
| BG143P | SEQ ID NO: 84 | Kappa chain | GACATTCAGATGACACAGAGCCCTAGCAGCCT |
| | | DNA | GTCCGCTAGCGTGGGCGACAGAGTGACCATCA |
| | | | CCTGCAAGGCTAGCCAAGACGCCGGGAGCGC |
| | | | CGTGGCCTGGTATCAGCAGAAGCCCGGCAAGG |
| | | | CCCCCAAGCTGCTGATCTACTGGGCTAGCACA |
| | | | AGACACACCGGCGTGCCTAGCAGATTCAGCGG |
| | | | CAGCGGCAGCGGCACCGACTTCACCCTGACCA |
| | | | TCAGCAGCCTGCAGCCCGAGGACTTCGCCACC |
| | | | TACTACTGTCAGCAGTACAGCAGCTACACCTTC |
| | | | GGCGGGGGCACCAAGCTGGAGATCAAGCGTA |
| | | | CGGTGGCTGCACCATCTGTCTTCATCTTCCCGC |
| | | | CATCTGATGAGCAGTTGAAATCTGGAACTGCCT |
| | | | CTGTTGTGTGCCTGCTGAATAACTTCTATCCCA |
| | | | GAGAGGCCAAAGTACAGTGGAAGGTGGATAA |
| | | | CGCCCTCCAATCGGGTAACTCCCAGGAGAGTG |
| | | | TCACAGAGCAGGACAGCAAGGACAGCACCTA |
| | | | CAGCCTCAGCAGCACCCTGACGCTGAGCAAAG |
| | | | CAGACTACGAGAAACACAAAGTCTACGCCTGC |
| | | | GAAGTCACCCATCAGGGCCTGAGCTCGCCCGT |
| | | | CACAAAGAGCTTCAACAGGGGAGAGTGTTGA |
| Linker | SEQ ID NO: 85 | AA | GGGGSGGGGSGGGGSGGGGS |
| Linker | SEQ ID NO: 86 | AA | GGGGSGGGGSGGGGS |
| CLDN6 | SEQ ID NO: 87 | AA | MASAGMQILGVVLTLLGWVNGLVSCALPMWK |
| | | | VTAFIGNSIVVAQVVWEGLWMSCVVQSTGQMQ |
| | | | CKVYDSLLALPQDLQAARALCVIALLVALFGLLV |
| | | | YLAGAKCTTCVEEKDSKARLVLTSGIVFVISGVL |
| | | | TLIPVCWTAHAIIRDFYNPLVAEAQKRELGASLYL |
| | | | GWAASGLLLLGGGLLCCTCPSGGSQGPSHYMAR |
| | | | YSTSAPAISRGPSEYPTKNYV |
| CLDN9 | SEQ ID NO: 88 | AA | MASTGLELLGMTLAVLGWLGTLVSCALPLWKV |
| | | | TAFIGNSIVVAQVVWEGLWMSCVVQSTGQMQC |
| | | | KVYDSLLALPQDLQAARALCVIALLLALLGLLV |
| | | | AITGAQCTTCVEDEGAKARIVLTAGVILLLAGILV |
| | | | LIPVCWTAHAIIQDFYNPLVAEALKRELGASLYL |
| | | | GWAAAALLMLGGGLLCCTCPPPQVERPRGPRLG |
| | | | YSIPSRSGASGLDKRDYV |

### EXAMPLES

### Example 1. Generation of Mouse Anti-CLDN6 Antibodies

To generate antibodies against CLDN6, cohorts of 20-25 BALB/C, SJL strains of inbred mice were immunized with human CLDN6 overexpression cells (L929/human CLDN6, internally prepared), each cohort being subjected to an immunization strategy comprising a unique combination of CLDN6 antigens, dose, injection route, adjuvant and immunization timing. A total of 5 animals in 4-5 cohorts were immunized. Animals received immunizations over varying periods between 0 and 56 days. To monitor immune responses, titrated serum was screened by FACS, typically after 21-56 days of 2-4 immunizations. Serum was screened for antibody binding to CLDN6 overexpression cells CHOK1/human CLDN6. CLDN6-specific antibody responses were measured in each animal, and animals with sufficient titers of anti-CLDN6 Ig were selected for final boosts for 4 days.

Lymphoid organs, including spleens and lymph nodes, were isolated from mice immunized as described above. Hybridomas were generated by fusions with immortalized mouse myeloma cells derived from the SP2/0 by PEG based fusion. The resulting cells were plated in 96 well cell culture plates using regular 1640 medium supplemented with HAT for selection of hybridomas.

### Example 2. Screening and Selection of Anti-CLDN6 Antibodies

Hybridomas were generated as described in Example 1. After 10-13 days of culture and growth media replacement, hybridoma culture supernatants were collected from individual wells and screened to identify wells with secreted CLDN6-specific antibodies. All supernatants were initially screened against at least two overexpression cell lines, including CHOK1/human CLDN6 and CHOK1/human CLDN9 (internally prepared). Antibody binding on overexpression cell lines were measured by FACS. Supernatants from over approximately 20000 culture wells in 4 hybridoma fusions were screened for CLDN6 antibodies. Briefly, 100 μL of hybridoma culture supernatant and a CLDN6-expressing cancer cell line (such as PA-1 or CHOK1/human CLDN6 stable cell lines) or control cells (such as parental CHOK1) were co-incubated for 30-60 min, washed, and incubated with anti-mouse IgG Fc secondary antibody conjugated to APC. After incubation and washing, fluorescence was measured by flow cytometry.

Hybridomas from positive wells were transferred to 24-well plates with fresh culture media to grow for 2-3 days before screening again by flow cytometry to confirm antibody binding to the cyno CLDN6 overexpression cell line and the human CLDN6 positive cancer cell line (PA-1). Antibody binding to the cyno CLDN6 overexpression cell line and the human CLDN6 positive cancer cell line (PA-1) was measured by flow cytometry. Briefly, 100 μL of hybridoma culture supernatant and CLDN-expressing cancer cell line (such as PA-1 or CHOK1/human CLDN6 and CHOK1/human CLDN9 stable cell lines) or control cells (such as parental CHOK1) were co-incubated for 30-60 min, washed, and incubated with anti-mouse IgG Fc secondary Ab conjugated to APC. After incubation and washing, fluorescence was measured by flow cytometry.

### Example 3. Subcloning of Selected CLDN6 Ab-Secreting Hybridomas

Selected CLDN6 antibody-secreting hybridomas were subcloned once or twice to ensure monoclonality. Briefly, approximately 80-100 viable hybridoma cells were plated in 3 mL of semi-solid methylcellulose medium (Stem Cell Technologies) in 6 well plate. After 7-10 days, hybridoma colonies arising from single cells as visible clones were picked to 96-well plate, and were further cultured for 2-4 days in fresh medium. Culture supernatant was screened by ELISA and flow cytometry as previously described to confirm human and cyno CLDN6 binding. Stable hybridoma subclones were cultured in vitro for cell cryopreservation, antibody producing and antibody VH and VL gene cloning and sequencing.

### Example 4. Determination of CLDN6 Binding EC50 Values for Mouse Anti-CLDN6 Antibodies

The selected anti-CLND6 antibody-secreting hybridomas after subcloning were plated in T75 flask with 40 ml fresh 1640 medium supplemented with 2% FBS for the antibody producing. After 7-10 days of culture, the hybridoma supernatants were harvested for the antibody purification using protein A column. Then binding activity of mouse anti-CLDN6 antibodies to CLDN6 positive cells were characterized using flow cytometry. The EC50 values of clone BG87P are presented in Tables 4-6. The data indicates that clone BG87P binds to human CLDN6 but not to human CLDN9. In addition, BG87P binds to mouse CLDN6 and cyno CLDN6.

TABLE 4
Binding activity of BG87P to CHOK1 stable cells expressing human CLDN6 and human CLDN9

| | CHOK1/human | CHOK1/human | CHOK1/human | CHOK1/human |
|---|---|---|---|---|
| | CLDN6-GFP binding | CLDN6-GFP binding | CLDN9-GFP binding | CLDN9-GFP binding |
| Clones | EC50 (nM) | Emax (MFI) | EC50 (nM) | Emax (MFI) |
| BG87P | 3.41 | 26576 | — | 30.7 |
| Human IgG1 | — | 31.1 | — | 26.9 |
| isotype Ctrl. | | | | |

TABLE 5
Cross-species binding activity of BG87P to mouse CLDN6 and cyno CLDN6

| | CHOK1/mouse | CHOK1/mouse | CHOK1/Cyno | CHOK1/Cyno |
|---|---|---|---|---|
| | CLDN6-GFP binding | CLDN6-GFP binding | CLDN6-GFP binding | CLDN6-GFP binding |
| Clones | EC50 (nM) | Emax (MFI) | EC50 (nM) | Emax (MFI) |
| BG87P | 94.69 | 27700 | 6.08 | 43400 |
| Human IgG1 | — | 41.8 | — | 225 |
| isotype Ctrl. | | | | |

TABLE 6
Binding activity of BG87P to cancer cell line
PA-1 with endogenous CLDN6 expression

| | PA-1 binding | PA-1 binding |
|---|---|---|
| Clones | EC50 (nM) | Emax (MFI) |
| BG87P | 3.38 | 74200 |
| Human IgG1 | — | 34 |
| isotype Ctrl. | | |

### Example 5. Antibody VH and VL Gene Cloning and Sequencing

After the removal of the supernatant, selected CLDN6 antibody-secreting hybridomas after subcloning were lysed by adding 100 mL RLT buffer in 96-well round bottom plates. The mRNA containing lysates were subsequently transferred to 96-well deep well plates for mRNA isolation, cDNA synthesis and DNA sequencing by standard sequencing techniques (Sanger sequencing and next generation sequencing). In general, total RNA of the cell lysates was prepared using the Total RNA Isolation Kit according to the manufacturer's instructions. The cDNA was generated by reverse transcription of the mRNA using the Super Script III first-strand synthesis SuperMix (Invitrogen®) according to the manufacturer's instructions. The nucleic acid and amino acid sequences of BG87P are shown in Table 1 (SEQ ID NO: 1-10).

Generation of Chimeric BG87P Antibody (chBG87P)

The ChBG87P antibody was generated by subcloning variable region of mouse BG87P (SEQ ID NO: 7 and 8) into an in-house developed expression vectors which contains constant regions of a human wildtype IgG1 and kappa chain. The antibody was expressed by co-transfection of the above two constructs into HEK293T cells and purified using a protein A column (Cat: 17-5438-02, GE Life Sciences®). The purified chimeric antibody was concentrated to 0.5-10 mg/ml in PBS and stored in aliquots in −80° C.-Freezer.

### Example 6. Humanization of Anti-CLDN6 Chimeric BG87P Antibody (chBG87P)

### Humanization Approach

For humanization of chBG87P, human germline IgG genes were searched for sequences that share high degrees of homology to the protein sequences of chBG87P variable regions by sequence comparison against the human immunoglobulin gene database in IMGT. The human IGHV and IGKV genes that are present in human antibody repertoires with high frequencies and highly homologous to chBG87P were selected as the templates for humanization.

### Design of Humanization Variants

Humanization was carried out by CDR-grafting following with critical back mutations incorporated. The humanized antibodies were engineered as human IgG1 wild type format by using an in-house developed expression vector. In the initial first round of humanization, mutations from murine variable region to human amino acid residues in framework regions were guides by 3D structures analysis and the murine framework residues with structural importance for maintaining the canonical structural of CDRs were retained in the first round of the humanization design. Five back mutations on heavy chain and three mutations on light chain were selected and performed single point mutation for exploration of critical back mutations: BG87P-Bz1 (VH SEQ ID NO: 15 and VL: SEQ ID NO: 14), BG87P-Bz2 (VH SEQ ID NO: 16 and VL: SEQ ID NO: 14), BG87P-Bz3 (VH SEQ ID NO: 17 and VL: SEQ ID NO: 14), BG87P-Bz4 (VH SEQ ID NO: 18 and VL: SEQ ID NO: 14), BG87P-Bz5 (VH SEQ ID NO: 19 and VL: SEQ ID NO: 14), BG87P-B26 (VH SEQ ID NO: 13 and VL: SEQ ID NO: 20), BG87P-Bz7 (VH SEQ ID NO: 13 and VL: SEQ ID NO: 20), and BG87P-Bz8 (VH SEQ ID NO: 13 and VL SEQ ID NO: 22). BG87P-Bz0 (VH: SEQ ID NO: 13 and VL: SEQ ID NO: 14) is the variant with all theoretical back mutations incorporated and the binding capacity of BG87P-B20 should be comparable to parental chBG87P. Comparison of the binding data reveals which back mutations affect binding significantly. Specifically, LCDRs of chBG87P (SEQ ID NO: 4 to 6) were grafted into the framework of human germline variable gene IGKV1-5 and 01-IGKJ4*01 with A43S, L78V and Y87F murine framework residues (resulted as SEQ ID NO: 14). HCDRs of chBG87P (SEQ ID NO: 1 to 3) were grafted into the framework of human germline variable gene IGHV1-3 and 01-JH6c with V21, T28S, 169L, R71V and Y91F murine framework residues retained (resulted as SEQ ID NO: 13); BG87P-20 (VH: SEQ ID NO: 11 and VL: SEQ ID NO: 12) is the resulted humanized variant with above HCDRs and LCDRs grafting however without any back mutations from murine VH and VL framework.

Expression and Purification of chBG87P and Humanized Antibodies

All first round of BG87P humanized variants (BG87P-20, BG87P-B20, BG87P-Bz1, BG87P-Bz2, BG87P-Bz3, BG87P-B24, BG87P-B25, BG87P-B26, BG87P-B27, and BG87P-Bz8) were constructed as a humanized full-length antibody using in-house developed expression vectors that contain constant regions of a human wildtype IgG1 and kappa chain, respectively, with easy adapting sub-cloning sites. All humanized variants were expressed by co-transfection of the above two constructs into HEK293T cells and purified using a protein A column (Cat: 17-5438-02, GE Life Sciences). The purified antibody was concentrated to 0.5-10 mg/ml in PBS and stored in aliquots in −80° C. Freezer.

Determination of Cell Binding Activity of 1^{st} Round of Humanized BG87P Variants (hBG87P) and PTM Removal Variants

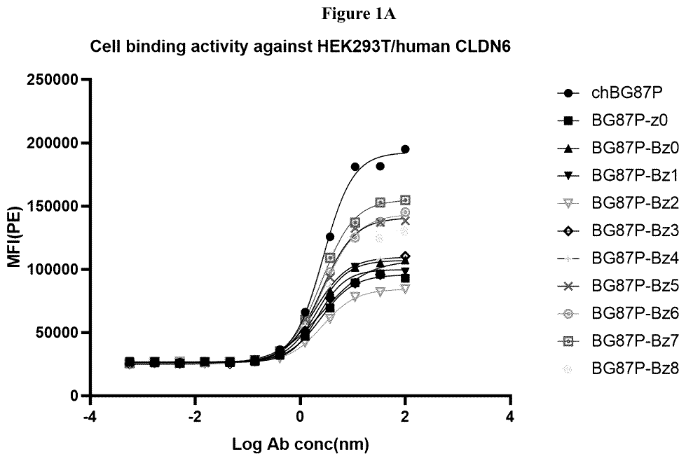

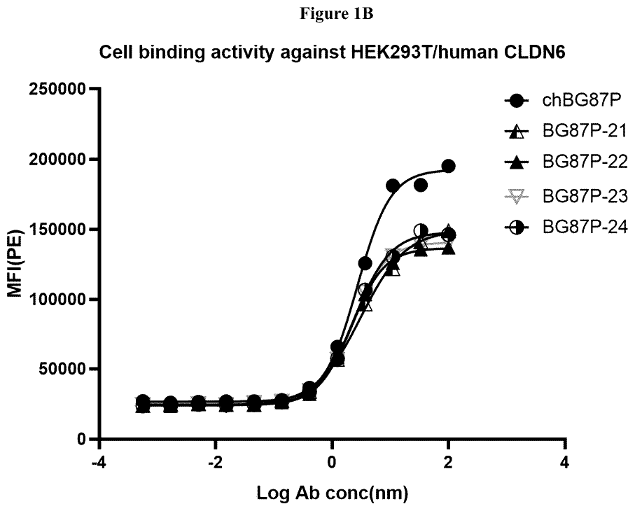

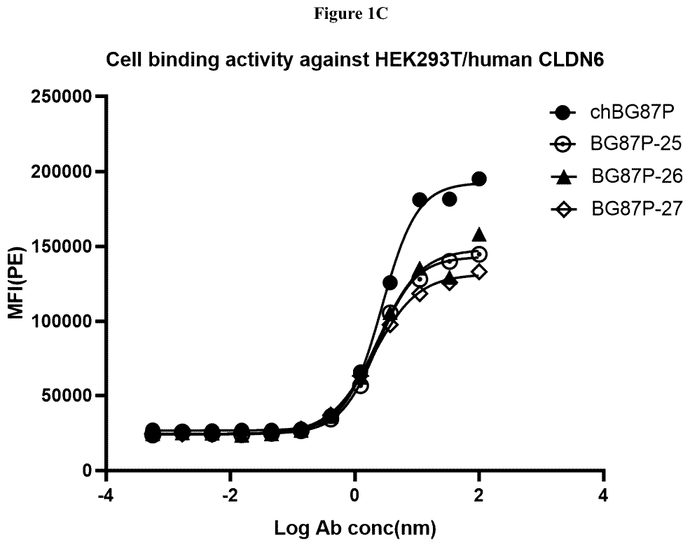

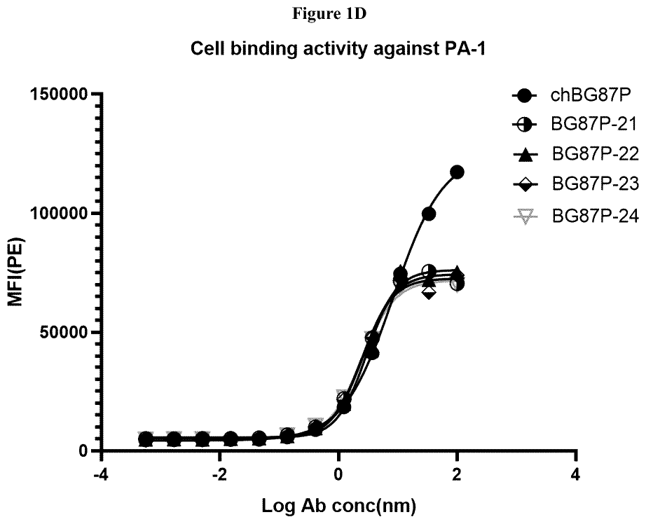

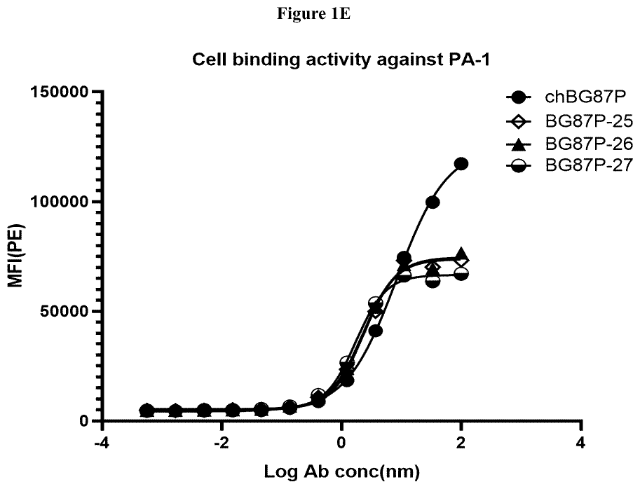

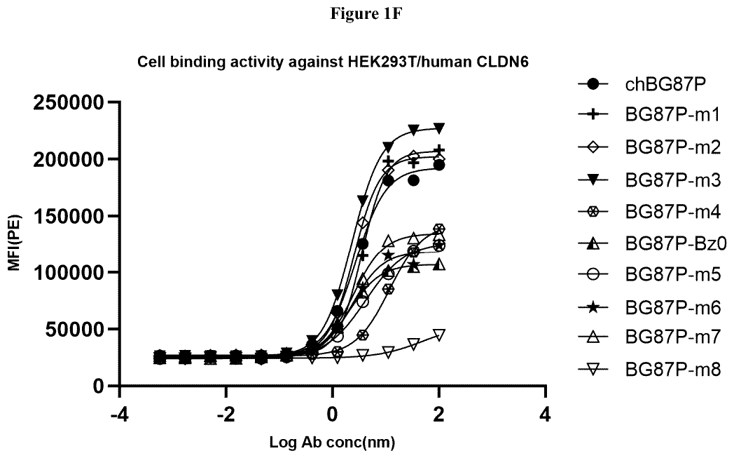

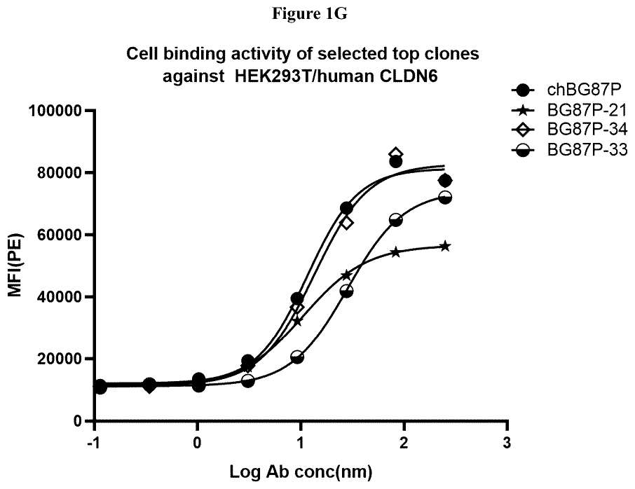

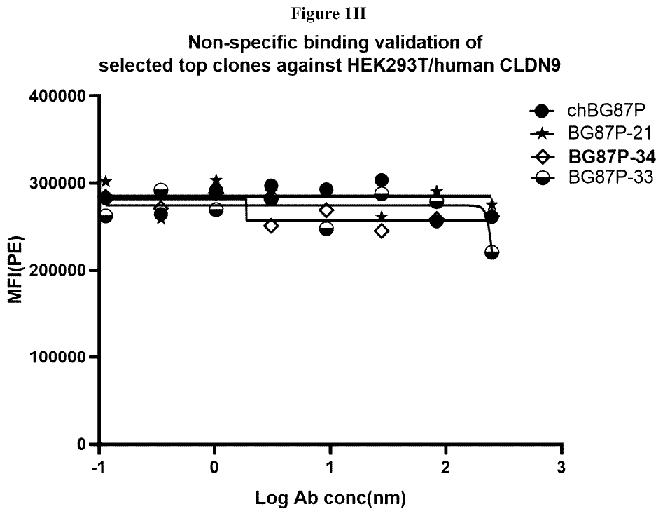

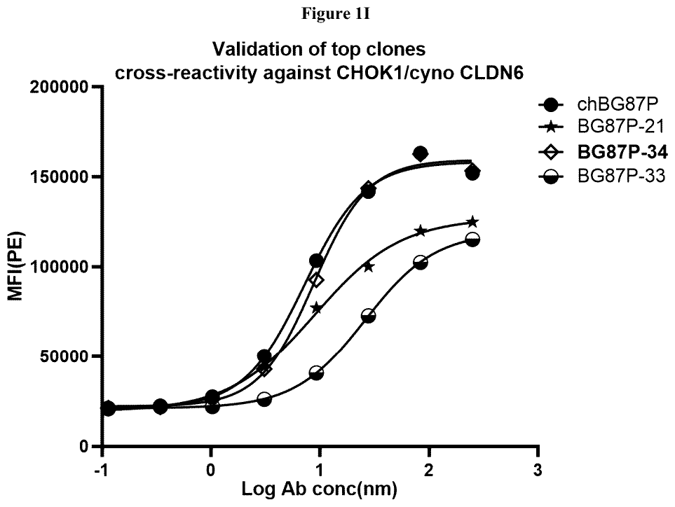

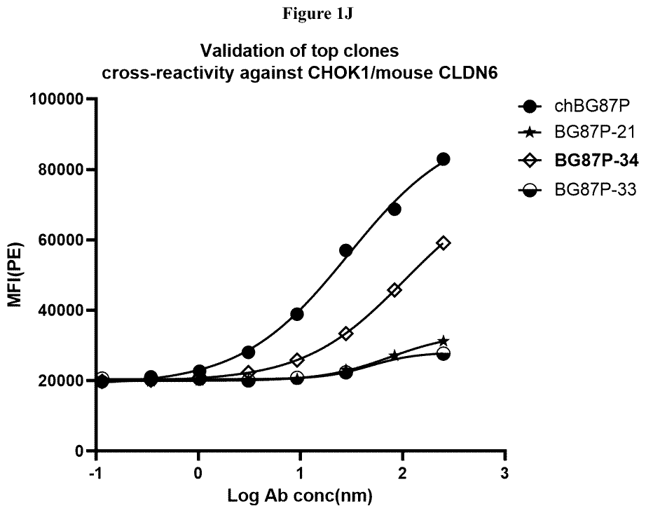

For affinity determination, CLDN6 over-expressing HEK293T cells and cancer cell line PA-1 which express high levels of human CLDN6 were used to evaluate the binding activity of BG87P related engineering variants. Live cells were seeded in 96-well plate, and were incubated with a series of dilutions of chBG87P and engineered variants thereof. Goat anti-human IgG was used as second antibody to detect antibody binding to the cell surface. EC50 values for dose-dependent binding to CLDN6 expressing cell lines were determined by fitting the dose-response data to the four-parameter logistic model with GraphPad Prism. Cell binding activity of 1^{st} round of BG87P humanization variants against HEK293T/human CLDN6 were compared to chBG87P and shown in FIG. 1A. Cell binding affinity (EC50) and Emax (MFI) of 1^{st} round of humanized variants was normalized to chBG87P for directly comparison and ranking (Table 7).

Beginning with the chBG87P antibody and BG87P-Bz0, several additional amino acid changes in the CDR regions of both VH and VL were made to further improve the biophysical properties for therapeutics use in human. The considerations included removing post translational modifications (PTM), improved heat stability (Tm), while maintaining binding activities, the resulted variants are BG87P-m1 (VH SEQ ID NO: 31 and VL SEQ ID NO: 8), BG87P-m2 (VH SEQ ID NO: 32 and VL SEQ ID NO: 8), BG87P-m3 (VH SEQ ID NO: 33 and VL SEQ ID NO: 8), and BG87P-m4 (VH SEQ ID NO: 34 and VL SEQ ID NO: 8) and BG87P-m5 (VH SEQ ID NO: 35 and VL SEQ ID NO: 14), BG87P-m6 (VH SEQ ID NO: 36 and VL SEQ ID NO 14), BG87P-m7 (VH SEQ ID NO: 37 and VL SEQ ID NO: 14), and BG87P-m8 (VH SEQ ID NO: 38 and VL SEQ ID NO: 14). Cell binding activity of chBG87P and BG87P-Bz0 related PTM removal variants against HEK293T/human CLDN6 was compared with chBG87P and BG87-Bz0, respectively (FIG. 1F). Cell binding affinity (EC50) and Emax (MFI) was normalized to chBG87P for directly comparison and ranking (Table 7). The results demonstrated that except for the mutation of H33A which resulted as BG87P-m4 and BG87P-m8, other replacements of potential detrimental residues maintained the binding capacity of respective parental ones.

TABLE 7
Summary of cell binding activity of humanized and PTM
removal variants against CLDN6 over-expressing HEK293T
HEK293T-human CLDN6

| | | | Ranking by Emax | | | |
|---|---|---|---|---|---|---|
| Ranking by Ec50 | | | | | | Emax |
| Antibody | Top MFI | | Antibody | Top MFI | | ratio to |
| name | Mean | Ec50 (nM) | name | Mean | Ec50 (nM) | BG87P |
| BG87P-Bz8 | 130650.51 | 1.71 | BG87P-m3 | 227257.19 | 2.32 | 1.18 |
| BG87P-Bz4 | 109529.57 | 1.88 | BG87P-m1 | 206987.02 | 3.56 | 1.08 |
| BG87P-27 | 131381.01 | 2 | BG87P-m2 | 202202.02 | 2.51 | 1.05 |
| BG87P-Bz0 | 106974.58 | 2.1 | chBG87P | 192332.83 | 2.72 | 1.00 |
| BG87P-22 | 136523.4 | 2.12 | BG87P-Bz7 | 154833.51 | 2.54 | 0.81 |
| BG87P-24 | 141013.73 | 2.16 | BG87P-21 | 149494.66 | 3.07 | 0.78 |
| BG87P-Bz1 | 99692.6 | 2.19 | BG87P-26 | 147913.6 | 2.3 | 0.77 |
| BG87P-26 | 147913.6 | 2.3 | BG87P-23 | 147866.77 | 2.43 | 0.77 |
| BG87P-m3 | 227257.19 | 2.32 | BG87P-Bz6 | 143672.43 | 2.79 | 0.75 |
| BG87P-25 | 142863.99 | 2.33 | BG87P-m4 | 143182.68 | 11.76 | 0.74 |
| BG87P-m6 | 118322.89 | 2.34 | BG87P-25 | 142863.99 | 2.33 | 0.74 |
| BG87P-m7 | 134492.69 | 2.43 | BG87P-24 | 141013.73 | 2.16 | 0.73 |
| BG87P-23 | 147866.77 | 2.43 | BG87P-Bz5 | 140664.98 | 2.55 | 0.73 |
| BG87P-z0 | 95645.46 | 2.45 | BG87P-22 | 136523.4 | 2.12 | 0.71 |
| BG87P-m2 | 202202.02 | 2.51 | BG87P-m7 | 134492.69 | 2.43 | 0.70 |
| BG87P-Bz7 | 154833.51 | 2.54 | BG87P-27 | 131381.01 | 2 | 0.68 |
| BG87P-Bz5 | 140664.98 | 2.55 | BG87P-Bz8 | 130650.51 | 1.71 | 0.68 |
| BG87P-Bz3 | 107318.29 | 2.63 | BG87P-m5 | 126460 | 4.19 | 0.66 |
| BG87P-Bz2 | 84347.71 | 2.65 | BG87P-m6 | 118322.89 | 2.34 | 0.62 |
| chBG87P | 192332.83 | 2.72 | BG87P-Bz4 | 109529.57 | 1.88 | 0.57 |
| BG87P-Bz6 | 143672.43 | 2.79 | BG87P-Bz3 | 107318.29 | 2.63 | 0.56 |
| BG87P-21 | 149494.66 | 3.07 | BG87P-Bz0 | 106974.58 | 2.1 | 0.56 |
| BG87P-m1 | 206987.02 | 3.56 | BG87P-Bz1 | 99692.6 | 2.19 | 0.52 |
| BG87P-m5 | 126460 | 4.19 | BG87P-z0 | 95645.46 | 2.45 | 0.50 |
| BG87P-m4 | 143182.68 | 11.76 | BG87P-Bz2 | 84347.71 | 2.65 | 0.44 |
| BG87P-m8 | 54013.49 | 48.74 | BG87P-m8 | 54013.49 | 48.74 | 0.28 |

TABLE 8
Cell binding activity of 2^{nd} round of humanized variants
which with PTM removal against cancer cell line PA-1
Cancer cell line: PA-1

| | | | Ranking by Emax | | | |
|---|---|---|---|---|---|---|
| Ranking by Ec50 | | | | | | Emax |
| Antibody | Top MFI | EC50 | Antibody | Top MFI | Ec50 | ratio to |
| name | Mean | (nM) | name | Mean | (nM) | BG87P |
| BG87P-27 | 66672.07 | 1.68 | BG87P | 124836.78 | 8.33 | 1.00 |
| BG87P-24 | 62436.19 | 2.27 | BG87P-21 | 76460.34 | 2.8 | 0.61 |
| BG87P-26 | 7BG143P0.80 | 2.30 | BG87P-25 | 73677.44 | 2.34 | 0.59 |
| BG87P-25 | 73677.44 | 2.34 | BG87P-23 | 72521.43 | 2.49 | 0.58 |
| BG87P-23 | 72521.43 | 2.49 | BG87P-26 | 68012.43 | 2.52 | 0.54 |
| BG87P-21 | 74335.52 | 2.62 | BG87P-22 | 67778.95 | 3.01 | 0.54 |
| BG87P-22 | 76120.75 | 3.00 | BG87P-27 | 66672.07 | 1.68 | 0.53 |
| BG87P-z0 | 53183.52 | 3.25 | BG87P-24 | 62436.19 | 2.27 | 0.50 |
| chBG87P | 124836.78 | 8.33 | BG87P-z0 | 53183.52 | 3.25 | 0.43 |

Determination of Cell Binding Activity of 2^{nd} Round of Critical Back Mutations Combining with PTM Removal Sites

After comprehensive analysis of EC50 and Emax of 1^{st} round humanization cell binding data (Table 7), four critical back mutation sites, VH: V21; VH: T28S; VH: 169L; VH: Y91F were identified and combined with PTM removal site for 2^{nd} round validation and determination of final humanization candidate. PTM removal mutation VH: V65G, which site with a higher popularity of G (G62%; V<1%) in human germline indicated a potential benefit in antibody framework stability, was involved in variant BG87P-m3, showed improved Emax and EC50 as compared to chBG87P in FIG. 1F and Table 7 which was incorporated in 2^{nd} round combination for further validation. The VH and VL sequences of resulted 2^{nd} round humanized variants BG87P-21, BG87P-22, BG87P-23, BG87P-24, BG87P-25, BG87P-26, and BG87P-27 are given in Table 1.

After identification of the cell binding activity of humanized combination variants as shown in FIG. 1B and FIG. 1C in HEK293T/human CLDN6 cells and FIG. 1D and FIG. 1E of PA-1 cells, BG87P-21 was selected as the top humanized candidate for further consideration (VH and VL amino acid sequences are SEQ ID NO: 24 and 12 respectively). BG87P-21 included critical back mutation site VH: T28S and PTM site VH: V65G, which revealed a comparable cell binding affinity as compared to chBG87P. The Emax was reduced of 22% in HEK293T/human CLDN6 and 40% in PA-1 (Table 7 and Table 8).

### Developability Evaluation of Humanized Anti-CLDN6 Antibodies

Biophysical properties were profiled for the identification of top humanized anti-CLDN6 antibody. The data indicates that BG87P-21 shows a moderate to high risk in hydrophobicity, subsequent of risk in self-interaction in PBS buffer which exhibited by the AC-SINS, B22KD and CIC readouts. (Table 9-Table 11).

TABLE 9
Biophysical properties analysis of BG87P-21 and chBG87P

| Sample name | | BG87P-21 | chBG87P | Threshold |
|---|---|---|---|---|
| Assay | HIC (min) | 21.9 | 25 | 21.1 |
| | CIC (min) | 15.5 | 14.7 | 14 |
| | KD in PBS (mL/g) | −20.1 | −15.6 | >−10 |
| | B22 in PBS | −3.10E−05 | −2.20E−05 | >0 |
| | AC-SINS in PBS | Δλ = 14.5 nm | Δλ = 6.0 nm | <10 nm |
| | Tm1 (° C.) | 69.26 | 70.17 | 65 |
| | Tagg (° C.) | 69.35 | 72.33 | 65 |
| | SEC-HPLC (%) | 98.42 | 92.52 | 0.95 |

For hydrophobicity assessment, 50 μg of sample at 1 mg/ml was diluted with a mobile phase A solution (1.5 M ammonium sulfate, 50 mM sodium phosphate, pH 7.0) to achieve a final ammonium sulfate concentration of about 1M before analysis. A MABPac HIC-10 column was used with a liner gradient of mobile phase A and mobile phase B solution (50 mM sodium phosphate, pH 7.0) over 29 min at a flow rate of 0.5 m1/min. Peak retention times were monitored at A280 absorbance. As indicated by Table 9, both chBG87P and BG87P-21 showed higher hydrophobicity performance and over the internal criterion 21.1 min in IgG format.

For thermostability assessment, the thermal stability of BG87P related engineering variants were described by the thermal unfolding transition midpoint Tm (° C.), which was measured by extrinsic fluorescence. The Tm was determined using QuantStudio™ 6 Flex System from Applied Biosystems. 20 μL of 1 mg/ml sample was mixed with 20 μL of 40XSYPRO orange. The plate was scanned from 25° C. to 95° C. at a rate of 0.9° C./min. The Tm was assigned using the first derivative of the raw data from the QuantStudio™ 6 Flex System Analysis software. The results were summarized in Table 9, which indicates both chBG87P and humanized variant BG87P-21 showed good thermostability.

For determination of the aggregation propensity of BG87P related engineering variants, static light scattering intensity was measured using a Uncle system (Unchained Labs). During the measurement, approximately 8.8 L protein sample at 1 mg/ml was loaded to the cuvette; the samples were held at 25° C. for 120 seconds, and then ramped to 95° C. at the rate of 0.3° C./min. The scattering data was collected at an angle of 90° with a laser wavelength of 266 nm. Tagg (aggregation temperature) were analyzed and calculated by Uncle Analysis Software. The results were summarized in Table 9. Both chBG87P and humanized variants showed acceptable Tagg.

CIC is a technique to identify antibody candidates with poor solubility or non-specific binding propensity. IgG from human serum or other ligands were chemically coupled to an NHS-activated chromatography resin. The retention times of proteins were tested on this resin using a HPLC to evaluate proteins solubility. After column coupling with IgG from human serum, The antibody sample and the sample buffer were diluted with mobile phase (PBS) to 0.1 mg/mL. The diluted sample and buffer were transferred to HPLC vial for LC-MS analysis. The result summarized in Table 9 indicates that both chBG87P and BG87P-21 show acceptable non-specific interaction with human IgG.

B22 and KD General description and intended use of the test method. The method is used to study weak protein-protein interactions, to predict the aggregation trend, to reveal the influence of formulation ingredients on intermolecular interactions and support the formulation buffer selection. Antibodies with buffer exchange samples were diluted to 1 mg/mL and centrifuge at 14000 rpm for 30 min, then check Tm, Tagg and DLS. Loading samples onto the Uni. 9 μL/well. Each sample set a duplicate hole. Set equipment parameters following the guide of Uncle and run the experiment. In this experiment we used B22 & Kd mode. Run information: Temperature (° C.): 25. Incubation Time (sec): 120. No. of Acquisitions: 4. Acquisition Time (sec): 5. Attenuator Control: Auto. Laser. Control: Auto Run. For kd: diffusion interaction parameter, if the interaction of proteins increases as the concentration increases (attracting each other), then the proteins behave as if they become larger and the diffusion coefficient (KD) decreases (negative slope). For B22: second virial coefficient, if protein interactions increase with increasing concentration (attracting each other), then proteins behave as if they are larger and 1/R90 decreasing (negative slope). The data indicated that in PBS, both chBG87P and humanized variants attract each other, tend to aggregate under this condition (Table 10).

AC-SINS is the assay to obtain the self-interaction for the sample to predict the aggregation possibility. It is based on concentrating antibodies from diluted solutions around gold nanoparticles pre-coated with polyclonal capture. Interactions between immobilized antibodies lead to reduced inter-particle distances and increased plasmon wavelengths (wavelength of maximum absorbance), which can be readily measured by optical means. Dilute the antibodies with offering buffer to 0.05 mg/mL, respectively. After gold nanoparticle preparation, a 9:1 volume ratio was used to mix gold nanoparticle solution with coating solution. After room temperature incubation for 1 h, thiolated PEG (final concentration 0.1 uM) was used to block empty sites in AuNP. Then incubation for another 1 h at room temperature. The particle solution was then centrifuged at 15000 rpm for 6 min. Upper solution discarded. Storage buffer at 1/10 of the starting volume was used to redissolve the particle. 10 μL of the concentrated coated particles was incubated with 100 μL of the test antibody solution at room temperature for 2 h in a polypropylene plate, then 90 μL of the resulting solution was transferred into a polystyrene UV transparent plate. The data indicated that both chBG87P and BG87P-21 showed suboptimal self-interaction propensity (Table 9).

TABLE 10
Self-interaction risk determination of
2^{nd} round humanized variants and chBG87P

| | kD | B₂₂ | k D Fit | B₂₂ Fit |
|---|---|---|---|---|
| Sample | (mL/g) | (mol*mL/g²) | Quality | Quality |
| BG87P-21 | −17.2 | −1.1E−05 | 0.97 | 0.96 |
| BG87P-22 | −22.7 | −2.5E−05 | 0.96 | 0.97 |
| BG87P-24 | −21.1 | −1.7E−05 | 0.95 | 0.94 |
| BG87P-26 | −17.5 | −2.0E−05 | 0.95 | 0.96 |
| chBG87P | −18.5 | 2.1E−05 | 0.98 | 0.97 |

TABLE 11
All the four humanized antibodies showed lower
hydrophobicity than chimeric antibody, however,
the hydrophobic risk was also relatively high

| Sample Name | Retention time(min) |
|---|---|
| BG87P-21 | 21.48 |
| BG87P-22 | 21.51 |
| BG87P-24 | 21.75 |
| BG87P-26 | 21.83 |
| chBG87P | 24.80 |

The hydrophobic patch causes the HIC retention time of the chBG87P to be over 25 minutes and the humanized BG87P-21 to be 21.9 minutes, both of them are higher than the acceptable threshold which is 21.1 minutes in IgG format. The root cause is the hydrophobic patches in HCDR3, in particular the 197-Y98-Y100-V100a part together with Y49-W50 (mainly W50) at the edge of FR2 (framework region 2) and LCDR2 of light chain (FIG. 2A). Automatic antibody pipeline analysis on aggregation of BG87P by Schrodinger also showed the higher aggregation risk (Table 12).

TABLE 12
Automatic antibody pipeline analysis on aggregation

| | chBG87P |
|---|---|
| Global AggScore | 164.53 |
| CDR Agg Score | 104.38 |
| CDRH1 AggScore | 0 |
| CDRH2 AggScore | 1.11 |
| CDRH3 AggScore | 89.7 |
| CDRL1 AggScore | 0 |
| CDRL2 AggScore | 13.53 |
| CDRL3 AggScore | 0 |

### Example 7. Solubility Engineering of Humanized Anti-CLDN6 Antibody

### Overall Strategy of Solubility Engineering of BG87P-21

In the aforesaid description, chBG87P has been engineered to a humanized antibody and we have identified BG87P-21 as a final top clone. However, a potential developability risk of a hydrophobic patch driven from HCDR3 of chBG87P has not been solved (FIG. 2). Considering that chBG87P shows promising binding activity and superior CLDN6 selectivity (FIG. 1A, FIG. 1B, FIG. 1C, FIG. 1D, FIG. 1E, FIG. 1F, FIG. 1G, and FIG. 1H), additional engineering of BG87P-21 has been done to remove the hydrophobic patch for optimal manufacturability and the mitigation of potential ADA risk.

Two main strategies were used to engineer the solubility issue of BG87P-21: single point mutation and framework swapping.

TABLE 13
Overall summary of solubility engineering of BG87P-21

| | | Screening | | |
|---|---|---|---|---|
| Design rationale | Assays | criteria | Top variants | Comment |
| 1st Round: | FACS | Improved | VK: Y49K/R | VK-Y49K is |
| Structure based mutagenesis to | HIC-RT | HIC-RT Or | VH: Y56H | abandoned |
| 1: Reduce exposed hydrophilicity | (no AC- | Improved | VH: W50Y/N | later since |
| 2: Mutate to human germline residues | SINS) | yield Or | VH: V5Q-V11T-P41S | it worsens |
| 3: Mutate to more popular residues in | | Improved | | AC-SINS |
| human Ab repertoire | | FACS Ec50 Or | | |
| 4: Improve hydrophobic core packing | | Improved | | |
| 5: Saturation mutation at critical | | FACS Emax Or | | |
| hydrophobic sites | | Improved | | |
| 2^{nd} Round: | | AC-SINS | VK: V29A/G | VH: Y59K |
| Extend structural based design | | (only in the last | VK: T31S/H | reduces yield |
| 1: Mutating residues adjacent to the | | round since the | VH: Y59K/E/Q/H/D/N | |
| hydrophobic patch | | assay was not in | | |
| (Since the results from 1^{st} round is less | | previous | | |
| than desired) | | discussions) | | |
| Combination of top point mutations | | | BG87P-31 | Both reduces |
| from the round 1^{st} and 2^{nd} | | | BG87P-32 | yield to |
| 1: Either improved HIC-RT | | | | BG87P-21; |
| 2: Or improved FACS Emax | | | | BG87P-31 |
| 3: Or improved FACS Ec50 | | | | reduces more |
| 4: Or improved yield or purity | | | | HIC-RT than |
| | | | | BG87P-32 |
| | | | | while with |
| | | | | inferior AC- |
| | | | | SINS value. |
| 3^{rd} Round: | FACS | | BG87P-33 | The new |
| Framework swapping for yield and self- | HIC-RT | | BG87P-34 | framework |
| interaction improvement | AC-SINS | | | will harbor |
| 1: The current human framework is | | | | more back |
| IGHV1-3 and IGKV1-5 | | | | mutations |
| 2: Test the additional framework of | | | | since it is less |
| IGHV3-23 and IGKV1-39 | | | | homologous |
| 3: Top point mutations on BG87P-21 are | | | | to the original |
| incorporated | | | | chimeric |
| 4: Rare amino acids are also mutated to | | | | sequence |
| popular amino acids | | | | |
| 5: Verification of charge distribution | | | | |
| influencing on self-interaction. | | | | |

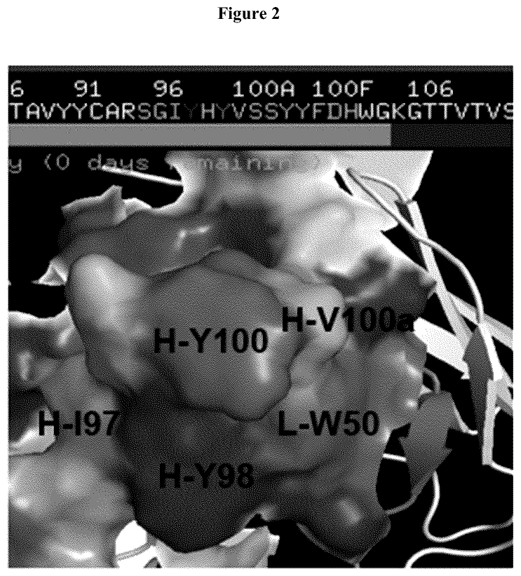

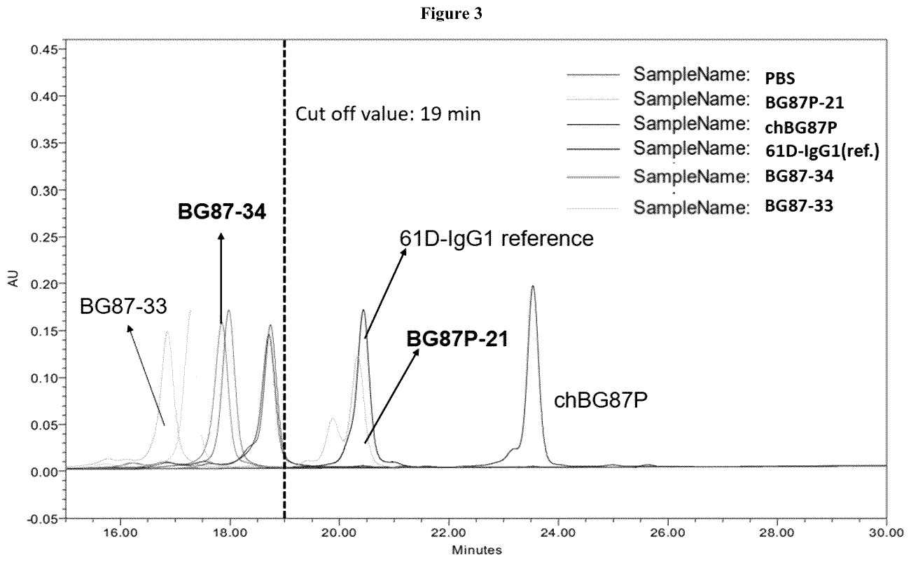

A large number of single point mutations were designed on two rationales: one is to substitute hydrophobic amino acids to more hydrophilic amino acids; and the other is to mutate rare to more popular amino acids at the same Kabat position in human antibody repertoire. 57 variants in 1^{st} round screening and 104 variants in 2^{nd} round screening were performed, seven locations were found can be replaced by other more hydrophilic amino acids with binding affinity comparable to parental BG87P-21 and slightly improved hydrophilicity (Table 14). Selected top mutations from 1^{st} and 2^{nd} round screening were combined to generate 56 variants for further validation. Combination variants BG87P-31 and BG87P-32 (VH and VL amino acid sequences are SEQ ID NO: 46 and 42 respectively) were selected as top candidates with comparable binding affinity to BG87P-21 and improved HIC-retention 17.4 min for BG87P-31 and 18.49 min for BG87P-32, both of them are superior to the parental BG87P-21 22.3 min (Table 14 and FIG. 3).

TABLE 14
Characterization of chBG87P solubility engineering variants

| | | | | BG87P- | BG87P- | BG87P- | BG87P- | BG87P- |
|---|---|---|---|---|---|---|---|---|
| Antibody name | | | chBG87P | 21 | 32 | 31 | 33 | 34 |
| Cell | FACS- | (HEK293- | 1 | 0.91 | 1.31 | 1.6 | 2.45 | 1.17 |
| binding | Ec50 | hCLDN6) | | | | | | |
| activity | ratio | | | | | | | |
| | to | | | | | | | |
| | BG87P | | | | | | | |

| | | | | | | | |
|---|---|---|---|---|---|---|---|
| | FACS Emax ratio | 1 | 0.66 | 0.63 | 0.76 | 0.91 | 1.03 |
| | to BG87P | | | | | | |
| | (HEK293T- | | | | | | |
| | hCLDN6) | | | | | | |
| | Ec50 to | 7.2 | 8.9 | No data | No data | 25.8 | 8.8 |
| | HEK293T_cyno- | | | | | | |
| | CLDN6 (nM) | | | | | | |
| | FACS Emax ratio | 1 | 0.8 | No data | No data | 0.75 | 1 |
| | to BG87P | | | | | | |
| | (HEK293T-cyno- | | | | | | |
| | CLDN6) | | | | | | |
| Biophysical | Tm1 (° C.) | 69.8 | 69.3 | 69.4 | 70.5 | 68.8 | 69.9 |
| properties | Tagg (° C.) | 65.6 | 65.4 | 69.86 | 69.16 | 62.3 | 65.2 |
| | AC-SINS DI (nm) | 12.85 | 21.95 | 10.09 | 22.2 | 7.7/11.4 | 8.05 |
| | HIC-RT (min) | 23.54 | 20.33 | 18.49 | 17.41 | 16.87 | 17.85 |
| | Purity (%) | 95.44 | 97.99 | 99.18 | 99.46 | 94.96 | 93.14 |
| | Yield (mg/L) | 242.87 | 119.57 | 30.06 | 34.02 | 111.65 | 221.07 |
| | Net charge (under | 2.9 | 8.8 | 9.3 | 9.59 | 5.8 | 4.8 |
| | pH 7.4) | | | | | | |

| Framework | Chimeric | IGHV1- | IGHV1- | IGHV1- | IGHV- | IGHV3- |
|---|---|---|---|---|---|---|
| | Ab | 3-01 & | 3-01 & | 3-01 & | 3-23 & | 23 & |
| | | IGKV1-5 | IGKV1-5 | IGKV1-5 | IGKV1-5 | IGKV1-39 |

However, even though the hydrophobicity was mitigated during single point mutation approach for solubility engineering, the self-association risk which determined by AC-SICNS was still showed from moderate to high. The reason for this unsolved issue was the hydrophobicity risk mainly reflected by the amount of the hydrophobic patches which actually exhibited on the surface of the antibody, while the reasons for inducing self-interaction also involved isoelectric point issues, uniform charge distribution and even some unknown specific interaction. (Doi.org/10.1021/mp200566k). Therefore, self-interaction risk triggering by above reasons cannot be mitigated only by substituting hydrophobic residues to hydrophilic ones. Moreover, we have found that the parental chBG87P which with higher HIC retention time 25 min while exhibited a lower self-interaction propensity with AC-SINS value around 12.85 nm in PBS buffer (Table 14). Another found appearance is that the calculated net charge of chBG87P is more less than BG87P-21 with 2.9 VS 8.8. Therefore, we hypothesize that framework or net charge may have an influence on self-association effect.

We tested the additional framework of IGHV3-23 and IGKV1-39, back mutations based on new paired framework and selected top point mutations on solubility engineering of BG87P-21 are both incorporated (Table 13). Final top candidate BG87P-34 showed no red flag in all routine biophysical properties (Table 14; VH and VL amino acid sequences are SEQ ID NO: 43 and 44 respectively).

Moreover, after framework swapping to IGHV3-23 and IGKV1-39, the lost Emax of BG87P-21 in original humanization procedures has been recovered (FIG. 1G). The principle for critical back mutation identification used in these two humanization procedures was the same therefore excluding the possibility that any back mutations were left out in the previous round of humanization to the Emax liability. One explanation for this recovered Emax in cell binding by framework swapping is that different paired framework may various in VH-VL angle, while a particular VH-VL angle may help to maintain Emax of the cell binding. Final lead clone BG87P-34 shows good cross-reactivity in different species (FIG. 1I and FIG. 1J). Non-specific binding to human CLDN9 was performed by using HEK293T/human CLDN9, the data indicated that BG87P-34 have a good selectivity to human CLDN6 over CLDN9 (FIG. 1H).

### Example 8. Humanization and scFv Engineering of the Anti-Human CD3 Antibody Sp34

The well reported mouse clone sp34 (Blumberg 1990 PNAS 87 (18):7220-24) was an optimal clone for developing anti-CD3 based therapeutics due to its cyno CD3 cross-reactivity. For humanization of sp34, human germline IgG genes were searched for sequences that share high degrees of homology to the protein sequences (SEQ ID NO: 48-57) of sp34 variable regions by blasting the human immunoglobulin gene database in IMGT (http://www(dot)imgt(dot)org/IMGT_vquest/share/textes/index(dot)html) and NCBI (http://www(dot)ncbi(dot)nlm(dot)nih(dot)gov/igblast/) websites. The human IGVH and IGVK genes that are present in human antibody repertoires with high frequencies (Glanville 2009 PNAS 106:20216-20221) and are homologous to sp34 were selected as the templates for humanization.

Humanization was carried out by CDR-grafting (Methods in Molecular Biology, Vol 248: Antibody Engineering, Methods and Protocols, Humana Press) and the humanization antibodies (hu-sp34) were engineered as the human IgG1 format using an in-house developed expression vector. In the initial round of humanization, mutations from murine to human amino acid residues in framework regions were guided by the simulated 3D structure, and the murine framework residues of structural importance for maintaining the canonical structures of CDRs were retained in the 1^{st} version of humanization antibody sp34. Specifically, CDRs of sp34 VL (SEQ ID NO: 51˜53) were grafted into the framework of human germline variable gene IGVκ3-15 with several murine framework residues (Q1, A2, V4, V36, E38, L43, F44, T45, G46, G49, L66, D69, A71, 185, and F87) retained. CDRs of sp34 VH (SEQ ID NO: 48-50) were grafted into the framework of human germline variable gene IGVH3-7 with several murine framework residues (D73, S76, M89, V93) retained.

Humanized sp34 (hu-sp34) and chimeric sp34 (ch-sp34) were constructed as human full-length antibody format using in-house developed expression vectors that contain constant regions of a human IgG1 and kappa chain, respectively, with easy adapting sub-cloning sites. Expression and preparation of humanized sp34 and chimeric sp34 antibodies was achieved by co-transfection of the heavy chain and corresponding light chain constructs into 293G cells (developed in house) and by purification using a protein A column. The purified antibodies were concentrated to 0.5-5 mg/mL in PBS and stored in aliquots in −80° C. freezer for using in the assays below.

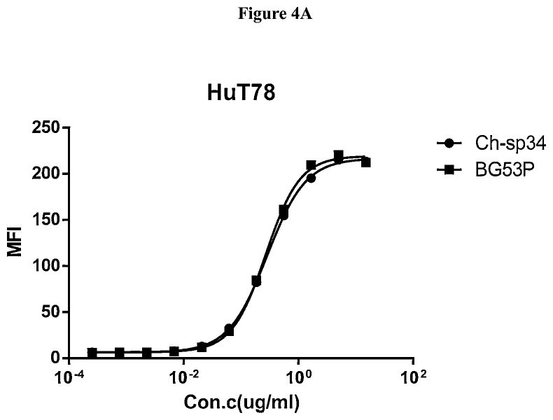

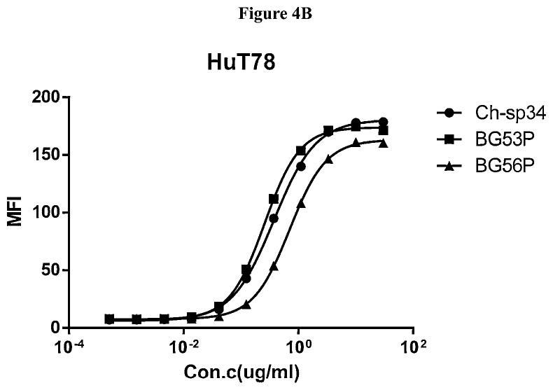

For affinity determination, antibodies were captured by anti-human Fc surface, and used in the affinity-assay based on surface plasmon resonance (SPR) technology. The binding activity of humanized sp34 to bind native CD3 on live cells was evaluated using HuT78 cells in a FACS based assay. Live HuT78 cells were seeded in 96-well plate, and were incubated with a series of dilutions of chimeric or humanized sp34. Mouse anti-Human IgG was used as second antibody to detect antibody binding to the cell surface. EC50 values for dose-dependent binding to human native CD3 were determined by fitting the dose-response data to the four-parameter logistic model with GraphPad Prism. The humanized sp34 BG53P (SEQ ID NO: 48-53 and 58-61) showed comparable binding affinity to ch-sp34 in both SPR assay and FACS assay (Table 15 and FIG. 4A).

TABLE 15
Comparison of binding affinities of hu-sp34 with ch-sp34 to CD3 by SPR and FACS

| | | | | | | | HuT78 |
|---|---|---|---|---|---|---|---|
| | | | | | | | Binding |
| | | | | | | | FACS |
| | SPR binding affinity to CD3_{ε} | | | SPR binding affinity to CD3_{ε}/δ | | | EC50 |
| Ab | kₒₙ (M⁻¹s⁻¹) | k_{off} (s⁻¹) | K_{D} (nM) | kₒₙ (M⁻¹s⁻¹) | k_{off} (s⁻¹) | K_{D} (nM) | (nM) |
| Ch-sp34 | 2.16E+05 | 4.96E−04 | 2.30E−09 | 1.09E+05 | 2.96E−03 | 2.71E−08 | 1.9 |
| BG53P | 1.98E+05 | 5.25E−04 | 2.65E−09 | 9.93E+04 | 2.52E−03 | 2.54E−08 | 1.8 |

Based on humanized sp34 BG53P template, we made several single-mutations converting the retained murine residues in framework region to corresponding human germline residues which include four retained murine residues (D73, S76, M89, V93) in VH and fifteen retained murine residues (Q1, A2, V4, V36, E38, LA3, F44, T45, G46, G49, L66, D69, A71, 185, and F87) in VL. All humanization mutations were made using primers containing mutations at specific positions and a site directed mutagenesis kit (Cat. No. FM111-02, TransGen, Beijing, China). The desired mutations were verified by sequencing analysis. These hu-sp34 variant antibodies were tested in binding assays as described previously. Comparing to hu-sp34-1A-1f, mutations of V36Y, G46L and G49Y (Kabat numbering) on VK significantly impaired binding affinities of the humanized variants and while the rest versions of hu-sp34 humanization variants had comparable binding activities to hu-sp34-1A-1f. D73N in VH significantly reduced expression level (data not shown).

Taken together, the well-engineered version of humanized monoclonal antibody, BG56P (SEQ ID NOS: 70-77, and 72-86) was derived from the mutation process described as above, and characterized in detail (Table 16 and FIG. 4B).

TABLE 16
Comparison of humanized sp34 binding affinities to CD3

| | | | | | | | HuT78 |
|---|---|---|---|---|---|---|---|
| | | | | | | | binding |
| | | | | | | | FACS |
| | SPR binding affinity to CD3_{ε} | | | SPR binding affinity to CD3_{ε}/δ | | | Ec50 |
| Ab | kₒₙ (M⁻1s⁻¹) | k_{off} (s⁻¹) | K_{D} (M) | kₒₙ (M⁻¹s⁻¹) | k_{off} (s⁻¹) | K_{D} (M) | (nM) |
| Ch-sp34 | 2.16E+05 | 4.96E−04 | 2.30E−09 | 1.09E+05 | 2.96E−03 | 2.71E−08 | 2.49 |
| BG53P | 1.06E+05 | 4.84E−04 | 4.58E−09 | 1.09E+05 | 1.05E−03 | 9.62E−09 | 1.72 |
| BG56P | 4.60E+04 | 3.25E−04 | 7.06E−09 | 6.16E+04 | 1.12E−03 | 1.82E−08 | 4.69 |

### Example 9. ScFv Engineering of Humanized Sp34

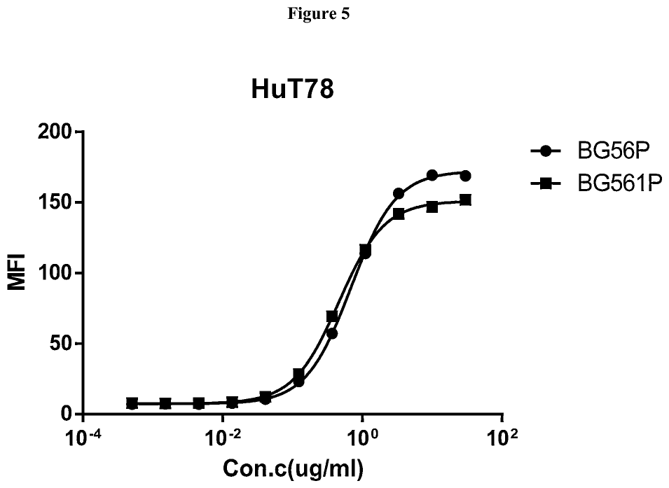

In order to generate a plug and play bispecific format and avoid the light chain-heavy chain mispairing, we reformatted the BG56P antibody into single-chain fragment variable (scFv) format with 3xG4S linker in between VH and Vκ. The reformatted scFv were fused to N-term of a human IgG1 Fc region into scFv-Fc format using in-house developed expression vectors with easy adapting sub-cloning sites. Expression and preparation of parental and re-engineered hu-sp34 scFv-Fc was achieved by transfection of the scFv-Fc construct into 293G cells (developed in house) and by purification using a protein A column. The purified scFv-Fc format antibodies were concentrated to 0.5-5 mg/mL in PBS and stored in aliquots in −80° C. freezer for using in the assays below. The scFv-ized BG56P (referred as BG561P, SEQ ID NO: 48-53 and 62-65) showed comparable binding affinities with that of antibody version of BG56P in SPR and FACS (Table 17 and FIG. 5).

TABLE 17
Comparison of affinities to CD3 of humanized sp34
and scFv humanized sp34 binding by SPR and FACS

| | | | | | | | HuT78 |
|---|---|---|---|---|---|---|---|
| | | | | | | | binding |
| | SPR binding affinity to CD3_{ε} | | | SPR binding affinity to CD3_{ε}/δ | | | FACS |
| Ab | kₒₙ (M⁻¹s⁻¹) | k_{off} (s⁻¹) | K_{D} (nM) | kₒₙ (M⁻¹s⁻¹) | k_{off} (s⁻¹) | K_{D} (nM) | EC50 (nM) |
| BG56P | 4.60E+04 | 3.25E−04 | 7.06E−09 | 6.16E+04 | 1.12E−03 | 1.82E−08 | 4.69 |
| BG561P | 4.42E+04 | 4.01E−04 | 9.08E−09 | 7.25E+04 | 1.88E−03 | 2.59E−08 | 4.35 |

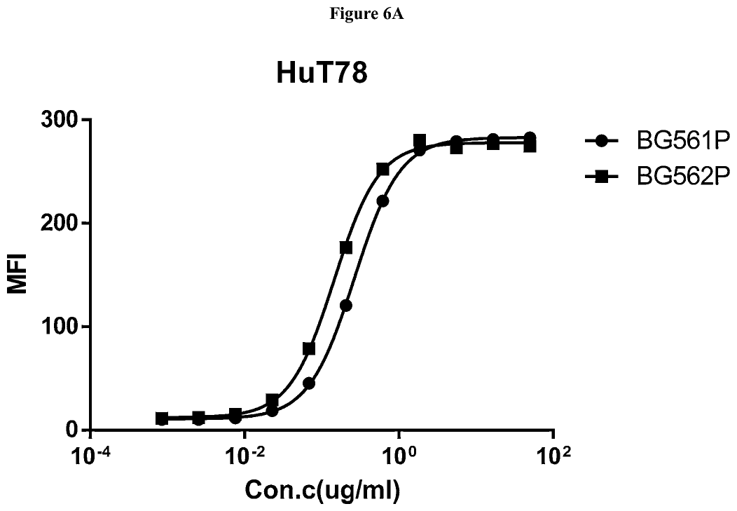

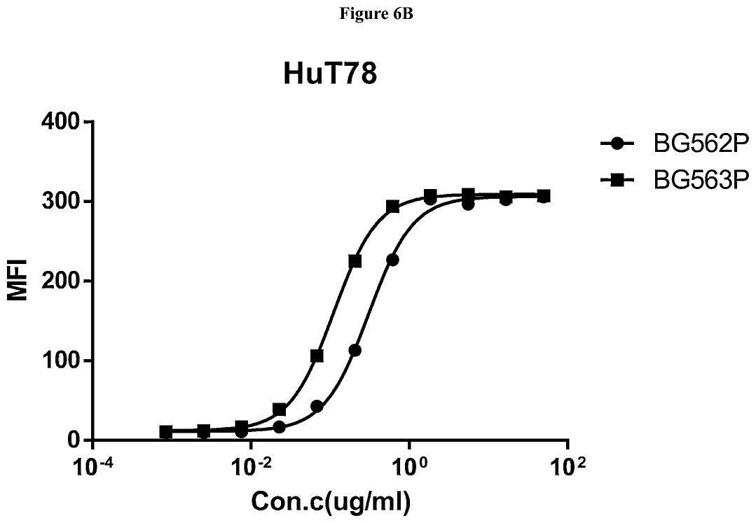

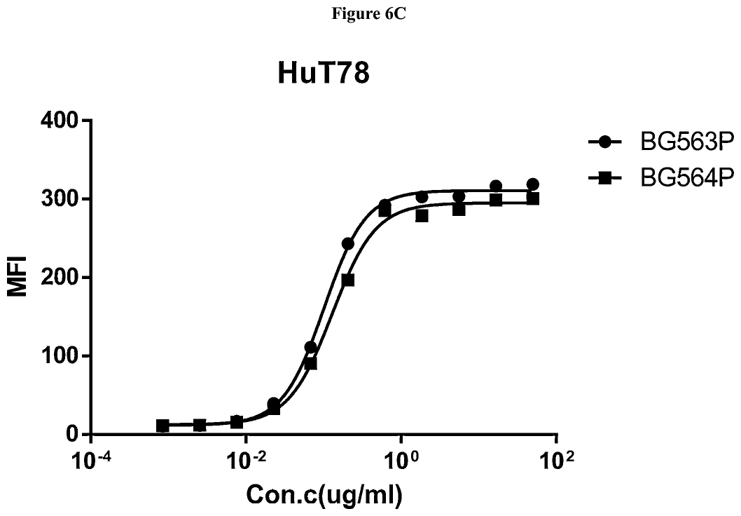

Based on BG561P, we made several mutations in the framework and the CDRs to remove potential PTM sites and improve thermal and colloidal stability for therapeutic use in human. Mutation of LAV in VL (the resulted humanized scFv is designated as BG562P, SEQ ID NO: 48-53 and 69-70) showed 5 degree improvement in aggregation temperature (Tagg). Combination of LAV in VL and A49G and D65G in VH (the resulted humanized scFv is designated as BG563P) (SEQ ID NO: 48, 71, 50, 51-53, 73, and 74) showed improved thermal and colloidal stabilities compared with BG561P while showed slightly improved binding affinity to human CD3 in FACS assay. The potential PTM sites include potential deamidation site N30 (NT) in the junction region of FR1 and HCDR1 (Kabat CDR definition) and N100 (NS) in HCDR3. Each of the N was mutated to S to remove the potential deamidation sites. All mutations were made using primers containing mutations at specific positions and a site directed mutagenesis kit (Cat. No. FM111-02, TransGen, Beijing, China). Taken together, the well-engineered version of humanized scFv, BG564P (SEQ ID NO: 48, 71, 75, 51-53, 77, and 78), was derived from the mutation process described as above, and characterized in detail. The results showed humanized scFv BG564P retained binding affinity to CD3 (Table 18-Table 20 and FIG. 6) and improved biophysical stabilities compared with humanized scFv BG561P (Table 20).

TABLE 18
Comparison of different versions of humanized
sp34 scFv-Fc binding affinities to CD3 by SPR

| | SPR binding affinity to CD3ε | | |
|---|---|---|---|
| Ab | kₒₙ (M⁻¹s⁻¹) | k_{off} (s⁻¹) | K_{D} (nM) |
| BG561P | 7.22E+04 | 3.60E−04 | 4.98E−09 |
| BG562P | 1.16E+05 | 3.67E−04 | 3.16E−09 |
| BG563P | 4.93E+05 | 7.72E−04 | 1.57E−09 |
| BG564P | 4.14E+05 | 5.76E−04 | 1.39E−09 |

TABLE 19
Comparison of humanized sp34 scFv-Fc
binding affinities to CD3 by FACS

| | HuT78 binding FACS | | |
|---|---|---|---|
| Ab | Test 1 Ec50 (nM) | Test 2 Ec50 (nM) | Test 3 Ec50 (nM) |
| BG561P | 2.46 | | |
| BG562P | 2.85 | 2.85 | |
| BG563P | | 0.94 | 0.94 |
| BG564P | | | 1.23 |

TABLE 20
Comparison of thermal stabilities and colloidal
stabilities of humanized sp34 scFc-Fc

| scFv-Fc | Tm (° C.) | Tagg (° C.) |
|---|---|---|
| BG561P | 58.1 | 45.5 |
| BG562P | 59.0 | 50.8 |
| BG563P | 59.9 | 50.9 |
| BG564P | 61.6 | 51.2 |

Melting temperature (Tm) was determined using a high throughput MicroCal™ VP-Capillary DSC (Malvern Instruments, Northampton, MA). Thermograms for each protein (350 μL at 0.5 mg/mL) were obtained from 20° C. to 100° C. using a scan rate of 90° C./hr. Thermograms of the buffer alone were subtracted from each protein sample. Obtained results shows the values for midpoint of transition temperatures (Tm) and the calorimetric enthalpy (ΔH) of the sample, which suggested the Tm of BG564P was improved compared with BG561P (Table 20).

The aggregation temperature Tagg (° C.) is representative of the colloidal stability of the samples and was obtained by monitoring the onset of aggregation by SLS266 using UNCLE™ (Unchained lab, Pleasanton, CA). Samples were loaded into Uni, and subjected to a temperature ramping from 15° C. to 95° C. The back-reflection optics cannot detect near UV light scattering by protein aggregates, and thus only non-scattered light reaches the detector. The reduction of back reflected light is therefore a direct measure for aggregation in the sample, which suggested the Tagg of BG564P was improved compared with BG561P (Table 20).

### Example 10. Generation of CLDN6×CD3 BsAb BG143P

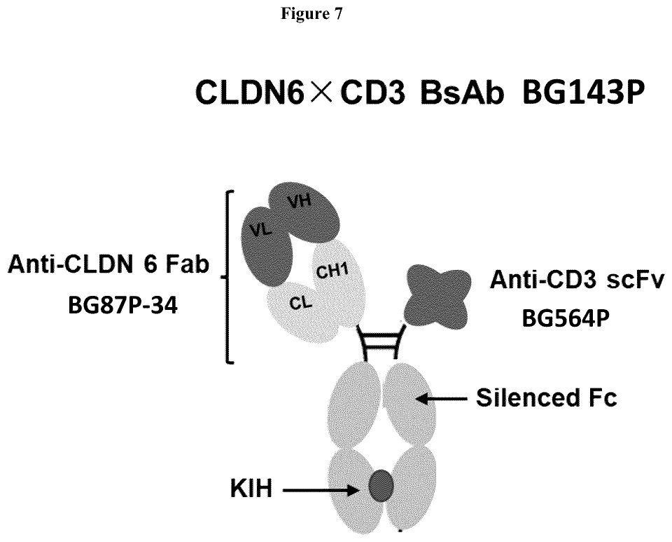

Agonistic anti-CD3 antibodies have demonstrated toxicity in the clinical setting, which may indicate that systemic FcγR cross-linking is not ideal for CD3 activation. The aim was to achieve potent CD3 stimulation specifically at the tumor site without systemic CD3 activation for a broad range of cancers. To overcome the dependency of FcγR cross-linking, we generated a CLDN6×CD3 BsAb BG143P with the following features as shown in FIG. 7. This specific construct BG143P includes an IgG-fusion like multi-specific antibody format with a module ratio of 1:1, a well-engineered Fab fragment BG87P-34 that binds to CLDN6 and a scFv of BG564P that binds to CD3 fusion at the N terminal of CH2, and a Fc null version of huIgG1, which has no FcγR binding but retains FcRn binding. Knob into Hole (KIH) was also introduced in Fc to increase heterodimerization. The sequence information of BG143P is listed in SEQ ID NO: 79-84.

### Example 11. Target-Binding Activity of CLDN6×CD3 BsAb BG143P

The binding kinetics of CLDN6×CD3 BsAb BG143P were measured using SPR. SPR was used to measure the on-rate constant (ka) and off-rate constant (ka) of the antibodies to recombinant proteins of CDεγ and then determined the affinity constant (K_{D}). The results showed that CLDN6×CD3 BsAb had a strong binding affinity to human CDεγ as shown in Table 21.

TABLE 21
Amino acid and DNA sequences of CLDN6 × CD3 BsAb BG143P

| Antibody | Antigen | ka (1/Ms) | kd (1/s) | K_{D} (M) |
|---|---|---|---|---|
| BG143P | Human CDεγ | 2.17E+06 | 1.81E−02 | 8.31E−09 |
| | Cyno CDεγ | 1.32E+06 | 5.99E−03 | 4.55E−09 |

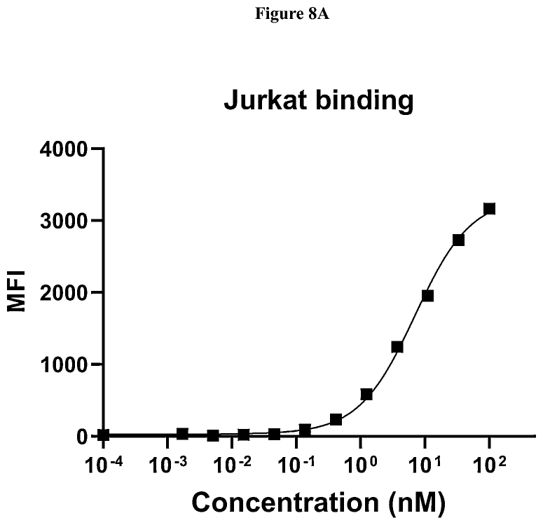

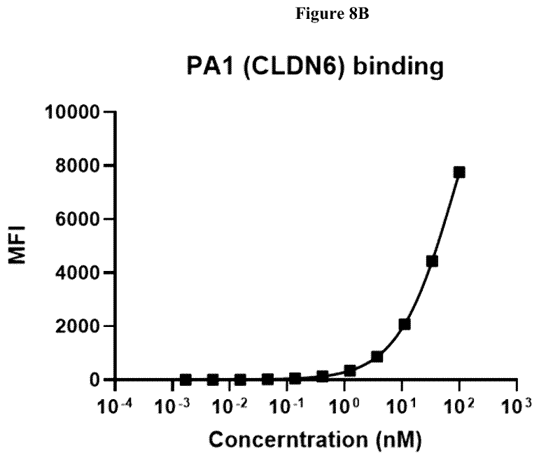

The FACS results further confirmed the binding activity of BG143P to CD3 and CLDN6. The BsAb showed strong binding activities to CD3-expressing Jurkat in a dose-responsive manner with EC50 of 6.98 nM. (FIG. 8A) Similarly, BG143P showed strong binding activities to CLDN6-expressing PA-1 in a dose-responsive manner with EC50 of 81.26 nM (FIG. 8B).

### Example 12. In Vitro Functional Activity of CLDN6×CD3 Antibody

### On-Target T Cell-Redirected Cytotoxicity and Cytokine Release

T cell-redirected cytotoxicity of BG143P against PA-1 (cancer cell line with high CLDN6 expression), Hutu80 (cancer cell line with medium CLDN6 expression), AGS (cancer cell line with low and heterogeneous CLDN6 expression) and NCI-H1299 (cancer cell line negative for CLDN6 expression) was evaluated using human PBMCs as effector cells. For measurement of cellular cytotoxicity, target cancer cell lines were engineered to express Nano-luciferase. Around 10000 target cells and 25000 human PBMCs (E/T=2.5) were seeded into each well of a 96-well U-bottom plate and incubated with various concentrations of antibody for 48 h at 37° C. and 5% CO₂. The supernatant was collected for cytokine detection. Target cell killing was measured by Nano-Glo detection kit (Promega). The cytotoxic activity (%) of antibody was calculated using the following formula. Cytotoxic activity (%)=(A−B)/(A−C)*100%. “A” represents the average luminescence signal of wells with untreated target cells only, “B” represents the average luminescence signal of wells with antibody and PBMCs, and “C” represents the average luminescence signal of wells with target cells totally lysed with Triton-X100. IFN-γ and IL-2 were detected in supernatants by HTRF kit (Cisbio).

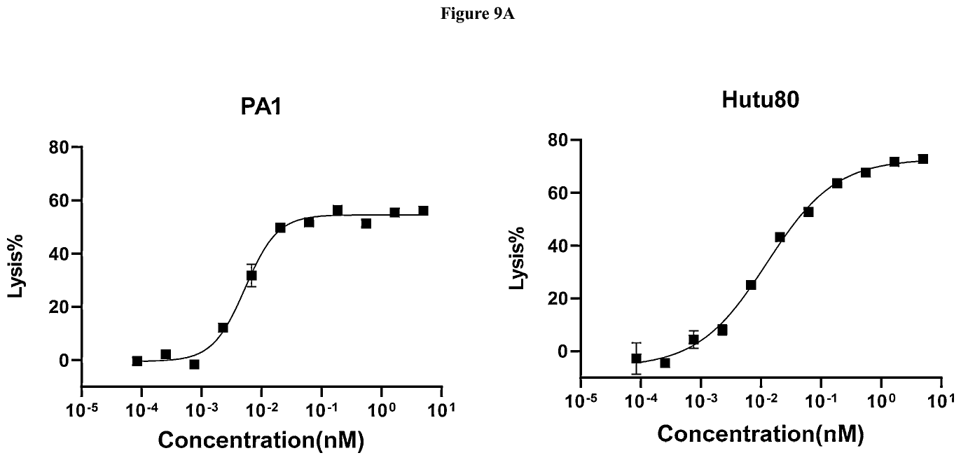

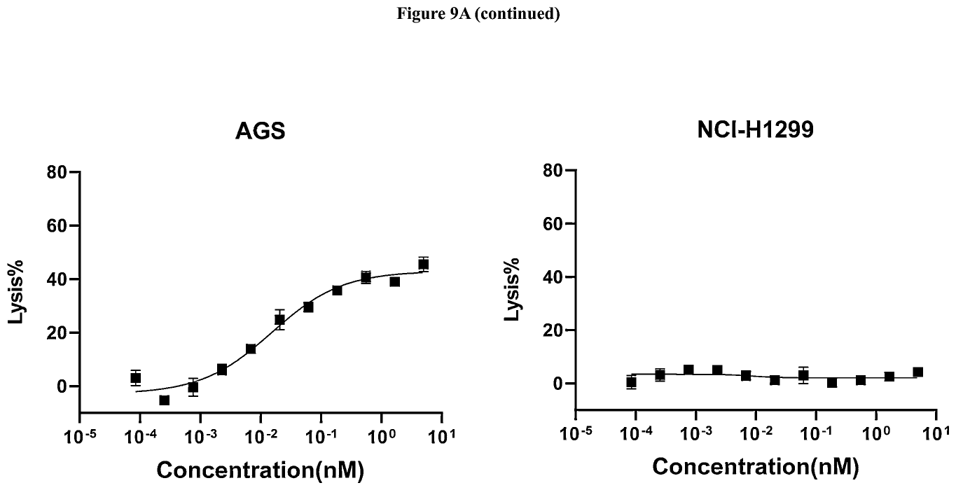

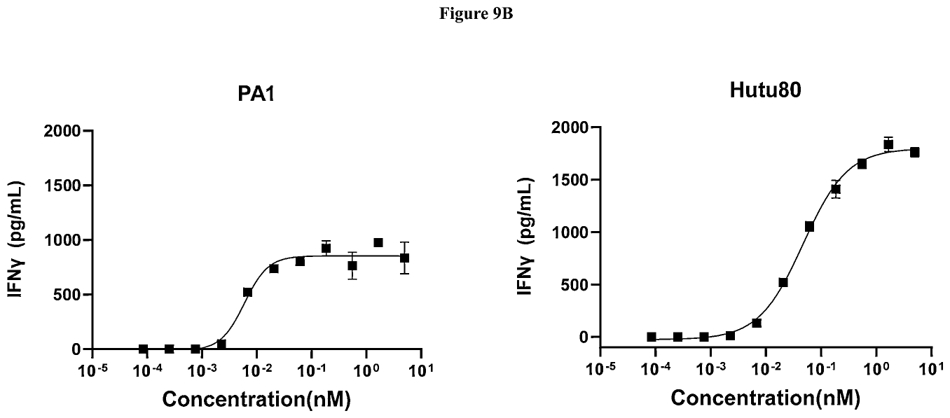

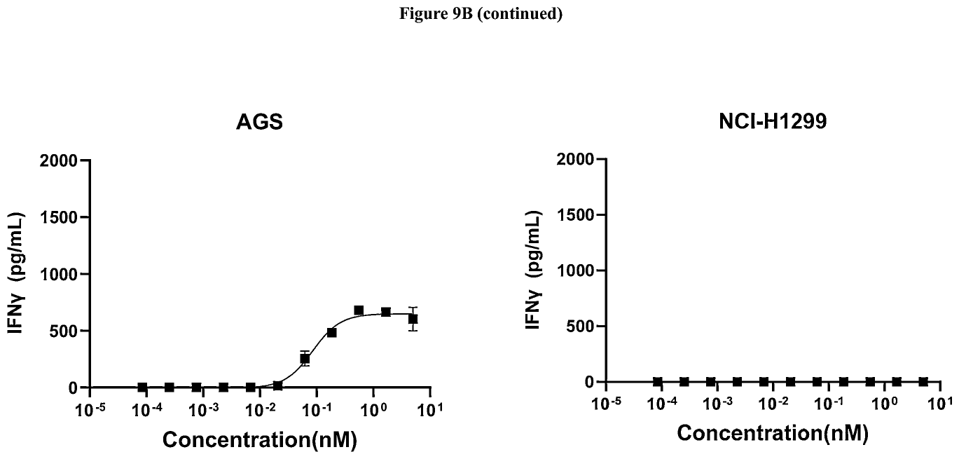

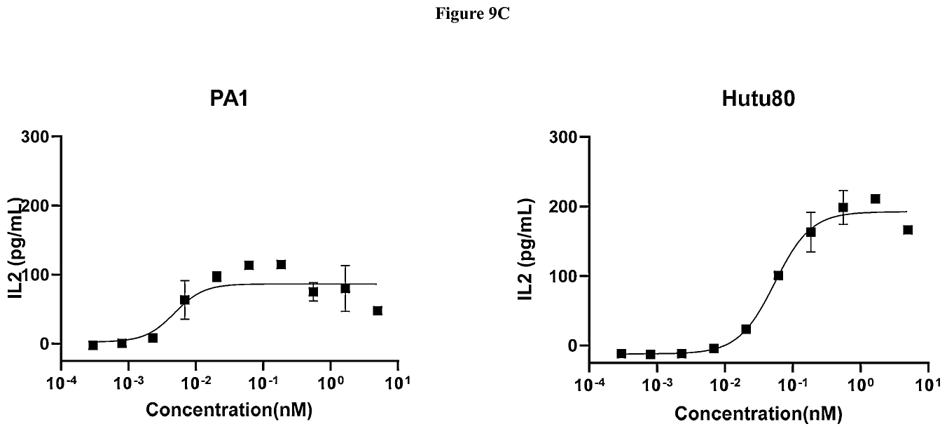

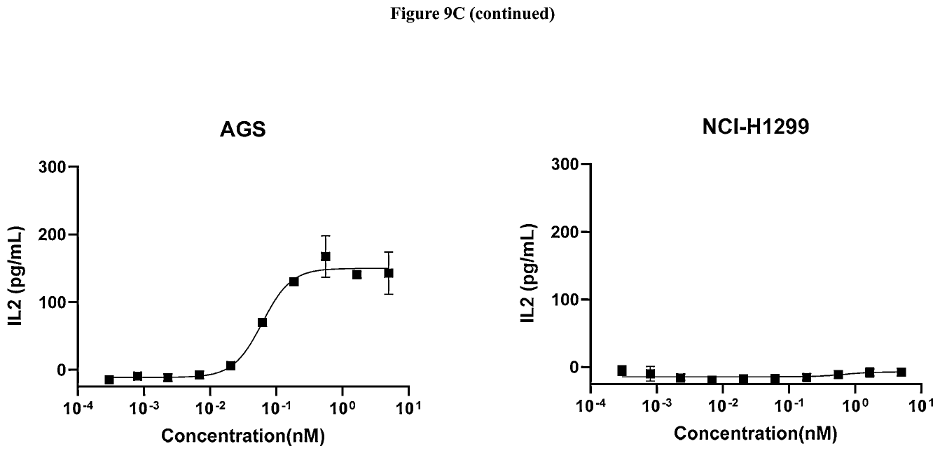

As shown in FIG. 9, BG143P showed potent T cell-redirected killing and cytokine release induction potency in a dose-dependent manner with pM EC50 levels.

### Functional Specificity Against Human CLDN6 and CLDN9

Amino acid sequence are highly conserved among human CLDN6 and CLDN9 with only 3 amino acids difference in the extracellular domain. CLDN9 is widely expressed in human normal tissues, thus the binding specificity between CLDN6 and CLDN9 is important and examined by FACS analysis.

Expressing vectors of human CLDN6 and CLDN9 were established by inserting synthesized cDNA coding corresponding sequences into mammalian expression vector. NCI-H1299 stable cells expressing human CLDN6 and CLDN9 were generated by transfection of corresponding plasmids. Cells were suspended at a concentration of 1×10⁶ cells in FACS buffer (2% FBS, 1×PBS), and the cell suspension was dispensed into a U-bottom 96-well plate (100 μL/well). Antibody was added thereto at final top concentration of 100 nM and 2× dilute for 11 dilutions, then mixed with cells and incubated at 4° C. for 1 hour. After centrifugation, the reaction solution was removed, and the cells were washed twice with 200 μL/well FACS buffer. Then, APC-anti human Fcγ was diluted 500-fold with FACS buffer and added as secondary antibodies to the cells. The cells were incubated at 4° C. for 30 mins, then washed twice as above, and suspended in 100 μL FACS buffer. The cell suspension was subjected to flow cytometry.

Killing of BG143P on NCI-H1299-CLDN6/CLDN9 was evaluated by Nano-Glo assay using human PBMCs, Around 10000 target cells and 25000 human PBMCs (E/T=2.5) were seeded into each well of a 96-well U-bottom plate and incubated with various concentrations of antibody for 48 h at 37° C. and 5% CO₂. The supernatant was collected for cytokine detection. Target cell killing was measured by Nano-Glo detection kit (Promega). The cytotoxic activity (%) of antibody was calculated using the following formula. Cytotoxic activity (%)=(A−B)/(A−C)*100%. “A” represents the average luminescence signal of wells with untreated target cells only, “B” represents the average luminescence signal of wells with antibody and PBMCs, and “C” represents the average luminescence signal of wells with target cells totally lysed with Triton-X100. IFN-γ was detected by HTRF kit (Cisbio).

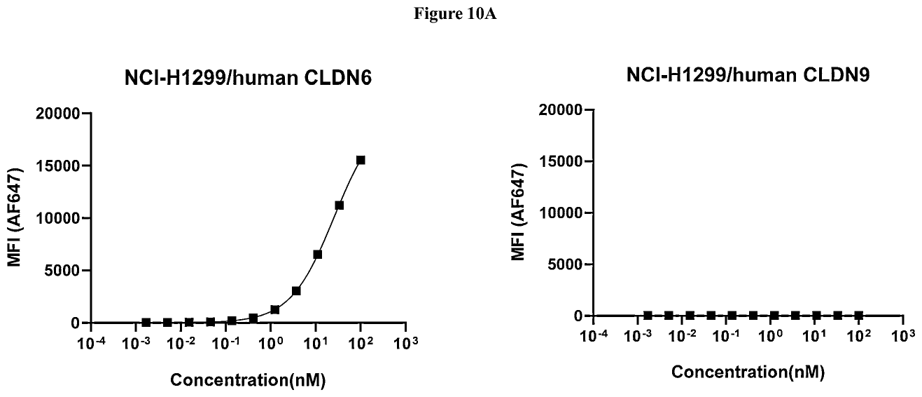

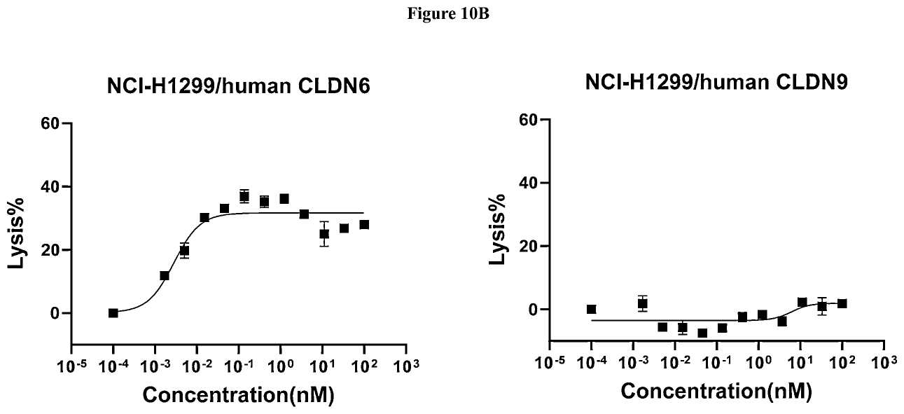

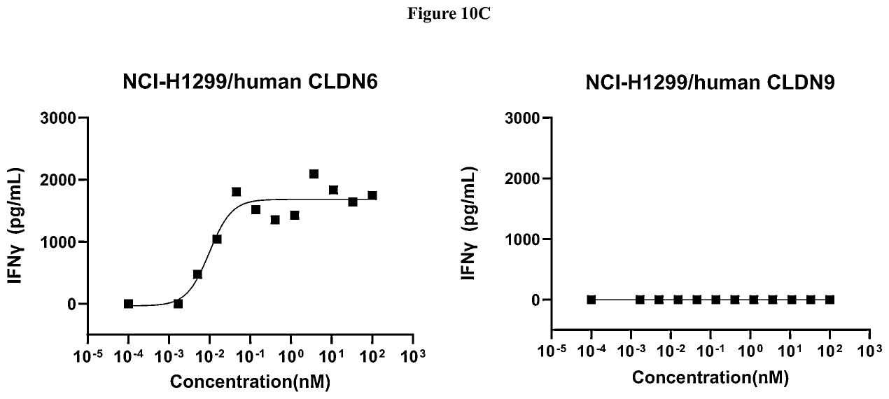

As shown in FIG. 10, BG143P was an antibody with specific binding (FIG. 10A), cell killing (e.g. lysis) (FIG. 10B) and IFN-γ induction activity (FIG. 10B) against human CLDN6, but not human CLDN9.

### Example 13. In Vivo Efficacy of CLDN6×CD3 BsAb BG143P in OV90 Xenograft Model

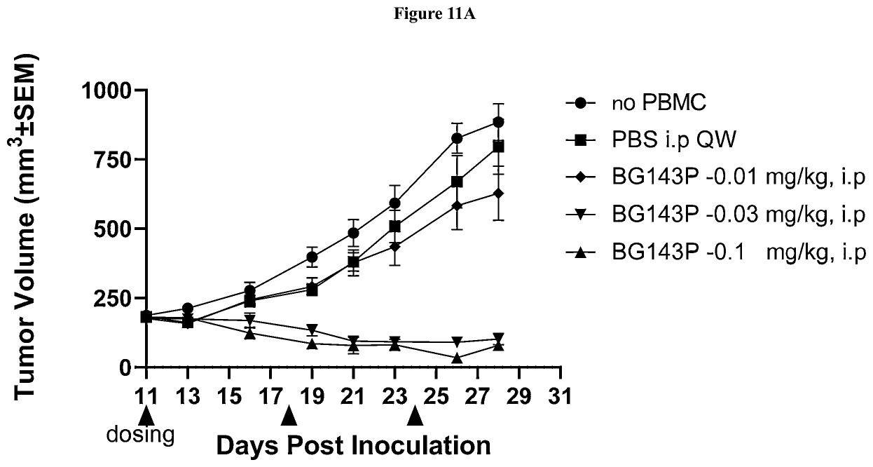

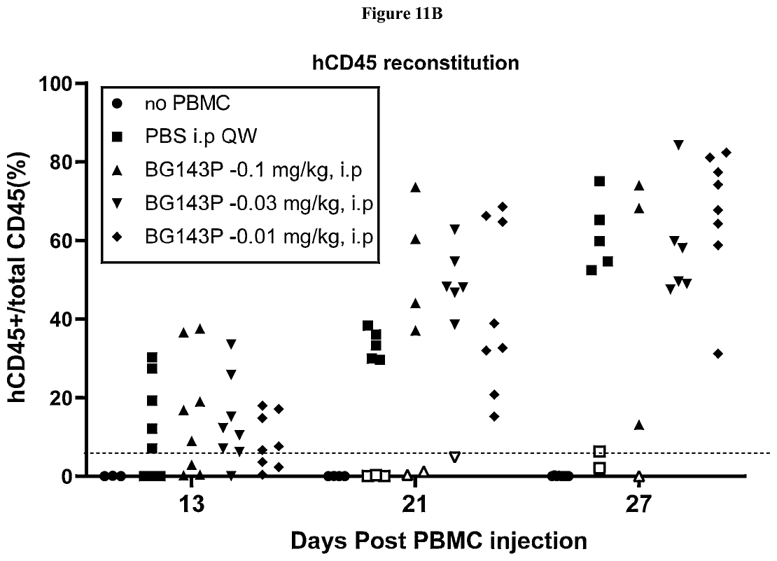

The in vivo anti-tumor efficacy of CLDN6×CD3 BsAb BG143P was evaluated in xenograft model in PBMC-humanized mice. The human ovarian cancer cell line OV-90 (ATCC) expressing human CLDN6 was subcutaneous inoculated into NCG (NOD/ShiLtJGpt-Prkdc^{em26Cd52}II2rg^{em26Cd22}/Gpt) mice, and human PBMCs were injected intravenously into the mice in the following day. Tumor bearing mice were randomized into treatment groups when tumor volume reached around 200 mm³, to receive an administration of the antibody or vehicle (PBS) as a control. Antibody/vehicle were administered once a week. The length (L) and width (W) of the tumor mass and body weight of each mouse were measured three times per week. And tumor volume (TV) was calculated as: TV=(L×W²)/2. FIG. 11A shows the in vivo anti-tumor efficacy of BG143P, which showed strong efficacy at 0.03 mg/kg and 0.1 mg/kg, with TGI % (Tumor Growth Inhibition Ratio, %) of 115.43% and 125.92%.

The reconstitution of hPBMC in the mouse was checked on week 2, week 3, and week 4, post PBMC injection. hCD45+ cells out of live cells in peripheral blood was 20% on week 2, and increased to 60% on week 4. FIG. 11B demonstrates hPBMC reconstitution.

### Example 14. In Vivo Efficacy of CLDN6×CD3 BsAb BG143P in B16F10−/hCLDN6 Syngeneic Model

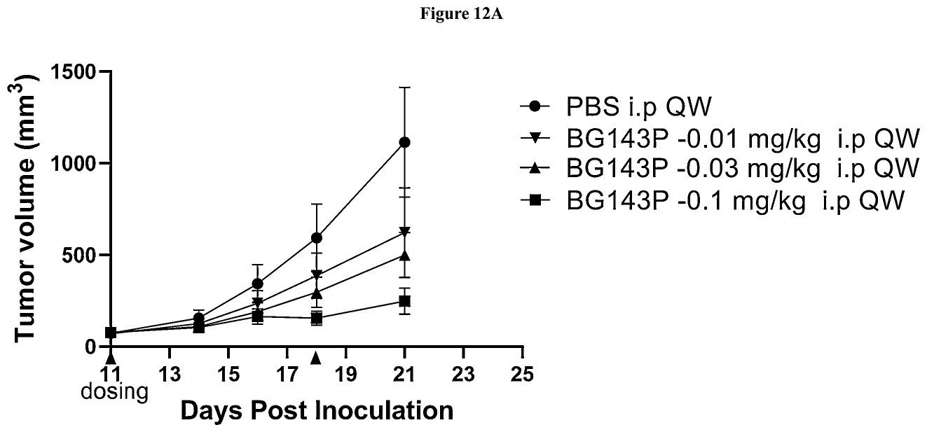

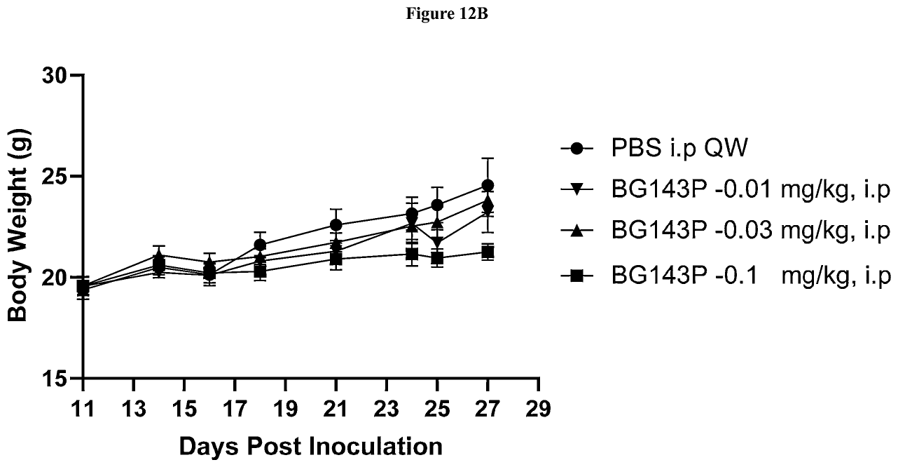

Another type of efficacy model was conducted to evaluate the in vivo efficacy of CLDN6×CD3 BsAb BG143P. Human CLDN6 expressing plasmid was constructed and stably transfected in B16F10 cell line, and the resulted B16F10/human CLDN6 cell line was confirmed to be able to growth in human CD3EDG-transgenic mice, with hCLDN6 expression preserved post tumor formation. To establish this model, B16F10/human CLDN6 cells were inoculated subcutaneously into hCD3EDG-transgenic mice, in which mouse CD3 genes were replaced with human counterparts. Mice were randomized after tumor volume reached around 100 mm³. Test article or PBS were intraperitoneally injected into the mice every week. The length (L) and width (W) of the tumor mass and body weight of each mouse were measured three times per week. And tumor volume (TV) was calculated as: TV=(L×W2)/2. BG143P at 0.1 mg/kg presented strong efficacy, with a TGI % of 93.54%, which is shown in FIG. 12A. No obvious body weight loss observed in the study, as illustrated in FIG. 12B.

### LITERATURES

ADDIN EN.REFLIST 1. Krause, G., et al., Structure and function of claudins. Biochimica et Biophysica Acta (BBA)—Biomembranes, 2008. 1778 (3): p. 631-645.
2. Günzel, D. and A. S. Yu, 8i Claudins and the modulation of tight junction permeability. Physiol Rev, 2013. 93 (2): p. 525-69.
3. Tsukita, S., H. Tanaka, and A. Tamura, The Claudins: From Tight Junctions to Biological Systems. Trends in Biochemical Sciences, 2019. 44 (2): p. 141-152.
4. Matter, K. and M. S. Balda, Signalling to and from tight junctions. Nat Rev Mol Cell Biol, 2003. 4 (3): p. 225-36.
5. Singh, A. B., S. B. Uppada, and P. Dhawan, Claudin proteins, outside-in signaling, and carcinogenesis. Pflugers Arch, 2017. 469 (1): p. 69-75.
6. Turksen, K. and T.-C. Troy, Claudin-6: A novel tight junction molecule is developmentally regulated in mouse embryonic epithelium. Developmental Dynamics, 2001. 222 (2): p. 292-300.
7. Du, H., et al., Claudin 6: Therapeutic prospects for tumours, and mechanisms of expression and regulation (Review). Mol Med Rep, 2021. 24 (3).
8. Ben-David, U., N. Nudel, and N. Benvenisty, Immunologic and chemical targeting of the tight-junction protein Claudin-6 eliminates tumorigenic human pluripotent stem cells. Nat Commun, 2013. 4: p. 1992.
9. Reinhard, K., et al., An RNA vaccine drives expansion and efficacy of claudin-CAR-T cells against solid tumors. Science, 2020. 367 (6476): p. 446-453.
10. Abuazza, G., et al., Claudins 6, 9, and 13 are developmentally expressed renal tight junction proteins. Am J Physiol Renal Physiol, 2006. 291 (6): p. F1132-41.
11. Hashizume, A., et al., Expression patterns of claudin family of tight junction membrane proteins in developing mouse submandibular gland. Dev Dyn, 2004. 231 (2): p. 425-31.
12. Kohmoto, T., et al., Claudin-6 is a single prognostic marker and functions as a tumor-promoting gene in a subgroup of intestinal type gastric cancer. Gastric Cancer, 2020. 23 (3): p. 403-417.
13. Kojima, M., et al., Prognostic Significance of Aberrant Claudin-6 Expression in Endometrial Cancer. Cancers (Basel), 2020. 12 (10).
14. Micke, P., et al., Aberrantly activated claudin 6 and 18.2 as potential therapy targets in non-small-cell lung cancer. Int J Cancer, 2014. 135 (9): p. 2206-14.
15. Sullivan, L. M., et al., Claudin-6 is a nonspecific marker for malignant rhabdoid and other pediatric tumors. Am J Surg Pathol, 2012. 36 (1): p. 73-80.
16. Ushiku, T., et al., Distinct expression pattern of claudin-6, a primitive phenotypic tight junction molecule, in germ cell tumours and visceral carcinomas. Histopathology, 2012. 61 (6): p. 1043-56.
17. Kamakura, D., R. Asano, and M. Yasunaga, T Cell Bispecific Antibodies: An Antibody-Based Delivery System for Inducing Antitumor Immunity. Pharmaceuticals (Basel), 2021. 14 (11).
18. Middelburg, J., et al., Overcoming Challenges for CD3-Bispecific Antibody Therapy in Solid Tumors. Cancers (Basel), 2021. 13 (2).
19. Singh, A., S. Dees, and I. S. Grewal, Overcoming the challenges associated with CD3+T-cell redirection in cancer. Br J Cancer, 2021. 124 (6): p. 1037-1048.
20. Baeuerle, P. A. and H. Wesche, T-cell-engaging antibodies for the treatment of solid tumors: challenges and opportunities. Current Opinion in Oncology, 2022. 34 (5).
21. Stadler, C. R., et al., Characterization of the first-in-class T-cell-engaging bispecific single-chain antibody for targeted immunotherapy of solid tumors expressing the oncofetal protein claudin 6. Oncoimmunology, 2016. 5 (3): p. e1091555.
22. Stadler, C. R., et al., Elimination of large tumors in mice by mRNA-encoded bispecific antibodies. Nat Med, 2017. 23 (7): p. 815-817.

1. A multi-specific antibody or antigen-binding fragment thereof, comprising a first antigen binding domain that specifically binds to human claudin 6 (CLDN6) and a second antigen binding domain that specifically binds to human cluster of differentiation 3 (CD3).

2. The multi-specific antibody or antigen-binding fragment of claim 1, wherein the first antigen binding domain does not bind to other claudin (CLDN) protein family members.

3. The multi-specific antibody or antigen-binding fragment of claim 2, wherein the first antigen binding domain does not bind to human claudin 9 (CLDN9).

4. The multi-specific antibody or antigen-binding fragment of claim 3, wherein the first antigen binding domain has high selectivity for human CLDN6 over human CLDN9.

5. The multi-specific antibody or antigen-binding fragment of claim 1, wherein the first antigen binding domain that specifically binds to human CLDN6 comprises:
(a) a heavy chain variable region that comprises (i) a heavy chain complementarity-determining region (HCDR)1 having an amino acid sequence of SEQ ID NO: 1, (ii) a HCDR2 having an amino acid sequence of SEQ ID NO: 39, and (iii) a HCDR3 having an amino acid sequence of SEQ ID NO: 3; and a light chain variable region that comprises: (iv) a light chain complementarity-determining region (LCDR)1 having an amino acid sequence of SEQ ID NO: 40, (v) a LCDR2 having an amino acid sequence of SEQ ID NO: 5, and (vi) a LCDR3 having an amino acid sequence of SEQ ID NO: 6;
(b) a heavy chain variable region that comprises: (i) a HCDR1 having an amino acid sequence of SEQ ID NO: 1, (ii) a HCDR2 having an amino acid sequence of SEQ ID NO: 2, and (iii) a HCDR3 having an amino acid sequence of SEQ ID NO: 3; and a light chain variable region that comprises:(iv) a LCDR1 having an amino acid sequence of SEQ ID NO: 4, (v) a LCDR2 having an amino acid sequence of SEQ ID NO: 5, and (vi) a LCDR3 having an amino acid sequence of SEQ ID NO: 6;
(c) a heavy chain variable region that comprises: (i) a HCDR1 having an amino acid sequence of SEQ ID NO: 1, (ii) a HCDR2 having an amino acid sequence of SEQ ID NO: 23, and (iii) a HCDR3 having an amino acid sequence of SEQ ID NO: 3; and a light chain variable region that comprises: (iv) a LCDR1 having an amino acid sequence of SEQ ID NO: 4, (v) a LCDR2 having an amino acid sequence of SEQ ID NO: 5, and (vi) a LCDR3 having an amino acid sequence of SEQ ID NO: 6; or
(d) a heavy chain variable region that comprises (i) a HCDR1 having an amino acid sequence of SEQ ID NO: 1, (ii) a HCDR2 having an amino acid sequence of SEQ ID NO: 45, and (iii) a HCDR3 having an amino acid sequence of SEQ ID NO: 3; and a light chain variable region that comprises: (iv) a LCDR1 having an amino acid sequence of SEQ ID NO: 40, (v) a LCDR2 having an amino acid sequence of SEQ ID NO: 5, and (vi) a LCDR3 having an amino acid sequence of SEQ ID NO: 6.

6. The multi-specific antibody or antigen-binding fragment of claim 1, wherein the first antigen binding domain comprises:
(a) a heavy chain variable region having an amino acid sequence at least 90, 91, 92, 93, 94, 95, 96, 97, 98 or 99% identical to SEQ ID NO: 43, and a light chain variable region having an amino acid sequence at least 90, 91, 92, 93, 94, 95, 96, 97, 98 or 99% identical to SEQ ID NO: 44;
(b) a heavy chain variable region having an amino acid sequence at least 90, 91, 92, 93, 94, 95, 96, 97, 98 or 99% identical to SEQ ID NO:7, and a light chain variable region having an amino acid sequence at least 90, 91, 92, 93, 94, 95, 96, 97, 98 or 99% identical to SEQ ID NO: 8;
(c) a heavy chain variable region having an amino acid sequence at least 90, 91, 92, 93, 94, 95, 96, 97, 98 or 99% identical to SEQ ID NO: 24, and a light chain variable region having an amino acid sequence at least 90, 91, 92, 93, 94, 95, 96, 97, 98 or 99% identical to SEQ ID NO: 12;
(d) a heavy chain variable region having an amino acid sequence at least 90, 91, 92, 93, 94, 95, 96, 97, 98 or 99% identical to SEQ ID NO: 41, and a light chain variable region having an amino acid sequence at least 90, 91, 92, 93, 94, 95, 96, 97, 98 or 99% identical to SEQ ID NO: 42;
(e) a heavy chain variable region having an amino acid sequence at least 90, 91, 92, 93, 94, 95, 96, 97, 98 or 99% identical to SEQ ID NO: 46, and a light chain variable region having an amino acid sequence at least 90, 91, 92, 93, 94, 95, 96, 97, 98 or 99% identical to SEQ ID NO: 47; or
(f) a heavy chain variable region having an amino acid sequence at least 90, 91, 92, 93, 94, 95, 96, 97, 98 or 99% identical to SEQ ID NO: 46, and a light chain variable region having an amino acid sequence at least 90, 91, 92, 93, 94, 95, 96, 97, 98 or 99% identical to SEQ ID NO: 42.

7. The multi-specific antibody or antigen-binding fragment of claim 6, wherein one, two, three, four, five, six, seven, eight, nine, or ten amino acids of SEQ ID NOS: 7, 8, 12, 24, 41, 42, 43, 44, 46, or 47 have been inserted, deleted or substituted.

8. The multi-specific antibody or antigen-binding fragment of claim 1, wherein the first antigen binding domain comprises:
(a) a heavy chain variable region having an amino acid sequence that comprises SEQ ID NO: 43, and a light chain variable region having an amino acid sequence that comprises SEQ ID NO: 44;
(b) a heavy chain variable region having an amino acid sequence that comprises SEQ ID NO:7, and a light chain variable region having an amino acid sequence that comprises SEQ ID NO: 8;
(c) a heavy chain variable region having an amino acid sequence that comprises SEQ ID NO: 24, and a light chain variable region having an amino acid sequence that comprises SEQ ID NO: 12;
(d) a heavy chain variable region having an amino acid sequence that comprises SEQ ID NO: 41, and a light chain variable region having an amino acid sequence that comprises SEQ ID NO: 42;
(e) a heavy chain variable region having an amino acid sequence that comprises SEQ ID NO: 46, and a light chain variable region having an amino acid sequence that comprises SEQ ID NO: 47; or
(f) a heavy chain variable region having an amino acid sequence that comprises SEQ ID NO: 46, and a light chain variable region having an amino acid sequence that comprises SEQ ID NO: 42.

9. The multi-specific antibody or antigen-binding fragment of claim 1, wherein the second antigen binding domain that specifically binds to human CD3 comprises:
(a) a heavy chain variable region that comprises (i) a HCDR1 having an amino acid sequence of SEQ ID NO: 48, (ii) a HCDR2 having an amino acid sequence of SEQ ID NO: 71, (iii) a HCDR3 having an amino acid sequence of SEQ ID NO: 75, and a light chain variable region that comprises: (iv) a LCDR1 having an amino acid sequence of SEQ ID NO: 51, (v) a LCDR2 having an amino acid sequence of SEQ ID NO: 52, and (vi) a LCDR3 having an amino acid sequence of SEQ ID NO: 53;
(b) a heavy chain variable region that comprises (i) a HCDR1 having an amino acid sequence of SEQ ID NO: 48, (ii) a HCDR2 having an amino acid sequence of SEQ ID NO: 49, and (iii) a HCDR3 having an amino acid sequence of SEQ ID NO: 50, and a light chain variable region that comprises: (iv) a LCDR1 having an amino acid sequence of SEQ ID NO: 51, (v) a LCDR2 having an amino acid sequence of SEQ ID NO: 52, and (vi) a LCDR3 having an amino acid sequence of SEQ ID NO: 53; or
(c) a heavy chain variable region that comprises (i) a HCDR1 having an amino acid sequence of SEQ ID NO: 48, (ii) a HCDR2 having an amino acid sequence of SEQ ID NO: 71, (iii) a HCDR3 having an amino acid sequence of SEQ ID NO: 50, and a light chain variable region that comprises: (iv) a LCDR1 having an amino acid sequence of SEQ ID NO: 51, (v) a LCDR2 having an amino acid sequence of SEQ ID NO: 52, and (vi) a LCDR3 having an amino acid sequence of SEQ ID NO: 53.

10. The multi-specific antibody or antigen-binding fragment of claim 1, wherein the second antigen binding domain comprises:
(a) a heavy chain variable region having an amino acid sequence at least 90, 91, 92, 93, 94, 95, 96, 97, 98 or 99% identical to SEQ ID NO: 76, and a light chain variable region having an amino acid sequence at least 90, 91, 92, 93, 94, 95, 96, 97, 98 or 99% identical to SEQ ID NO: 68;
(b) a heavy chain variable region having an amino acid sequence at least 90, 91, 92, 93, 94, 95, 96, 97, 98 or 99% identical to SEQ ID NO: 54, and a light chain variable region having an amino acid sequence at least 90, 91, 92, 93, 94, 95, 96, 97, 98 or 99% identical to SEQ ID NO: 55;
(v) a heavy chain variable region having an amino acid sequence at least 90, 91, 92, 93, 94, 95, 96, 97, 98 or 99% identical to SEQ ID NO: 58, and a light chain variable region having an amino acid sequence at least 90, 91, 92, 93, 94, 95, 96, 97, 98 or 99% identical to SEQ ID NO: 59;
(d) a heavy chain variable region having an amino acid sequence at least 90, 91, 92, 93, 94, 95, 96, 97, 98 or 99% identical to SEQ ID NO: 62, and a light chain variable region having an amino acid sequence at least 90, 91, 92, 93, 94, 95, 96, 97, 98 or 99% identical to SEQ ID NO: 63;
(e) a heavy chain variable region having an amino acid sequence at least 90, 91, 92, 93, 94, 95, 96, 97, 98 or 99% identical to SEQ ID NO: 62, and a light chain variable region having an amino acid sequence at least 90, 91, 92, 93, 94, 95, 96, 97, 98 or 99% identical to SEQ ID NO: 68; or
(f) a heavy chain variable region having an amino acid sequence at least 90, 91, 92, 93, 94, 95, 96, 97, 98 or 99% identical to SEQ ID NO: 72, and a light chain variable region having an amino acid sequence at least 90, 91, 92, 93, 94, 95, 96, 97, 98 or 99% identical to SEQ ID NO: 68.

11. The multi-specific antibody or antigen-binding fragment of claim 10, wherein one, two, three, four, five, six, seven, eight, nine, or ten amino acids of SEQ ID NOS: 54, 55, 58, 59, 62, 68, 72 or 76 have been inserted, deleted or substituted.

12. The multi-specific antibody or antigen-binding fragment of claim 1, wherein the second antigen binding domain comprises:
(a) a heavy chain variable region having an amino acid sequence that comprises SEQ ID NO: 76, and a light chain variable region having an amino acid sequence that comprises SEQ ID NO: 68;
(b) a heavy chain variable region having an amino acid sequence that comprises SEQ ID NO: 54, and a light chain variable region having an amino acid sequence that comprises SEQ ID NO: 55;
(c) a heavy chain variable region having an amino acid sequence that comprises SEQ ID NO: 58, and a light chain variable region having an amino acid sequence that comprises SEQ ID NO: 59;
(d) a heavy chain variable region having an amino acid sequence that comprises SEQ ID NO: 62, and a light chain variable region having an amino acid sequence that comprises SEQ ID NO: 63;
(e) a heavy chain variable region having an amino acid sequence that comprises SEQ ID NO: 62, and a light chain variable region having an amino acid sequence that comprises SEQ ID NO: 68; or
(f) a heavy chain variable region having an amino acid sequence that comprises SEQ ID NO: 72, and a light chain variable region having an amino acid sequence that comprises SEQ ID NO: 68.

13. The multi-specific antibody or antigen-binding fragment of claim 1, wherein the second antigen binding domain comprises:
(a) a single chain variable fragment (scFv) having an amino acid sequence that comprises SEQ ID NO: 77;
(b) a single chain variable fragment (scFv) having an amino acid sequence that comprises SEQ ID NO: 66;
(c) a single chain variable fragment (scFv) having an amino acid sequence that comprises SEQ ID NO: 69; or
(d) a single chain variable fragment (scFv) having an amino acid sequence that comprises SEQ ID NO: 73.

14. The multi-specific antibody or antigen-binding fragment of claim 1, wherein:
(a) the first antigen binding domain that specifically binds to human CLDN6 comprises: a heavy chain variable region that comprises (i) a HCDR1 having an amino acid sequence of SEQ ID NO: 1, (ii) a HCDR2 having an amino acid sequence of SEQ ID NO: 2, and (iii) a HCDR3 having an amino acid sequence of SEQ ID NO: 3, and a light chain variable region that comprises: (iv) a LCDR1 having an amino acid sequence of SEQ ID NO: 4, (v) a LCDR2 having an amino acid sequence of SEQ ID NO: 5, and (vii) a LCDR3 having an amino acid sequence of SEQ ID NO: 6; and the second antigen binding domain that specifically binds to human CD3 comprises: a heavy chain variable region that comprises (i) a HCDR1 having an amino acid sequence of SEQ ID NO: 48, (ii) a HCDR2 having an amino acid sequence of SEQ ID NO: 49, and (iii) a HCDR3 having an amino acid sequence of SEQ ID NO: 50, and a light chain variable region that comprises: (iv) a LCDR1 having an amino acid sequence of SEQ ID NO: 51, (v) a LCDR2 having an amino acid sequence of SEQ ID NO: 52, and (vi) a LCDR3 having an amino acid sequence of SEQ ID NO: 53;
(b) the first antigen binding domain that specifically binds to human CLDN6 comprises: a heavy chain variable region that comprises (i) a HCDR1 having an amino acid sequence of SEQ ID NO: 1, (ii) a HCDR2 having an amino acid sequence of SEQ ID NO: 2, and (iii) a HCDR3 having an amino acid sequence of SEQ ID NO: 3, and a light chain variable region that comprises: (iv) a LCDR1 having an amino acid sequence of SEQ ID NO: 4, (v) a LCDR2 having an amino acid sequence of SEQ ID NO: 5, and (vi) a LCDR3 having an amino acid sequence of SEQ ID NO: 6; and the second antigen binding domain that specifically binds to human CD3 comprises: a heavy chain variable region that comprises (i) a HCDR1 having an amino acid sequence of SEQ ID NO: 48, (ii) a HCDR2 having an amino acid sequence of SEQ ID NO: 71, and (iii) a HCDR3 having an amino acid sequence of SEQ ID NO: 50, and a light chain variable region that comprises: (iv) a LCDR1 having an amino acid sequence of SEQ ID NO: 51, (v) a LCDR2 having an amino acid sequence of SEQ ID NO: 52, and (vi) a LCDR3 having an amino acid sequence of SEQ ID NO: 53; or
(c) the first antigen binding domain that specifically binds to human CLDN6 comprises: a heavy chain variable region that comprises (i) a HCDR1 having an amino acid sequence of SEQ ID NO: 1, (ii) a HCDR2 having an amino acid sequence of SEQ ID NO: 2, and (iii) a HCDR3 having an amino acid sequence of SEQ ID NO: 3, and a light chain variable region that comprises: (iv) a LCDR1 having an amino acid sequence of SEQ ID NO: 4, (v) a LCDR2 having an amino acid sequence of SEQ ID NO: 5, and (vi) a LCDR3 having an amino acid sequence of SEQ ID NO: 6; and the second antigen binding domain that specifically binds to human CD3 comprises: a heavy chain variable region that comprises (i) a HCDR1 having an amino acid sequence of SEQ ID NO: 48, (ii) a HCDR2 having an amino acid sequence of SEQ ID NO: 71, (iii) a HCDR3 having an amino acid sequence of SEQ ID NO: 75, and a light chain variable region that comprises: (iv) a LCDR1 having an amino acid sequence of SEQ ID NO: 51, (v) a LCDR2 having an amino acid sequence of SEQ ID NO: 52, and (vi) a LCDR3 having an amino acid sequence of SEQ ID NO: 53.

15. The multi-specific antibody or antigen-binding fragment of claim 1, wherein:
(a) the first antigen binding domain that specifically binds to human CLDN6 comprises: a heavy chain variable region that comprises (i) a HCDR1 having an amino acid sequence of SEQ ID NO: 1, (ii) a HCDR2 having an amino acid sequence of SEQ ID NO: 23, and (iii) a HCDR3 having an amino acid sequence of SEQ ID NO: 3, and a light chain variable region that comprises: (iv) a LCDR1 having an amino acid sequence of SEQ ID NO: 4, (v) a LCDR2 having an amino acid sequence of SEQ ID NO: 5, and (vi) a LCDR3 having an amino acid sequence of SEQ ID NO: 6; and the second antigen binding domain that specifically binds to human CD3 comprises: a heavy chain variable region that comprises (i) a HCDR1 having an amino acid sequence of SEQ ID NO: 48, (ii) a HCDR2 having an amino acid sequence of SEQ ID NO: 49, and (iii) a HCDR3 having an amino acid sequence of SEQ ID NO: 50, and a light chain variable region that comprises: (iv) a LCDR1 having an amino acid sequence of SEQ ID NO: 51, (v) a LCDR2 having an amino acid sequence of SEQ ID NO: 52, and (vi) a LCDR3 having an amino acid sequence of SEQ ID NO: 53;
(b) the first antigen binding domain that specifically binds to human CLDN6 comprises: a heavy chain variable region that comprises (i) a HCDR1 having an amino acid sequence of SEQ ID NO: 1, (ii) a HCDR2 having an amino acid sequence of SEQ ID NO: 23, and (iii) a HCDR3 having an amino acid sequence of SEQ ID NO: 3, and a light chain variable region that comprises: (iv) a LCDR1 having an amino acid sequence of SEQ ID NO: 4, (v) a LCDR2 having an amino acid sequence of SEQ ID NO: 5, and (vi) a LCDR3 having an amino acid sequence of SEQ ID NO: 6; and the second antigen binding domain that specifically binds to human CD3 comprises: a heavy chain variable region that comprises (i) a HCDR1 having an amino acid sequence of SEQ ID NO: 48, (ii) a HCDR2 having an amino acid sequence of SEQ ID NO: 71, and (iii) a HCDR3 having an amino acid sequence of SEQ ID NO: 50, and a light chain variable region that comprises: (iv) a LCDR1 having an amino acid sequence of SEQ ID NO: 51, (v) a LCDR2 having an amino acid sequence of SEQ ID NO: 52, and (vi) a LCDR3 having an amino acid sequence of SEQ ID NO: 53; or
(c) the first antigen binding domain that specifically binds to human CLDN6 comprises: a heavy chain variable region that comprises (i) a HCDR1 having an amino acid sequence of SEQ ID NO: 1, (ii) a HCDR2 having an amino acid sequence of SEQ ID NO: 23, and (iii) a HCDR3 having an amino acid sequence of SEQ ID NO: 3, and a light chain variable region that comprises: (iv) a LCDR1 having an amino acid sequence of SEQ ID NO: 4, (v) a LCDR2 having an amino acid sequence of SEQ ID NO: 5, and (vi) a LCDR3 having an amino acid sequence of SEQ ID NO: 6; and the second antigen binding domain that specifically binds to human CD3 comprises: a heavy chain variable region that comprises (i) a HCDR1 having an amino acid sequence of SEQ ID NO: 48, (ii) a HCDR2 having an amino acid sequence of SEQ ID NO: 71, and (iii) a HCDR3 having an amino acid sequence of SEQ ID NO: 75 and a light chain variable region that comprises: (iv) a LCDR1 having an amino acid sequence of SEQ ID NO: 51, (v) a LCDR2 having an amino acid sequence of SEQ ID NO: 52, and (vi) a LCDR3 having an amino acid sequence of SEQ ID NO: 53.

16. The multi-specific antibody or antigen-binding fragment of claim 1, wherein:
(a) the first antigen binding domain that specifically binds to human CLDN6 comprises: a heavy chain variable region that comprises (i) a HCDR1 having an amino acid sequence of SEQ ID NO: 1, (ii) a HCDR2 having an amino acid sequence of SEQ ID NO: 39, and (iii) a HCDR3 having an amino acid sequence of SEQ ID NO: 3, and a light chain variable region that comprises: (iv) a LCDR1 having an amino acid sequence of SEQ ID NO: 40, (v) a LCDR2 having an amino acid sequence of SEQ ID NO: 5, and (vi) a LCDR3 having an amino acid sequence of SEQ ID NO: 6; and the second antigen binding domain that specifically binds to human CD3 comprises: a heavy chain variable region that comprises (i) a HCDR1 having an amino acid sequence of SEQ ID NO: 48, (ii) a HCDR2 having an amino acid sequence of SEQ ID NO: 49, and (iii) a HCDR3 having an amino acid sequence of SEQ ID NO: 50, and a light chain variable region that comprises: (iv) a LCDR1 having an amino acid sequence of SEQ ID NO: 51, (v) a LCDR2 having an amino acid sequence of SEQ ID NO: 52, and (vi) a LCDR3 having an amino acid sequence of SEQ ID NO: 53;
(b) the first antigen binding domain that specifically binds to human CLDN6 comprises: a heavy chain variable region that comprises (i) a HCDR1 having an amino acid sequence of SEQ ID NO: 1, (ii) a HCDR2 having an amino acid sequence of SEQ ID NO: 39, and (iii) a HCDR3 having an amino acid sequence of SEQ ID NO: 3, and a light chain variable region that comprises: (iv) a LCDR1 having an amino acid sequence of SEQ ID NO: 40, (v) a LCDR2 having an amino acid sequence of SEQ ID NO: 5, and (vi) a LCDR3 having an amino acid sequence of SEQ ID NO: 6; and the second antigen binding domain that specifically binds to human CD3 comprises: a heavy chain variable region that comprises (i) a HCDR1 having an amino acid sequence of SEQ ID NO: 48, (ii) a HCDR2 having an amino acid sequence of SEQ ID NO: 71, and (iii) a HCDR3 having an amino acid sequence of SEQ ID NO: 50, and a light chain variable region that comprises: (iv) a LCDR1 having an amino acid sequence of SEQ ID NO: 51, (v) a LCDR2 having an amino acid sequence of SEQ ID NO: 52, and (f) a LCDR3 having an amino acid sequence of SEQ ID NO: 53; or
(c) the first antigen binding domain that specifically binds to human CLDN6 comprises: a heavy chain variable region that comprises (i) a HCDR1 having an amino acid sequence of SEQ ID NO: 1, (ii) a HCDR2 having an amino acid sequence of SEQ ID NO: 39, and (iii) a HCDR3 having an amino acid sequence of SEQ ID NO: 3, and a light chain variable region that comprises: (iv) a LCDR1 having an amino acid sequence of SEQ ID NO: 40, (v) a LCDR2 having an amino acid sequence of SEQ ID NO: 5, and (vi) a LCDR3 having an amino acid sequence of SEQ ID NO: 6; and the second antigen binding domain that specifically binds to human CD3 comprises: a heavy chain variable region that comprises (i) a HCDR1 having an amino acid sequence of SEQ ID NO: 48, (ii) a HCDR2 having an amino acid sequence of SEQ ID NO: 71, and (iii) a HCDR3 having an amino acid sequence of SEQ ID NO: 75, and a light chain variable region that comprises: (iv) a LCDR1 having an amino acid sequence of SEQ ID NO: 51, (v) a LCDR2 having an amino acid sequence of SEQ ID NO: 52, and (vi) a LCDR3 having an amino acid sequence of SEQ ID NO: 53.

17. The multi-specific antibody or antigen-binding fragment of claim 16, wherein: the first antigen binding domain that specifically binds to human CLDN6 comprises: a heavy chain variable region that comprises (i) a HCDR1 having an amino acid sequence of SEQ ID NO: 1, (ii) a HCDR2 having an amino acid sequence of SEQ ID NO: 39, (iii) a HCDR3 having an amino acid sequence of SEQ ID NO: 3 and a light chain variable region that comprises: (iv) a LCDR1 having an amino acid sequence of SEQ ID NO: 40, (v) a LCDR2 having an amino acid sequence of SEQ ID NO: 5, and (vii) a LCDR3 having an amino acid sequence of SEQ ID NO: 6; and the second antigen binding domain that specifically binds to human CD3 comprises: a heavy chain variable region that comprises (i) a HCDR1 having an amino acid sequence of SEQ ID NO: 48, (ii) a HCDR2 having an amino acid sequence of SEQ ID NO: 71, (iii) a HCDR3 having an amino acid sequence of SEQ ID NO: 75 and a light chain variable region that comprises: (iv) a LCDR1 having an amino acid sequence of SEQ ID NO: 51, (v) a LCDR2 having an amino acid sequence of SEQ ID NO: 52, and (vi) a LCDR3 having an amino acid sequence of SEQ ID NO: 53.

18. The multi-specific antibody or antigen-binding fragment of claim 1, wherein:
(a) the first antigen binding domain that specifically binds to human CLDN6 comprises: a heavy chain variable region that comprises (i) a HCDR1 having an amino acid sequence of SEQ ID NO: 1, (ii) a HCDR2 having an amino acid sequence of SEQ ID NO: 45, (iii) a HCDR3 having an amino acid sequence of SEQ ID NO: 3 and a light chain variable region that comprises: (ivd) a LCDR1 having an amino acid sequence of SEQ ID NO: 40, (v) a LCDR2 having an amino acid sequence of SEQ ID NO: 5, and (vi) a LCDR3 having an amino acid sequence of SEQ ID NO: 6; and the second antigen binding domain that specifically binds to human CD3 comprises: a heavy chain variable region that comprises (i) a HCDR1 having an amino acid sequence of SEQ ID NO: 48, (ii) a HCDR2 having an amino acid sequence of SEQ ID NO: 49, (iii) a HCDR3 having an amino acid sequence of SEQ ID NO: 50 and a light chain variable region that comprises: (iv) a LCDR1 having an amino acid sequence of SEQ ID NO: 51, (v) a LCDR2 having an amino acid sequence of SEQ ID NO: 52, and (vi) a LCDR3 having an amino acid sequence of SEQ ID NO: 53;
(b) the first antigen binding domain that specifically binds to human CLDN6 comprises: a heavy chain variable region that comprises (i) a HCDR1 having an amino acid sequence of SEQ ID NO: 1, (ii) a HCDR2 having an amino acid sequence of SEQ ID NO: 45, (iii) a HCDR3 having an amino acid sequence of SEQ ID NO: 3 and a light chain variable region that comprises: (iv) a LCDR1 having an amino acid sequence of SEQ ID NO: 40, (v) a LCDR2 having an amino acid sequence of SEQ ID NO: 5, and (vi) a LCDR3 having an amino acid sequence of SEQ ID NO: 6; and the second antigen binding domain that specifically binds to human CD3 comprises: a heavy chain variable region that comprises (i) a HCDR1 having an amino acid sequence of SEQ ID NO: 48, (ii) a HCDR2 having an amino acid sequence of SEQ ID NO: 71, (iii) a HCDR3 having an amino acid sequence of SEQ ID NO: 50 and a light chain variable region that comprises: (iv) a LCDR1 having an amino acid sequence of SEQ ID NO: 51, (v) a LCDR2 having an amino acid sequence of SEQ ID NO: 52, and (vi) a LCDR3 having an amino acid sequence of SEQ ID NO: 53; or
(c) the first antigen binding domain that specifically binds to human CLDN6 comprises: a heavy chain variable region that comprises (i) a HCDR1 having an amino acid sequence of SEQ ID NO: 1, (ii) a HCDR2 having an amino acid sequence of SEQ ID NO: 45, (iii) a HCDR3 having an amino acid sequence of SEQ ID NO: 3 and a light chain variable region that comprises: (iv) a LCDR1 having an amino acid sequence of SEQ ID NO: 40, (v) a LCDR2 having an amino acid sequence of SEQ ID NO: 5, and (vi) a LCDR3 having an amino acid sequence of SEQ ID NO: 6; and the second antigen binding domain that specifically binds to human CD3 comprises: a heavy chain variable region that comprises (i) a HCDR1 having an amino acid sequence of SEQ ID NO: 48, (ii) a HCDR2 having an amino acid sequence of SEQ ID NO: 71, (iii) a HCDR3 having an amino acid sequence of SEQ ID NO: 75 and a light chain variable region that comprises: (iv) a LCDR1 having an amino acid sequence of SEQ ID NO: 51, (v) a LCDR2 having an amino acid sequence of SEQ ID NO: 52, and (vi) a LCDR3 having an amino acid sequence of SEQ ID NO: 53.

19. The multi-specific antibody or antigen-binding fragment of claim 18, wherein:
the first antigen binding domain that specifically binds to human CLDN6 comprises: a heavy chain variable region that comprises (i) a HCDR1 having an amino acid sequence of SEQ ID NO: 1, (ii) a HCDR2 having an amino acid sequence of SEQ ID NO: 45, (iii) a HCDR3 having an amino acid sequence of SEQ ID NO: 3 and a light chain variable region that comprises: (iv) a LCDR1 having an amino acid sequence of SEQ ID NO: 40, (v) a LCDR2 having an amino acid sequence of SEQ ID NO: 5, and (vi) a LCDR3 having an amino acid sequence of SEQ ID NO: 6;
and the second antigen binding domain that specifically binds to human CD3 comprises: a heavy chain variable region that comprises (i) a HCDR1 having an amino acid sequence of SEQ ID NO: 48, (ii) a HCDR2 having an amino acid sequence of SEQ ID NO: 71, (iii) a HCDR3 having an amino acid sequence of SEQ ID NO: 75 and a light chain variable region that comprises: (iv) a LCDR1 having an amino acid sequence of SEQ ID NO: 51, (v) a LCDR2 having an amino acid sequence of SEQ ID NO: 52, and (vi) a LCDR3 having an amino acid sequence of SEQ ID NO: 53.

20. The multi-specific antibody or antigen-binding fragment of claim 1, wherein:
the first antigen binding domain that specifically binds to human CLDN6 comprises:
a) a heavy chain variable region that comprises (i) a HCDR1 having an amino acid sequence of SEQ ID NO: 1, (ii) a HCDR2 having an amino acid sequence of SEQ ID NO: 2, (iii) a HCDR3 having an amino acid sequence of SEQ ID NO: 3 and a light chain variable region that comprises: (iv) a LCDR1 having an amino acid sequence of SEQ ID NO: 4, (v) a LCDR2 having an amino acid sequence of SEQ ID NO: 5, and (vi) a LCDR3 having an amino acid sequence of SEQ ID NO: 6;
b) a heavy chain variable region that comprises (i) a HCDR1 having an amino acid sequence of SEQ ID NO: 1, (ii) a HCDR2 having an amino acid sequence of SEQ ID NO: 23, (iii) a HCDR3 having an amino acid sequence of SEQ ID NO: 3 and a light chain variable region that comprises: (iv) a LCDR1 having an amino acid sequence of SEQ ID NO: 4, (v) a LCDR2 having an amino acid sequence of SEQ ID NO: 5, and (vi) a LCDR3 having an amino acid sequence of SEQ ID NO: 6;
c) a heavy chain variable region that comprises (i) a HCDR1 having an amino acid sequence of SEQ ID NO: 1, (ii) a HCDR2 having an amino acid sequence of SEQ ID NO: 39, (iii) a HCDR3 having an amino acid sequence of SEQ ID NO: 3 and a light chain variable region that comprises: (iv) a LCDR1 having an amino acid sequence of SEQ ID NO: 40, (v) a LCDR2 having an amino acid sequence of SEQ ID NO: 5, and (vi) a LCDR3 having an amino acid sequence of SEQ ID NO: 6; or
d) the first antigen binding domain that specifically binds to human CLDN6 comprises: a heavy chain variable region that comprises (i) a HCDR1 having an amino acid sequence of SEQ ID NO: 1, (ii) a HCDR2 having an amino acid sequence of SEQ ID NO: 45, (iii) a HCDR3 having an amino acid sequence of SEQ ID NO: 3 and a light chain variable region that comprises: (iv) a LCDR1 having an amino acid sequence of SEQ ID NO: 40, (v) a LCDR2 having an amino acid sequence of SEQ ID NO: 5, and (vi) a LCDR3 having an amino acid sequence of SEQ ID NO: 6; and
the second antigen binding domain that specifically binds to human CD3 comprises:
a) a heavy chain variable region (VH) having an amino acid sequence that comprises SEQ ID NO: 54, and a light chain variable region (VL) having an amino acid sequence that comprises SEQ ID NO: 55;
b) a heavy chain variable region (VH) having an amino acid sequence that comprises SEQ ID NO: 58, and a light chain variable region (VL) having an amino acid sequence that comprises SEQ ID NO: 59;
c) a heavy chain variable region (VH) having an amino acid sequence that comprises SEQ ID NO: 62, and a light chain variable region (VL) having an amino acid sequence that comprises SEQ ID NO: 63;
d) a heavy chain variable region (VH) having an amino acid sequence that comprises SEQ ID NO: 62, and a light chain variable region (VL) having an amino acid sequence that comprises SEQ ID NO: 68;
e) a heavy chain variable region (VH) having an amino acid sequence that comprises SEQ ID NO: 72, and a light chain variable region (VL) having an amino acid sequence that comprises SEQ ID NO: 68; or
f) a heavy chain variable region (VH) having an amino acid sequence that comprises SEQ ID NO: 76, and a light chain variable region (VL) having an amino acid sequence that comprises SEQ ID NO: 68.

21. The multi-specific antibody or antigen-binding fragment of claim 1, wherein:
the first antigen binding domain that specifically binds to human CLDN6 comprises:
a) a heavy chain variable region that comprises (i) a HCDR1 having an amino acid sequence of SEQ ID NO: 1, (ii) a HCDR2 having an amino acid sequence of SEQ ID NO: 39, (iii) a HCDR3 having an amino acid sequence of SEQ ID NO: 3 and a light chain variable region that comprises: (iv) a LCDR1 having an amino acid sequence of SEQ ID NO: 40, (v) a LCDR2 having an amino acid sequence of SEQ ID NO: 5, and (vi) a LCDR3 having an amino acid sequence of SEQ ID NO: 6; or
and
the second antigen binding domain that specifically binds to human CD3 comprises:
b) a heavy chain variable region (VH) having an amino acid sequence that comprises SEQ ID NO: 54, and a light chain variable region (VL) having an amino acid sequence that comprises SEQ ID NO: 55;
c) a heavy chain variable region (VH) having an amino acid sequence that comprises SEQ ID NO: 58, and a light chain variable region (VL) having an amino acid sequence that comprises SEQ ID NO: 59;
d) a heavy chain variable region (VH) having an amino acid sequence that comprises SEQ ID NO: 62, and a light chain variable region (VL) having an amino acid sequence that comprises SEQ ID NO: 63;
e) a heavy chain variable region (VH) having an amino acid sequence that comprises SEQ ID NO: 62, and a light chain variable region (VL) having an amino acid sequence that comprises SEQ ID NO: 68;
f) a heavy chain variable region (VH) having an amino acid sequence that comprises SEQ ID NO: 72, and a light chain variable region (VL) having an amino acid sequence that comprises SEQ ID NO: 68; or
g) a heavy chain variable region (VH) having an amino acid sequence that comprises SEQ ID NO: 76, and a light chain variable region (VL) having an amino acid sequence that comprises SEQ ID NO: 68.

22. The multi-specific antibody or antigen-binding fragment of claim 1, wherein:
the first antigen binding domain that specifically binds to human CLDN6 comprises:
(a) a heavy chain variable region that comprises (i) a HCDR1 having an amino acid sequence of SEQ ID NO: 1, (ii) a HCDR2 having an amino acid sequence of SEQ ID NO: 23, (iii) a HCDR3 having an amino acid sequence of SEQ ID NO: 3 and a light chain variable region that comprises: (iv) a LCDR1 having an amino acid sequence of SEQ ID NO: 4, (v) a LCDR2 having an amino acid sequence of SEQ ID NO: 5, and (vi) a LCDR3 having an amino acid sequence of SEQ ID NO: 6; or
(b) a heavy chain variable region that comprises (i) a HCDR1 having an amino acid sequence of SEQ ID NO: 1, (ii) a HCDR2 having an amino acid sequence of SEQ ID NO: 39, (iii) a HCDR3 having an amino acid sequence of SEQ ID NO: 3 and a light chain variable region that comprises: (iv) a LCDR1 having an amino acid sequence of SEQ ID NO: 40, (v) a LCDR2 having an amino acid sequence of SEQ ID NO: 5, and (vi) a LCDR3 having an amino acid sequence of SEQ ID NO: 6; or
(c) a heavy chain variable region that comprises (i) a HCDR1 having an amino acid sequence of SEQ ID NO: 1, (ii) a HCDR2 having an amino acid sequence of SEQ ID NO: 45, (iii) a HCDR3 having an amino acid sequence of SEQ ID NO: 3 and a light chain variable region that comprises: (iv) a LCDR1 having an amino acid sequence of SEQ ID NO: 40, (v) a LCDR2 having an amino acid sequence of SEQ ID NO: 5, and (vi) a LCDR3 having an amino acid sequence of SEQ ID NO: 6;
and
the second antigen binding domain that specifically binds to human CD3 comprises:
(i) a single chain variable fragment (scFv) having an amino acid sequence that comprises SEQ ID NO: 66;
(ii) a single chain variable fragment (scFv) having an amino acid sequence that comprises SEQ ID NO: 69;
(iii) a single chain variable fragment (scFv) having an amino acid sequence that comprises SEQ ID NO: 73; or
(iv) a single chain variable fragment (scFv) having an amino acid sequence that comprises SEQ ID NO: 77.

23. The multi-specific antibody or antigen-binding fragment of claim 1, wherein:
the first antigen binding domain that specifically binds to human CLDN6 comprises:
(i) a heavy chain variable region (VH) having an amino acid sequence that comprises SEQ ID NO: 24, and a light chain variable region (VL) having an amino acid sequence that comprises SEQ ID NO: 12;
(ii) a heavy chain variable region (VH) having an amino acid sequence that comprises SEQ ID NO: 41, and a light chain variable region (VL) having an amino acid sequence that comprises SEQ ID NO: 42;
(iii) a heavy chain variable region (VH) having an amino acid sequence that comprises SEQ ID NO: 43, and a light chain variable region (VL) having an amino acid sequence that comprises SEQ ID NO: 44;
(iv) a heavy chain variable region (VH) having an amino acid sequence that comprises SEQ ID NO: 46, and a light chain variable region (VL) having an amino acid sequence that comprises SEQ ID NO: 47; or
(v) a heavy chain variable region (VH) having an amino acid sequence that comprises SEQ ID NO: 46, and a light chain variable region (VL) having an amino acid sequence that comprises SEQ ID NO: 42;
and
the second antigen binding domain that specifically binds to human CD3 comprises:
(i) a heavy chain variable region (VH) having an amino acid sequence that comprises SEQ ID NO: 54, and a light chain variable region (VL) having an amino acid sequence that comprises SEQ ID NO: 55;
(ii) a heavy chain variable region (VH) having an amino acid sequence that comprises SEQ ID NO: 58, and a light chain variable region (VL) having an amino acid sequence that comprises SEQ ID NO: 59;
(iii) a heavy chain variable region (VH) having an amino acid sequence that comprises SEQ ID NO: 62, and a light chain variable region (VL) having an amino acid sequence that comprises SEQ ID NO: 63;
(iv) a heavy chain variable region (VH) having an amino acid sequence that comprises SEQ ID NO: 62, and a light chain variable region (VL) having an amino acid sequence that comprises SEQ ID NO: 68;
(v) a heavy chain variable region (VH) having an amino acid sequence that comprises SEQ ID NO: 72, and a light chain variable region (VL) having an amino acid sequence that comprises SEQ ID NO: 68; or
(vi) a heavy chain variable region (VH) having an amino acid sequence that comprises SEQ ID NO: 76, and a light chain variable region (VL) having an amino acid sequence that comprises SEQ ID NO: 68.

24. The multi-specific antibody or antigen-binding fragment of claim 1, wherein:
the first antigen binding domain that specifically binds to human CLDN6 comprises:
(i) a heavy chain variable region (VH) having an amino acid sequence that comprises SEQ ID NO: 24, and a light chain variable region (VL) having an amino acid sequence that comprises SEQ ID NO: 12;
(ii) a heavy chain variable region (VH) having an amino acid sequence that comprises SEQ ID NO: 41, and a light chain variable region (VL) having an amino acid sequence that comprises SEQ ID NO: 42;
(iii) a heavy chain variable region (VH) having an amino acid sequence that comprises SEQ ID NO: 43, and a light chain variable region (VL) having an amino acid sequence that comprises SEQ ID NO: 44;
(iv) a heavy chain variable region (VH) having an amino acid sequence that comprises SEQ ID NO: 46, and a light chain variable region (VL) having an amino acid sequence that comprises SEQ ID NO: 47; or
(v) a heavy chain variable region (VH) having an amino acid sequence that comprises SEQ ID NO: 46, and a light chain variable region (VL) having an amino acid sequence that comprises SEQ ID NO: 42;
and
the second antigen binding domain that specifically binds to human CD3 comprises: a heavy chain variable region (VH) having an amino acid sequence that comprises SEQ ID NO: 72, and a light chain variable region (VL) having an amino acid sequence that comprises SEQ ID NO: 68.

25. The multi-specific antibody or antigen-binding fragment of claim 1, wherein:
the first antigen binding domain that specifically binds to human CLDN6 comprises:
(i) a heavy chain variable region (VH) having an amino acid sequence that comprises SEQ ID NO: 24, and a light chain variable region (VL) having an amino acid sequence that comprises SEQ ID NO: 12;
(ii) a heavy chain variable region (VH) having an amino acid sequence that comprises SEQ ID NO: 41, and a light chain variable region (VL) having an amino acid sequence that comprises SEQ ID NO: 42;
(iii) a heavy chain variable region (VH) having an amino acid sequence that comprises SEQ ID NO: 43, and a light chain variable region (VL) having an amino acid sequence that comprises SEQ ID NO: 44;
(iv) a heavy chain variable region (VH) having an amino acid sequence that comprises SEQ ID NO: 46, and a light chain variable region (VL) having an amino acid sequence that comprises SEQ ID NO: 47; or
(v) a heavy chain variable region (VH) having an amino acid sequence that comprises SEQ ID NO: 46, and a light chain variable region (VL) having an amino acid sequence that comprises SEQ ID NO: 42;
and
the second antigen binding domain that specifically binds to human CD3 comprises: a heavy chain variable region (VH) having an amino acid sequence that comprises SEQ ID NO: 76, and a light chain variable region (VL) having an amino acid sequence that comprises SEQ ID NO: 68.

26. The multi-specific antibody or antigen-binding fragment of claim 1, wherein:
the first antigen binding domain that specifically binds to human CLDN6 comprises: a heavy chain variable region (VH) having an amino acid sequence that comprises SEQ ID NO: 43, and a light chain variable region (VL) having an amino acid sequence that comprises SEQ ID NO: 44;
and the second antigen binding domain that specifically binds to human CD3 comprises:
(i) a heavy chain variable region (VH) having an amino acid sequence that comprises SEQ ID NO: 54, and a light chain variable region (VL) having an amino acid sequence that comprises SEQ ID NO: 55;
(ii) a heavy chain variable region (VH) having an amino acid sequence that comprises SEQ ID NO: 58, and a light chain variable region (VL) that comprises SEQ ID NO: 59;
(iii) a heavy chain variable region (VH) that comprises SEQ ID NO: 62, and a light chain variable region (VL) having an amino acid sequence that comprises SEQ ID NO: 63;
(iv) a heavy chain variable region (VH) having an amino acid sequence that comprises SEQ ID NO: 62, and a light chain variable region (VL) having an amino acid sequence that comprises SEQ ID NO: 68;
(v) a heavy chain variable region (VH) having an amino acid sequence that comprises SEQ ID NO: 72, and a light chain variable region (VL) having an amino acid sequence that comprises SEQ ID NO: 68; or
(vi) a heavy chain variable region (VH) having an amino acid sequence that comprises SEQ ID NO: 76, and a light chain variable region (VL) having an amino acid sequence that comprises SEQ ID NO: 68.

27. The multi-specific antibody or antigen-binding fragment of claim 26, wherein: the first antigen binding domain that specifically binds to human CLDN6 comprises: a heavy chain variable region (VH) having an amino acid sequence that comprises SEQ ID NO: 43, and a light chain variable region (VL) having an amino acid sequence that comprises SEQ ID NO: 44; and the second antigen binding domain that specifically binds to human CD3 comprises: a heavy chain variable region (VH) having an amino acid sequence that comprises SEQ ID NO: 72, and a light chain variable region (VL) having an amino acid sequence that comprises SEQ ID NO: 68.

28. The multi-specific antibody or antigen-binding fragment of claim 26, wherein: the first antigen binding domain that specifically binds to human CLDN6 comprises: a heavy chain variable region (VH) having an amino acid sequence that comprises SEQ ID NO: 43, and a light chain variable region (VL) having an amino acid sequence that comprises SEQ ID NO: 44; and the second antigen binding domain that specifically binds to human CD3 comprises: a heavy chain variable region (VH) having an amino acid sequence that comprises SEQ ID NO: 76, and a light chain variable region (VL) having an amino acid sequence that comprises SEQ ID NO: 68.

29. The multi-specific antibody or antigen-binding fragment of claim 1, wherein:
the first antigen binding domain that specifically binds to human CLDN6 comprises:
(i) a heavy chain variable region (VH) having an amino acid sequence that comprises SEQ ID NO: 24, and a light chain variable region (VL) having an amino acid sequence that comprises SEQ ID NO: 12;
(ii) a heavy chain variable region (VH) having an amino acid sequence that comprises SEQ ID NO: 41, and a light chain variable region (VL) having an amino acid sequence that comprises SEQ ID NO: 42;
(iii) a heavy chain variable region (VH) having an amino acid sequence that comprises SEQ ID NO: 43, and a light chain variable region (VL) having an amino acid sequence that comprises SEQ ID NO: 44;
(iv) a heavy chain variable region (VH) having an amino acid sequence that comprises SEQ ID NO: 46, and a light chain variable region (VL) having an amino acid sequence that comprises SEQ ID NO: 47; or
(v) a heavy chain variable region (VH) having an amino acid sequence that comprises SEQ ID NO: 46, and a light chain variable region (VL) having an amino acid sequence that comprises SEQ ID NO: 42.
and
the second antigen binding domain that specifically binds to human CD3 comprises:
(i) a single chain variable fragment (scFv) having an amino acid sequence that comprises SEQ ID NO: 66;
(ii) a single chain variable fragment (scFv) having an amino acid sequence that comprises SEQ ID NO: 69;
(iii) a single chain variable fragment (scFv) having an amino acid sequence that comprises SEQ ID NO: 73; or
(iv) a single chain variable fragment (scFv) having an amino acid sequence that comprises SEQ ID NO: 77.

30. The multi-specific antibody or antigen-binding fragment of claim 1, wherein:
the first antigen binding domain that specifically binds to human CLDN6 comprises: a heavy chain variable region (VH) having an amino acid sequence that comprises SEQ ID NO: 43, and a light chain variable region (VL) having an amino acid sequence that comprises SEQ ID NO: 44;
and the second antigen binding domain that specifically binds to human CD3 comprises:
(a) a single chain variable fragment (scFv) having an amino acid sequence that comprises SEQ ID NO: 66;
(b) a single chain variable fragment (scFv) having an amino acid sequence that comprises SEQ ID NO: 69;
(c) a single chain variable fragment (scFv) having an amino acid sequence that comprises SEQ ID NO: 73; or
(d) a single chain variable fragment (scFv) having an amino acid sequence that comprises SEQ ID NO: 77.

31. The multi-specific antibody or antigen-binding fragment of claim 30, wherein: the first antigen binding domain that specifically binds to human CLDN6 comprises: a heavy chain variable region (VH) having an amino acid sequence that comprises SEQ ID NO: 43, and a light chain variable region (VL) having an amino acid sequence that comprises SEQ ID NO: 44; and the second antigen binding domain that specifically binds to human CD3 comprises: a single chain variable fragment (scFv) having an amino acid sequence that comprises SEQ ID NO: 66.

32. The multi-specific antibody or antigen-binding fragment of claim 30, wherein: the first antigen binding domain that specifically binds to human CLDN6 comprises: a heavy chain variable region (VH) having an amino acid sequence that comprises SEQ ID NO: 43, and a light chain variable region (VL) having an amino acid sequence that comprises SEQ ID NO: 44; and the second antigen binding domain that specifically binds to human CD3 comprises: a single chain variable fragment (scFv) having an amino acid sequence that comprises SEQ ID NO: 69.

33. The multi-specific antibody or antigen-binding fragment of claim 30, wherein: the first antigen binding domain that specifically binds to human CLDN6 comprises: a heavy chain variable region (VH) having an amino acid sequence that comprises SEQ ID NO: 43, and a light chain variable region (VL) having an amino acid sequence that comprises SEQ ID NO: 44; and the second antigen binding domain that specifically binds to human CD3 comprises: a single chain variable fragment (scFv) having an amino acid sequence that comprises SEQ ID NO: 73.

34. The multi-specific antibody or antigen-binding fragment of claim 30, wherein: the first antigen binding domain that specifically binds to human CLDN6 comprises: a heavy chain variable region (VH) having an amino acid sequence that comprises SEQ ID NO: 43, and a light chain variable region (VL) having an amino acid sequence that comprises SEQ ID NO: 44; and the second antigen binding domain that specifically binds to human CD3 comprises: a single chain variable fragment (scFv) having an amino acid sequence that comprises SEQ ID NO: 77.

35. The multi-specific antibody or antigen-binding fragment of claim 1, which is a monoclonal antibody, a chimeric antibody, a humanized antibody, a human engineered antibody, a single chain antibody (scFv), a Fab fragment, a Fab′ fragment, or a F(ab′)2 fragment.

36. The multi-specific antibody or antigen-binding fragment of claim 1, wherein the multi-specific antibody is a bispecific antibody.

37. The multi-specific antibody or antigen-binding fragment of claim 36, wherein the bispecific antibody is BG143P.

38. The multi-specific antibody or antigen-binding fragment of claim 1, wherein the antibody or antigen-binding fragment thereof has antibody dependent cellular cytotoxicity (ADCC) or complement dependent cytotoxicity (CDC).

39. The multi-specific antibody or antigen-binding fragment of claim 1, wherein the antibody or antigen-binding fragment thereof has reduced glycosylation or no glycosylation or is hypofucosylated.

40. The multi-specific antibody or antigen-binding fragment of claim 1, wherein the antibody or antigen-binding fragment thereof comprises increased bisecting GlcNac structures.

41. The multi-specific antibody or antigen-binding fragment of claim 1, wherein the antibody or antigen-binding fragment comprises a Fc domain that is an IgG1 with reduced effector function.

42. The multi-specific antibody or antigen-binding fragment of claim 1, wherein the antibody or antigen-binding fragment comprises a Fc domain that is an IgG4.

43. A pharmaceutical composition comprising the multi-specific antibody or antigen-binding fragment of claim 1 and a pharmaceutically acceptable carrier.

44. The pharmaceutical composition of claim 43, further comprising a histidine/histidine HCl, a trehalose dihydrate, and/or a polysorbate 20.

45. A method of treating cancer comprising administering to a patient in need an effective amount of the multi-specific antibody or antigen-binding fragment of claim 1.

46-52. (canceled)

53. An isolated nucleic acid that encodes the multi-specific antibody or antigen-binding fragment of claim 1.

54. A vector comprising the nucleic acid of claim 53.

55. A host cell comprising the vector of claim 54.

56. A process for producing a multi-specific antibody or antigen-binding fragment thereof comprising cultivating the host cell of claim 55 in culture and recovering the antibody or antigen-binding fragment from the culture.

57-59. (canceled)
